(19) United States
(12) Patent Application Publication
(10) Pub. No.: US 2023/0277682 A1
(43) Pub. Date: Sep. 7, 2023

(54) VERRUCARIN A DERIVATIVES AND ANTIBODY DRUG CONJUGATES THEREOF

(71) Applicant: Regeneron Pharmaceuticals, Inc., Tarrytown, NY (US)

(72) Inventor: Thomas Nittoli, Orangeburg, NY (US)

(21) Appl. No.: 18/154,262

(22) Filed: Jan. 13, 2023

Related U.S. Application Data
(60) Provisional application No. 63/367,108, filed on Jun. 27, 2022.
(60) Provisional application No. 63/299,824, filed on Jan. 14, 2022.

(51) Int. Cl. A61K 47/68; C07D 493/20; A61K 47/65; C07D 493/22; A61P 31/14

(52) U.S. Cl. CPC A61K 47/6841; C07D 493/20; A61K 47/65; A61K 47/6803; C07D 493/22; A61P 31/14

(57) ABSTRACT

Provided herein are verrucarin A derivatives, linker-verrucarin A derivatives and antibody drug conjugates thereof. In one embodiment, the verrucarin A derivatives, linker-verrucarin A derivatives and antibody drug conjugates are useful in treating viral diseases.

### RELATED APPLICATIONS

This application claims priority under 35 USC 119(e) to U.S. Provisional Patent Applications 63/299,824, filed Jan. 14, 2022, and 63/367,108, filed Jun. 27, 2022. The contents of each of the above applications are incorporated herein by reference in its entirety.

### STATEMENT REGARDING FEDERALLY SPONSORED RESEARCH

This invention was made with U.S. government support under OTA Contract #HHS0100201700020C awarded by The Biomedical Advanced Research and Development Authority (BARDA). The U.S. government has certain rights in the invention.

### SEQUENCE LISTING

This application contains a Sequence Listing, which is being submitted herewith as an XML file named “10968US01.xml”, created on Jan. 11, 2023, size 457,094 bytes, which is incorporated by reference herein in its entirety.

### FIELD

Provided herein are verrucarin A derivatives and antibody drug conjugates (ADCs) thereof. The verrucarin A derivatives and ADCs provided herein are useful, inter alia, in the treatment of viral diseases.

### BACKGROUND

Verrucarin A is a trichothecene toxin that has been studied in the treatment of various cancers. However, the toxicity of verrucarin A is too high to provide a therapeutic window for its use as a therapy. Thus, ADCs of verrucarin A have been proposed to solve this problem (U.S. Pat. Nos. 4,744,981, 10,232,051 and 10,985,112; U.S. Patent Application Publication No. US 2015/0250896). Verrucarin A has also been shown to have significant antiviral activity against, e.g., vaccinia virus strain DII and Newcastle disease virus strain Miyadera (NDV) (Tamura et al. J. Anitbiot. (Tokyo) 1968, 21(2):160-161) and arenavirus Junin (JUNV) (Garcia et al. Planta Med. 2002, 68(3):209-212).

ADCs combine the power of antibody specificity with the ability to site specifically target a particular type of cell or tissue with a payload. Such site-specificity allows use of toxic payloads that would otherwise lack a sufficient therapeutic window. Research in this area has drawn significant interest and has led to marketed pharmaceutical products, including ADCETRIS® (brentuximab vedotin) and KADCYLA™ (ado-trastuzumab emtansine).

With few exceptions, current antiviral therapies merely treat symptoms of the disease or inhibit viral replication. Such treatments may slow the progression of the disease until either the natural immune response becomes effective or viral stasis is reached. Furthermore, there are few, if any, efficacious broad antiviral therapeutics. Thus, there is a significant unmet need for treatment and prophylaxis of virus infections.

Furthermore, development of antivirals that utilize alternative mechanisms of action is critical for reducing the risk of viruses developing resistance to existing therapeutics. Utilization of ADCs containing a broad-spectrum payload may facilitate use of a broad range of antiviral antibodies which often lack sufficient neutralizing activity to be efficacious on their own. Thus, there is a continuing need for efficient, site-specific methods for treating viral infections.

### SUMMARY

Provided herein are verrucarin A derivatives and ADCs thereof. In one embodiment, the verrucarin A derivatives for use in the compositions and methods provided herein have Formula I:

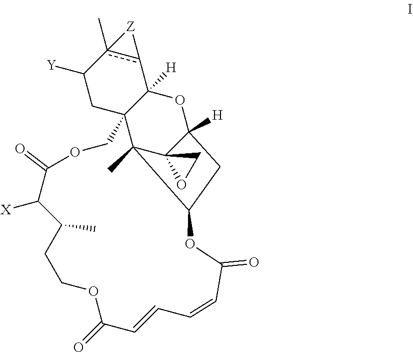

or a pharmaceutically acceptable derivative thereof, wherein X, Y and Z are as defined elsewhere herein.

In another embodiment, the verrucarin A derivatives for use in the compositions and methods provided herein have Formula II:

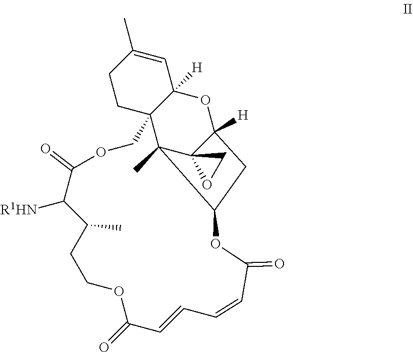

or a pharmaceutically acceptable derivative thereof, wherein R¹ is as defined elsewhere herein.

In another embodiment, provided are linker-verrucarin A derivatives of Formula III for use in the compositions and methods provided herein:

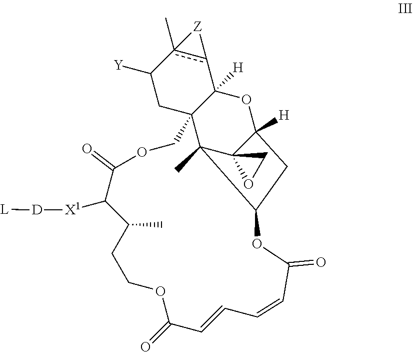

or a pharmaceutically acceptable derivative thereof, wherein X¹, D, L, Y, and Z are as defined elsewhere herein.

In another embodiment, provided are linker-verrucarin A derivatives of Formula IV for use in the compositions and methods provided herein:

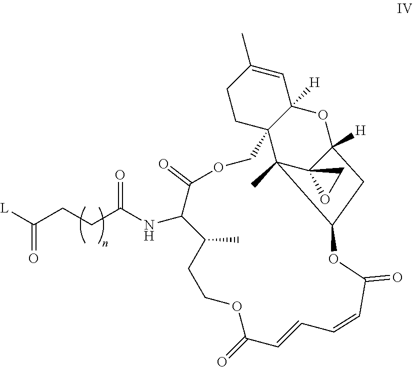

or a or a pharmaceutically acceptable derivative thereof, wherein L and n are as defined elsewhere herein.

In another embodiment, provided are ADCs of Formula V for use in the compositions and methods provided herein:

Z-(L-X)ᵥ V

or a pharmaceutically acceptable derivative thereof, wherein:

Z is an anti-viral antigen-binding domain;

L is a linking group as defined elsewhere herein;

X is a verrucarin A derivative; and

v is an integer from 1 to 12.

In another embodiment, the verrucarin A derivatives and ADCs thereof provided herein are useful in methods of treatment of viral diseases. In one embodiment, the viral disease is influenza, COVID-19 or ebola.

### DETAILED DESCRIPTION

### I. Definitions

To facilitate understanding of the disclosure set forth herein, a number of terms are defined below.

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

The singular forms “a,” “an,” and “the” include plural references, unless the context clearly dictates otherwise.

As used herein “subject” is an animal, such as a mammal, including human, such as a patient.

As used herein, biological activity refers to the in vivo activities of a compound or physiological responses that result upon in vivo administration of a compound, composition or other mixture. Biological activity, thus, encompasses therapeutic effects and pharmacokinetic behavior of such compounds, compositions and mixtures. Biological activities can be observed in in vitro systems designed to test for such activities.

As used herein, “influenza” means influenza A, B and C. “Influenza A” means a virus including eighteen subtypes defined by their hemagglutinin proteins (H1-H18) and eleven subtypes defined by their neuraminidase proteins (N1-N11). All combinations of these subtypes are possible and included within the scope of influenza A in this disclosure. Subtypes that affect humans include H1, H2, H3, H5, H6, H7, H9 and H10; and N1, N2, N6, N7, N8 and N9. Thus, in certain embodiments, the influenza A has a combination of these subtypes. In certain embodiments, influenza A may be an H1N1, H3N2, etc., subtype.

As used herein, “SARS-CoV-2” means the SARS-CoV-2 virus, including variants thereof (e.g., variants being monitored, variants of interest, variants of concern, and variants of high consequence). As of June 2022, variants being monitored include: Alpha (B.1.1.7 and Q lineages), Beta (B.1.351 and descendent lineages), Gamma (P.1 and descendent lineages), Delta (B.1.617.2 and AY lineages), Epsilon (B.1.427 and B.1.429), Eta (B.1.525), Iota (B.1.526), Kappa (B.1.617.1), 1.617.3, Mu (B.1.621 and B.1.621.1), and Zeta (P.2). As of June 2022, variants of concern include Omicron (B.1.1.529, BA.1, BA.1.1, BA.2, BA.3, BA.4 and BA.5 lineages).

As used herein, “antigen-binding domain” means any peptide, polypeptide, nucleic acid molecule, scaffold-type molecule, peptide display molecule, or polypeptide-containing construct that is capable of specifically binding a particular antigen of interest. As used herein, “antigen-binding domain” includes antibodies and antigen-binding fragments of antibodies. All references to proteins, polypeptides and protein fragments herein are intended to refer to the human version of the respective protein, polypeptide or protein fragment unless explicitly specified as being from a non-human species.

As used herein, the term “specifically binds” or the like means that the antigen-binding domain forms a complex with a particular antigen characterized by a dissociation constant (K₀) of 500 μM or less, and does not bind other unrelated antigens under ordinary test conditions.

As used herein, “unrelated antigens” are proteins, peptides or polypeptides that have less than 95% amino acid identity to one another.

The term “antibody,” as used herein, means any antigen-binding molecule or molecular complex comprising at least one complementarity determining region (CDR) that specifically binds to or interacts with a particular antigen (e.g., on or in an influenza viral particle or a SARS-CoV-2 viral particle). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, CH1, CH2 and CH3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

As used herein, the term “antigen-binding fragment” of an antibody means any naturally occurring, enzymatically obtainable, synthetic, or genetically engineered polypeptide or glycoprotein that specifically binds an antigen to form a complex.

As used herein, the term “human antibody” means antibodies having variable and constant regions derived from human germline immunoglobulin sequences. Human antibodies may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

As used herein, the term “recombinant human antibody”, means all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see, e.g., Taylor et al. (1992) Nucl. Acids Res. 20:6287-6295) or antibodies prepared, expressed, created or isolated by any other means that involves splicing of human immunoglobulin gene sequences to other DNA sequences.

As used herein in the context of amino acid sequences, the term “substantial identity” or “substantially identical” means that two amino acid sequences, when optimally aligned, such as by the programs GAP or BESTFIT using default gap weights, share at least 95%, 98% or 99% sequence identity.

As used herein, the term “surface plasmon resonance”, refers to an optical phenomenon that allows for the analysis of real-time interactions by detection of alterations in protein concentrations within a biosensor matrix, for example using the BIAcore™ system (Biacore Life Sciences division of GE Healthcare, Piscataway, N.J.).

As used herein, the term “K_{D}”, means the equilibrium dissociation constant of a particular protein-protein interaction (e.g., antibody-antigen interaction). Unless indicated otherwise, the K_{D} values disclosed herein refer to K_{D} values determined by surface plasmon resonance assay at 25° C.

As used herein, pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and inorganic salts, such as but not limited to, sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates, mesylates, and fumarates.

As used herein, treatment means any manner in which one or more of the symptoms of a disease or disorder are ameliorated or otherwise beneficially altered. Treatment also encompasses any pharmaceutical use of the compositions herein, such as use for treating viral infections.

As used herein, amelioration of the symptoms of a particular disorder by administration of a particular compound or pharmaceutical composition refers to any lessening, whether permanent or temporary, lasting or transient that can be attributed to or associated with administration of the compound or pharmaceutical composition.

As used herein, the IC₅₀ refers to an amount, concentration or dosage of a particular test compound that achieves a 50% inhibition of a maximal response in an assay that measures such response.

Where moieties are specified by their conventional chemical formulae, written from left to right, they equally encompass the chemically identical moieties that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain saturated hydrocarbon radical. The term “alkylene” by itself or as part of another substituent means a divalent radical derived from an alkyl. Typically, an alkyl (or alkylene) group will have from 1 to 24 carbon atoms, including those groups having 10 or fewer carbon atoms. A “lower alkyl” or “lower alkylene” is a shorter chain alkyl or alkylene group, generally having six or fewer carbon atoms. Examples of alkyl groups include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

The term “alkenyl,” by itself or as part of another substituent, means, unless otherwise stated, a straight (i.e., unbranched) or branched chain hydrocarbon radical having one or more carbon-carbon double bonds. The term “alkenylene” by itself or as part of another substituent means a divalent radical derived from an alkenyl. Typically, an alkenyl (or alkenylene) group will have from 1 to 24 carbon atoms, including those groups having 10 or fewer carbon atoms. A “lower alkenyl” or “lower alkenylene” is a shorter chain alkenyl or alkenylene group, generally having six or fewer carbon atoms. Examples of alkenyl groups include, but are not limited to, vinyl (i.e., ethenyl), 2-propenyl, crotyl, 2-isopentenyl, 2-(butadienyl), 2,4-pentadienyl, 3-(1,4-pentadienyl), and the higher homologs and isomers.

The term “alkynyl,” by itself or as part of another substituent, means, unless otherwise stated, a straight (i.e., unbranched) or branched chain hydrocarbon radical having one or more carbon-carbon triple bonds, which can include di- and multivalent radicals, having the number of carbon atoms designated (i.e., C₁-C₁₀ means one to ten carbons). Examples of alkynyl groups include, but are not limited to, ethynyl, 1- and 3-propynyl, 3-butynyl, and the higher homologs and isomers.

The terms “alkoxy,” “alkylamino,” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a straight or branched chain hydrocarbon radical, consisting of a heteroatom in the chain selected from the group consisting of O, N, P, Si and S, and wherein the nitrogen and sulfur atoms may optionally be oxidized and the nitrogen atom may have an alkyl substituent to fulfill valency and/or may optionally be quaternized. The heteroatom(s) O, N, P, Si and S may be placed at any interior position of the heteroalkyl group. Examples include, but are not limited to, —CH₂—CH₂—O—CH₃, —CH₂—CH₂—NH—CH₃, —CH₂—CH₂—N(CH₃)—CH₃, —CH₂—CH₂—S(O)—CH₃, —CH₂—CH₂—S(O)₂—CH₃, —CH═CH—O—CH₃, OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively, including bicyclic, tricyclic and bridged bicyclic groups. Additionally, for heterocycloalkyl, a heteroatom can occupy the position at which the heterocycle is attached to the remainder of the molecule. The terms “cycloalkylene” and “heterocycloalkylene” by themselves or as part of another substituent means a divalent radical derived from a cycloalkyl or heterocycloalkyl. Examples of cycloalkyl include, but are not limited to, cyclopentyl, cyclohexyl, 1-cyclohexenyl, 3-cyclohexenyl, cycloheptyl, norbornanyl, bicyclo(2.2.2)octanyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, 1- or 2-azabicyclo(2.2.2)octanyl, and the like.

The term “aryl” means, unless otherwise stated, a polyunsaturated, aromatic, hydrocarbon substituent which can be a single ring or multiple rings (in one embodiment from 1 to 3 rings) which are fused together or linked covalently. The term “heteroaryl” refers to aryl groups that contain from one to four heteroatoms selected from N, O, and S in the ring(s), wherein the nitrogen and sulfur atoms are optionally oxidized, and the nitrogen atom(s) are optionally quaternized. A heteroaryl group can be attached to the remainder of the molecule through a carbon or heteroatom. The terms “arylene” and “heteroarylene” by themselves or as part of another substituent means a divalent radical derived from a aryl or heteroaryl. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. The term “heteroarylium” refers to a heteroaryl group that is positively charged on one or more of the heteroatoms.

Each of the above terms are meant to include both substituted and unsubstituted forms of the indicated radical. Non-limiting examples of substituent moieties for each type of radical are provided below.

Substituent moieties for alkyl, heteroalkyl, alkylene, alkenyl, heteroalkylene, heteroalkenyl, alkynyl, cycloalkyl, heterocycloalkyl, cycloalkenyl, and heterocycloalkenyl groups are, in one embodiment, selected from, deuterium, —OR′, ═O, ═NR′, ═N—OR′, —NR′R″, —SR′, halo, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to the number of hydrogen atoms in such radical. In one embodiment, substituent moieties for cycloalkyl, heterocycloalkyl, cycloalkenyl, and heterocycloalkenyl groups also include substituted and unsubstituted alkyl, substituted and unsubstituted alkenyl, and substituted and unsubstituted alkynyl. R′, R″, R′″ and R″″ each in one embodiment independently are hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound provided herein includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituent moieties, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Substituent moieties for aryl and heteroaryl groups are, in one embodiment, selected from deuterium, halo, substituted and unsubstituted alkyl, substituted and unsubstituted alkenyl, and substituted and unsubstituted alkynyl, —OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of hydrogens on the aromatic ring system; and where R′, R″, R′″ and R″″ are, in one embodiment, independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound provided herein includes more than one R group, for example, each of the R groups is independently selected as are each R′, R″, R′″ and R″″ groups when more than one of these groups is present.

Two of the substituent moieties on adjacent atoms of an aryl or heteroaryl ring may optionally form a ring of the formula -Q′-C(O)—(CRR′)_{q}-Q″-, wherein Q′ and Q″ are independently —NR—, —O—, —CRR′— or a single bond, and q is an integer of from 0 to 3. Alternatively, two of the substituent moieties on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituent moieties on adjacent atoms of the aryl or heteroaryl ring may optionally be replaced with a substituent of the formula —(CRR′)ₛ—X′—(CR″R′″)_{d}—, where s and d are independently integers of from 0 to 3, and X is —O—, —NR′—, —S—, —S(O)—, —S(O)₂—, or —S(O)₂NR′—. The substituent moieties R, R′, R″ and R′″ are, in one embodiment, independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

The terms “halo,” by itself or as part of another substituent, means, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is meant to include, but not be limited to, trifluoromethyl, 2,2,2-trifluoroethyl, 4-chlorobutyl, 3-bromopropyl, and the like.

The term “oxo” as used herein means an oxygen atom that is double bonded to a carbon atom.

As used herein, the term “heteroatom” or “ring heteroatom” is meant to include oxygen (O), nitrogen (N), sulfur (S), phosphorus (P), and silicon (Si).

Certain verrucarin A derivatives or ADCs provided herein possess asymmetric carbon atoms (optical centers) or double bonds; the racemates, diastereomers, tautomers, geometric isomers and individual isomers are encompassed within the scope of the present disclosure. The ADCs provided herein do not include those which are known in the art to be too unstable to synthesize and/or isolate.

### II. Verrucarin A Derivatives

In one embodiment, provided herein are verrucarin A derivatives of Formula I for use in the compositions and methods provided herein:

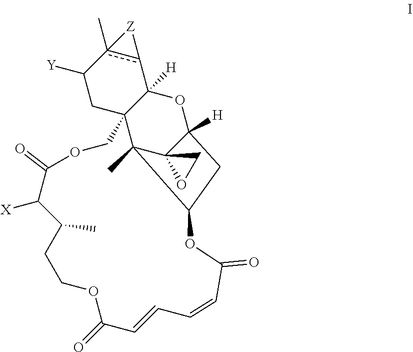

or a pharmaceutically acceptable derivative thereof, wherein:

X is NR¹R², OR³ or SR⁴;

R¹ and R² are each independently H, alkyl, OR⁵ or COR⁶, or together with the nitrogen atom to which they are attached form heterocycloalkyl;

R³ is alkyl or COR⁷;

R⁴ is H, alkyl or COR⁸;

R⁵ is H or alkyl;

R⁶ is R⁹, OR¹⁹ or NR¹¹R¹²;

R⁷-R⁹ are each independently alkyl or aralkyl;

R¹⁰-R¹² are each independently H, alkyl or aralkyl;

Y is H or OH; and

(i) Z is O and [symbol] is a single bond; or (ii) Z is absent and [symbol] is a double bond.

In another embodiment, R¹ and R² are each independently H, lower alkyl or COR⁶, or together with the nitrogen atom to which they are attached form heterocycloalkyl.

In another embodiment, R¹ and R² are each independently H, methyl, ethyl, C(O)-alkylene-CO—W where W is OR¹³ or NR¹⁴R¹⁵, or C(O)—CH(V)—CH₃ where V is OR¹⁶ or NR¹⁷R¹⁸, or together with the nitrogen atom to which they are attached form piperazinyl, piperidinyl, pyrrolidinyl, imidazolidinyl or azepinyl; R¹³ and R¹⁶ are each independently H or alkyl; and R¹⁴, R¹⁵, R¹⁷ and R¹⁸ are each independently H, alkyl, hydroxy or alkoxy.

In another embodiment, R¹ and R² are each independently H, methyl, ethyl, C(O)—(C₂₋₄alkylene)-CO—W where W is OR¹³ or NR¹⁴R¹⁵, or C(O)—CH(V)—CH₃ where V is OR¹⁶ or NR¹⁷R¹⁸, or together with the nitrogen atom to which they are attached form piperazinyl; R¹³ and R¹⁶ are each independently H or methyl; and R¹⁴, R¹⁵, R¹⁷ and R¹⁸ are each independently H, methyl, hydroxy or methoxy.

In another embodiment, R¹ is H, methyl, ethyl, C(O)CH₂CH₂COOH, C(O)CH₂CH₂CH₂COOH, C(O)CH₂CH₂CH₂CH₂COOH, C(O)CH₂CH₂CH₂COOMe, C(O)CH₂CH₂CH₂CONHOH, C(O)CH₂CH₂CH₂CONHOMe, C(O)—CH(OH)—CH₃, C(O)—CH(NH₂)—CH₃ or C(O)—CH(NMe₂)-CH₃.

In another embodiment, R² is H or methyl.

In another embodiment, R¹ and R² together with the nitrogen atom to which they are attached form 4-methyl-1-piperazinyl.

In another embodiment, R³ is methyl or C(O)-alkyl, where the alkyl is optionally substituted. In another embodiment, R³ is methyl or C(O)—CH(NR¹⁹R²⁰)—CHs, where R¹⁹ and R²⁰ are each independently H, alkyl or CO-alkyl. In another embodiment, R³ is methyl, C(O)—CH(NHMe)-CH₃ or C(O)—CH(NHAc)—CH₃.

In another embodiment, R⁴ is COR⁸. In another embodiment, R⁴ is C(O)Me.

In another embodiment, R⁵ is H or methyl.

In another embodiment, R⁶ is R⁹, where R⁹ is alkylene-CO—W, where W is OR¹³ or NR¹⁴R¹⁵, R¹³ is H or methyl; and R¹⁴ and R¹⁵ are each independently H, methyl, hydroxy or methoxy. In another embodiment, R⁶ is CH₂CH₂COOH, CH₂CH₂CH₂COOH, CH₂CH₂CH₂CH₂COOH, CH₂CH₂CH₂COOMe, CH₂CH₂CH₂CONHOH or CH₂CH₂CH₂CONHOMe.

In another embodiment, R⁷ and R⁸ are each independently alkyl. In another embodiment, R⁷ and R⁸ are each independently methyl.

In another embodiment, R¹⁰-R¹² are each independently H or alkyl. In another embodiment, R¹⁰-R¹² are each independently H or methyl.

In another embodiment, Y is H, Z is absent and [symbol] is a double bond. In another embodiment, Y is OH, Z is absent and [symbol] is a double bond. In another embodiment, Y is H, Z is O and [symbol] is a single bond.

In one embodiment, the verrucarin A derivatives of Formula I for use in the compositions and methods provided herein have Formula Ia:

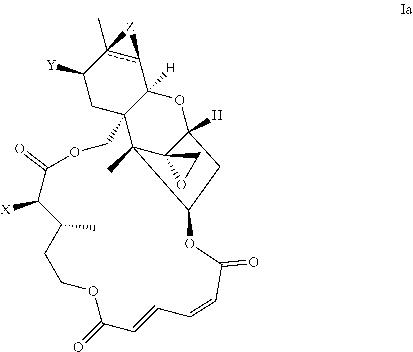

or a pharmaceutically acceptable derivative thereof, wherein the variables are as defined elsewhere herein.

In another embodiment, the verrucarin A derivatives of Formula I for use in the compositions and methods provided herein have Formula Ib:

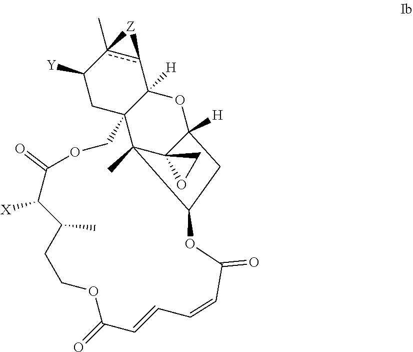

or a pharmaceutically acceptable derivative thereof, wherein the variables are as defined elsewhere herein.

In one embodiment, provided herein are verrucarin A derivatives of Formula II for use in the compositions and methods provided herein:

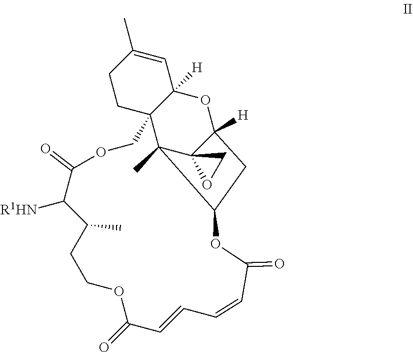

or a pharmaceutically acceptable derivative thereof, wherein:

R¹ is H or —COR⁶; and

R⁶ is -alkylene-COOH.

In another embodiment, R¹ is H. In another embodiment, R¹ is —COR⁶.

In another embodiment, R⁶ is —(CR²¹R²²)ₘCOOH, where R²¹ and R²² are each independently H or alkyl; and m is an integer from 0-6. In another embodiment, R²¹ and R²² are each independently H or methyl. In another embodiment, R²¹ and R²² are each H.

In another embodiment, m is 2, 3 or 4. In another embodiment, m is 2. In another embodiment, m is 3. In another embodiment, m is 4.

In another embodiment, R is —COR¹, where R¹ is —(CH₂)ₘCOOH where m is 2, 3 or 4. In another embodiment, R¹ is —CO—(CH₂)₂COOH. In another embodiment, R¹ is —CO—(CH₂)₃COOH. In another embodiment, R¹ is —CO—(CH₂)₄COOH.

In another embodiment, the verrucarin A derivatives of Formula II for use in the compositions and methods provided herein have Formula IIa:

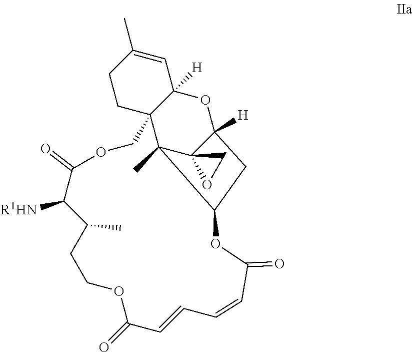

or a pharmaceutically acceptable derivative thereof, where R¹ is as defined elsewhere herein.

In another embodiment, the verrucarin A derivatives of Formula II for use in the compositions and methods provided herein have Formula IIb:

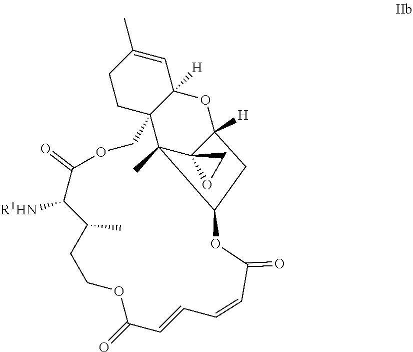

or a pharmaceutically acceptable derivative thereof, where R¹ is as defined elsewhere herein.

In one embodiment, the verrucarin A derivatives for use in the compositions and methods provided herein are selected from:

| Compound | Structure |
|---|---|
| 4 (R)-amino-verrucarin A | 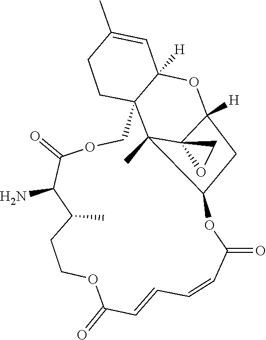 [SMILES: CC1=C[C@H]2O[C@@H]3C[C@H]4OC(=O)/C=C\C=C\C(=O)OCC[C@@H](C)[C@@H](N)C(=O)OC[C@@]2(CC1)[C@]4(C)[C@]31CO1] |
| 5 | 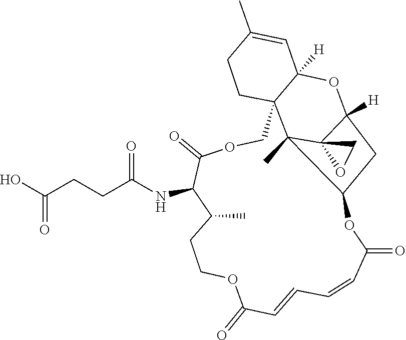 [SMILES: CC1=C[C@H]2O[C@@H]3C[C@H]4OC(=O)/C=C\C=C\C(=O)OCC[C@@H](C)[C@@H](NC(=O)CCC(=O)O)C(=O)OC[C@@]2(CC1)[C@]4(C)[C@]31CO1] |
| 6 | 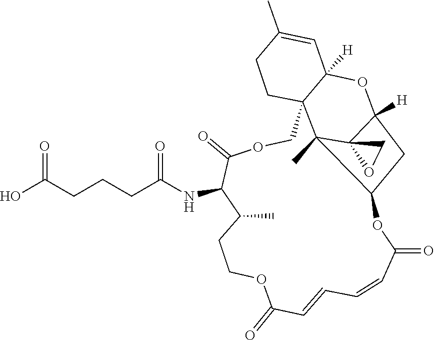 [SMILES: CC1=C[C@H]2O[C@@H]3C[C@H]4OC(=O)/C=C\C=C\C(=O)OCC[C@@H](C)[C@@H](NC(=O)CCCC(=O)O)C(=O)OC[C@@]2(CC1)[C@]4(C)[C@]31CO1] |
| 7 | 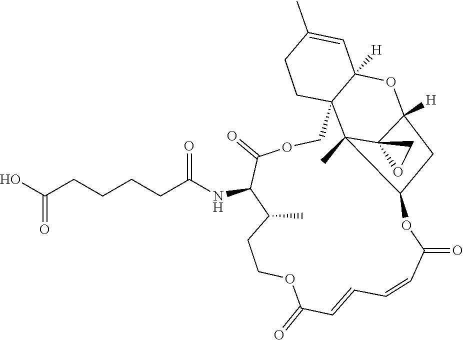 [SMILES: CC1=C[C@H]2O[C@@H]3C[C@H]4OC(=O)/C=C\C=C\C(=O)OCC[C@@H](C)[C@@H](NC(=O)CCCCC(=O)O)C(=O)OC[C@@]2(CC1)[C@]4(C)[C@]31CO1] |
| 22 | 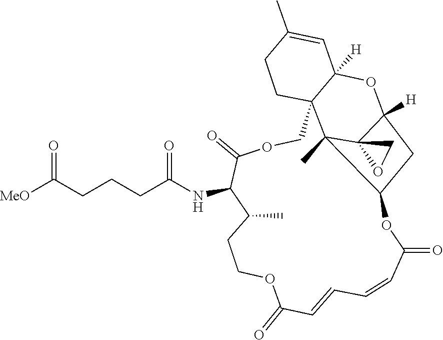 [SMILES: COC(=O)CCCC(=O)N[C@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1] |
| 24 | 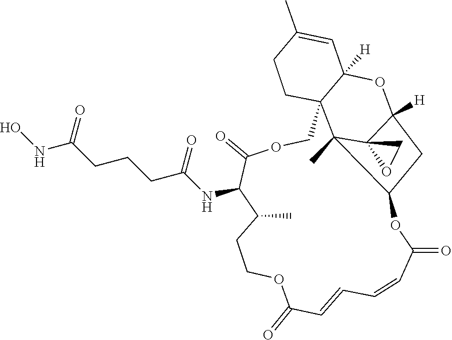 [SMILES: CC1=C[C@H]2O[C@@H]3C[C@H]4OC(=O)/C=C\C=C\C(=O)OCC[C@@H](C)[C@@H](NC(=O)CCCC(=O)NO)C(=O)OC[C@@]2(CC1)[C@]4(C)[C@]31CO1] |
| 25 | 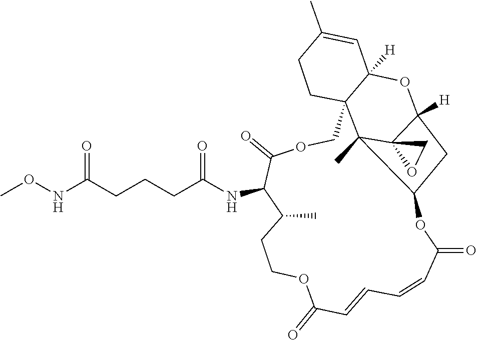 [SMILES: CONC(=O)CCCC(=O)N[C@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1] |
| 54 | 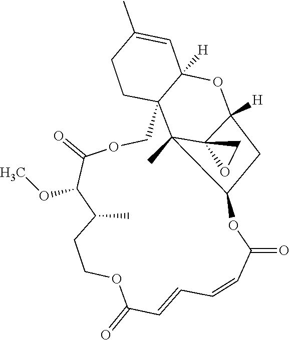 [SMILES: CO[C@@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1] |
| 55 | 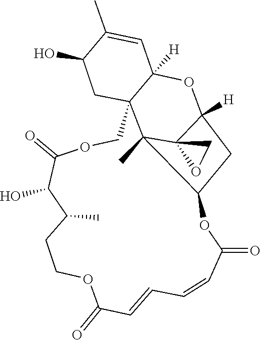 [SMILES: CC1=C[C@H]2O[C@@H]3C[C@H]4OC(=O)/C=C\C=C\C(=O)OCC[C@@H](C)[C@H](O)C(=O)OC[C@@]2(C[C@H]1O)[C@]4(C)[C@]31CO1] |
| 56 | 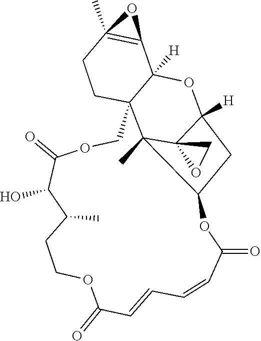 |
| 57 | 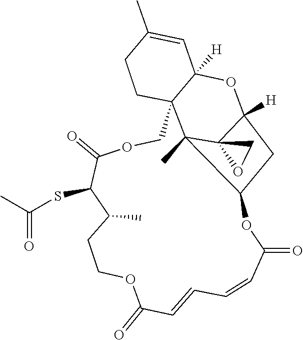 [SMILES: CC(=O)S[C@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1] |
| 58 | 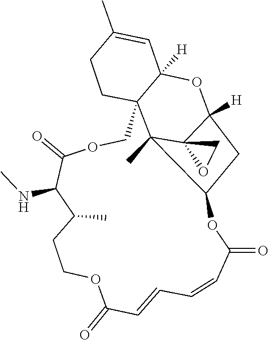 [SMILES: CN[C@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1] |
| 59 | 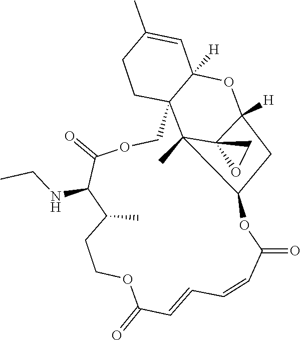 [SMILES: CCN[C@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1] |
| 60 | 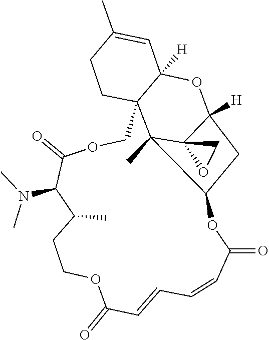 [SMILES: CC1=C[C@H]2O[C@@H]3C[C@H]4OC(=O)/C=C\C=C\C(=O)OCC[C@@H](C)[C@@H](N(C)C)C(=O)OC[C@@]2(CC1)[C@]4(C)[C@]31CO1] |
| 61 | 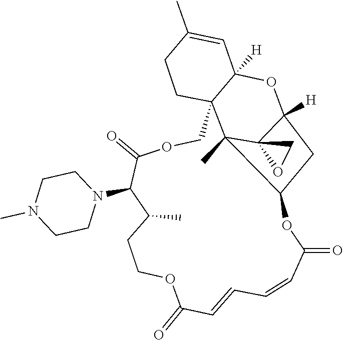 [SMILES: CC1=C[C@H]2O[C@@H]3C[C@H]4OC(=O)/C=C\C=C\C(=O)OCC[C@@H](C)[C@@H](N5CCN(C)CC5)C(=O)OC[C@@]2(CC1)[C@]4(C)[C@]31CO1] |
| 64 | 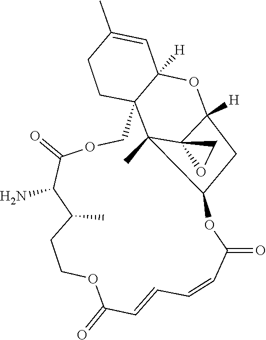 [SMILES: CC1=C[C@H]2O[C@@H]3C[C@H]4OC(=O)/C=C\C=C\C(=O)OCC[C@@H](C)[C@H](N)C(=O)OC[C@@]2(CC1)[C@]4(C)[C@]31CO1] |
| 65 | 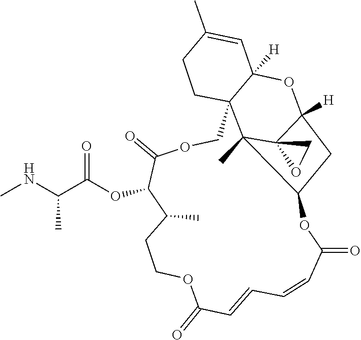 [SMILES: CN[C@@H](C)C(=O)O[C@@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1] |
| 66 | 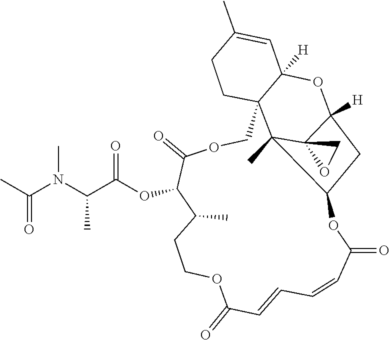 [SMILES: CC(=O)N(C)[C@@H](C)C(=O)O[C@@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1] |
| 78 | 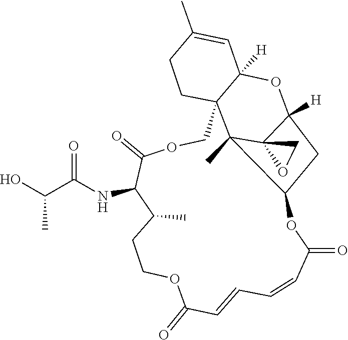 [SMILES: CC1=C[C@H]2O[C@@H]3C[C@H]4OC(=O)/C=C\C=C\C(=O)OCC[C@@H](C)[C@@H](NC(=O)[C@H](C)O)C(=O)OC[C@@]2(CC1)[C@]4(C)[C@]31CO1] |
| 80 | 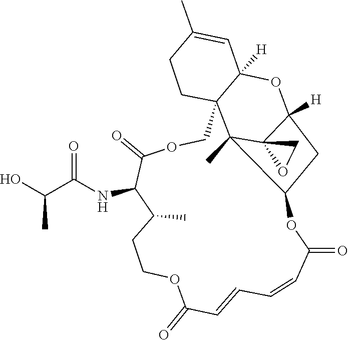 [SMILES: CC1=C[C@H]2O[C@@H]3C[C@H]4OC(=O)/C=C\C=C\C(=O)OCC[C@@H](C)[C@@H](NC(=O)[C@@H](C)O)C(=O)OC[C@@]2(CC1)[C@]4(C)[C@]31CO1] |
| 82 | 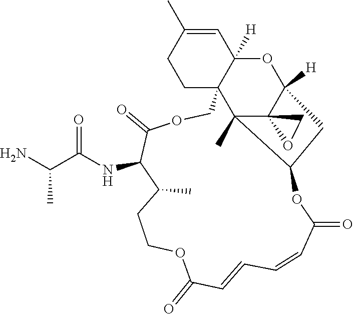 [SMILES: CC1=C[C@H]2O[C@@H]3C[C@H]4OC(=O)/C=C\C=C\C(=O)OCC[C@@H](C)[C@@H](NC(=O)[C@H](C)N)C(=O)OC[C@@]2(CC1)[C@]4(C)[C@]31CO1] |
| 84 | 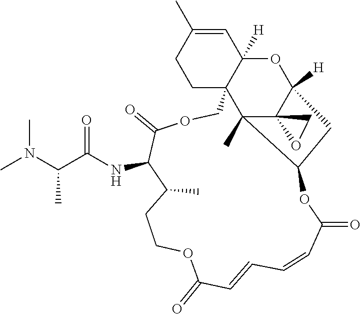 [SMILES: CC1=C[C@H]2O[C@@H]3C[C@H]4OC(=O)/C=C\C=C\C(=O)OCC[C@@H](C)[C@@H](NC(=O)[C@H](C)N(C)C)C(=O)OC[C@@]2(CC1)[C@]4(C)[C@]31CO1] |

In another embodiment, the verrucarin A derivatives for use in the compositions and methods provided herein are selected from:

| Compound | Structure |
|---|---|
| 4 (R)-amino-verrucarin A | 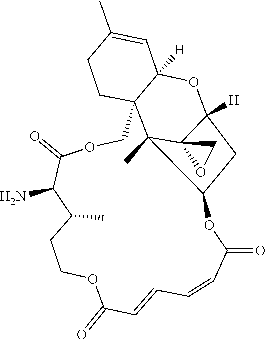 [SMILES: CC1=C[C@H]2O[C@@H]3C[C@H]4OC(=O)/C=C\C=C\C(=O)OCC[C@@H](C)[C@@H](N)C(=O)OC[C@@]2(CC1)[C@]4(C)[C@]31CO1] |
| 5 | 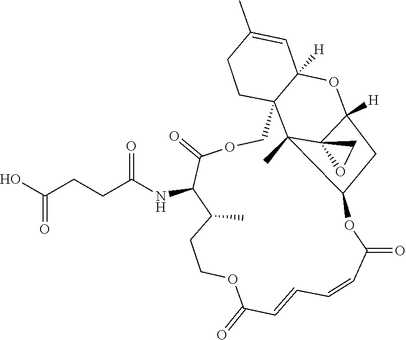 [SMILES: CC1=C[C@H]2O[C@@H]3C[C@H]4OC(=O)/C=C\C=C\C(=O)OCC[C@@H](C)[C@@H](NC(=O)CCC(=O)O)C(=O)OC[C@@]2(CC1)[C@]4(C)[C@]31CO1] |
| 6 | 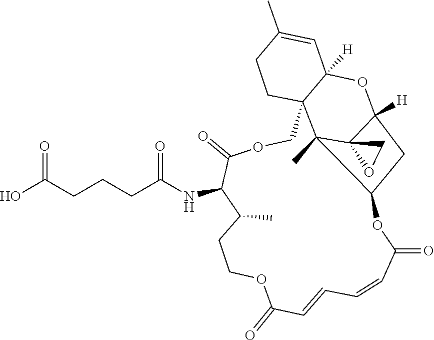 [SMILES: CC1=C[C@H]2O[C@@H]3C[C@H]4OC(=O)/C=C\C=C\C(=O)OCC[C@@H](C)[C@@H](NC(=O)CCCC(=O)O)C(=O)OC[C@@]2(CC1)[C@]4(C)[C@]31CO1] |
| 7 | 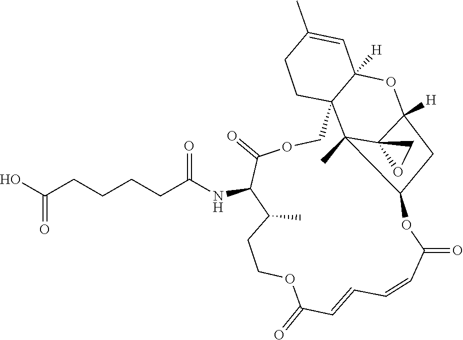 [SMILES: CC1=C[C@H]2O[C@@H]3C[C@H]4OC(=O)/C=C\C=C\C(=O)OCC[C@@H](C)[C@@H](NC(=O)CCCCC(=O)O)C(=O)OC[C@@]2(CC1)[C@]4(C)[C@]31CO1] |

### III. Synthesis of Verrucarin A Derivatives

The verrucarin A derivatives provided herein may be prepared by methods well known to those of skill in the art. For example, the free hydroxyl group of verrucarin A may be derivatized as a leaving group, e.g., a triflate using standard methods, e.g., triflic anhydride and pyridine. The leaving group may be displaced by a nucleophile, e.g., an amine or protected amine, an azide, etc. The resultant amino or azido derivative may then be deprotected or reduced, e.g., with triphenylphosphine, to provide an amino-verrucarin A, e.g., compound 4.

The amino-verrucarin A is then acylated under standard conditions, e.g., with an alkylene dicarboxylic acid anhydride and tertiary amine, to provide a HOOC-alkylene-CO-verrucarin A amide, e.g., compounds 5, 6, and 7.

### IV. Linker-Verrucarin A Derivatives

In one embodiment, provided herein are linker-verrucarin A derivatives of Formula III for use in the compositions and methods provided herein:

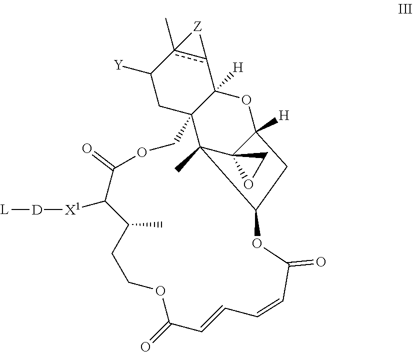

or a pharmaceutically acceptable derivative thereof, wherein X¹ is O or NH; D is absent or is —C(O)—(CH₂)₂₋₅—C(O)— or —O—NH—C(O)—(CH₂)₂₋₅—C(O)—; L is a linking group; Y is H or OH; and (i) Z is O and [symbol] is a single bond; or (ii) Z is absent and [symbol] is a double bond.

In one embodiment, Y is H, Z is absent and [symbol] is a double bond. In another embodiment, Y is OH, Z is absent and [symbol] is a double bond. In another embodiment, Y is H, Z is I and [symbol] is a single bond.

In another embodiment, X is O. In another embodiment, X is NH.

In another embodiment, D is absent. In another embodiment, D is —C(O)—(CH₂)₂₋₅—C(O)—. In another embodiment, D is —O—NH—C(O)—(CH₂)₂₋₅—C(O)—. In another embodiment, D is —C(O)—(CH₂)₂—C(O)—. In another embodiment, D is —C(O)—(CH₂)₃—C(O)—. In another embodiment, D is —C(O)—(CH₂)₄—C(O)—. In another embodiment, D is —O—NH—C(O)—(CH₂)₃—C(O)—.

In one embodiment, the linker-verrucarin A derivatives of Formula III have Formula IIIa:

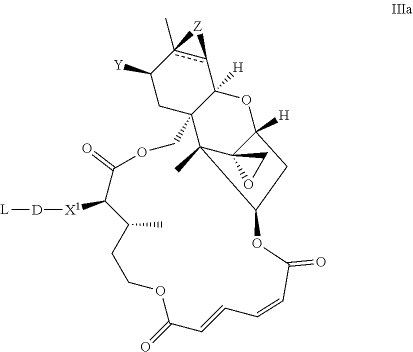

or a pharmaceutically acceptable derivative thereof, where the variables are as defined elsewhere herein.

In one embodiment, the linker-verrucarin A derivatives of Formula III have Formula IIIb:

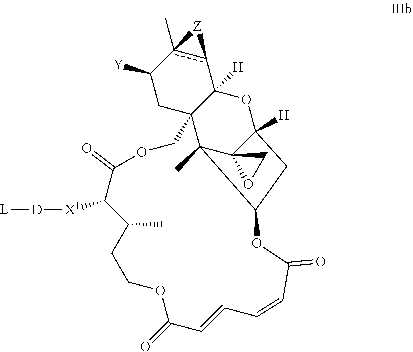

or a pharmaceutically acceptable derivative thereof, where the variables are as defined elsewhere herein.

In one embodiment, the linker-verrucarin A derivatives for use in the compositions and methods provided herein have Formula IV:

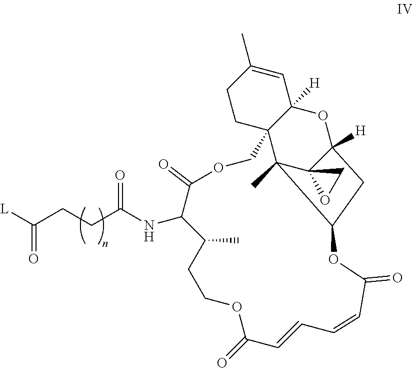

or a pharmaceutically acceptable derivative thereof, wherein L is a linking group and n is an integer from 1 to 4.

In another embodiment, n is 1, 2 or 3. In another embodiment, n is 1. In another embodiment, n is 2. In another embodiment, n is 3.

In one embodiment, the linker-verrucarin A derivatives of Formula IV have Formula IVa:

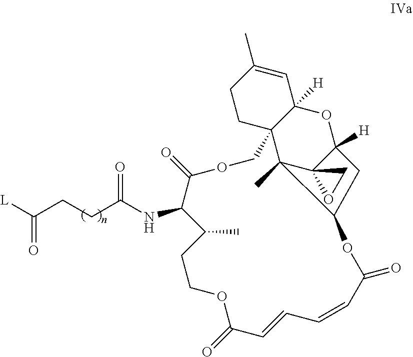

or a pharmaceutically acceptable derivative thereof, wherein the variables are as defined herein.

In another embodiment, the linker-verrucarin A derivatives of Formula IV have Formula IVb:

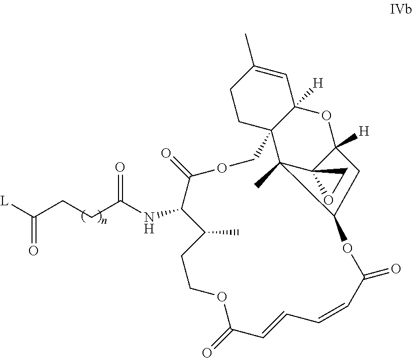

or a pharmaceutically acceptable derivative thereof, wherein the variables are as defined herein.

In certain embodiments, L is any group or moiety that links, connects, or bonds the selenium with a payload. Suitable linkers may be found, for example, in Antibody-Drug Conjugates and Immunotoxins, Phillips, G. L., Ed.; Springer Verlag: New York, 2013; Antibody-Drug Conjugates, Ducry, L., Ed.; Humana Press, 2013; Antibody-Drug Conjugates, Wang, J., Shen, W.-C., and Zaro, J. L., Eds.; Springer International Publishing, 2015. In certain embodiments, the L group for the verrucarin A derivatives and ADCs provided herein is sufficiently stable to exploit the circulating half-life of the antigen binding domain and, at the same time, capable of releasing its payload after antigen-mediated internalization of the ADC. Linker L can be cleavable or non-cleavable. Cleavable linkers for use as L herein include linkers that are cleaved by intracellular metabolism following internalization, e.g., cleavage via hydrolysis, reduction, or enzymatic reaction. Non-cleavable linkers for use as L herein include linkers that release an attached payload via lysosomal degradation of the antigen binding domain following internalization. Suitable L linkers include, but are not limited to, acid-labile linkers, hydrolysis-labile linkers, enzymatically cleavable linkers, reduction labile linkers, self-immolative linkers, and non-cleavable linkers. Suitable L linkers also include, but are not limited to, those that are or comprise peptides, carbohydrates, glucuronides, polyethylene glycol (PEG) units, hydrazones, mal-caproyl units, dipeptide units, valine-citruline units, and para-aminobenzyloxy (PAB) units.

Any linker molecule or linker technology known in the art can be used as L in the verrucarin A derivatives and ADCs provided herein. In certain embodiments, the L linker is a cleavable linker. In other embodiments, the L linker is a non-cleavable linker. In certain embodiments, L linkers that can be used in the ADCs provided herein include linkers that comprise or consist of, e.g., MC (6-maleimidocaproyl), MP (maleimidopropanoyl), val-cit (valine-citrulline), val-ala (valine-alanine), dipeptide site in protease-cleavable linkers, ala-phe (alanine-phenylalanine), dipeptide site in protease-cleavable linkers, PAB (p-aminobenzyloxy), and variants and combinations thereof. Additional examples of L linkers that can be used in the verrucarin A derivatives and ADCs provided herein are disclosed, e.g., in U.S. Pat. No. 7,754,681 and in Ducry, Bioconjugate Chem., 2010, 21:5-13, and the references cited therein.

In certain embodiments, the L linkers are stable in physiological conditions. In certain embodiments, the L linkers are cleavable, for instance, able to release at least the payload portion in the presence of an enzyme or at a particular pH range or value. In some embodiments, an L linker comprises an enzyme-cleavable moiety. In one embodiment, enzyme-cleavable L linkers include, but are not limited to, peptide bonds, ester linkages, and hydrazones. In some embodiments, the L linker comprises a cathepsin-cleavable linker.

In some embodiments, the L linker comprises a non-cleavable moiety.

In some embodiments, the L linker comprises one or more amino acids. Suitable amino acids include natural, non-natural, standard, non-standard, proteinogenic, non-proteinogenic, and L- or D-α-amino acids. In some embodiments, the L linker comprises alanine, valine, glycine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or combination thereof. In certain embodiments, one or more side chains of the amino acids is linked to a side chain group, described below. In some embodiments, the linker comprises valine and citrulline. In some embodiments, the L linker comprises lysine, valine, and citrulline. In some embodiments, the L linker comprises lysine, valine, and alanine. In some embodiments, the L linker comprises valine and alanine. In some embodiments, the linker is a peptide comprising or consisting of the amino acids alanine and alanine, or divalent -AA-. In some embodiments, the linker is a peptide comprising or consisting of the amino acids glutamic acid and alanine, or -EA-. In some embodiments, the linker is a peptide comprising or consisting of the amino acids glutamic acid and glycine, or -EG-. In some embodiments, the linker is a peptide comprising or consisting of the amino acids glycine and glycine, or -GG-. In some embodiments, the linker is a peptide comprising or consisting of the amino acids glutamine, valine, and citrulline, or -Q-V-Cit- or -QVCit-. In some embodiments, the linker is a peptide comprising or consisting of the amino acids glutamic acid, valine, and citrulline, or -E-V-Cit- or -EVCit-. In some embodiments, the linker is a peptide comprising or consisting of the amino acids -GGGGS-. In some embodiments, the linker is a peptide comprising or consisting of the amino acids -GGGGG-. In some embodiments, the linker is a peptide comprising or consisting of the amino acids -GGGGK-. In some embodiments, the linker is a peptide comprising or consisting of the amino acids -GFGG-. In some embodiments, the linker is a peptide comprising or consisting of the amino acids lysine, valine, and citrulline, or -KVCit-. In some embodiments, the linker is a peptide comprising or consisting of the amino acids -KVA-. In some embodiments, the linker is a peptide comprising or consisting of the amino acids -VA-.

In some embodiments, the L linker comprises a self-immolative group. The self-immolative group can be any such group known to those of skill in the art. In particular embodiments, the self-immolative group is p-aminobenzyl (PAB), or a derivative thereof. Useful derivatives include p-aminobenzyloxycarbonyl (PABC) and p-amino-α-methylbenzyl (MePAB). Those of skill in the art will recognize that a self-immolative group is capable of carrying out a chemical reaction which releases the remaining atoms of an L linker from a payload.

In other embodiments, the L group can be modified with one or more enhancement groups. In certain embodiments, the enhancement group can be linked to the side chain of any amino acid in L. In one embodiment, amino acids for linking enhancement groups include lysine, asparagine, aspartate, glutamine, glutamate, and citrulline. The link to the enhancement group can be a direct bond to the amino acid side chain, or the link can be indirect via a spacer and/or reactive group. In one embodiment, spacers and reactive groups include any described herein. In certain embodiments, the enhancement group can be any group that imparts a beneficial effect to the payload, linker payload, or ADC including, but not limited to, biological, biochemical, synthetic, solubilizing, imaging, detecting, and reactivity effects, and the like. In certain embodiments, the enhancement group is a hydrophilic group. In certain embodiments, the enhancement group is a cyclodextrin. In certain embodiments, the enhancement group is an alkyl, heteroalkyl, alkenyl, heteroalkenyl sulfonic acid, heteroalkenyl taurine, heteroalkenyl phosphoric acid or phosphate, heteroalkenyl amine (e.g., quaternary amine), or heteroalkenyl sugar. In certain embodiments, sugars include, without limitation, monosaccharides, disaccharides, and polysaccharides. Exemplary monosaccharides include glucose, ribose, deoxyribose, xylose, arabinose, mannose, galactose, fructose, and the like. In certain embodiments, sugars include sugar acids such as glucuronic acid, further including conjugated forms such as glucuronides (i.e., via glucuronidation). Exemplary disaccharides include maltose, sucrose, lactose, lactulose, trehalose, and the like. Exemplary polysaccharides include amylose, amylopectin, glycogen, inulin, cellulose, and the like. The cyclodextrin can be any cyclodextrin known to those of skill. In certain embodiments, the cyclodextrin is alpha cyclodextrin, beta cyclodextrin, or gamma cyclodextrin, or mixtures thereof. In certain embodiments, the cyclodextrin is alpha cyclodextrin. In certain embodiments, the cyclodextrin is beta cyclodextrin. In certain embodiments, the cyclodextrin is gamma cyclodextrin. In certain embodiments, the enhancement group is capable of improving solubility of the remainder of the ADC. In certain embodiments, the alkyl, heteroalkyl, alkenyl, or heteroalkenyl sulfonic acid is substituted or non-substituted. In certain embodiments, the alkyl, heteroalkyl, alkenyl, or heteroalkenyl sulfonic acid is —(CH₂)_{1_5}SO₃H, —(CH₂)ₓ—NH—(CH₂)₁₋₅SO₃H, —(CH₂)ₓ—C(O)NH—(CH₂)₁₋₅SO₃H, —(CH₂CH₂O)_{y}—C(O)NH—(CH₂)₁₋₅SO₃H, —(CH₂)ₓ—N((CH₂)₁₋₅C(O)NH(CH₂)₁₋₅SO₃H)₂, —(CH₂)ₓ—C(O)N((CH₂)_{1_5}C(O)NH(CH₂)₁₋₅SO₃H)₂, or —(CH₂CH₂O)_{y}—C(O)N((CH₂)₁₋₅C(O)NH(CH₂)₁₋₅SO₃H)₂, wherein x is 1, 2, 3, 4, or 5, and y is 1, 2, 3, 4, or 5. In one embodiment, the alkyl or alkenyl sulfonic acid is —(CH₂)₁₋₅ SO₃H. In another embodiment, the heteroalkyl or heteroalkenyl sulfonic acid is —(CH₂)ₓ—NH—(CH₂)₁₋₅SO₃H, wherein x is 1, 2, 3, 4, or 5. In another embodiment, the alkyl, heteroalkyl, alkenyl, or heteroalkenyl sulfonic acid is —(CH₂)ₓ—C(O)NH—(CH₂)₁₋₅SO₃H, wherein x is 1, 2, 3, 4, or 5. In another embodiment, the alkyl, heteroalkyl, alkenyl, or heteroalkenyl sulfonic acid is —(CH₂CH₂O)_{y}—C(O)NH—(CH₂)₁₋₅SO₃H, wherein y is 1, 2, 3, 4, or 5. In another embodiment, the alkyl, heteroalkyl, alkenyl, or heteroalkenyl sulfonic acid is —(CH₂)ₓ—N((CH₂)₁₋₅C(O)NH(CH₂)₁₋₅SO₃H)₂, wherein x is 1, 2, 3, 4, or 5. In another embodiment, the alkyl, heteroalkyl, alkenyl, or heteroalkenyl sulfonic acid is —(CH₂)ₓ—C(O)N((CH₂)₁₋₅C(O)NH(CH₂)₁₋₅SO₃H)₂, wherein x is 1, 2, 3, 4, or 5. In another embodiment, the alkyl, heteroalkyl, alkenyl, or heteroalkenyl sulfonic acid is (CH₂CH₂O)_{y}—C(O)N((CH₂)₁₋₅C(O)NH(CH₂)₁₋₅SO₃H)₂, wherein y is 1, 2, 3, 4, or 5.

In some embodiments, the linker is:

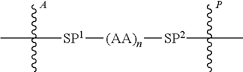

wherein:
SP¹ is a spacer;
SP² is a spacer;

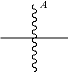

is one or more bonds to the antigen-binding domain Z;

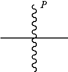

is one or more bonds to the verrucarin A derivative;
each AA is an amino acid residue; and
n is an integer from zero to ten.

The SP¹ spacer is a moiety that connects the (AA)ₙ moiety or residue to the antigen-binding domain Z or to a reactive group residue which is bonded to Z. Suitable SP¹ spacers include, but are not limited to, those comprising alkylene or polyether, or both. The ends of the spacers, for example, the portion of the spacer bonded to the Z or an AA, can be moieties derived from reactive moieties that are used for purposes of coupling the antibody or an AA to the spacer during chemical synthesis of the conjugate. In certain embodiments, n is 0, 1, 2, 3, or 4 (i.e., when n is 0, AA is absent). In one embodiment, n is 2. In another embodiment, n is 3. In another embodiment, n is 4.

In some embodiments, the SP¹ spacer comprises an alkylene. In some embodiments, the SP¹ spacer comprises a C₅₋₇ alkylene. In some embodiments, the SP¹ spacer comprises a polyether. In some embodiments, the SP¹ spacer comprises a polymer of ethylene oxide such as polyethylene glycol (PEG). Polymeric units of polyethylene glycol are commonly represented as —(OCH₂CH₂)ₚ—, where p is an integer from one to one hundred. For example, —(OCH₂CH₂)₂— can also be represented as —OCH₂CH₂—OCH₂CH₂— or PEG₂. In certain embodiments, the polyethylene glycol is PEG₁. In certain embodiments, the polyethylene glycol is PEG₂. In certain embodiments, the polyethylene glycol is PEG₃. In certain embodiments, the polyethylene glycol is PEG₄. In certain embodiments, the polyethylene glycol is PEG₅. In certain embodiments, the polyethylene glycol is PEG₆. In certain embodiments, the polyethylene glycol is PEG₇. In certain embodiments, the polyethylene glycol is PEG₈. In certain embodiments, the polyethylene glycol is PEG₉. In certain embodiments, the polyethylene glycol is PEG₁₀. In certain embodiments, the polyethylene glycol is PEG₁₁. In certain embodiments, the polyethylene glycol is PEG₁₂. In certain embodiments, the polyethylene glycol is PEG₁₃. In certain embodiments, the polyethylene glycol is PEG₁₄. In certain embodiments, the polyethylene glycol is PEG₁₅. In certain embodiments, the polyethylene glycol is PEG₁₆. In certain embodiments, the polyethylene glycol is PEG₁₇. In certain embodiments, the polyethylene glycol is PEG₁₈. In certain embodiments, the polyethylene glycol is PEG₁₉. In certain embodiments, the polyethylene glycol is PEG₂₀. In certain embodiments, the polyethylene glycol is PEG₂₁. In certain embodiments, the polyethylene glycol is PEG₂₂. In certain embodiments, the polyethylene glycol is PEG₂₃. In certain embodiments, the polyethylene glycol is PEG₂₄. In certain embodiments, the polyethylene glycol is PEG₂₅. In certain embodiments, the polyethylene glycol is PEG₂₆. In certain embodiments, the polyethylene glycol is PEG₂₇. In certain embodiments, the polyethylene glycol is PEG₂₈. In certain embodiments, the polyethylene glycol is PEG₂₉. In certain embodiments, the polyethylene glycol is PEG₃₀. In certain embodiments, the polyethylene glycol is PEG₃₁. In certain embodiments, the polyethylene glycol is PEG₃₂. In certain embodiments, the polyethylene glycol is PEG₃₃. In certain embodiments, the polyethylene glycol is PEG₃₄. In certain embodiments, the polyethylene glycol is PEG₃₅. In certain embodiments, the polyethylene glycol is PEG₃₆. In certain embodiments, the polyethylene glycol is PEG₃₇. In certain embodiments, the polyethylene glycol is PEG₃₈. In certain embodiments, the polyethylene glycol is PEG₃₉. In certain embodiments, the polyethylene glycol is PEG₄₀. In certain embodiments, the polyethylene glycol is PEG₄₁. In certain embodiments, the polyethylene glycol is PEG₄₂. In certain embodiments, the polyethylene glycol is PEG₄₃. In certain embodiments, the polyethylene glycol is PEG₄₄. In certain embodiments, the polyethylene glycol is PEG₄₅. In certain embodiments, the polyethylene glycol is PEG₄₆. In certain embodiments, the polyethylene glycol is PEG₄₇. In certain embodiments, the polyethylene glycol is PEG₄₈. In certain embodiments, the polyethylene glycol is PEG₄₉. In certain embodiments, the polyethylene glycol is PEG₅₀. In certain embodiments, the polyethylene glycol is PEG₅₁. In certain embodiments, the polyethylene glycol is PEG₅₂. In certain embodiments, the polyethylene glycol is PEG₅₃. In certain embodiments, the polyethylene glycol is PEG₅₄. In certain embodiments, the polyethylene glycol is PEG₅₅. In certain embodiments, the polyethylene glycol is PEG₅₆. In certain embodiments, the polyethylene glycol is PEG₅₇. In certain embodiments, the polyethylene glycol is PEG₅₈. In certain embodiments, the polyethylene glycol is PEG₅₉. In certain embodiments, the polyethylene glycol is PEG₆₀. In certain embodiments, the polyethylene glycol is PEG₆₁. In certain embodiments, the polyethylene glycol is PEG₆₂. In certain embodiments, the polyethylene glycol is PEG₆₃. In certain embodiments, the polyethylene glycol is PEG₆₄. In certain embodiments, the polyethylene glycol is PEG₆₅. In certain embodiments, the polyethylene glycol is PEG₆₆. In certain embodiments, the polyethylene glycol is PEG₆₇. In certain embodiments, the polyethylene glycol is PEG₆₈. In certain embodiments, the polyethylene glycol is PEG₆₉. In certain embodiments, the polyethylene glycol is PEG₇₀. In certain embodiments, the polyethylene glycol is PEG₇₁. In certain embodiments, the polyethylene glycol is PEG₇₂. In certain embodiments, the polyethylene glycol is PEG₇₃. In certain embodiments, the polyethylene glycol is PEG₇₄. In certain embodiments, the polyethylene glycol is PEG₇₅. In certain embodiments, the polyethylene glycol is PEG₇₆. In certain embodiments, the polyethylene glycol is PEG₇₇. In certain embodiments, the polyethylene glycol is PEG₇₇. In certain embodiments, the polyethylene glycol is PEG₇₉. In certain embodiments, the polyethylene glycol is PEG₈₀. In certain embodiments, the polyethylene glycol is PEG₈₁. In certain embodiments, the polyethylene glycol is PEG₈₂. In certain embodiments, the polyethylene glycol is PEG₈₃. In certain embodiments, the polyethylene glycol is PEG₈₄. In certain embodiments, the polyethylene glycol is PEG₈₅. In certain embodiments, the polyethylene glycol is PEG₈₆. In certain embodiments, the polyethylene glycol is PEG₈₇. In certain embodiments, the polyethylene glycol is PEG₈₈. In certain embodiments, the polyethylene glycol is PEG₈₉. In certain embodiments, the polyethylene glycol is PEG₉₀. In certain embodiments, the polyethylene glycol is PEG₉₁. In certain embodiments, the polyethylene glycol is PEG₉₂.

In some embodiments, the SP¹ spacer is:

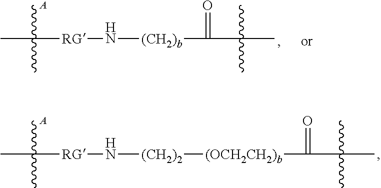

wherein:
RG′ is a reactive group residue following reaction of a reactive group RG with a binding agent;

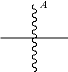

is a bond to the antigen-binding domain Z;

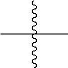

is a bond to (AA)ₙ;
n is an integer from zero to ten; and
b is, independently, an integer from 1 to 92.

The reactive group RG can be any reactive group known to those of skill in the art to be capable of forming one or more bonds to the antigen-binding domain Z. The reactive group RG is a moiety comprising a portion in its structure that is capable of reacting with the binding agent (e.g., reacting with an antibody at its cysteine or lysine residues, or at an azide moiety, for example, a PEG-N₃ functionalized antibody at one or more glutamine residues; or at an amino moiety, for example, a PEG-NH₂ functionalized antibody at one or more glutamine residues) to form antibody-drug conjugates described herein. Following conjugation to the antigen-binding domain, the reactive group becomes the reactive group residue (RG′). Illustrative reactive groups include, but are not limited to, those that comprise haloacetyl, isothiocyanate, succinimide, N-hydroxysuccinimide, or maleimide portions that are capable of reacting with the binding agent.

The SP² spacer, when present, is a moiety that connects the (AA)ₙ moiety to the payload. Suitable spacers include, but are not limited to, those described above as SP¹ spacers. Further suitable SP² spacers include, but are not limited to, those comprising alkylene or polyether, or both. The ends of the SP² spacers, for example, the portion of the spacer directly bonded to the verrucarin A derivative or an AA, can be moieties derived from reactive moieties that are used for purposes of coupling the verrucarin A derivative or AA to the SP² spacer during the chemical synthesis of the conjugate. In some examples, the ends of the SP² spacers, for example, the portion of the SP² spacer directly bonded to the verrucarin A derivative or an AA, can be residues of reactive moieties that are used for purposes of coupling the verrucarin A derivative or an AA to the spacer during the chemical synthesis of the conjugate.

In some embodiments, the SP² spacer, when present, is selected from the group consisting of —NH-(p-C₆H₄)—CH₂—, —NH-(p-C₆H₄)—CH₂OC(O)—, an amino acid, a dipeptide, a tripeptide, an oligopeptide

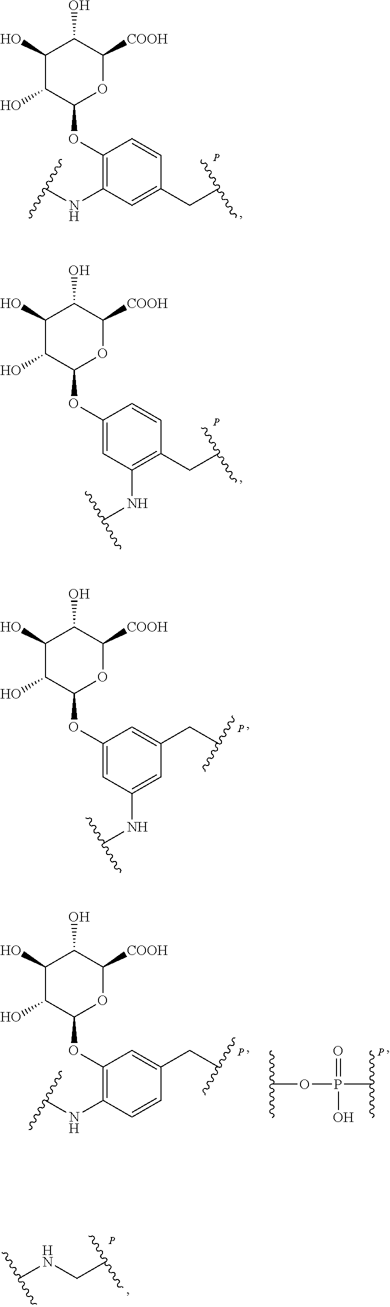

and any combinations thereof. In certain embodiments, each

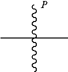

is a bond to the payload, and each

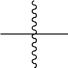

is a bond to (AA)ₙ or absent if n=0.

In the above formulae, each (AA)ₙ is an amino acid or, optionally, a p-aminobenzyl-oxycarbonyl residue (PABC). n can be 0; if so, (AA)ₙ is absent. In one embodiment, if PABC is present, only one PABC is present. In another embodiment, the PABC residue, if present, is bonded to a terminal AA in the (AA)ₙ group, proximal to the payload. Suitable amino acids for each AA include natural, non-natural, standard, non-standard, proteinogenic, non-proteinogenic, and L- or D-α-amino acids. In some embodiments, the AA comprises alanine, valine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or any combinations thereof (e.g., dipeptides, tripeptides, and oligopeptides, and the like). In certain embodiments, one or more side chains of the amino acids is linked to a side chain group, described below. In some embodiments, n is two. In some embodiments, the (AA)ₙ is valine-citrulline. In some embodiments, (AA)ₙ is citrulline-valine. In some embodiments, (AA)ₙ is valine-alanine. In some embodiments, (AA)ₙ is alanine-valine. In some embodiments, (AA)ₙ is valine-glycine. In some embodiments, (AA)ₙ is glycine-valine. In some embodiments, the (AA)ₙ is valine-citrulline-PABC. In some embodiments, (AA)ₙ is citrulline-valine-PABC. In some embodiments, n is three. In some embodiments, (AA)ₙ is glutamate-valine-citrulline. In some embodiments, (AA)ₙ is glutamine-valine-citrulline. In some embodiments, (AA)ₙ is lysine-valine-alanine. In some embodiments, (AA)ₙ is lysine-valine-citrulline. In some embodiments, n is four. In some embodiments, (AA)ₙ is glutamate-valine-citrulline-PAB. In some embodiments, (AA)ₙ is glutamine-valine-citrulline-PABC.

In another embodiment, L is a cleavable linker.

In another embodiment, L is an acid-labile linker, a hydrolysis-labile linker, an enzymatically cleavable linker, a reduction labile linker, or a self-immolative linker.

In another embodiment, L includes or is a peptide, a carbohydrate, an N-hydroxysuccinimidyl ester, a glucuronide, one or more polyethylene glycol units, a hydrazone, a mal-caproyl unit, a dipeptide unit, a valine-citruline unit, or a para-aminobenzyl unit, or combinations thereof.

In another embodiment, L includes or is a peptide, a carbohydrate, a glucuronide, one or more polyethylene glycol units, a hydrazone, a mal-caproyl unit, a dipeptide unit, a valine-citruline unit, or a para-aminobenzyl unit, or combinations thereof.

In another embodiment, L includes or is MC (6-maleimidocaproyl), MP (maleimidopropanoyl), val-cit (valine-citrulline), val-ala (valine-alanine), isoleucine-phenylalanine-citrulline, a dipeptide site in protease-cleavable linker, ala-phe (alanine-phenylalanine), or a PAB (p-aminobenzyloxy), or combinations thereof.

In another embodiment, L includes or is of MC (6-maleimidocaproyl), MP (maleimidopropanoyl), val-cit (valine-citrulline), val-ala (valine-alanine), a dipeptide site in protease-cleavable linker, ala-phe (alanine-phenylalanine), or a PAB (p-aminobenzyloxy), or combinations thereof.

In another embodiment, L includes val-cit. In another embodiment, L comprises or consists of val-cit-PAB. In another embodiment, L comprises or consists of val-cit-MePAB (val-cit-(p-amino-α-benzyloxy)). In another embodiment, L includes or is AC (6-aminocaproyl). In another embodiment, L includes or is MC (6-maleimidocaproyl). In another embodiment, L includes or is MC-val-cit-PAB. In another embodiment, L includes or is AC-val-cit-PAB. In another embodiment, L includes or is MC-val-cit-MePAB. In another embodiment, L includes or is AC-val-cit-MePAB. In another embodiment, L includes or is AC-GGFG-CH₂—.

In certain embodiments, the linker-verrucarin A derivative provided herein has one of the formulae:

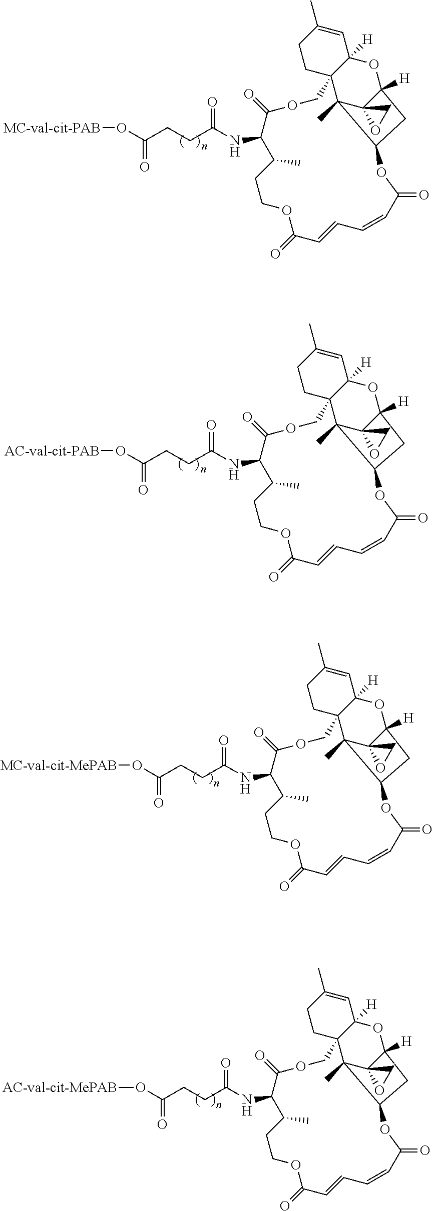

wherein n is an integer from 1-4, or, in another embodiment, n is 1, 2 or 3.

In one embodiment, the linker-verrucarin A derivative for use in the compositions and methods provided herein is selected from:

| Compound | Structure |
|---|---|
| 10a (n = 1) | 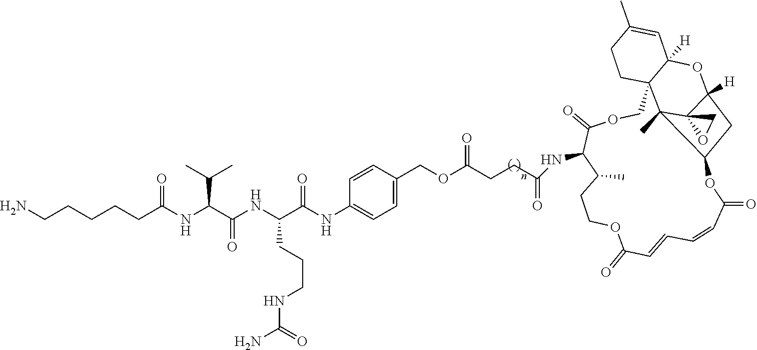 [SMILES: CC1=C[C@H]2O[C@@H]3C[C@H]4OC(=O)/C=C\C=C\C(=O)OCC[C@@H](C)[C@@H](NC(=O)CCC(=O)OCc5ccc(NC(=O)[C@H](CCCNC(N)=O)NC(=O)[C@@H](NC(=O)CCCCCN)C(C)C)cc5)C(=O)OC[C@@]2(CC1)[C@]4(C)[C@]31CO1] |
| 10b (n = 2) | 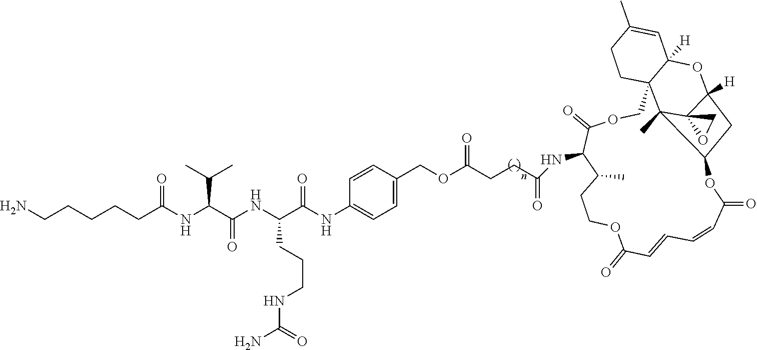 [SMILES: CC1=C[C@H]2O[C@@H]3C[C@H]4OC(=O)/C=C\C=C\C(=O)OCC[C@@H](C)[C@@H](NC(=O)CCC(=O)OCc5ccc(NC(=O)[C@H](CCCNC(N)=O)NC(=O)[C@@H](NC(=O)CCCCCN)C(C)C)cc5)C(=O)OC[C@@]2(CC1)[C@]4(C)[C@]31CO1] |
| 10c (n = 3) | 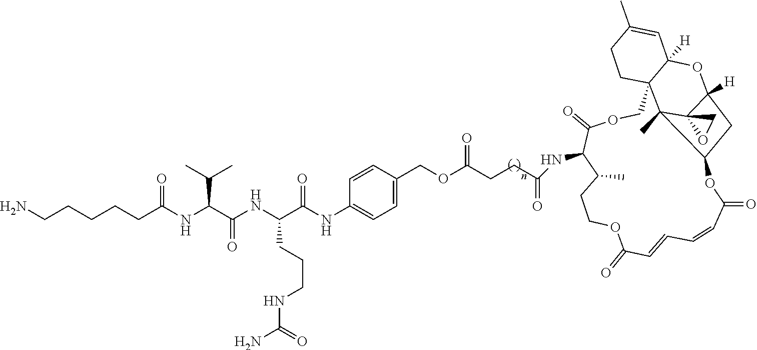 [SMILES: CC1=C[C@H]2O[C@@H]3C[C@H]4OC(=O)/C=C\C=C\C(=O)OCC[C@@H](C)[C@@H](NC(=O)CCC(=O)OCc5ccc(NC(=O)[C@H](CCCNC(N)=O)NC(=O)[C@@H](NC(=O)CCCCCN)C(C)C)cc5)C(=O)OC[C@@]2(CC1)[C@]4(C)[C@]31CO1] |
| 12a (n = 1) | 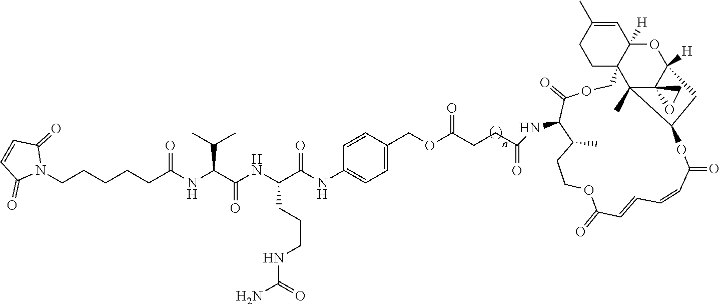 [SMILES: CC1=C[C@H]2O[C@@H]3C[C@H]4OC(=O)/C=C\C=C\C(=O)OCC[C@@H](C)[C@@H](NC(=O)CCC(=O)OCc5ccc(NC(=O)[C@H](CCCNC(N)=O)NC(=O)[C@@H](NC(=O)CCCCCN6C(=O)C=CC6=O)C(C)C)cc5)C(=O)OC[C@@]2(CC1)[C@]4(C)[C@]31CO1] |
| 12b (n = 2) | 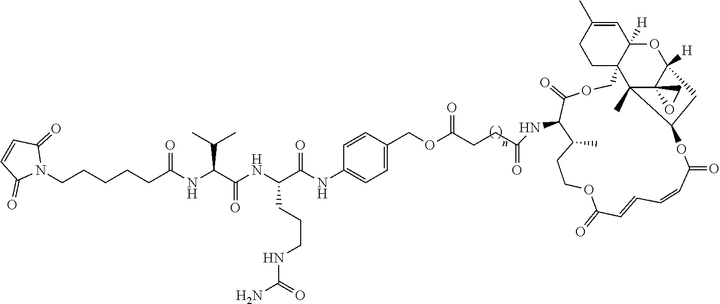 [SMILES: CC1=C[C@H]2O[C@@H]3C[C@H]4OC(=O)/C=C\C=C\C(=O)OCC[C@@H](C)[C@@H](NC(=O)CCC(=O)OCc5ccc(NC(=O)[C@H](CCCNC(N)=O)NC(=O)[C@@H](NC(=O)CCCCCN6C(=O)C=CC6=O)C(C)C)cc5)C(=O)OC[C@@]2(CC1)[C@]4(C)[C@]31CO1] |
| 12c (n = 3) | 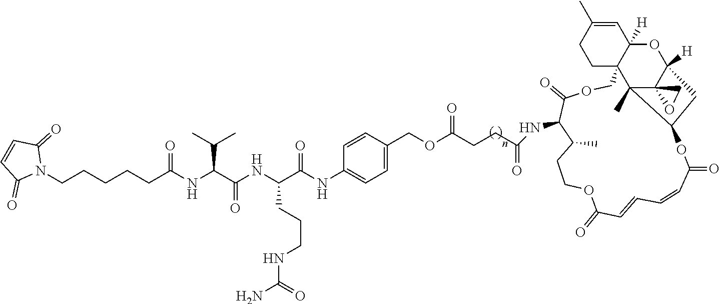 [SMILES: CC1=C[C@H]2O[C@@H]3C[C@H]4OC(=O)/C=C\C=C\C(=O)OCC[C@@H](C)[C@@H](NC(=O)CCC(=O)OCc5ccc(NC(=O)[C@H](CCCNC(N)=O)NC(=O)[C@@H](NC(=O)CCCCCN6C(=O)C=CC6=O)C(C)C)cc5)C(=O)OC[C@@]2(CC1)[C@]4(C)[C@]31CO1] |
| 19a (n = 1) | 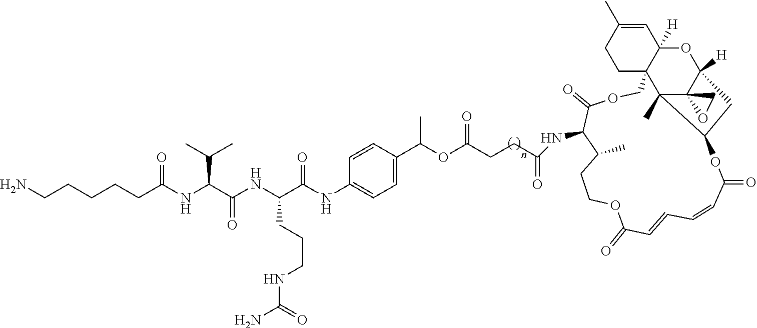 [SMILES: CC1=C[C@H]2O[C@@H]3C[C@H]4OC(=O)/C=C\C=C\C(=O)OCC[C@@H](C)[C@@H](NC(=O)CCC(=O)OC(C)c5ccc(NC(=O)[C@H](CCCNC(N)=O)NC(=O)[C@@H](NC(=O)CCCCCN)C(C)C)cc5)C(=O)OC[C@@]2(CC1)[C@]4(C)[C@]31CO1] |
| 19b (n = 2) | 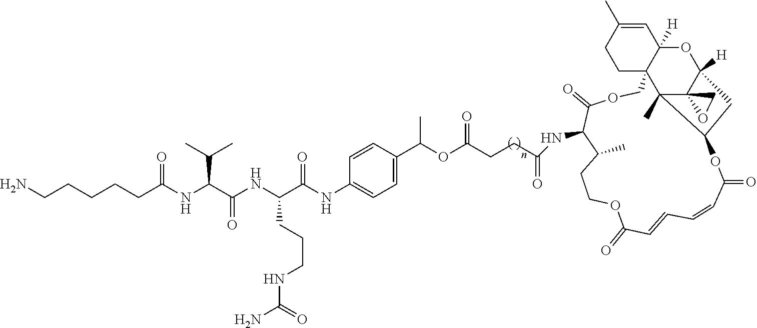 [SMILES: CC1=C[C@H]2O[C@@H]3C[C@H]4OC(=O)/C=C\C=C\C(=O)OCC[C@@H](C)[C@@H](NC(=O)CCC(=O)OC(C)c5ccc(NC(=O)[C@H](CCCNC(N)=O)NC(=O)[C@@H](NC(=O)CCCCCN)C(C)C)cc5)C(=O)OC[C@@]2(CC1)[C@]4(C)[C@]31CO1] |
| 19c (n = 3) | 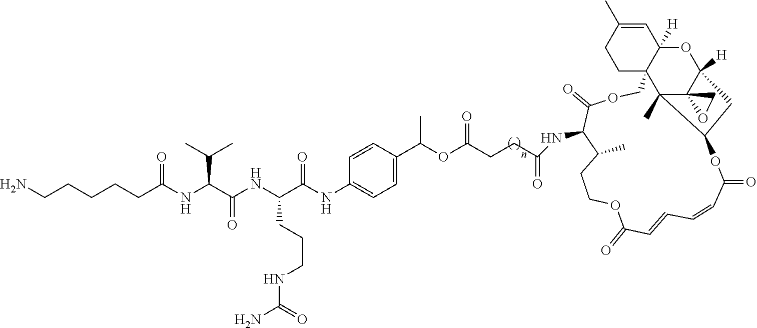 [SMILES: CC1=C[C@H]2O[C@@H]3C[C@H]4OC(=O)/C=C\C=C\C(=O)OCC[C@@H](C)[C@@H](NC(=O)CCC(=O)OC(C)c5ccc(NC(=O)[C@H](CCCNC(N)=O)NC(=O)[C@@H](NC(=O)CCCCCN)C(C)C)cc5)C(=O)OC[C@@]2(CC1)[C@]4(C)[C@]31CO1] |
| 21a (n = 1) | 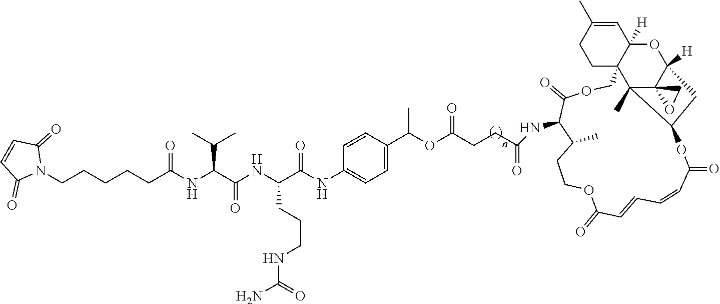 [SMILES: CC1=C[C@H]2O[C@@H]3C[C@H]4OC(=O)/C=C\C=C\C(=O)OCC[C@@H](C)[C@@H](NC(=O)CCC(=O)OC(C)c5ccc(NC(=O)[C@H](CCCNC(N)=O)NC(=O)[C@@H](NC(=O)CCCCCN6C(=O)C=CC6=O)C(C)C)cc5)C(=O)OC[C@@]2(CC1)[C@]4(C)[C@]31CO1] |
| 21b (n = 2) | 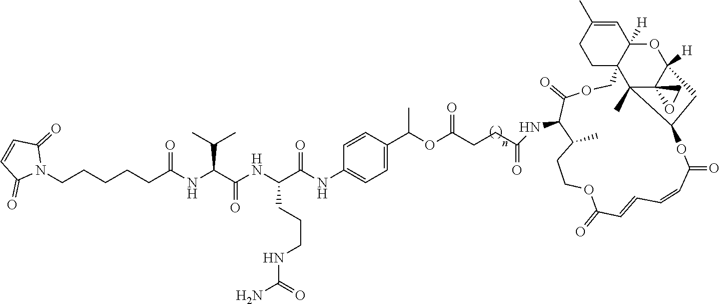 [SMILES: CC1=C[C@H]2O[C@@H]3C[C@H]4OC(=O)/C=C\C=C\C(=O)OCC[C@@H](C)[C@@H](NC(=O)CCC(=O)OC(C)c5ccc(NC(=O)[C@H](CCCNC(N)=O)NC(=O)[C@@H](NC(=O)CCCCCN6C(=O)C=CC6=O)C(C)C)cc5)C(=O)OC[C@@]2(CC1)[C@]4(C)[C@]31CO1] |
| 21c (n = 3) | 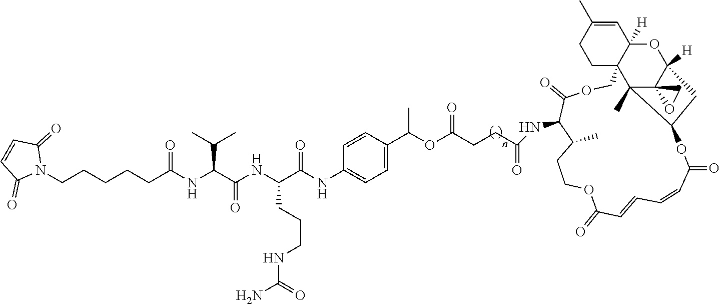 [SMILES: CC1=C[C@H]2O[C@@H]3C[C@H]4OC(=O)/C=C\C=C\C(=O)OCC[C@@H](C)[C@@H](NC(=O)CCC(=O)OC(C)c5ccc(NC(=O)[C@H](CCCNC(N)=O)NC(=O)[C@@H](NC(=O)CCCCCN6C(=O)C=CC6=O)C(C)C)cc5)C(=O)OC[C@@]2(CC1)[C@]4(C)[C@]31CO1] |
| 37 | 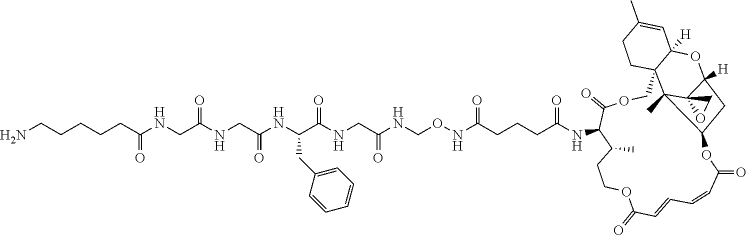 [SMILES: CC1=C[C@H]2O[C@@H]3C[C@H]4OC(=O)/C=C\C=C\C(=O)OCC[C@@H](C)[C@@H](NC(=O)CCCC(=O)NOCNC(=O)CNC(=O)[C@H](Cc5ccccc5)NC(=O)CNC(=O)CNC(=O)CCCCCN)C(=O)OC[C@@]2(CC1)[C@]4(C)[C@]31CO1] |
| 46 | 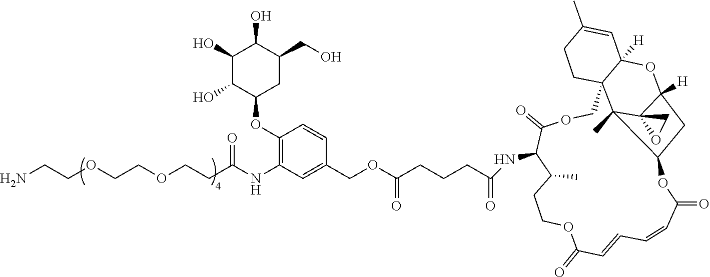 |
| 53 | 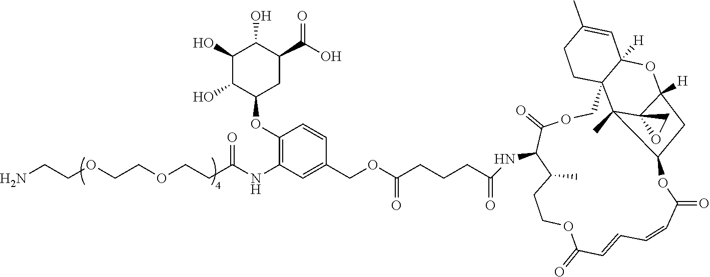 |
| 70a | 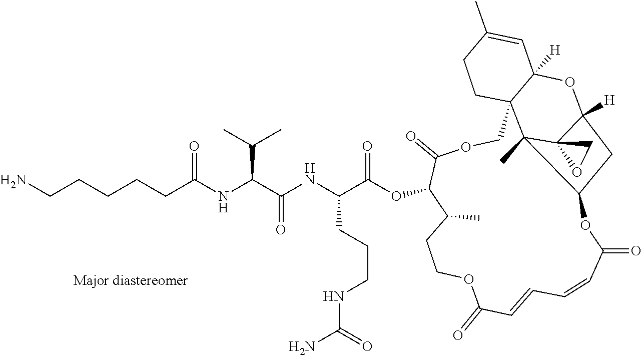 [SMILES: CC1=C[C@H]2O[C@@H]3C[C@H]4OC(=O)/C=C\C=C\C(=O)OCC[C@@H](C)[C@H](OC(=O)[C@H](CCCNC(N)=O)NC(=O)[C@@H](NC(=O)CCCCCN)C(C)C)C(=O)OC[C@@]2(CC1)[C@]4(C)[C@]31CO1] |
| 70b | 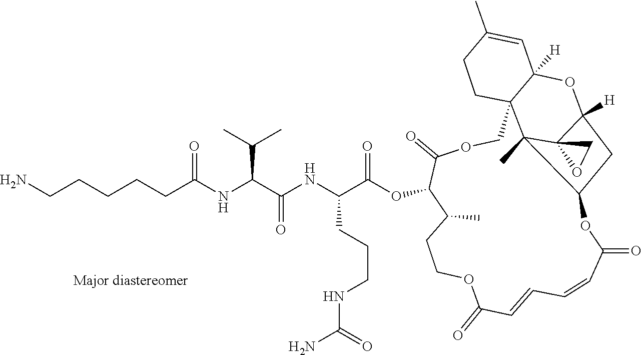 [SMILES: CC1=C[C@H]2O[C@@H]3C[C@H]4OC(=O)/C=C\C=C\C(=O)OCC[C@@H](C)[C@H](OC(=O)[C@H](CCCNC(N)=O)NC(=O)[C@@H](NC(=O)CCCCCN)C(C)C)C(=O)OC[C@@]2(CC1)[C@]4(C)[C@]31CO1] |
| 74 | 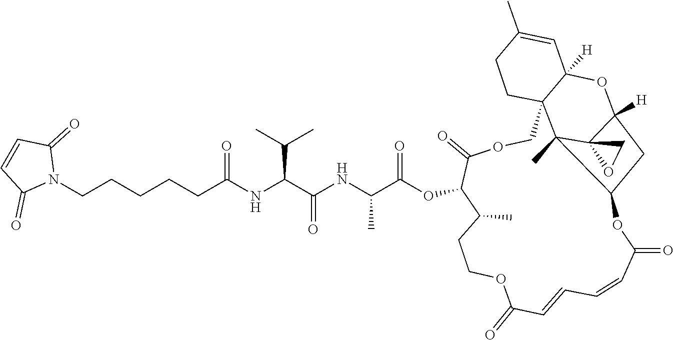 [SMILES: CC1=C[C@H]2O[C@@H]3C[C@H]4OC(=O)/C=C\C=C\C(=O)OCC[C@@H](C)[C@H](OC(=O)[C@H](C)NC(=O)[C@@H](NC(=O)CCCCCN5C(=O)C=CC5=O)C(C)C)C(=O)OC[C@@]2(CC1)[C@]4(C)[C@]31CO1] |
| 75 | 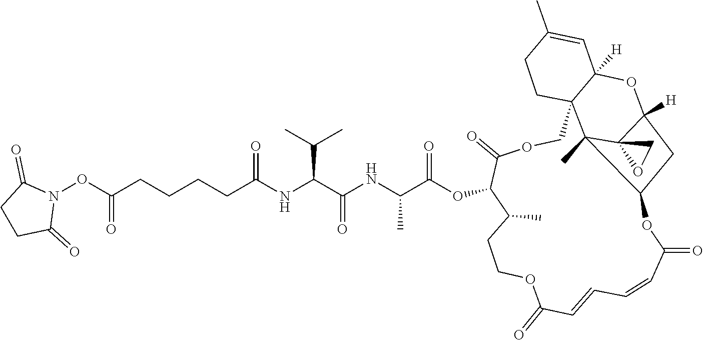 [SMILES: CC1=C[C@H]2O[C@@H]3C[C@H]4OC(=O)/C=C\C=C\C(=O)OCC[C@@H](C)[C@H](OC(=O)[C@H](C)NC(=O)[C@@H](NC(=O)CCCCC(=O)ON5C(=O)CCC5=O)C(C)C)C(=O)OC[C@@]2(CC1)[C@]4(C)[C@]31CO1] |
| 76 | 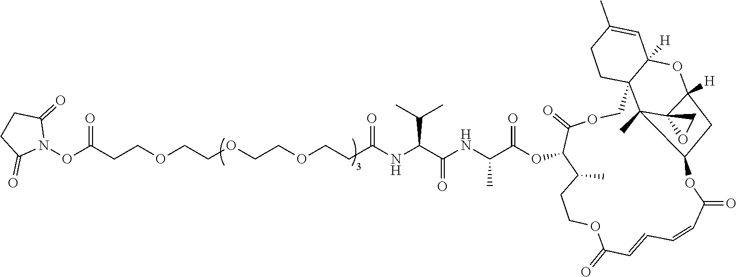 |
| 88 | 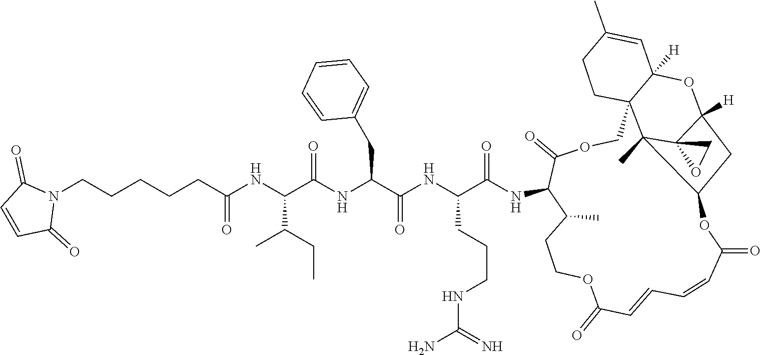 [SMILES: CCC(C)[C@H](NC(=O)CCCCCN1C(=O)C=CC1=O)C(=O)N[C@@H](Cc1ccccc1)C(=O)N[C@@H](CCCNC(=N)N)C(=O)N[C@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1] |
| 91 | 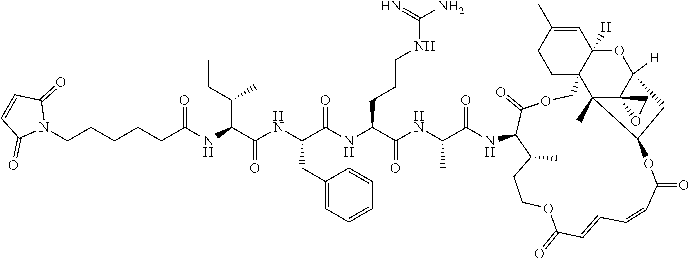 [SMILES: CC[C@H](C)[C@H](NC(=O)CCCCCN1C(=O)C=CC1=O)C(=O)N[C@@H](Cc1ccccc1)C(=O)N[C@@H](CCCNC(=N)N)C(=O)N[C@@H](C)C(=O)N[C@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1] |
| 97 | 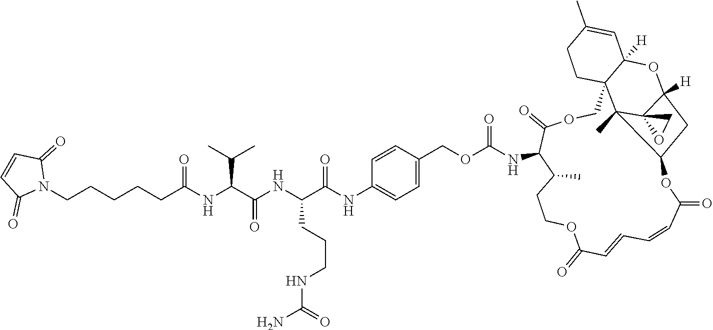 [SMILES: CC1=C[C@H]2O[C@@H]3C[C@H]4OC(=O)/C=C\C=C\C(=O)OCC[C@@H](C)[C@@H](NC(=O)OCc5ccc(NC(=O)[C@H](CCCNC(N)=O)NC(=O)[C@@H](NC(=O)CCCCCN6C(=O)C=CC6=O)C(C)C)cc5)C(=O)OC[C@@]2(CC1)[C@]4(C)[C@]31CO1] |
| 95 | 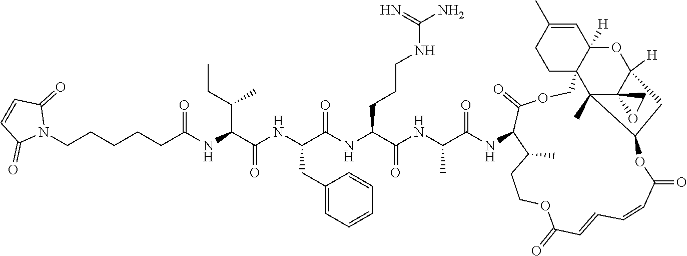 [SMILES: CC[C@H](C)[C@H](NC(=O)CCCCCN1C(=O)C=CC1=O)C(=O)N[C@@H](Cc1ccccc1)C(=O)N[C@@H](CCCNC(=N)N)C(=O)N[C@@H](C)C(=O)N[C@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1] |
| 101 | 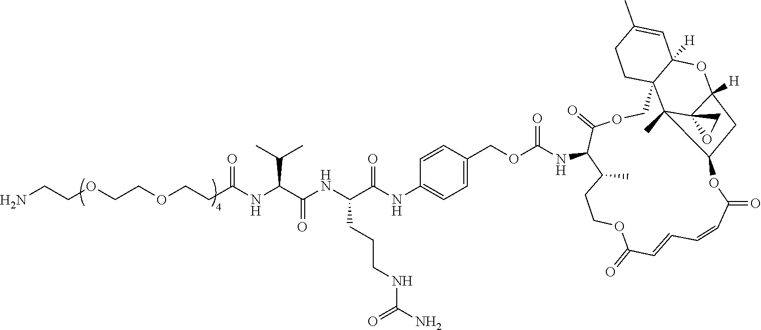 |
| 106 | 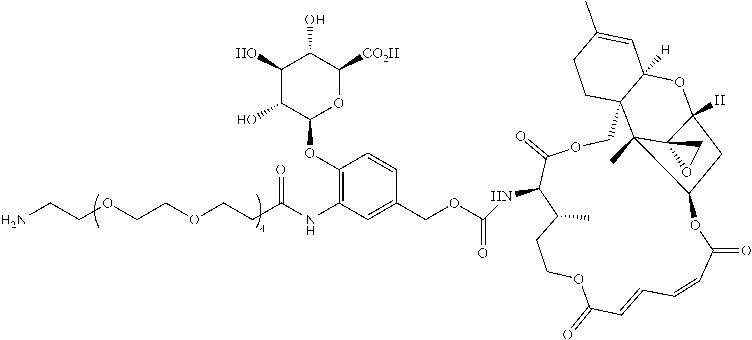 |
| 109 | 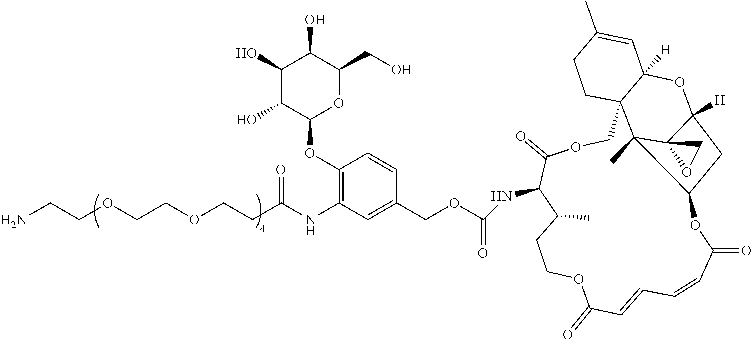 |
| 114 | 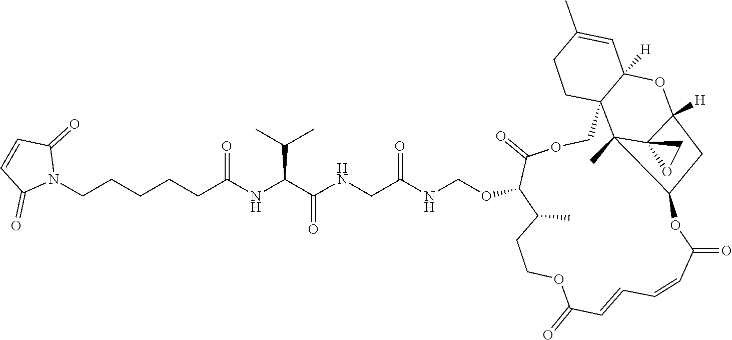 [SMILES: CC1=C[C@H]2O[C@@H]3C[C@H]4OC(=O)/C=C\C=C\C(=O)OCC[C@@H](C)[C@H](OCNC(=O)CNC(=O)[C@@H](NC(=O)CCCCCN5C(=O)C=CC5=O)C(C)C)C(=O)OC[C@@]2(CC1)[C@]4(C)[C@]31CO1] |
| 118 | 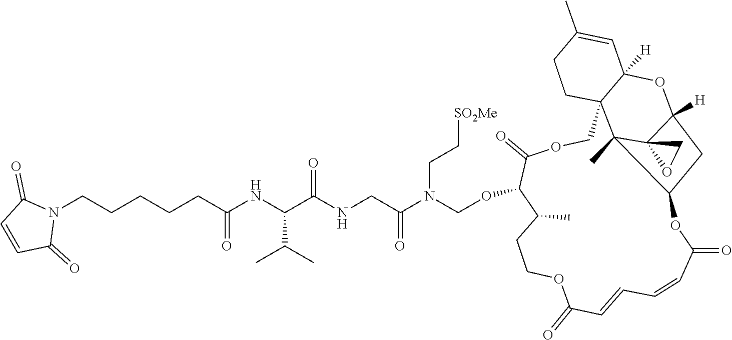 [SMILES: CC1=C[C@H]2O[C@@H]3C[C@H]4OC(=O)/C=C\C=C\C(=O)OCC[C@@H](C)[C@H](OCN(CCS(C)(=O)=O)C(=O)CNC(=O)[C@@H](NC(=O)CCCCCN5C(=O)C=CC5=O)C(C)C)C(=O)OC[C@@]2(CC1)[C@]4(C)[C@]31CO1] |
| 121 | 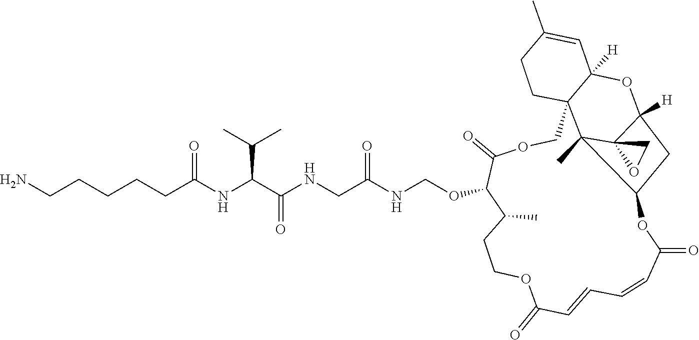 [SMILES: CC1=C[C@H]2O[C@@H]3C[C@H]4OC(=O)/C=C\C=C\C(=O)OCC[C@@H](C)[C@H](OCNC(=O)CNC(=O)[C@@H](NC(=O)CCCCCN)C(C)C)C(=O)OC[C@@]2(CC1)[C@]4(C)[C@]31CO1] |
| 124 | 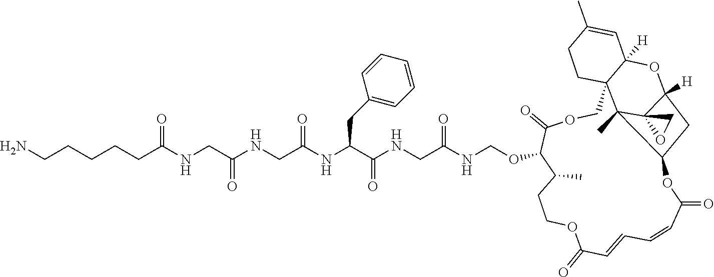 [SMILES: CC1=C[C@H]2O[C@@H]3C[C@H]4OC(=O)/C=C\C=C\C(=O)OCC[C@@H](C)[C@H](OCNC(=O)CNC(=O)[C@H](Cc5ccccc5)NC(=O)CNC(=O)CNC(=O)CCCCCN)C(=O)OC[C@@]2(CC1)[C@]4(C)[C@]31CO1] |
| 126 | 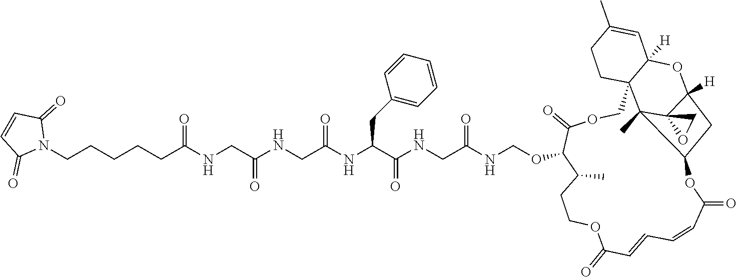 [SMILES: CC1=C[C@H]2O[C@@H]3C[C@H]4OC(=O)/C=C\C=C\C(=O)OCC[C@@H](C)[C@H](OCNC(=O)CNC(=O)[C@H](Cc5ccccc5)NC(=O)CNC(=O)CNC(=O)CCCCCN5C(=O)C=CC5=O)C(=O)OC[C@@]2(CC1)[C@]4(C)[C@]31CO1] |

In one embodiment, the linker-verrucarin A derivative for use in the compositions and methods provided herein is selected from:

| Compound | Structure |
|---|---|
| 10a (n = 1) | 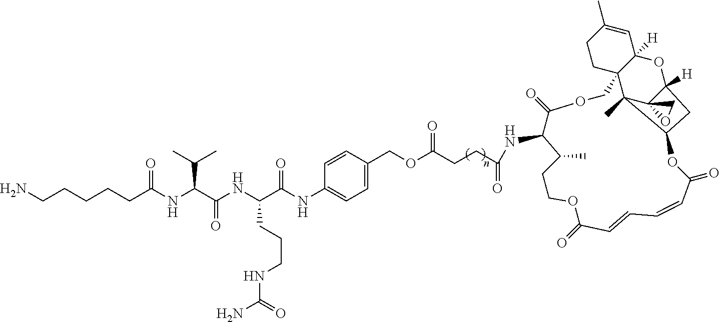 |
| 10b (n = 2) | 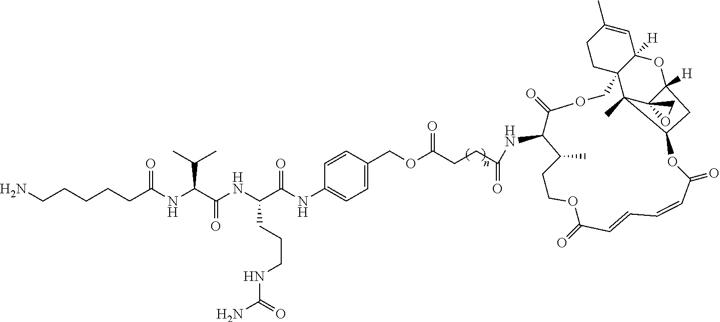 |
| 10c (n = 3) | 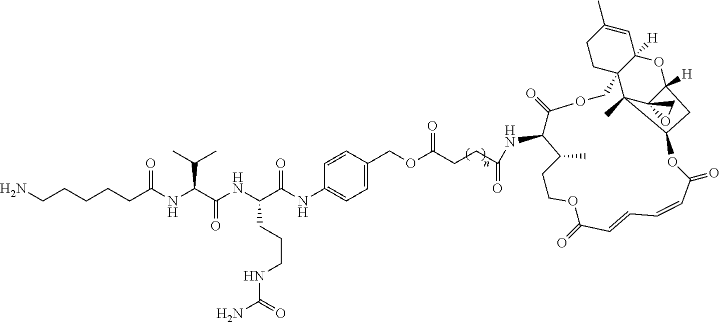 |
| 12a (n = 1) | 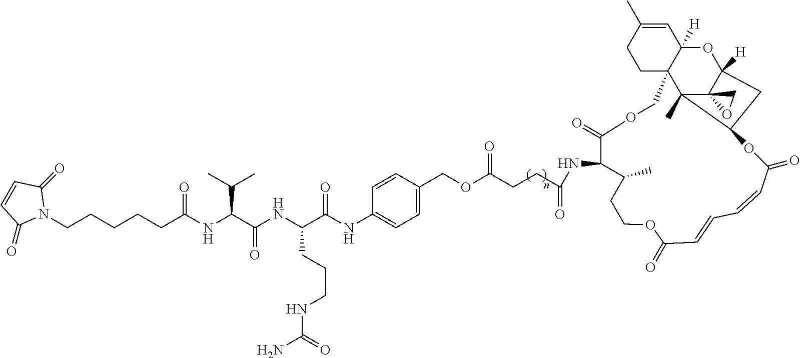 |
| 12b (n = 2) | 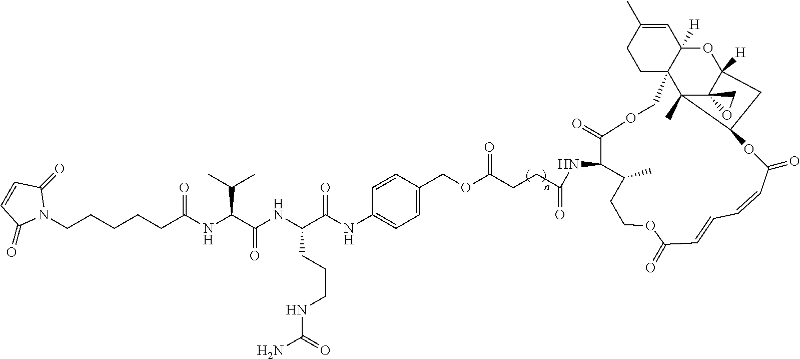 |
| 12c (n = 3) | 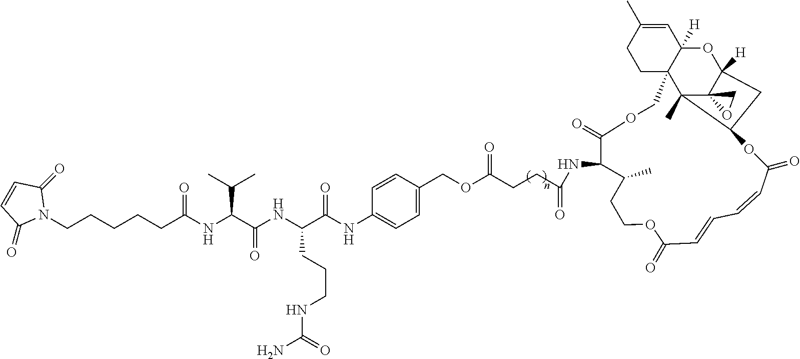 |
| 19a (n = 1) | 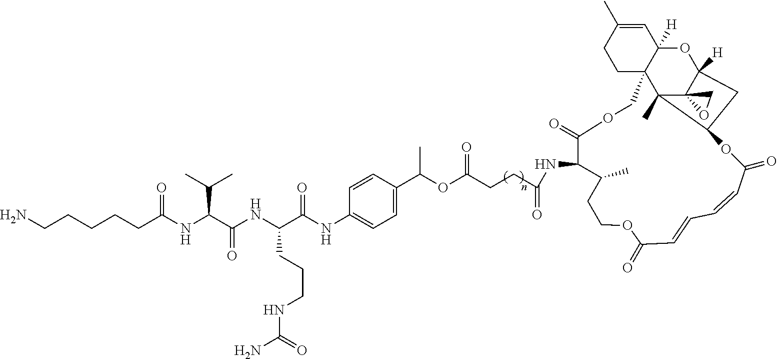 |
| 19b (n = 2) | 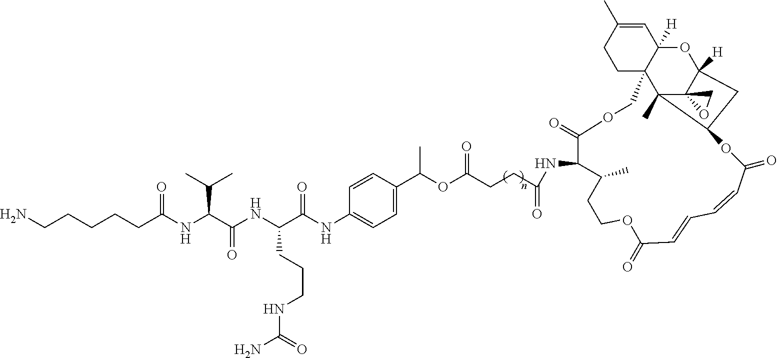 |
| 19c (n = 3) | 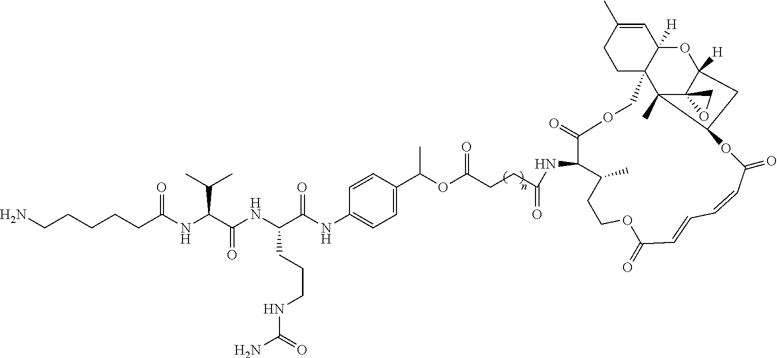 |
| 21a (n = 1) | 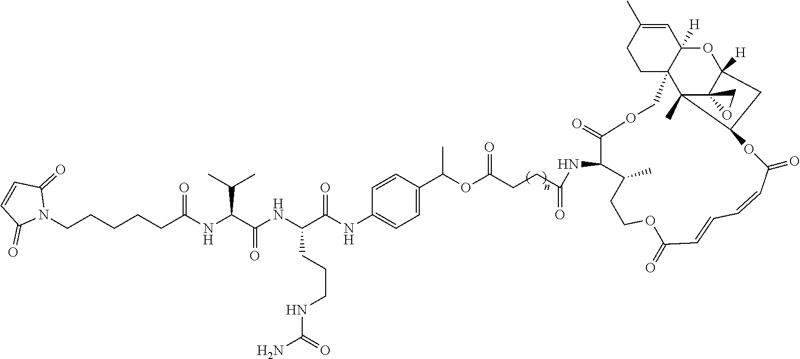 |
| 21b (n = 2) | 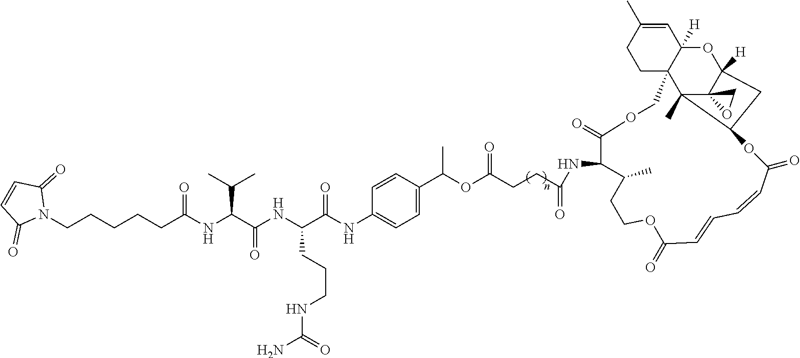 |
| 21c (n = 3) | 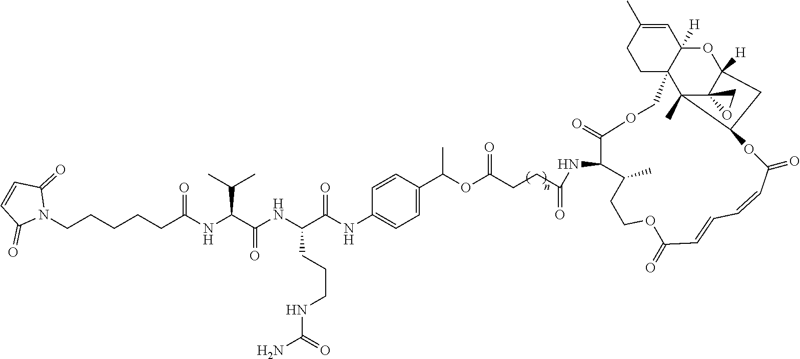 |

### V. Synthesis of Linker-Verrucarin A Derivatives

The linker-verrucarin A derivatives provided herein may be prepared according to methods well known to those of skill in the art. For example, the terminal carboxylic acid in the verrucarin A derivatives provided herein may then be esterified under standard conditions, e.g., with a p-aminobenzyl alcohol derivative, to provide verrucarin A-linker compounds, e.g., compounds 10a-c, 12a-c, 19a-c and 21a-c.

### VI. ADCs for Use in Compositions and Methods

In one embodiment, provided herein are ADCs for use in the compositions and methods provided herein having Formula V:

Z-(L-X)ᵥ V

or a pharmaceutically acceptable derivative thereof, wherein:

Z is an anti-influenza antigen-binding domain, an anti-SARS-CoV-2 antigen-binding domain, or an anti-ebola antigen-binding domain;

L is a linking group as defined herein;

X is a verrucarin A derivative; and

v is an integer from 1 to 12.

In one embodiment, X is a verrucarin A derivative as described herein. In another embodiment, v is an integer from 1 to 10, from 1 to 8, from 1 to 6 or from 1 to 4. In another embodiment, v is 1, 2, 3, 4, 5, 6, 7, 8, 9 or 10. In another embodiment, v is 1, 2, 3, 4, 5, 6, 7 or 8. In another embodiment, v is about 2 or is 2.

A. Antigen-Binding Domains Z

Suitable antigen-binding domains Z for any of the ADCs provided herein include, but are not limited to, antibodies, viral receptors, or any other cell binding or peptide binding molecules or substances. In other embodiments, antigen-binding domains that can be used in the ADCs provided herein include antibodies, antigen-binding fragments of antibodies, peptides that specifically interact with a particular antigen (e.g., peptibodies), receptor molecules that specifically interact with a particular antigen, proteins comprising a ligand-binding portion of a receptor that specifically binds a particular antigen, antigen-binding scaffolds (e.g., DARPins, HEAT repeat proteins, ARM repeat proteins, tetratricopeptide repeat proteins, and other scaffolds based on naturally occurring repeat proteins, etc., (see, e.g., Boersma and Pluckthun, 2011, Curr. Opin. Biotechnol. 22:849-857, and references cited therein)), and aptamers or portions thereof. The full-length amino acid sequence of an exemplary influenza hemagglutinin (HA) is shown in GenBank as accession number ACP44150.1.

In one embodiment, antigen-binding domains Z include antibodies (e.g., fully human antibodies) and antigen-binding fragments thereof that specifically bind to influenza virus proteins, such as the surface proteins hemagglutinin (HA), neuraminidase (NA), and Matrix-2 (M2). In some embodiments, these antigen-binding domains Z modulate the interaction of influenza virus with host cells. In some embodiments, the antibodies or antigen-binding fragments thereof bind to mature hemagglutinin. In some embodiments, the antibodies or antigen-binding fragments thereof bind to an HA0 hemagglutinin precursor protein. The anti-influenza HA antibodies may bind to the influenza virus HA with high affinity. In certain embodiments, the antibodies herein are blocking antibodies wherein the antibodies may bind to influenza HA and block the attachment to and/or entry of the virus into host cells. In some embodiments, the blocking antibodies herein may block the binding of influenza virus to cells and as such may inhibit or neutralize viral infectivity of host cells. In some embodiments, the blocking antibodies may be useful for treating a subject suffering from an influenza virus infection. The antibodies when administered to a subject in need thereof may reduce the infection by a virus such as influenza in the subject. The antibodies may be used to decrease viral loads in a subject. The antibodies may be used alone or as adjunct therapy with other therapeutic moieties or modalities known in the art for treating a viral infection. In certain embodiments, the antibodies may bind to an epitope in the stem region of the viral HA, the head region of the viral HA, or both. Furthermore, the antibodies can be used prophylactically (before infection) to protect a mammal from infection, or can be used therapeutically (after infection is established) to ameliorate a previously established infection, or can be used to ameliorate at least one symptom associated with the infection.

In one embodiment, antigen-binding domains Z include antibodies (e.g., fully human antibodies) and antigen-binding fragments thereof that specifically bind to SARS-CoV-2 proteins, such as the spike glycoprotein (which may also be referred to as the spike protein, or as SARS-CoV-2-S). In some embodiments, these antigen-binding domains Z modulate the interaction of SARS-CoV-2 with host cells. In some embodiments, the antibodies or antigen-binding fragments thereof bind to mature spike glycoprotein. In some embodiments, the antibodies or antigen-binding fragments thereof bind to a spike precursor protein. The anti-SARS-CoV-2-S antibodies may bind to the spike glycoprotein with high affinity. In certain embodiments, the antibodies herein are blocking antibodies wherein the antibodies may bind to SARS-CoV-2-S and block the attachment to and/or entry of the virus into host cells, for example by blocking the interaction between the spike glycoprotein and its receptor ACE2. In some embodiments, the blocking antibodies herein may block the binding of SARS-CoV-2 to cells and as such may inhibit or neutralize viral infectivity of host cells. In some embodiments, the blocking antibodies may be useful for treating a subject suffering from an SARS-CoV-2 infection and/or experiencing COVID-19 symptoms. The antibodies when administered to a subject in need thereof may reduce the infection by a virus such as SARS-CoV-2 in the subject. The antibodies may be used to decrease viral loads in a subject. The antibodies may be used alone or as adjunct therapy with other therapeutic moieties or modalities known in the art for treating a viral infection. In certain embodiments, the antibodies may bind to an epitope in the receptor-binding domain of the spike glycoprotein. Furthermore, the antibodies can be used prophylactically (before infection) to protect a mammal from infection, or can be used therapeutically (after infection is established) to ameliorate a previously established infection, or can be used to ameliorate at least one symptom associated with the infection.

In certain embodiments, the antibodies are obtained from mice immunized with a primary immunogen, such as a full-length influenza HA, SARS-CoV-2-S or ebola virus GP, or with a recombinant form of influenza HA, SARS-CoV-2-S or ebola virus GP, or fragments thereof followed by immunization with a secondary immunogen, or with an immunogenically active fragment of influenza HA, SARS-CoV-2-S or ebola virus GP. In certain embodiments, the antibodies are obtained from mice immunized with an influenza, SARS-CoV-2 or ebola vaccine composition followed by booster immunization with one or more recombinantly produced influenza HA, SARS-CoV-2-S, or ebola peptides, respectively. In certain embodiments, the antibodies are obtained from humans. In certain embodiments, the antibodies are obtained from mammals (e.g., non-human mammals). In certain embodiments, the antibodies are obtained from non-human primates.

The immunogen may be a biologically active and/or immunogenic fragment of influenza HA, SARS-CoV-2-S, or ebola virus, or DNA encoding the active fragment thereof. For influenza, the fragment may be derived from the stem region of the HA protein (see, e.g., Sui et al. Nature Struct. and Mol. Biol., published online 22 Feb. 2009; pp, 1-9), the head region of the HA protein, or a combination thereof. For SARS-CoV-2, the fragment may be derived from the full-length SARS-CoV-2-S protein or from the receptor binding domain (RBD). For ebola, the fragment may be derived from full-length ebola protein or from the ebola virus GP, including the amino-terminal fragment (e.g. GP1), or the carboxy-terminal fragment (e.g. GP2).

The antigen-binding domains Z may be modified to include addition or substitution of certain residues for tagging or for purposes of conjugation to carrier molecules, such as, keyhole limpet hemocyanin (KLH). For example, a cysteine may be added at either the N-terminal or C-terminal end of a peptide, or a linker sequence may be added to prepare the peptide for conjugation to, for example, KLH for immunization.

Certain anti-influenza antibodies, anti-influenza-HA antibodies, or ADCs provided herein have antiviral activity, such as being able to bind to and neutralize the activity of influenza-HA, as determined by in vitro or in vivo assays. Certain anti-influenza antibodies, anti-influenza-HA antibodies, or ADCs provided herein are able to bind to HA but do not have neutralizing activity, as determined by in vitro or in vivo assays. The ability of the antibodies or ADCs herein to bind to and neutralize the activity of influenza-HA and thus the attachment and/or entry of the virus into a host cell followed by the ensuing viral infection, may be measured using any standard method known to those skilled in the art, including binding assays, or activity assays, as described herein.

Certain anti-SARS-CoV-2 antibodies, anti-SARS-CoV-2-S antibodies, or ADCs provided herein have antiviral activity, such as being able to bind to and neutralize the activity of SARS-CoV-2-S, as determined by in vitro or in vivo assays. Certain anti-SARS-CoV-2 antibodies, anti-SARS-CoV-2-S antibodies, or ADCs provided herein are able to bind to SARS-CoV-2-S but do not have neutralizing activity, as determined by in vitro or in vivo assays. The ability of the antibodies or ADCs herein to bind to and neutralize the activity of SARS-CoV-2-S and thus the attachment and/or entry of the virus into a host cell followed by the ensuing viral infection, may be measured using any standard method known to those skilled in the art, including binding assays, or activity assays, as described herein.

Certain anti-ebola antibodies or ADCs provided herein have antiviral activity, such as being able to bind to and neutralize the activity of ebola virus, as determined by in vitro or in vivo assays. Certain anti-ebola antibodies or ADCs provided herein are able to bind to ebola but do not have neutralizing activity, as determined by in vitro or in vivo assays. The ability of the antibodies or ADCs herein to bind to and neutralize the activity of ebola virus and thus the attachment and/or entry of the virus into a host cell followed by the ensuing viral infection, may be measured using any standard method known to those skilled in the art, including binding assays, or activity assays, as described herein.

The antigen-binding domains Z, such as antibodies, or ADCs specific for influenza-HA, SARS-CoV-2-S or ebola virus may contain no additional labels or moieties, or they may contain an N-terminal or C-terminal label or moiety. In one embodiment, the label or moiety is biotin. In a binding assay, the location of a label (if any) may determine the orientation of the peptide relative to the surface upon which the peptide is bound. For example, if a surface is coated with avidin, a peptide containing an N-terminal biotin will be oriented such that the C-terminal portion of the peptide will be distal to the surface. In one embodiment, the label may be a radionuclide, a fluorescent dye, or an MRI-detectable label. In certain embodiments, such labeled antibodies may be used in diagnostic assays including imaging assays. In one embodiment, the additional moiety is a peptide tag. In one embodiment, an ADC includes an antibody heavy chain and further includes a peptide tag at the C-terminus of the antibody heavy chain. In one embodiment, an ADC includes an antibody heavy chain and further includes a peptide tag at the C-terminus of the antibody heavy chain, wherein the peptide tag is ELQRP, LLQG, LLQGG, LLQLLQG, LLQYQG, LLQGA, LLQGSG, SLLQG, LQG, LLQLQ, LLQLLQ, LLQGR, LLQYQGA, LQGG, LGQG or LLQLLQGA. See, e.g., WO 2012/059882, U.S. Pat. No. 9,676,871 and U.S. Patent Application Publication No. US 2003/0138785. In one embodiment, the ADCs provided herein include an antibody heavy chain and further includes a peptide tag (e.g., a pentapeptide) at the C-terminus of the antibody heavy chain, wherein the peptide tag is the pentapeptide sequence LLQGA (e.g., for use in conjugating a linker-payload via transglutaminase). In one embodiment, the ADCs provided herein include an antibody heavy chain and further includes a peptide tag (e.g., a pentapeptide) at the C-terminus of the antibody heavy chain, wherein the peptide tag is the pentapeptide sequence ELQGP (e.g., for use in conjugating a linker-payload via transglutaminase). In one embodiment, an ADC includes two antibody heavy chains and further includes a peptide tag at the C-terminus of each antibody heavy chain. In one embodiment, an ADC includes two antibody heavy chains and further includes a peptide tag at the C-terminus of each antibody heavy chain, wherein the peptide tag is the pentapeptide sequence LLQGA. In one embodiment, an ADC includes two antibody heavy chains and further includes a peptide tag at the C-terminus of each antibody heavy chain, wherein the peptide tag is the pentapeptide sequence ELQGP.

In certain embodiments, the antibody comprises a light chain. In certain embodiments, the light chain is a kappa light chain. In certain embodiments, the light chain is a lambda light chain. In certain embodiments, the antibody comprises a heavy chain. In some embodiments, the heavy chain is an IgA. In some embodiments, the heavy chain is an IgD. In some embodiments, the heavy chain is an IgE. In some embodiments, the heavy chain is an IgG. In some embodiments, the heavy chain is an IgM. In some embodiments, the heavy chain is an IgGY. In some embodiments, the heavy chain is any class, e.g., IgG1, IgG2, IgG3, IgG4, IgA1, IgA2, or subclass. In some embodiments, the heavy chain is an IgG1. In some embodiments, the heavy chain is an IgG2. In some embodiments, the heavy chain is an IgG3. In some embodiments, the heavy chain is an IgG4. In some embodiments, the heavy chain is an IgA1. In some embodiments, the heavy chain is an IgA2. In some embodiments, Z has a molecular weight of at least 500, 600, 700, 800, 900, 1000, 10000, 50000 or 100000 Daltons.

In some embodiments, the antibody is an antibody fragment. Non-limiting examples of antigen-binding fragments for use in the ADCs provided herein include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. In other embodiments, an antigen-binding fragment of an antibody includes other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains. In some embodiments, the antibody fragment is an Fv fragment. In some embodiments, the antibody fragment is a Fab fragment. In some embodiments, the antibody fragment is a F(ab′)₂ fragment. In some embodiments, the antibody fragment is a Fab′ fragment. In some embodiments, the antibody fragment is an scFv (sFv) fragment. In some embodiments, the antibody fragment is an scFv-Fc fragment.

In some embodiments, the antibody is a monoclonal antibody. In some embodiments, the antibody is a polyclonal antibody. In some embodiments, the antibody is a bispecific antibody including a first antigen-binding domain, and a second antigen-binding domain.

In some embodiments, the antibody is a chimeric antibody. In some embodiments, the antibody is a humanized antibody. In some embodiments, the antibody is a human antibody.

In certain embodiments the antibody can be engineered to comprise a glutamine residue. Techniques for modifying an antibody sequence to include a glutamine residue are within the skill of those in the art (see, e.g., Ausubel et al. Current Protoc. Mol. Biol.). In one embodiment, the antibody includes an antibody heavy chain and further includes a peptide tag at the C-terminus of the antibody heavy chain. In one embodiment, the antibody includes an antibody heavy chain and further includes a peptide tag, e.g., transglutaminase recognition sequence or pentapeptide tag, at the C-terminus of the antibody heavy chain, wherein the peptide tag is the pentapeptide sequence LLQGA or ELQGP.

B. Preparation of Human Antibodies

Methods for generating human antibodies in transgenic mice are known in the art. Any such known methods can be used in the context of this disclosure to make human antibodies that specifically bind to influenza-HA, SARS-CoV-2-S or ebola virus. An immunogen comprising any one of the following can be used to generate antibodies to influenza-HA, SARS-CoV-2-S or ebola virus. In certain embodiments, the antibodies herein are obtained from mice immunized with a full length, native influenza-HA (See, e.g., GenBank accession number FJ966082.1), with full length SARS-CoV-2-S, or with the SARS-CoV-2-S receptor binding domain, or with a live attenuated or inactivated virus, or with DNA encoding the protein or fragment thereof. Alternatively, the influenza-HA, SARS-CoV-2-S or ebola protein or a fragment thereof may be produced using standard biochemical techniques and modified and used as immunogen. In one embodiment, the immunogen is a recombinantly produced influenza-HA protein or SARS-CoV-2-S, or a fragment thereof. In certain embodiments herein, the immunogen may be an influenza virus vaccine or a SARS-CoV-2 vaccine. In certain embodiments, one or more influenza, SARS-CoV-2 or ebola booster injections may be administered. In certain embodiments, the influenza booster injections may comprise one or more influenza virus strains, or hemagglutinins derived from these strains, e.g., see Protein Sciences H1 A/New Caledonia/20/1999, H5 A/Indonesia/05/2005, H3 A/Victoria/361/2011, H7 A/Netherlands/219/2003, or H9 A/Hong Kong/1073/1988, or the influenza B virus strains B/Victoria/2/87, B/Nanchang/3451/93, B/Singapore/11/1994, B/Florida/4/2006, or BNamagata/16/88. In certain embodiments, the booster injections may contain a 1:1 mixture of the influenza strains, or a 1:1 mixture of the hemagglutinins derived from the strains. In certain embodiments, the immunogen may be a recombinant influenza-HA peptide expressed in E. coli or in any other eukaryotic or mammalian cells such as Chinese hamster ovary (CHO) cells or influenza virus itself.

Using VELOCIMMUNE® technology (see, e.g., U.S. Pat. No. 6,596,541) or any other known method for generating monoclonal antibodies, high affinity chimeric antibodies to influenza-HA, SARS-CoV-2-S or ebola are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. As described in WO 2016/100807 or US 2016/0176953 A1, each of which are incorporated by reference in their entirety, the antibodies are characterized and selected for desirable characteristics, including affinity, selectivity, epitope, etc. The mouse constant regions are replaced with a desired human constant region to generate the fully human antibody herein, for example, wild-type or modified IgG1 or IgG4. While the constant region selected may vary according to specific use, high affinity antigen-binding and target specificity characteristics reside in the variable region.

C. Bioequivalents

The antigen-binding domains Z, including anti-influenza-HA antibodies and antibody fragments, anti-SARS-CoV-2-S antibodies and antibody fragments, and anti-ebola antibodies and antibody fragments, provided for use in the ADCs herein encompass proteins having amino acid sequences that vary from those of the described antibodies, but that retain the ability to bind Influenza-HA, SARS-CoV-2-S, or ebola, respectively. Such variant antibodies and antibody fragments comprise one or more additions, deletions, or substitutions of amino acids when compared to parent sequence, but exhibit biological activity that is essentially equivalent to that of the described antibodies. Likewise, the antibody-encoding DNA sequences of the present disclosure encompass sequences that comprise one or more additions, deletions, or substitutions of nucleotides when compared to the disclosed sequence, but that encode an antibody or antibody fragment that is essentially bioequivalent to an antibody or antibody fragment herein. Other bioequivalent anti-influenza-HA antibodies and antibody fragments are as described in WO 2016/100807 or US 2016/0176953 A1, each of which are incorporated by reference in their entirety. Other bioequivalent anti-SARS-CoV-2-S antibodies and antibody fragments are described in, e.g., U.S. Pat. No. 10,787,501. Other bioequivalent anti-ebola antibodies and antibody fragments are described in, e.g., U.S. Pat. Nos. 11,530,255 and 9,771,414.

D. Biological Characteristics of the Antibodies

In general, the antigen-binding domains Z, including antibodies, provided herein function by binding to influenza-HA, SARS-CoV-2-S, or ebola virus. For example, provided herein are antibodies and antigen-binding fragments of antibodies that bind influenza-HA, SARS-CoV-2-S or ebola virus (e.g., at 25° C. or at 37° C.) with a K_{D} of less than 10 nM, as measured by real-time bio-layer interferometer-based biosensor (Octet HTX assay), or by surface plasmon resonance. In certain embodiments, the antibodies or antigen-binding fragments thereof bind influenza-HA, SARS-CoV-2-S or ebola with a K_{D} of less than about 5 nM, less than about 2 nM, less than about 1 nM, less than about 500 pM, less than 250 pM, or less than 100 pM, as measured by surface plasmon resonance, e.g., using the assay format as described in WO 2016/100807 or US 2016/0176953 A1, each of which are incorporated by reference in their entirety, or a substantially similar assay.

Non-limiting, exemplary in vitro assays for measuring binding activity are illustrated in Example 3 of WO 2016/100807 or US 2016/0176953 A1, each of which is incorporated herein by reference in their entirety. In WO 2016/100807 or US 2016/0176953 A1 Example 3, the binding affinity and dissociation constants of anti-influenza-HA antibodies for influenza-HA were determined by real-time bio-layer interferometer-based biosensor (Octet HTX assay). In Examples 4 and 5 of WO 2016/100807 or US 2016/0176953 A1, neutralization assays were used to determine infectivity of diverse group 1 strains of influenza virus. In Example 6 of WO 2016/100807 or US 2016/0176953 A1, certain antibodies were shown to mediate complement dependent cytotoxicity (CDC) of virus-infected cells in vitro. Examples 7 and 10 of WO 2016/100807 or US 2016/0176953 A1 demonstrate that certain antibodies of the disclosure are capable of neutralizing an influenza A infection in vivo when administered either prophylactically or therapeutically.

In one embodiment, the antigen-binding domains Z, including antibodies and antigen-binding fragments thereof, for use in the ADCs provided herein are those that bind influenza-HA, SARS-CoV-2-S, or ebola virus with a dissociative half-life (t %) of greater than about 100 minutes as measured by surface plasmon resonance at 25° C., e.g., using an assay format as defined in WO 2016/100807 or US 2016/0176953 A1, each of which are incorporated herein by reference in their entirety, or a substantially similar assay. In certain embodiments, the antibodies or antigen-binding fragments for use in the ADCs provided herein bind influenza-HA, SARS-CoV-2-S, or ebola virus with a t % of greater than about 200 minutes, greater than about 300 minutes, greater than about 400 minutes, greater than about 500 minutes, greater than about 600 minutes, greater than about 700 minutes, greater than about 800 minutes, greater than about 900 minutes, or greater than about 1000 minutes as measured by surface plasmon resonance at 25° C., e.g., using an assay format as defined in WO 2016/100807 or US 2016/0176953 A1, each of which is incorporated herein by reference in their entirety (e.g., imAb-capture or antigen-capture format), or a substantially similar assay. In one embodiment, the antibodies and antigen-binding fragments herein bind influenza-HA, SARS-CoV-2-S or ebola virus with a dissociative half-life (t %) of greater than 300 minutes. In one embodiment, an antibody herein provides for about a 1.5 to 2-fold increase in dissociative half-life as compared to a comparator antibody designated Control I mAb, when tested in monkeys and mice.

In another embodiment, the antigen-binding domains Z, including antibodies or antigen-binding fragments thereof, for use in the ADCs provided herein are those that neutralize the infectivity of influenza virus, SARS-CoV-2 or ebola virus, for its host cells. In some embodiments, the antibodies exhibit a neutralization potency against various representative group 1 influenza viruses (H1N1 A/Puerto Rico/08/1934; H5N1 A/Vietnam/1203/2004; H1N1 A California/07/2009; H1N1 A/Wisconsin/1933; H1N1 A/Brisbane/59/1997, H9N2 A Hong Kong/33982/2009, H13N6 a/gull/Maryland/704/1977 and H16N3 A/shorebird/Delaware/172/2006) with an IC₅₀ ranging from about 1.6 nM to about 130 nM in a microneutralization assay, e.g., as shown in Examples 4 and 5 of WO 2016/100807 or US 2016/0176953 A1, each of which is incorporated herein by reference in their entirety, or a substantially similar assay. In one embodiment, the antibodies or antigen-binding fragments thereof that neutralize the infectivity of influenza virus for its host cells do so with an IC₅₀ of less than 130 nM. In some embodiments, the antibodies exhibit a neutralization potency against any of the SARS-CoV-2 variants described herein, including but not limited to delta and omicron variants. In some embodiments, the antibodies exhibit a neutralization potency against any ebola virus variant.

In other embodiments, the antigen-binding domains Z, including antibodies or antigen-binding fragments thereof, for use in the ADCs provided herein are those that mediate complement dependent cytotoxicity of infected cells, with an EC₅₀ ranging from about 20 nM to about 66 nM (see example 6 in WO 2016/100807 or US 2016/0176953 A1, each of which is incorporated herein by reference in their entirety). In one embodiment, the antibodies or antigen-binding fragments thereof mediate complement-dependent cytotoxicity of infected cells, with an EC₅₀ less than 66 nM.

In another embodiment, the anti-influenza-A HA antibodies for use in the ADCs provided herein demonstrate an increase in protection, or neutralization of influenza A infection in vivo, as compared to a control antibody. In another embodiment, the anti-SARS-CoV-2-5 antibodies for use in the ADCs provided herein demonstrate an increase in protection, or neutralization of SARS-CoV-2 infection in vivo, as compared to a control antibody. In another embodiment, the anti-ebola antibodies for use in the ADCs provided herein demonstrate an increase in protection, or neutralization of ebola infection in vivo, as compared to a control antibody. Certain antibodies show neutralization when administered either prophylactically (prior to infection) or therapeutically (after infection); see example 7 in WO 2016/100807 or US 2016/0176953 A1, each of which is incorporated herein by reference in their entirety.

In one embodiment, the antigen-binding domains Z, including isolated recombinant antibodies or antigen-binding fragments thereof, bind specifically to influenza-HA, wherein the antibody or fragment thereof exhibits two or more of the following characteristics: (a) is a fully human monoclonal antibody; (b) binds to influenza-HA with a dissociation constant (K_{D}) of less than 10⁻⁹ M, as measured in a surface plasmon resonance assay; (c) demonstrates a dissociative half-life (t %) ranging from about 370 minutes to greater than 1000 minutes; (d) demonstrates neutralization of group 1 influenza A viruses selected from H1N1, H5N1, H9N2, H13N6, and H16N3, with an IC₅₀ ranging from about 1.6 nM to about 130 nM; (e) demonstrates complement mediated lysis of influenza virus infected cells with an EC₅₀ of about 20 nM to about 66 nM; or (f) demonstrates protection, as measured by increased survival in an animal model of influenza virus infection when administered either before or after virus challenge.

In one embodiment, the antigen-binding domains Z, including isolated recombinant antibodies or antigen-binding fragments thereof, bind specifically a SARS-CoV-2 omicron variant, wherein the antibody or fragment thereof exhibits two or more of the following characteristics: (a) is a fully human monoclonal antibody; (b) binds to a SARS-CoV-2 omicron variant with a dissociation constant (K₅₀) of less than 10⁻⁹ M, as measured in a surface plasmon resonance assay; (c) demonstrates a dissociative half-life (t %) ranging from about 370 minutes to greater than 1000 minutes; (d) demonstrates neutralization of SARS-CoV-2 viruses selected from omicron B.1.1.529, BA.1, BA.1.1, BA.2, BA.3, BA.4 and BA.5 lineages, with an IC₅₀ ranging from about 1.6 nM to about 130 nM; (e) demonstrates complement mediated lysis of SARS-CoV-2 virus infected cells with an EC₅₀ of about 20 nM to about 66 nM; or (f) demonstrates protection, as measured by increased survival in an animal model of SARS-CoV-2 infection when administered either before or after virus challenge.

In one embodiment, the antigen-binding domains Z, including isolated recombinant antibodies or antigen-binding fragments thereof, bind specifically an ebola virus, wherein the antibody or fragment thereof exhibits two or more of the following characteristics: (a) is a fully human monoclonal antibody; (b) binds to an ebola virus with a dissociation constant (K₅₀) of less than 10⁻⁹ M, as measured in a surface plasmon resonance assay; (c) demonstrates a dissociative half-life (t %) ranging from about 370 minutes to greater than 1000 minutes; (d) demonstrates neutralization of ebola viruses, with an IC₅₀ ranging from about 1.6 nM to about 130 nM; (e) demonstrates complement mediated lysis of ebola virus infected cells with an EC₅₀ of about 20 nM to about 66 nM; or (f) demonstrates protection, as measured by increased survival in an animal model of ebola infection when administered either before or after virus challenge.

The antigen-binding domains Z, including antibodies and antigen-binding fragments thereof, for use in the ADCs herein may possess two or more of the aforementioned biological characteristics, or any combinations thereof. Other biological characteristics of the antigen-binding domains Z, including antibodies and antigen-binding fragments thereof, for use in the ADCs herein will be evident to a person of ordinary skill in the art from a review of the present disclosure including the working Examples herein.

E. Heavy and Light Chain Variable Region Amino Acid and Nucleotide Sequences

In some embodiments, the antigen-binding domain Z, conjugated to the linker-payload or payload can be an antibody that targets influenza-HA. Exemplary influenza-HA antibodies can be found, for example, in WO 2016/100807 or US 2016/0176953 A1, each of which are incorporated herein by reference in their entirety. In some embodiments, an influenza-HA antibody comprises a heavy chain complementarity determining region (HCDR)-1 comprising SEQ ID NO: 20; an HCDR2 comprising SEQ ID NO: 22; an HCDR3 comprising SEQ ID NO: 24; a light chain complementarity determining region (LCDR)-1 comprising SEQ ID NO: 28; an LCDR2 comprising SEQ ID NO: 30; and an LCDR3 comprising SEQ ID NO: 32. In some embodiments, an influenza-HA antibody comprises a heavy chain variable region (HCVR) comprising SEQ ID NO: 18 and a light chain variable region (LCVR) comprising SEQ ID NO: 26. In any of the foregoing embodiments, the influenza-HA antibody can be prepared by site-directed mutagenesis to insert a glutamine residue at a site without resulting in disabled antibody function or binding. In one embodiment, the antibody includes a HCVR and further includes a peptide tag at the C-terminus of the HCVR. In one embodiment, the antibody includes a HCVR and further includes a peptide tag at the C-terminus of the HCVR, wherein the peptide tag is the pentapeptide sequence LLQGA. In one embodiment, the antibody includes a HCVR and further includes a peptide tag at the C-terminus of the HCVR. In one embodiment, the antibody includes a HCVR and further includes a peptide tag at the C-terminus of the HCVR, wherein the peptide tag is the pentapeptide sequence ELQGP. In one embodiment, the antibody includes two HCVRs and further includes a peptide tag at the C-terminus of each HCVR. In one embodiment, the antibody includes two HCVRs and further includes a peptide tag at the C-terminus of the HCVRs, wherein each peptide tag is independently the pentapeptide sequence LLQGA or the pentapeptide sequence ELQGP.

In some embodiments, the antigen-binding domain Z, conjugated to the linker-payload or payload can be an antibody that targets SARS-CoV-2. Exemplary SARS-CoV-2 antibodies can be found, for example, in U.S. Pat. No. 10,787,501, which is incorporated herein by reference in its entirety. In some embodiments, a SARS-CoV-2 antibody comprises a heavy chain complementarity determining region (HCDR)-1 comprising SEQ ID NO: 315 or 335; an HCDR2 comprising SEQ ID NO: 317 or 337; an HCDR3 comprising SEQ ID NO: 319 or 339; a light chain complementarity determining region (LCDR)-1 comprising SEQ ID NO: 323 or 343; an LCDR2 comprising SEQ ID NO: 325 or 345; and an LCDR3 comprising SEQ ID NO: 327 or 347. In some embodiments, a SARS-CoV-2 antibody comprises a heavy chain variable region (HCVR) comprising SEQ ID NO: 313 or 333 and a light chain variable region (LCVR) comprising SEQ ID NO: 321 or 341. In any of the foregoing embodiments, the SARS-CoV-2 antibody can be prepared by site-directed mutagenesis to insert a glutamine residue at a site without resulting in disabled antibody function or binding. In one embodiment, the antibody includes a HCVR and further includes a peptide tag at the C-terminus of the HCVR. In one embodiment, the antibody includes a HCVR and further includes a peptide tag at the C-terminus of the HCVR, wherein the peptide tag is the pentapeptide sequence LLQGA. In one embodiment, the antibody includes a HCVR and further includes a peptide tag at the C-terminus of the HCVR. In one embodiment, the antibody includes a HCVR and further includes a peptide tag at the C-terminus of the HCVR, wherein the peptide tag is the pentapeptide sequence ELQGP. In one embodiment, the antibody includes two HCVRs and further includes a peptide tag at the C-terminus of each HCVR. In one embodiment, the antibody includes two HCVRs and further includes a peptide tag at the C-terminus of the HCVRs, wherein each peptide tag is independently the pentapeptide sequence LLQGA or the pentapeptide sequence ELQGP.

In some embodiments, the antigen-binding domain Z, conjugated to the linker-payload or payload can be an antibody that targets ebola. Exemplary ebola antibodies can be found, for example, in U.S. Pat. Nos. 9,771,414 and 11,530,255, which are incorporated herein by reference in its entirety. In some embodiments, an ebola antibody comprises a heavy chain complementarity determining region (HCDR)-1 comprising SEQ ID NO: 355; an HCDR2 comprising SEQ ID NO: 357; an HCDR3 comprising SEQ ID NO: 359; a light chain complementarity determining region (LCDR)-1 comprising SEQ ID NO: 363; an LCDR2 comprising SEQ ID NO: WAS; and an LCDR3 comprising SEQ ID NO: 365. In some embodiments, a SARS-CoV-2 antibody comprises a heavy chain variable region (HCVR) comprising SEQ ID NO: 353 and a light chain variable region (LCVR) comprising SEQ ID NO: 361. In any of the foregoing embodiments, the SARS-CoV-2 antibody can be prepared by site-directed mutagenesis to insert a glutamine residue at a site without resulting in disabled antibody function or binding. In one embodiment, the antibody includes a HCVR and further includes a peptide tag at the C-terminus of the HCVR. In one embodiment, the antibody includes a HCVR and further includes a peptide tag at the C-terminus of the HCVR, wherein the peptide tag is the pentapeptide sequence LLQGA. In one embodiment, the antibody includes a HCVR and further includes a peptide tag at the C-terminus of the HCVR. In one embodiment, the antibody includes a HCVR and further includes a peptide tag at the C-terminus of the HCVR, wherein the peptide tag is the pentapeptide sequence ELQGP. In one embodiment, the antibody includes two HCVRs and further includes a peptide tag at the C-terminus of each HCVR. In one embodiment, the antibody includes two HCVRs and further includes a peptide tag at the C-terminus of the HCVRs, wherein each peptide tag is independently the pentapeptide sequence LLQGA or the pentapeptide sequence ELQGP.

Table 1 sets forth the amino acid sequence identifiers of the heavy and light chain variable regions and CDRs of selected anti-influenza-HA, anti-SARS-CoV-2 and ebola antibodies. The corresponding nucleic acid sequence identifiers are set forth in Table 2.

TABLE 1
Amino Acid Sequence Identifiers

| Antibody | SEQ ID Nos: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAb11723 | 2 | 4 | 6 | 8 | 10 | 12 | 14 | 16 |
| mAb11729 | 18 | 20 | 22 | 24 | 26 | 28 | 30 | 32 |
| mAb11820 | 34 | 36 | 38 | 40 | 42 | 44 | 46 | 48 |
| mAb11829 | 50 | 52 | 54 | 56 | 58 | 60 | 62 | 64 |
| mAb11829* | 50 | 52 | 54 | 56 | 66 | 68 | 70 | 72 |
| mAb11829 | 50 | 52 | 54 | 56 | 58 | 60 | 62 | 64 |
| mAb11830 | 74 | 76 | 78 | 80 | 82 | 84 | 86 | 88 |
| mAb11830* | 74 | 76 | 78 | 80 | 66 | 68 | 70 | 72 |
| mAb11903 | 90 | 92 | 94 | 96 | 98 | 100 | 102 | 104 |
| mAb14571 | 106 | 108 | 110 | 112 | 114 | 116 | 118 | 120 |
| mAb14571 | 106 | 108 | 110 | 112 | 114 | 116 | 118 | 120 |
| mAb11704 | 122 | 124 | 126 | 128 | 130 | 132 | 134 | 136 |
| mAb11711 | 138 | 140 | 142 | 144 | 146 | 148 | 150 | 152 |
| mAb11714 | 154 | 156 | 158 | 160 | 162 | 164 | 166 | 168 |
| mAb11717 | 170 | 172 | 174 | 176 | 178 | 180 | 182 | 184 |
| mAb11724 | 186 | 188 | 190 | 192 | 194 | 196 | 198 | 200 |
| mAb11727 | 202 | 204 | 206 | 208 | 210 | 212 | 214 | 216 |
| mAb11730* | 218 | 220 | 222 | 224 | 226 | 228 | 230 | 232 |
| mAb11731* | 234 | 236 | 238 | 240 | 66 | 68 | 70 | 72 |
| mAb11734* | 242 | 244 | 246 | 248 | 66 | 68 | 70 | 72 |
| mAb11736* | 250 | 252 | 254 | 256 | 66 | 68 | 70 | 72 |
| mAb11742* | 258 | 260 | 262 | 264 | 66 | 68 | 70 | 72 |
| mAb11744* | 266 | 268 | 270 | 272 | 66 | 68 | 70 | 72 |
| mAb11745* | 274 | 276 | 278 | 280 | 66 | 68 | 70 | 72 |
| mAb11747* | 282 | 284 | 286 | 288 | 66 | 68 | 70 | 72 |
| mAb11748* | 290 | 292 | 294 | 296 | 66 | 68 | 70 | 72 |
| mAb5385 | 298 | 299 | 300 | 301 | 302 | 303 | 304 | 305 |
| mAb10985** | 313 | 315 | 317 | 319 | 321 | 323 | 325 | 327 |
| mAb10987*** | 333 | 335 | 337 | 339 | 341 | 343 | 345 | 347 |
| mAb3471^{A} | 353 | 355 | 357 | 359 | 361 | 363 | **** | 365 |
| 4A8^{B} | 370 | — | — | — | 372 | — | — | — |

*mAbcontains one or more mutations in the constant region
**HC is SIN: 329; LC is SIN: 331
***HC is SIN: 349; LC is SIN: 351
****LCDR2 is WAS
^{A}HC is SEQ ID NO: 367, LC is SEQ ID NO: 369
^{B}hlgG1 constant HC is SEQ ID NO: 371, constant LC is SEQ ID NO: 373

SEQ ID NO: 14 is Lys Ala Ser; SEQ ID NO: 30 is Ala Ala Ser; SEQ ID NO: 46 is Lys Ala Ser; SEQ ID NO: 62 is Ala Ala Ser; SEQ ID NO: 70 is Ala Ala Ser; SEQ ID NO: 86 is Thr Ala Ser; SEQ ID NO: 102 is Gly Ala Ser; SEQ ID NO: 118 is Lys Ile Ser; SEQ ID NO: 134 is Ala Thr Ser; SEQ ID NO: 150 is Ala Ala Ser; SEQ ID NO: 166 is Ala Ala Ser; SEQ ID NO: 182 is Ala Ala Ser; SEQ ID NO: 198 is Ala Ala Ser; SEQ ID NO: 214 is Lys Ala Ser; SEQ ID NO: 230 is Gly Ala Ser; SEQ ID NO: 304 is Gly Asn Ser; SEQ ID NO: 325 is Gly Asn Ser; SEQ ID NO: 345 is Asp Val Ser. The remaining SEQ ID NOs from Table 1 are shown in the Sequence Listing XML, incorporated by reference herein.

TABLE 2
Nucleic Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAb11723 | 1 | 3 | 5 | 7 | 9 | 11 | 13 | 15 |
| mAb11729 | 17 | 19 | 21 | 23 | 25 | 27 | 29 | 31 |
| mAb11820 | 33 | 35 | 37 | 39 | 41 | 43 | 45 | 47 |
| mAb11829 | 49 | 51 | 53 | 55 | 57 | 59 | 61 | 63 |
| mAb11829* | 49 | 51 | 53 | 55 | 65 | 67 | 69 | 71 |
| mAb11829 | 49 | 51 | 53 | 55 | 57 | 59 | 61 | 63 |
| mAb11830 | 73 | 75 | 77 | 79 | 81 | 83 | 85 | 87 |
| mAb11830* | 73 | 75 | 77 | 79 | 65 | 67 | 69 | 71 |
| mAb11903 | 89 | 91 | 93 | 95 | 97 | 99 | 101 | 103 |
| mAb14571 | 105 | 107 | 109 | 111 | 113 | 115 | 117 | 119 |
| mAb14571 | 105 | 107 | 109 | 111 | 113 | 115 | 117 | 119 |
| mAb11704 | 121 | 123 | 125 | 127 | 129 | 131 | 133 | 135 |
| mAb11711 | 137 | 139 | 141 | 143 | 145 | 147 | 149 | 151 |
| mAb11714 | 153 | 155 | 157 | 159 | 161 | 163 | 165 | 167 |
| mAb11717 | 169 | 171 | 173 | 175 | 177 | 179 | 181 | 183 |
| mAb11724 | 185 | 187 | 189 | 191 | 193 | 195 | 197 | 199 |
| mAb11727 | 201 | 203 | 205 | 207 | 209 | 211 | 213 | 215 |
| mAb11730* | 217 | 219 | 221 | 223 | 225 | 227 | 229 | 231 |
| mAb11731* | 233 | 235 | 237 | 239 | 65 | 67 | 69 | 71 |
| mAb11734* | 241 | 243 | 245 | 247 | 65 | 67 | 69 | 71 |
| mAb11736* | 249 | 251 | 253 | 255 | 65 | 67 | 69 | 71 |
| mAb11742* | 257 | 259 | 261 | 263 | 65 | 67 | 69 | 71 |
| mAb11744* | 265 | 267 | 269 | 271 | 65 | 67 | 69 | 71 |
| mAb11745* | 273 | 275 | 277 | 279 | 65 | 67 | 69 | 71 |
| mAb11747* | 281 | 283 | 285 | 287 | 65 | 67 | 69 | 71 |
| mAb11748* | 289 | 291 | 293 | 295 | 65 | 67 | 69 | 71 |
| mAb10985** | 312 | 324 | 316 | 318 | 320 | 322 | 324 | 326 |
| mAb10987*** | 332 | 334 | 336 | 338 | 340 | 342 | 344 | 346 |
| mAb3471^{A} | 352 | 354 | 356 | 358 | 360 | 362 | **** | 364 |

* mAbcontains one or more mutations in the constant region.
**HC is SEQ ID NO: 328; LC is SEQ ID NO: 330
***HC is SEQ ID NO: 348; LC is SEQ ID NO: 350
****LCDR2 is TGG GCA TCT
^{A}HC is SEQ ID NO: 366, LC is SEQ ID NO: 369
^{B}hlgG1 constant HC is SEQ ID NO: 371, constant LC is SEQ IS NO: 373

SEQ ID NO: 13 is aaggcgtct; SEQ ID NO: 29 is gctgcgtcc; SEQ ID NO: 45 is aaggcgtct; SEQ ID NO: 61 is gctgcatcc; SEQ ID NO: 69 is gctgcatcc; SEQ ID NO: 85 is actgcatcc; SEQ ID NO: 101 is ggtgcatcc; SEQ ID NO: 117 is aagatttct; SEQ ID NO: 133 is gctacatcc; SEQ ID NO: 149 is gctgcatcc; SEQ ID NO: 165 is gctgcatcc; SEQ ID NO: 181 is gctgcatcc; SEQ ID NO: 197 is gctgcatcc; SEQ ID NO: 213 is aaggcgtct; SEQ ID NO: 229 is ggtgcatcc; SEQ ID NO: 324 is ggtaacagc; SEQ ID NO: 344 is gatgtcagt. The remaining SEQ ID NOs from Table 2 are shown in the Sequence Listing XML, incorporated by reference herein.

4A8 hIgG1 HC nucleic acid sequence is SEQ ID NO: 374; hIgG1-HC-Cterm-LLQGA HC amino acid sequence is SEQ ID NO: 375 and nucleic acid sequence is SEQ ID NO: 376; and hIgG1-HC-Cterm-ELQRP HC amino acid sequence is SEQ ID NO: 377 and nucleic acid sequence is SEQ ID NO: 378.

F. Conjugation of Linker-Verrucarin A Derivatives -L-X to Antigen-Binding Domains Z

The linker-verrucarin A derivatives -L-X can be conjugated to the antigen-binding domain Z, e.g., antibody or antigen-binding fragment, through an attachment at a particular amino acid within the antibody or antigen-binding fragment. In one embodiment, amino acid attachments that can be used herein include, e.g., lysine (see, e.g., U.S. Pat. No. 5,208,020; US 2010/0129314; Hollander et al., Bioconjugate Chem., 2008, 19:358-361; WO 2005/089808; U.S. Pat. No. 5,714,586; US 2013/0101546; and US 2012/0585592), cysteine (see, e.g., US 2007/0258987; WO 2013/055993; WO 2013/055990; WO 2013/053873; WO 2013/053872; WO 2011/130598; US 2013/0101546; and U.S. Pat. No. 7,750,116), selenocysteine (see, e.g., WO 2008/122039; and Hofer et al., Proc. Natl. Acad. Sci., USA, 2008, 105:12451-12456), formyl glycine (see, e.g., Carrico et al., Nat. Chem. Biol., 2007, 3:321-322; Agarwal et al., Proc. Natl. Acad. Sci., USA, 2013, 110:46-51, and Rabuka et al., Nat. Protocols, 2012, 10:1052-1067), non-natural amino acids (see, e.g., WO 2013/068874, and WO 2012/166559), and acidic amino acids (see, e.g., WO 2012/05982). Linkers L can also be conjugated to an antigen-binding domain Z via attachment to carbohydrates (see, e.g., US 2008/0305497, WO 2014/065661, Ryan et al., Food & Agriculture Immunol., 2001, 13:127-130, and Jeger et al., Angew Chem Int Ed Engl., 2010, 49:9995-9997).

In some embodiments, the antigen-binding domain Z is an antibody or antigen-binding fragment, which is bonded to the L group of a linker-verrucarin A derivative -L-X through a lysine residue. In some embodiments, the antigen-binding domain Z is an antibody or antigen-binding fragment, which is bonded to the L group of a linker-verrucarin A derivative -L-X through a cysteine residue.

In certain embodiments herein, the L group of a linker-verrucarin A derivative can be conjugated to one or more glutamine residues in the antigen-binding domain Z via transglutaminase-based chemo-enzymatic conjugation (see, e.g., Jeger et al., Angew Chem Int Ed Engl., 2010, 49:9995-9997 and Dennler et al., Bioconjugate Chem. 2014, 25:569-578). For example, in the presence of transglutaminase, one or more glutamine residues of an antibody can be coupled to a primary amine compound. Primary amine compounds include, e.g., verrucarin A derivatives X and linker-verrucarin A derivatives -L-X, which directly provide antibody drug conjugates via transglutaminase-mediated coupling. Antibodies comprising glutamine residues can be isolated from natural sources or engineered to comprise one or more glutamine residues. Techniques for engineering glutamine residues into an antibody polypeptide chain (glutaminyl-modified antibodies or antigen-binding fragments) are within the skill of the practitioners in the art. In certain embodiments, the antibody is aglycosylated. In certain embodiments, the antibody is glycosylated.

In certain embodiments, the antibody or a glutaminyl-modified antibody or antigen-binding fragment comprises at least one glutamine residue in at least one polypeptide chain sequence. In certain embodiments, the antibody or a glutaminyl-modified antibody or antigen-binding fragment comprises two heavy chain polypeptides, each with one Gln295 or Q295 residue. In further embodiments, the antibody or a glutaminyl-modified antibody or antigen-binding fragment comprises one or more glutamine residues at a site other than a heavy chain 295. Included herein are antibodies of this section bearing N297Q mutation(s) described herein. In another embodiment, the antibodies can also be conjugated in the presence of the carbohydrates at N297 using the method described in Dickdiesser, et al., Bioconjugate Chem. 2020, 31, 1070-1076.

In one embodiment, the antibody or a glutaminyl-modified antibody or antigen-binding fragment includes an antibody heavy chain and further includes a peptide tag at the C-terminus of the antibody heavy chain. In one embodiment, the antibody or a glutaminyl-modified antibody or antigen-binding fragment includes an antibody heavy chain and further includes a peptide tag at the C-terminus of the antibody heavy chain, wherein the peptide tag is the pentapeptide sequence LLQGA or ELQGP. In one embodiment, the antibody or a glutaminyl-modified antibody or antigen-binding fragment includes two antibody heavy chains and further includes a peptide tag at the C-terminus of each antibody heavy chain. In one embodiment, the antibody or a glutaminyl-modified antibody or antigen-binding fragment includes two antibody heavy chains and further includes a peptide tag at the C-terminus of each antibody heavy chain, wherein the peptide tag is the pentapeptide sequence LLQGA or ELQGP.

In another embodiment, provided herein are ADC compounds having one of the following formulae:

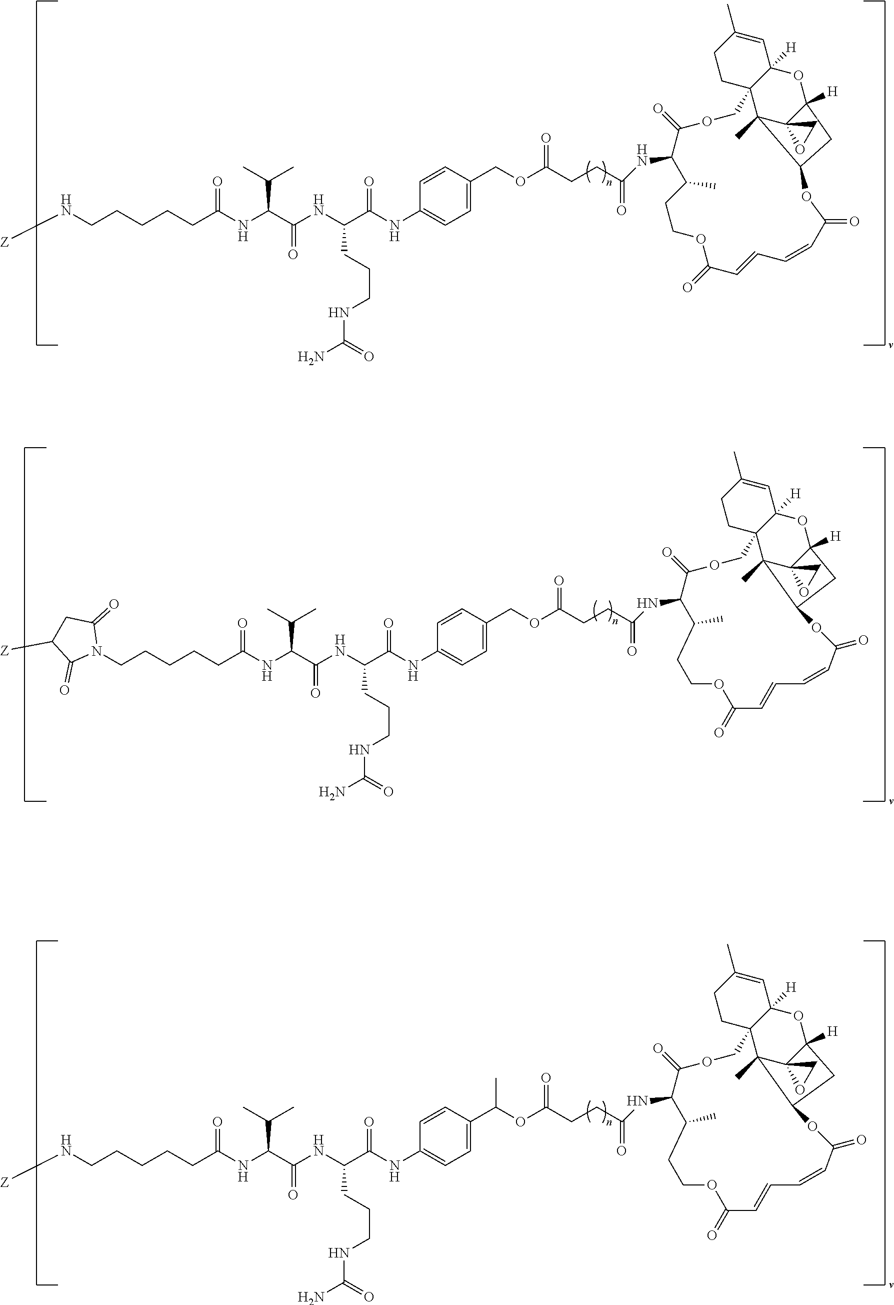

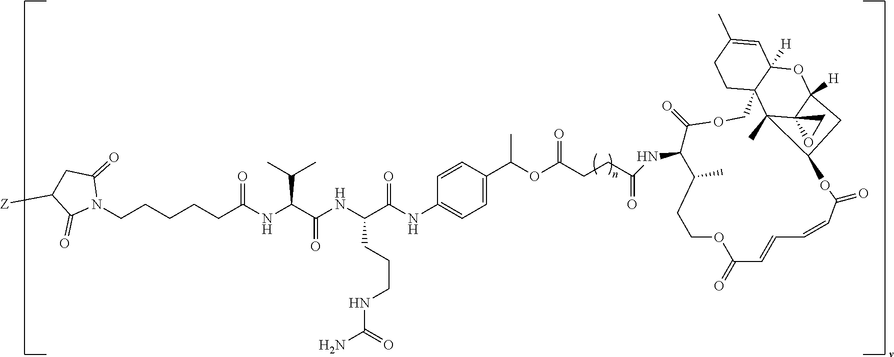

where n, v and Z are as defined herein.

### VII. Synthesis of the ADCs

Also provided herein is a method of synthesizing the ADCs provided herein. The ADCs provided herein may be prepared according to standard methods well known to those of skill in the art.

For example, antibodies with an engineered conjugation site at the C-terminus, e.g., a polypeptide or “Q-tag”, may be reacted with a linker-verrucarin A derivative having a primary amine in the presence of transglutaminase, e.g., bacterial transglutaminase, to give the ADCs provided herein.

Alternatively, non-engineered antibodies may be treated with excess TCEP (tris(2-carboxyethyl)phosphine) to reduce interchain disulfide bonds. The reduced antibody is then reacted with a linker-verrucarin A derivative having a maleimido group to give the ADCs provided herein.

In both cases, the ADCs may be purified by standard techniques, e.g., size exclusion chromatography in PBS/5% glycerol.

### VIII. Pharmaceutical Compositions

The pharmaceutical compositions provided herein contain therapeutically effective amounts of one or more of the verrucarin A derivatives, linker-verrucarin A derivatives and/or ADCs provided herein and a pharmaceutically acceptable carrier.

The verrucarin A derivatives, linker-verrucarin A derivatives and/or ADCs can be formulated into suitable pharmaceutical preparations. Typically, the verrucarin A derivatives, linker-verrucarin A derivatives and/or ADCs described above are formulated into pharmaceutical compositions using techniques and procedures well known in the art (see, e.g., Ansel Introduction to Pharmaceutical Dosage Forms, Seventh Edition 1999).

In the compositions, effective concentrations of one or more of the verrucarin A derivatives, linker-verrucarin A derivatives and/or ADCs or pharmaceutically acceptable salts is (are) mixed with a suitable pharmaceutical carrier. In certain embodiments, the concentrations of the verrucarin A derivatives, linker-verrucarin A derivatives and/or ADCs in the compositions are effective for delivery of an amount, upon administration, that treats, prevents, or ameliorates one or more of the symptoms and/or progression of a disease or disorder disclosed herein.

Typically, the compositions are formulated for single dosage administration. To formulate a composition, the weight fraction of the verrucarin A derivatives, linker-verrucarin A derivatives and/or ADC is dissolved, suspended, dispersed or otherwise mixed in a selected carrier at an effective concentration such that the treated condition is relieved or ameliorated. Pharmaceutical carriers suitable for administration of the verrucarin A derivatives, linker-verrucarin A derivatives and/or ADCs provided herein include any such carriers known to those skilled in the art to be suitable for the particular mode of administration.

In some embodiments, the verrucarin A derivative, linker-verrucarin A derivative and/or ADC is included in the pharmaceutically acceptable carrier in an amount sufficient to exert a therapeutically useful effect in the absence of undesirable side effects on the subject treated. The therapeutically effective concentration may be determined empirically by testing the compounds in in vitro and in vivo systems described herein and well known to those of skill in the art, and then extrapolated therefrom for dosages for humans. In some embodiments, the verrucarin A derivative, linker-verrucarin A derivative and/or ADC is administered in a method to achieve a therapeutically effective concentration of the payload. In some embodiments, a companion diagnostic (see, e.g., Olsen D and Jorgensen J T, Front. Oncol., 2014 May 16, 4:105, doi: 10.3389/fonC.2014.00105) is used to determine the therapeutic concentration and safety profile of the verrucarin A derivative, linker-verrucarin A derivative and/or ADC in specific subjects or subject populations.

The concentration of the verrucarin A derivative, linker-verrucarin A derivative and/or ADC in the pharmaceutical composition will depend on absorption, tissue distribution, inactivation and excretion rates of the verrucarin A derivative, linker-verrucarin A derivative and/or ADC, the physicochemical characteristics of the verrucarin A derivative, linker-verrucarin A derivative and/or ADC, the dosage schedule, and amount administered as well as other factors known to those of skill in the art. For example, the amount that is delivered is sufficient to ameliorate one or more of the symptoms of a disease or disorder disclosed herein.

The compositions may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions.

The compositions may include other active compounds to obtain desired combinations of properties. The verrucarin A derivatives, linker-verrucarin A derivatives and/or ADCs provided herein, or pharmaceutically acceptable salts thereof as described herein, may also be advantageously administered for therapeutic or prophylactic purposes together with another pharmacological agent known in the general art to be of value in treating one or more of the diseases or medical conditions referred to herein. It is to be understood that such combination therapy constitutes a further aspect of the compositions and methods of treatment provided herein.

### IX. Dosing

The verrucarin A derivatives, linker-verrucarin A derivatives and/or ADCs provided herein, and pharmaceutical compositions provided herein, may be dosed in certain therapeutically or prophylactically effective amounts, certain time intervals, certain dosage forms, and certain dosage administration methods as described below.

The methods provided herein encompass treating a patient regardless of subject's age, although some diseases or disorders are more common in certain age groups.

The verrucarin A derivative, linker-verrucarin A derivative and/or ADC provided herein, or a pharmaceutically acceptable salt thereof, can be administered repeatedly if necessary, for example, until the subject experiences stable disease or regression, or until the subject experiences disease progression or unacceptable toxicity.

The verrucarin A derivative, linker-verrucarin A derivative and/or ADC provided herein, or a pharmaceutically acceptable salt thereof, can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound, such as the verrucarin A derivative, linker-verrucarin A derivative and/or ADC provided herein, or a pharmaceutically acceptable salt thereof, is administered once or more than once each day, for example, for a period of time. The term “continuous” is intended to mean that a therapeutic compound, such as the verrucarin A derivative, linker-verrucarin A derivative and/or ADC provided herein, or a pharmaceutically acceptable salt thereof, is administered daily for an uninterrupted period of at least 10 days to 52 weeks. The term “intermittent” or “intermittently” as used herein is intended to mean stopping and starting at either regular or irregular intervals. For example, intermittent administration of the verrucarin A derivative, linker-verrucarin A derivative and/or ADC provided herein, or a pharmaceutically acceptable salt thereof, is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as the verrucarin A derivative, linker-verrucarin A derivative and/or ADC provided herein, or a pharmaceutically acceptable salt thereof, is administered daily or continuously but with a rest period. In some such embodiments, administration is once a day for two to six days, then a rest period with no administration for five to seven days.

### X. Methods of Treatment

In another embodiment, a method of treating a subject with a verrucarin A derivative, linker-verrucarin A derivative and/or ADC provided herein, or a pharmaceutically acceptable salt thereof, is provided. In another embodiment, a method of treating a subject with a pharmaceutical composition comprising a verrucarin A derivative, linker-verrucarin A derivative and/or ADC provided herein, or a pharmaceutically acceptable salt thereof, is provided. The pharmaceutical composition comprises any of the verrucarin A derivatives, linker-verrucarin A derivatives and/or ADCs disclosed herein, or a pharmaceutically acceptable salt thereof, and a pharmaceutically acceptable carrier.

In one embodiment, the verrucarin A derivatives, linker-verrucarin A derivatives and/or ADCs provided herein are useful in treating viral diseases. In one embodiment, the verrucarin A derivatives, linker-verrucarin A derivatives and/or ADCs provided herein are useful in treating COVID-19. SARS-CoV-2, the viral cause of COVID-19, has shown a remarkable ability to mutate into additional pathogenic variants, e.g., beta, delta, omicron, etc. The verrucarin A derivatives, linker-verrucarin A derivatives and/or ADCs provided herein are useful in treating some or all such SARS-CoV-2 variants. For example, the antibody portion of ADCs provided herein can be generated or modified to overcome viral resistance in the event that a SARS-CoV-2 variant develops resistance to the existing antibody portion. In another embodiment, the verrucarin A derivatives, linker-verrucarin A derivatives and/or ADCs provided herein are useful in treating Ebola.

In certain embodiments, the verrucarin A derivatives, linker-verrucarin A derivatives and/or ADCs provided herein are useful in treating influenza including influenza A, B and C. In another embodiment, provided is a method of treating influenza in a subject by administering a verrucarin A derivative, linker-verrucarin A derivative and/or an ADC provided herein. In another embodiment, provided is a method of treating influenza A in a subject by administering a verrucarin A derivative, linker-verrucarin A derivative and/or an ADC provided herein. In another embodiment, provided is a method of treating influenza A in a subject by administering a verrucarin A derivative, linker-verrucarin A derivative and/or an ADC provided herein, wherein the influenza A is any of subtypes H1, H2, H3, H5, H6, H7, H9 and H10; and N1, N2, N6, N7, N8 and N9. In another embodiment, provided is a method of treating H1N1 influenza A in a subject by administering a verrucarin A derivative, linker-verrucarin A derivative and/or an ADC provided herein. In another embodiment, provided is a method of treating H3N2 influenza A in a subject by administering a verrucarin A derivative, linker-verrucarin A derivative and/or an ADC provided herein.

In certain embodiments, the verrucarin A derivatives, linker-verrucarin A derivatives and/or ADCs provided herein are useful in treating COVID-19 including COVID-19 caused by SARS-CoV-2, including Alpha (B.1.1.7 and Q lineages), Beta (B.1.351 and descendent lineages), Gamma (P.1 and descendent lineages), Delta (B.1.617.2 and AY lineages), Epsilon (B.1.427 and B.1.429), Eta (B.1.525), Iota (B.1.526), Kappa (B.1.617.1), 1.617.3, Mu (B.1.621 and B.1.621.1), Zeta (P.2) and Omicron (B.1.1.529, BA.1, BA.1.1, BA.2, BA.3, BA.4 and BA.5 lineages) variants. In another embodiment, provided is a method of treating COVID-19 in a subject by administering a verrucarin A derivative, linker-verrucarin A derivative and/or an ADC provided herein. In another embodiment, provided is a method of treating COVID-19 caused by an omicron variant of SARS-CoV-2 in a subject by administering a verrucarin A derivative, linker-verrucarin A derivative and/or an ADC provided herein. In another embodiment, provided is a method of treating COVID-19 caused by an omicron variant of SARS-CoV-2 in a subject by administering a verrucarin A derivative, linker-verrucarin A derivative and/or an ADC provided herein, wherein the COVID-19 caused by an omicron variant of SARS-CoV-2 is any of subtypes B.1.1.529, BA.1, BA.1.1, BA.2, BA.3, BA.4 and BA.5. In another embodiment, provided is a method of treating COVID-19 caused by the Omicron BA.4 variant of SARS-CoV-2 in a subject by administering a verrucarin A derivative, linker-verrucarin A derivative and/or an ADC provided herein. In another embodiment, provided is a method of treating COVID-19 caused by the Omicron BA.5 variant of SARS-CoV-2 in a subject by administering a verrucarin A derivative, linker-verrucarin A derivative and/or an ADC provided herein.

In certain embodiments, the verrucarin A derivatives, linker-verrucarin A derivatives and/or ADCs provided herein are useful in treating ebola, including ameliorating or reducing the severity of at least one symptom of ebola virus infection including, but not limited to fever, headache, fatigue, loss of appetite, myalgia, diarrhea, vomiting, abdominal pain, dehydration and unexplained bleeding. In another embodiment, the verrucarin A derivatives, linker-verrucarin A derivatives and/or ADCs provided herein are useful prophylactically in subjects at risk for developing an Ebola virus infection.

In the context of the methods of treatment provided herein, the verrucarin A derivatives, linker-verrucarin A derivatives and/or ADCs may be administered as a monotherapy (i.e., as the only therapeutic agent) or in combination with one or more additional therapeutic agents (examples of which are described elsewhere herein).

### XI. Combination Therapy with a Second Active Agent

Provided herein are compositions comprising any of the verrucarin A derivatives, linker-verrucarin A derivatives and/or ADCs provided herein in combination with one or more additional therapeutically active components, and methods of treatment comprising administering such combinations to a subject.

The verrucarin A derivatives, linker-verrucarin A derivatives and/or ADCs provided herein may be co-formulated with and/or administered in combination with one or more additional therapeutically active component(s) selected from oseltamivir, zanamivir, peramivir, baloxavir, amantadine and rimantadine. The verrucarin A derivatives, linker-verrucarin A derivatives and/or ADCs provided herein also may be co-formulated with and/or administered in combination with one or more additional therapeutically active component(s) selected from molnupiravir, remdesivir, baricitinib, nirmatrelvir, ritonavir, bebtelovimab, tocilizumab, casirivimab and imdevimab. In another embodiment, the verrucarin A derivatives, linker-verrucarin A derivatives and/or ADCs provided herein may be co-formulated with and/or administered in combination with one or more additional therapeutically active component(s) selected from an anti-viral drug, an anti-inflammatory drug (such as corticosteroids, and non-steroidal anti-inflammatory drugs), a anti-ebola antibody, a vaccine for ebola virus, TKM ebola (small interfering RNAs that target viral RNA polymerase), brincidofovir (CMX-001), favipiravir (T-705), BCX-4430, AVI-7537 (antisense phosphorodiamidate morpholino oligomers that target ebola virus VP24 gene), interferons, or any other palliative therapy to treat an ebola virus infection.

The additional therapeutically active component(s), e.g., any of the agents listed above or derivatives thereof, may be administered just prior to, concurrent with, or shortly after the administration of a verrucarin A derivative, linker-verrucarin A derivative and/or ADC provided herein. In another embodiment, provided are pharmaceutical compositions in which a verrucarin A derivative, linker-verrucarin A derivative and/or ADC provided herein is co-formulated with one or more of the additional therapeutically active component(s) as described herein.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a verrucarin A derivative and/or ADC provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

Administration of the verrucarin A derivatives, linker-verrucarin A derivatives and/or ADCs provided herein provided herein, or a derivative thereof and one or more second active agents to a subject can occur simultaneously or sequentially by the same or different routes of administration. The suitability of a particular route of administration employed for a particular active agent will depend on the active agent itself (e.g., whether it can be administered orally without decomposing prior to entering the blood stream).

### XII. Examples

The examples below are meant to illustrate certain embodiments provided herein, and not to limit the scope of this disclosure.

Verrucarin A derivatives, linker-verrucarin A derivatives and ADCs provided herein were synthesized as indicated below. All the solvents used were used as is and purchased either from Sigma Aldrich or Fisher Scientific. ¹H spectra were recorded on Varian Inova 300 MHz and 500 MHz NMR instruments. The chemical shifts (δ) were reported in ppm with respect to the NMR solvents used for analysis and were reported as s—singlet, d—doublet, t—triplet, q—quartet, dd—doublet of doublet, dt—doublet of triplet, dq—doublet of quartet, and m—multiplet. Coupling constants (J) were reported in hertz (Hz). Chromatographic purities were determined on an Agilent 1100, 1260 Infinity with 6130 Quadrupole LC/MS, or 1200 Series LC/MS systems using Chromolith® FastGradient RP-18e analytical columns (50×2 mm, Merck KGaA, P/N 1.52007.0001) and the following analytical HPLC method: injection volume 2-10 μL; flow rate 1 mL/min; 5-95% acetonitrile in water over 4 min; Agilent diode array detector at λ=254 nm; room temperature. Low resolution mass spectrometry was performed on Agilent systems using electrospray ionization sources and analyzed with either single quadrupole or ion trap mass detectors.

### Example 1

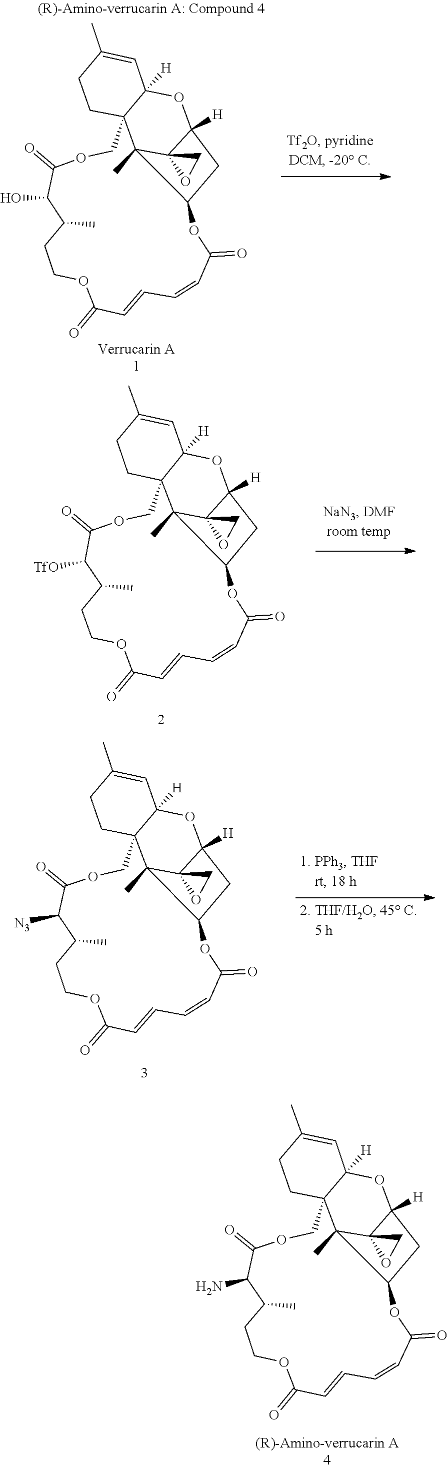 [SMILES: CC1=C[C@H]2O[C@@H]3C[C@H]4OC(=O)/C=C\C=C\C(=O)OCC[C@@H](C)[C@H](O)C(=O)OC[C@@]2(CC1)[C@]4(C)[C@]31CO1 ; CC1=C[C@H]2O[C@@H]3C[C@H]4OC(=O)/C=C\C=C\C(=O)OCC[C@@H](C)[C@H](OS(=O)(=O)C(F)(F)F)C(=O)OC[C@@]2(CC1)[C@]4(C)[C@]31CO1 ; CC1=C[C@H]2O[C@@H]3C[C@H]4OC(=O)/C=C\C=C\C(=O)OCC[C@@H](C)[C@@H](N=[N+]=[N-])C(=O)OC[C@@]2(CC1)[C@]4(C)[C@]31CO1 ; CC1=C[C@H]2O[C@@H]3C[C@H]4OC(=O)/C=C\C=C\C(=O)OCC[C@@H](C)[C@@H](N)C(=O)OC[C@@]2(CC1)[C@]4(C)[C@]31CO1]

Compound 2: To a solution of Verrucarin A (1, 5 mg, 0.01 mmol) in DCM (1.5 mL) at −20° C., pyridine (0.3 mL) was added followed by slow addition of trifluoromethanesulfonic anhydride (3.4 μL, 0.02 mmol). The reaction was stirred at the same temperature for 1 hour, at which time it was complete by LCMS analysis. The reaction was diluted with water (2 mL) and extracted with DCM (3×2 mL). The combined organic layers were washed with water and dried over anhydrous sodium sulfate, filtered, and concentrated to obtain 2 as colorless oil, which was used in the next step without purification. MS (ESI, pos.): calc'd for C₂₈H₃₃F₃O₁₁S, 634.2; found 635.1 (M+H), 657.1 (M+Na).

Compound 3: To a solution of crude 2 (0.01 mmol) in an anhydrous DMF (0.5 mL) at room temperature, sodium azide (2 mg, 0.03 mmol) was added and the mixture was vigorously stirred for 45 min, at which time the reaction was complete by LCMS analysis. The reaction was diluted with water (10 mL) and extracted with ethyl acetate (3×3 mL). The combined organic layers were dried over anhydrous sodium sulfate, filtered, and concentrated. Residual solvents were removed under high vacuum, affording 3, which was used in the next step without purification. MS (ESI, pos.): calc'd for C₂₇H₃₃N₃O₈, 527.2; found 528.3 (M+H), 550.2 (M+Na).

Compound 4: To a THF (1 mL) solution of 3 (0.01 mmol), triphenylphosphine (6 mg, 0.02 mmol) was added at room temperature and the reaction was stirred for 16 hours. DI water (0.2 mL) was added and the mixture was heated to 50° C. for 8 hours. The reaction was concentrated in vacuo, and the residue was dissolved in DMSO (0.6 mL) and injected into ISCO (5.5 g C18 Aq using 5-95% MeCN/water both having 0.05% AcOH) for purification. Pure fractions were combined and lyophilized to obtain compound 4 as off-fluffy solid (3.8 mg, 77% over three steps). MS (ESI, pos.): calc'd for C₂₇H₃₅NO₈, 501.2; found 502.3 (M+H). ¹H-NMR (500 MHz; CDCl₃): δ 8.07 (dd, J=15.7, 11.7 Hz, 1H), 6.68 (t, J=11.4 Hz, 1H), 6.14 (d, J=11.1 Hz, 1H), 6.05 (d, J=15.7 Hz, 1H), 5.80 (dd, J=8.1, 4.0 Hz, 1H), 5.45 (d, J=4.6 Hz, 1H), 4.53-4.47 (m, 2H), 4.27 (d, J=12.3 Hz, 1H), 4.05 (td, J=11.5, 3.2 Hz, 1H), 3.88 (d, J=5.1 Hz, 1H), 3.62 (d, J=5.1 Hz, 1H), 3.18 (d, J=9.1 Hz, 1H), 3.14 (d, J=3.9 Hz, 1H), 2.83 (d, J=3.9 Hz, 1H), 2.53 (dd, J=15.5, 8.2 Hz, 1H), 2.39-2.32 (m, 1H), 2.24-2.19 (m, 1H), 2.09-1.95 (m, 5H), 1.81-1.76 (m, 2H), 1.76 (s, 3H), 1.35-1.30 (m, 1H), 1.03 (d, J=6.7 Hz, 3H), 0.91 (s, 3H).

### Example 2

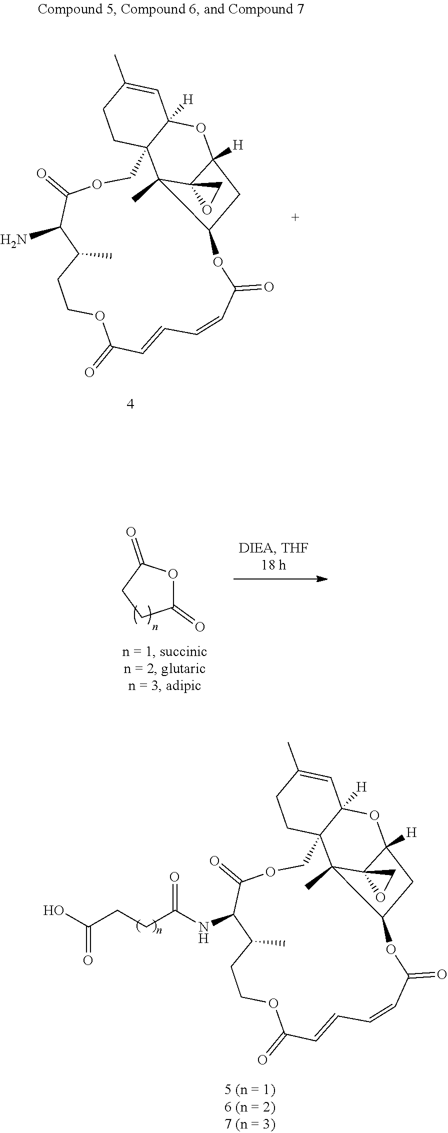

Compound 5: To a solution of (R)-amino-verrucarin A (4, 7 mg, 0.014 mmol) and succinic anhydride (2 mg, 0.02 mmol) in an anhydrous THF (0.6 mL) at room temperature N,N-diisopropylethylamine (8 μL, 0.042 mmol) was added and the reaction was stirred for 18 h. Volatiles were removed in vacuo and the residue was dissolved in DMF (0.5 mL) and purified by Teledyne ISCO EZ prep on 30×150 mm Gemini column using gradient elution 5-95% MeCN/H₂O (both having 0.05% AcOH). Pure fractions were combined and lyophilized to obtain compound 5 (7.8 mg, 93%) as an off-white fluffy solid. MS (ESI, pos.): calc'd for C₃₁H₃₉NO₁₁, 601.3; found 602.3 (M+H). ¹H-NMR (500 MHz; CDCl₃): δ 8.15 (dd, J=15.7, 11.7 Hz, 1H), 6.70 (t, J=11.4 Hz, 1H), 6.15 (d, J=11.0 Hz, 1H), 6.07-6.04 (m, 2H), 5.80 (dd, J=7.9, 4.0 Hz, 1H), 5.41 (d, J=4.5 Hz, 1H), 4.50 (dt, J=10.8, 3.6 Hz, 1H), 4.40 (d, J=12.3 Hz, 1H), 4.33-4.28 (m, 2H), 4.01 (td, J=11.2, 2.8 Hz, 1H), 3.86 (d, J=5.0 Hz, 1H), 3.59 (d, J=5.0 Hz, 1H), 3.13 (d, J=3.8 Hz, 1H), 2.83 (d, J=3.8 Hz, 1H), 2.75-2.64 (m, 2H), 2.56-2.42 (m, 2H), 2.21 (td, J=9.9, 5.2 Hz, 2H), 2.02-1.88 (m, 5H), 1.73 (s, 3H), 1.68-1.63 (m, 1H), 1.49-1.44 (m, 1H), 1.08 (d, J=6.7 Hz, 3H), 0.85 (s, 3H).

Compound 6: Using the same method and scale as for compound 5, compound 6 was prepared utilizing glutaric anhydride. Isolated yield was 8.0 mg (94% yield). MS (ESI, pos.): calc'd for C₃₂H₄₁NO₁₁, 615.3; found 616.3 (M+H). ¹H-NMR (500 MHz; CDCl₃): δ 8.14 (dd, J=15.7, 11.7 Hz, 1H), 6.70 (t, J=11.3 Hz, 1H), 6.15 (d, J=11.0 Hz, 1H), 6.06 (d, J=15.8 Hz, 1H), 5.85 (d, J=8.2 Hz, 1H), 5.79 (dd, J=7.7, 3.8 Hz, 1H), 5.41 (d, J=4.6 Hz, 1H), 4.49-4.46 (m, 1H), 4.39 (d, J=12.4 Hz, 1H), 4.30 (q, J=7.6 Hz, 2H), 4.02 (td, J=11.2, 2.0 Hz, 1H), 3.86 (d, J=5.0 Hz, 1H), 3.58 (d, J=5.0 Hz, 1H), 3.12 (d, J=3.8 Hz, 1H), 2.84 (d, J=3.7 Hz, 1H), 2.53-2.40 (m, 3H), 2.29 (t, J=7.3 Hz, 2H), 2.21 (td, J=9.9, 5.8 Hz, 2H), 1.99-1.88 (m, 6H), 1.73 (s, 3H), 1.65 (d, J=6.2 Hz, 1H), 1.49 (t, J=12.6 Hz, 1H), 1.09 (d, J=6.5 Hz, 3H), 0.84 (s, 3H).

Compound 7: Using the same method and scale as for compound 5, compound 7 was prepared utilizing adipic anhydride. Isolated yield was 5.6 mg (63% yield). MS (ESI, pos.): calc'd for C33H₄₃NO₁₁, 629.3; found 630.3 (M+H). ¹H-NMR (500 MHz; CDCl₃): δ 8.16 (dd, J=15.6, 11.7 Hz, 1H), 6.70 (t, J=11.4 Hz, 1H), 6.15 (d, J=11.1 Hz, 1H), 6.06 (d, J=15.7 Hz, 1H), 5.88 (d, J=8.0 Hz, 1H), 5.80 (dd, J=8.2, 4.0 Hz, 1H), 5.41 (d, J=5.1 Hz, 1H), 4.52 (dt, J=11.0, 3.9 Hz, 1H), 4.40 (d, J=12.3 Hz, 1H), 4.30 (t, J=10.2 Hz, 2H), 4.03 (td, J=11.4, 3.0 Hz, 1H), 3.86 (d, J=5.0 Hz, 1H), 3.58 (d, J=5.2 Hz, 1H), 3.13 (d, J=3.8 Hz, 1H), 2.83 (d, J=3.9 Hz, 1H), 2.51 (dd, J=15.5, 8.1 Hz, 1H), 2.38-2.36 (m, 2H), 2.23-2.18 (m, 4H), 2.06-1.87 (m, 5H), 1.73 (s, 3H), 1.68-1.62 (m, 4H), 1.47 (dd, J=14.0, 11.1 Hz, 1H), 1.10 (d, J=6.7 Hz, 3H), 0.83 (s, 3H).

### Example 3

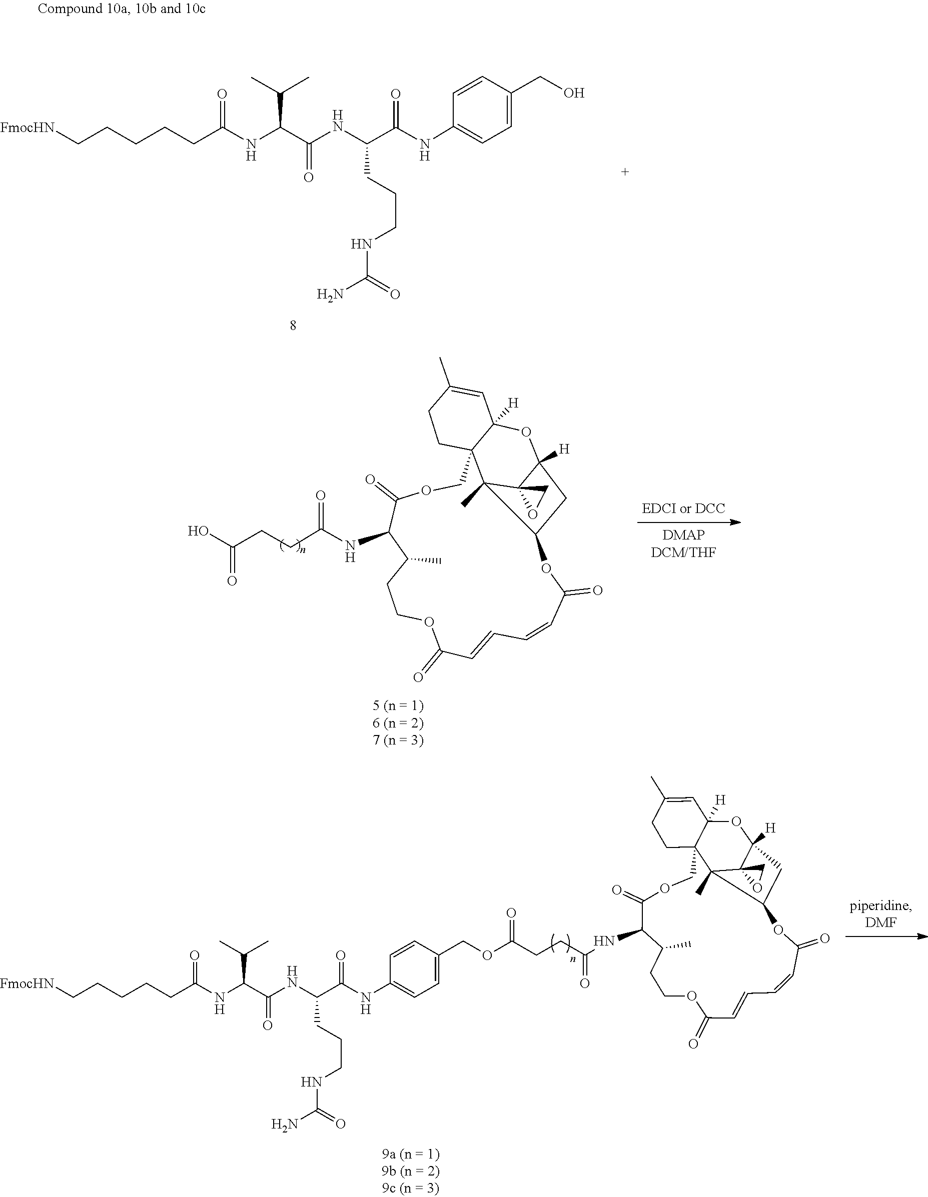

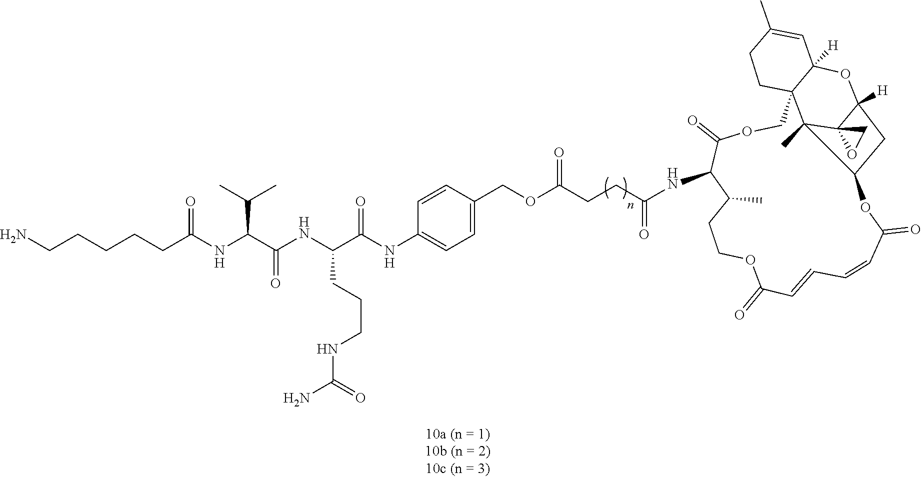

Compound 9a: To a mixture of compound 8 (9 mg, 0.011 mmol) and compound 5 (4.5 mg, 0.007 mmol) in THF/DCM (1+1 mL), were added EDCI (2.3 mg, 0.011 mmol) and DMAP (0.5 mg, 0.004 mmol) and the reaction was stirred for 18 h. Volatiles were removed under reduced pressure and the residue was purified on an ISCO 30 g C18Aq column using 5-95% MeCN/H₂O (both having 0.05% AcOH). Pure fractions were combined and lyophilized to obtain compound 9b (7 mg, 77%) as a fluffy white solid. MS (ESI, pos.): calc'd for C₇₀H₈₇N₇O₁₇, 1297.6; found 1298.5 (M+H).

Compound 9b: Compound 9b was prepared following the general procedure using acid 6. Yield=76%. MS (ESI, pos.): calc'd for C₇₁H₈₉N₇O₁₇, 1311.6; found 1312.5 (M+H).

Compound 9c: Compound 9c was prepared following the general procedure using acid 7. Yield=71%. MS (ESI, pos.): calc'd for C₇₂H₉₁N₇O₁₇, 1325.6; found 1326.5 (M+H).

Compound 10a: To a solution of compound 9a (7 mg 0.0053 mmol) in DMF (0.8 mL), was added 5% piperidine in DMF (0.5 mL) and the reaction was stirred for 60 min then purified on a 30×150 mm Gemini column using 5-95% MeCN/H₂O (both having 0.05% AcOH). Pure fractions were combined and lyophilized to obtain compound 10a (4.8 mg, 84%) as a fluffy white solid. MS (ESI, pos.): calc'd for C₅₅H₇₇N₇O₁₅, 1075.5; found 1076.4 (M+H). ¹H-NMR (500 MHz; DMSO-d₆): δ 10.01 (s, 1H), 8.22 (d, J=5.8 Hz, 1H), 8.16-8.13 (m, 1H), 7.96-7.91 (m, 1H), 7.88-7.83 (m, 1H), 7.59 (d, J=7.9 Hz, 2H), 7.27 (d, J=8.1 Hz, 2H), 6.87 (t, J=11.3 Hz, 1H), 6.24 (t, J=12.3 Hz, 2H), 6.04-6.00 (m, 1H), 5.76-5.74 (m, 1H), 5.41 (s, 2H), 5.26 (d, J=3.4 Hz, 1H), 5.00 (s, 2H), 4.40-4.35 (m, 2H), 4.18-4.11 (m, 2H), 4.02-3.99 (m, 1H), 3.95-3.90 (m, 1H), 3.74-3.71 (m, 1H), 3.68-3.65 (m, 2H), 3.03-2.94 (m, 5H), 2.74 (d, J=3.3 Hz, 1H), 2.43-2.40 (m, 2H), 2.19-2.07 (m, 4H), 1.98-1.92 (m, 2H), 1.82 (s, 2H), 1.65-1.57 (m, 8H), 1.47 (dd, J=4.2, 3.1 Hz, 3H), 1.36-1.34 (m, 3H), 1.24-1.23 (m, 4H), 0.91 (d, J=6.4 Hz, 2H), 0.83 (dd, J=11.9, 6.8 Hz, 7H), 0.74 (s, 3H).

Compound 10b was prepared from compound 9b following the same procedure. Yield=68%. MS (ESI, pos.): calc'd for C₅₆H₇₉N₇O₁₅, 1089.6; found 1090.6 (M+H). ¹H-NMR (500 MHz; DMSO-d₆): δ 10.01 (s, 1H), 8.15 (d, J=6.3 Hz, 1H), 7.94 (ddd, J=15.6, 11.6, 0.9 Hz, 1H), 7.61 (d, J=8.6 Hz, 2H), 7.27 (d, J=8.6 Hz, 2H), 6.87 (t, J=11.7 Hz, 1H), 6.24 (dd, J=13.3, 11.1 Hz, 2H), 5.76 (dd, J=8.0, 3.7 Hz, 1H), 5.44-5.43 (m, 1H), 5.27 (d, J=2.3 Hz, 1H), 5.01 (s, 2H), 4.42-4.38 (m, 1H), 4.33-4.31 (m, 1H), 4.11 (d, J=12.2 Hz, 2H), 4.02 (d, J=12.3 Hz, 1H), 3.92 (td, J=11.3, 4.2 Hz, 1H), 3.73 (dd, J=10.0, 6.1 Hz, 1H), 3.68 (d, J=5.2 Hz, 1H), 3.65 (d, J=5.3 Hz, 1H), 3.02 (d, J=4.0 Hz, 1H), 2.99-2.93 (m, 3H), 2.74 (d, J=4.1 Hz, 1H), 2.63 (dt, J=3.7, 1.9 Hz, 1H), 2.60 (dd, J=14.4, 7.1 Hz, 2H), 2.43-2.39 (m, 2H), 2.37-2.33 (m, 4H), 2.21-2.16 (m, 5H), 2.01-1.92 (m, 2H), 1.87 (s, 3H), 1.74-1.71 (m, 3H), 1.65 (m, 2H), 1.60 (s, 3H), 1.50-1.36 (m, 7H), 1.28-1.23 (m, 4H), 0.91 (d, J=6.6 Hz, 3H), 0.84 (t, J=6.4 Hz, 6H), 0.74 (s, 3H).

Compound 10c was prepared from compound 9c following the same procedure. Yield=56%. MS (ESI, pos.): calc'd for C₅₇H₈₁N₇O₁₅, 1103.6; found 1104.4 (M+H). ¹H-NMR (500 MHz; DMSO-d₆): δ 10.00 (s, 1H), 8.10 (d, J=6.3 Hz, 1H), 7.96-7.90 (m, 2H), 7.59 (d, J=8.4 Hz, 2H), 7.27 (d, J=8.6 Hz, 2H), 6.87 (t, J=11.5 Hz, 1H), 6.61-6.59 (m, 1H), 6.24 (t, J=12.6 Hz, 3H), 5.77-5.74 (m, 1H), 5.42-5.41 (m, 2H), 5.28-5.27 (m, 1H), 5.00 (s, 2H), 4.41-4.33 (m, 3H), 4.13-4.10 (m, 2H), 4.01 (d, J=12.4 Hz, 1H), 3.95-3.90 (m, 2H), 3.74 (dd, J=9.7, 6.3 Hz, 1H), 3.68-3.65 (m, 2H), 3.01 (d, J=4.0 Hz, 2H), 2.99-2.94 (m, 2H), 2.73 (d, J=3.9 Hz, 1H), 2.63 (t, J=1.6 Hz, 1H), 2.45-2.40 (m, 1H), 2.35-2.32 (m, 3H), 2.20-2.14 (m, 5H), 1.99-1.92 (m, 2H), 1.84 (s, 1H), 1.78-1.72 (m, 1H), 1.66-1.61 (m, 4H), 1.50-1.46 (m, 4H), 1.39-1.34 (m, 3H), 0.91 (d, J=6.6 Hz, 3H), 0.85-0.82 (m, 6H), 0.74 (s, 3H).

### Example 4

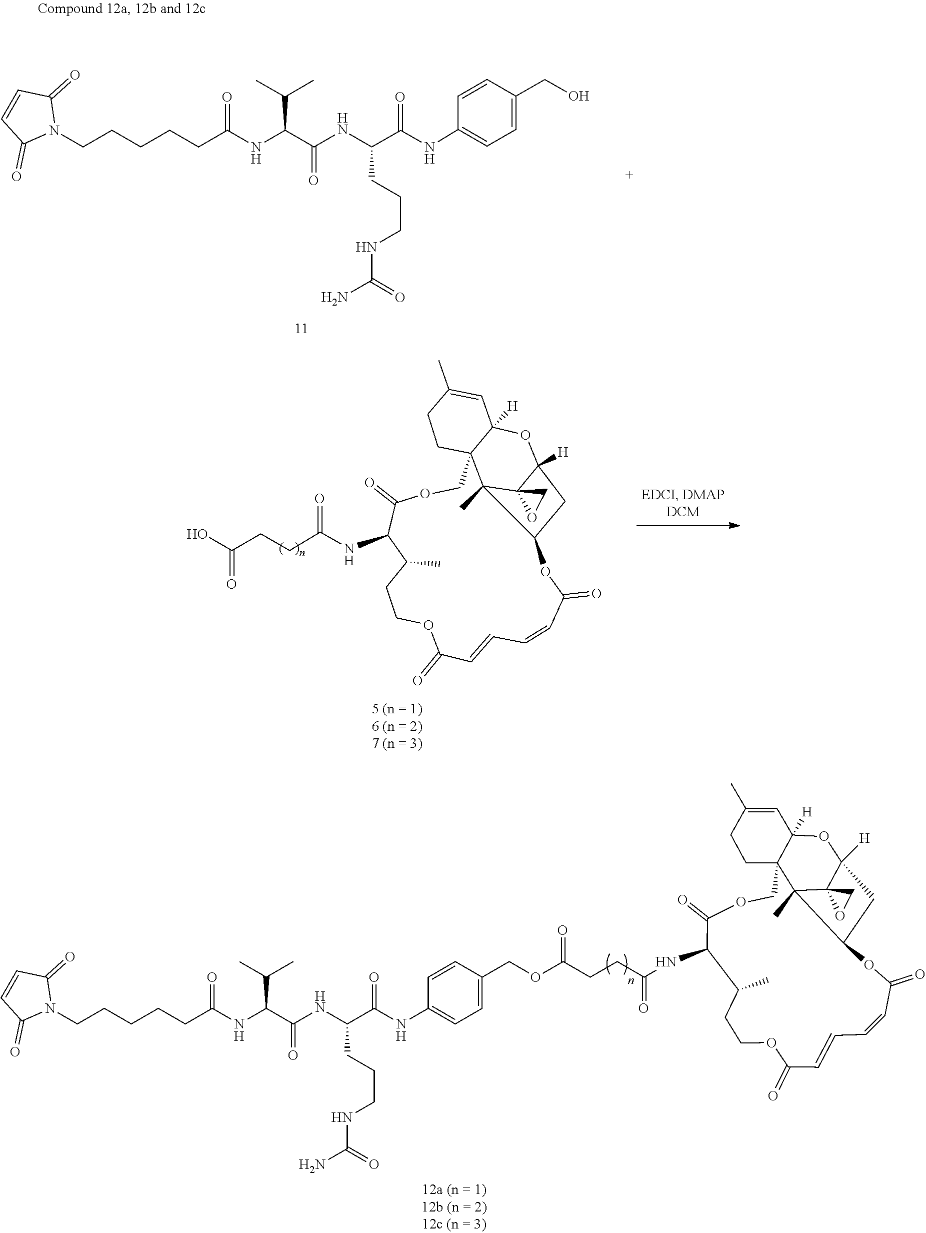

Compound 12a: Compound 12a was prepared from acid 5 and alcohol 11 using the same procedure as for compound 9a. Yield=13% after 2 purifications. MS (ESI, pos.): calc'd for C₅₉H₇₇N₇O₁₇, 1155.5; found 1156.3 (M+H). ¹H NMR (500 MHz; DMSO-d₆) δ 10.03 (s, 1H), 8.23-8.20 (m, 1H), 8.15-8.12 (m, 1H), 7.93 (dd, J=12.0, 15.5 Hz, 1H), 7.82 (d, J=9.0 Hz, 1H), 7.58 (d, J=8.5 Hz, 2H), 7.26 (d, J=8.5 Hz, 2H), 7.16 (br m, 3H), 6.99 (s, 2H), 6.86 (t, J=11.5 Hz, 1H), 6.26-6.21 (m, 2H), 6.01-5.98 (br m, 1H), 5.76-5.74 (m, 1H), 5.42-5.38 (m, 2H), 5.26 (d, J=4.5 Hz, 1H), 5.02-4.97 (m, 2H), 4.42-4.34 (m, 2H), 4.17 (dd, J=8.5, 7.0 Hz, 1H), 4.12 (d, J=12.5 Hz, 1H), 4.00 (d, J=12.5, 1H), 3.95-3.89 (m, 1H), 3.73 (dd, J=6.5, 10.0 Hz, 1H), 3.67 (d, J=5.0 Hz, 2H), 3.03 (d, J=4.0 Hz, 2H), 3.00-2.91 (m, 1H) 2.73 (d, J=4.0 Hz, 1H), 2.62 (dt, J=3.5, 2.0 Hz, 1H), 2.45-2.38 (m, 1H), 2.35 (m, 2H), 2.20-2.06 (m, 2H), 1.98-1.91 (m, 2H), 1.77-1.58 (m, 4H), 1.58-1.52 (m, 9H), 1.47-1.40 (m, 2H), 1.37-1.30 (m, 1H), 1.30-1.08 (m, 4H), 0.90 (d, J=6.5 Hz, 3H), 0.82 (dd, J=15.5, 7.0 Hz, 6H), 0.74 (s, 3H).

Compound 12b: Compound 12b was prepared from acid 6 and alcohol 11 using the same procedure as for compound 9a. Yield=41%. MS (ESI, pos.): calc'd for C₆₀H₇₉N₇O₁₇, 1169.6; found 1170.3 (M+H). ¹H-NMR (500 MHz; DMSO-d₆): δ 9.98 (s, 1H), 8.15 (d, J=6.2 Hz, 1H), 8.06 (d, J=7.4 Hz, 1H), 7.94 (ddd, J=15.6, 11.7, 0.9 Hz, 1H), 7.78 (d, J=8.6 Hz, 1H), 7.58 (d, J=8.6 Hz, 2H), 7.27 (d, J=8.6 Hz, 2H), 6.99 (s, 2H), 6.87 (t, J=11.5 Hz, 1H), 6.54-6.48 (m, 3H), 6.24 (t, J=12.4 Hz, 2H), 5.95 (t, J=5.6 Hz, 1H), 5.76 (dd, J=8.1, 3.8 Hz, 1H), 5.38 (dd, J=1.6, 1.1 Hz, 2H), 5.27 (d, J=4.7 Hz, 1H), 5.00 (s, 2H), 4.42-4.34 (m, 2H), 4.18 (dd, J=8.6, 6.9 Hz, 1H), 4.11 (d, J=12.2 Hz, 1H), 4.02 (d, J=12.3 Hz, 1H), 3.92 (td, J=11.4, 4.2 Hz, 1H), 3.73 (dd, J=9.9, 6.2 Hz, 1H), 3.67 (dd, J=11.1, 5.1 Hz, 2H), 3.01 (dd, J=12.7, 5.4 Hz, 2H), 2.93 (dt, J=13.0, 6.5 Hz, 1H), 2.74 (d, J=4.0 Hz, 1H), 2.63 (dt, J=3.6, 1.8 Hz, 1H), 2.42 (dd, J=15.1, 8.2 Hz, 1H), 2.35 (td, J=7.5, 2.8 Hz, 2H), 2.18 (dt, J=11.4, 7.1 Hz, 4H), 2.13-2.06 (m, 2H), 1.98-1.92 (m, 2H), 1.90 (d, J=9.2 Hz, 1H), 1.73 (dd, J=14.1, 8.3 Hz, 3H), 1.67-1.56 (m, 6H), 1.47 (dq, J=14.8, 7.4 Hz, 4H), 1.27-1.23 (m, 2H), 1.17 (dt, J=15.3, 7.7 Hz, 2H), 0.91 (d, J=6.6 Hz, 3H), 0.82 (dd, J=15.5, 6.8 Hz, 6H), 0.74 (s, 3H).

Compound 12c: Compound 12c was prepared from acid 7 and alcohol 11 using the same procedure as for compound 9a. Yield=23%. MS (ESI, pos.): calc'd for C₆₁H₈₁N₇O₁₇, 1183.6; found 1184.4 (M+H). ¹H NMR (500 MHz; DMSO-d6) δ 10.00 (s, 1H), 8.12-8.07 (m, 2H), 7.93 (dd, J=12.5, 16 Hz, 1H), 7.83-7.78 (br s, 1H), 7.58 (d, J=8.0 Hz, 2H), 7.26 (d, J=8.0 Hz, 2H), 6.98 (s, 2H), 6.86 (t, J=11.5 Hz, 1H), 6.82-6.72 (br s, 1H), 6.26-6.20 (m, 2H), 6.00-5.96 (br s, 1H), 5.77-5.73 (m, 1H), 5.42-5.37 (m, 2H), 5.35-5.30 (br s, 1H), 5.29-5.26 (m, 1H), 5.01-4.97 (br s, 2H), 4.40-4.33 (m, 2H), 4.18-4.15 (m, 1H), 4.11 (d, J=12.5 Hz, 1H), 4.03-3.98 (m, 1H), 3.95-3.89 (m, 1H), 3.74 (dd, J=6.5, 10.0 Hz, 1H), 3.67 (d, J=4.5 Hz, 1H), 3.66-3.63 (m, 1H), 3.05-2.87 (m, 4H), 2.73 (d, J=3.5 Hz, 1H), 2.63-2.58 (m, 1H), 2.46-2.39 (m, 2H), 2.37-2.30 (m, 2H), 2.22-2.05 (m, 4H), 1.99-1.91 (m, 2H), 1.81-1.69 (m, 1H), 1.69-1.63 (m, 3H), 1.63-1.54 (m, 4H), 1.53-1.37 (m, 8H), 1.37-1.31 (m, 1H), 1.28-1.13 (m, 4H), 0.90 (d, J=6.5 Hz, 3H), 0.82 (dd, J=7, 15 Hz, 6H), 0.73 (s, 3H).

### Example 5

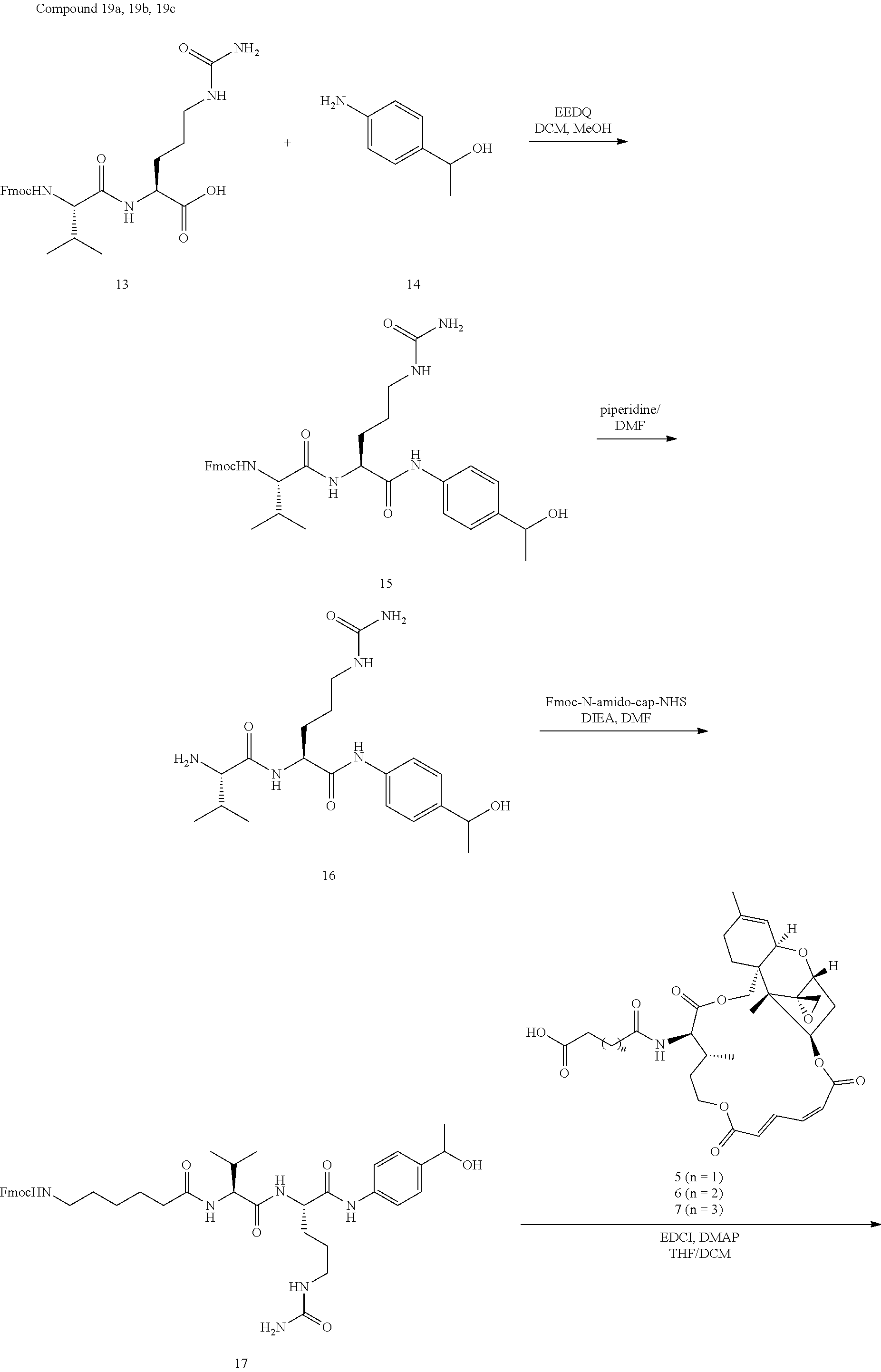

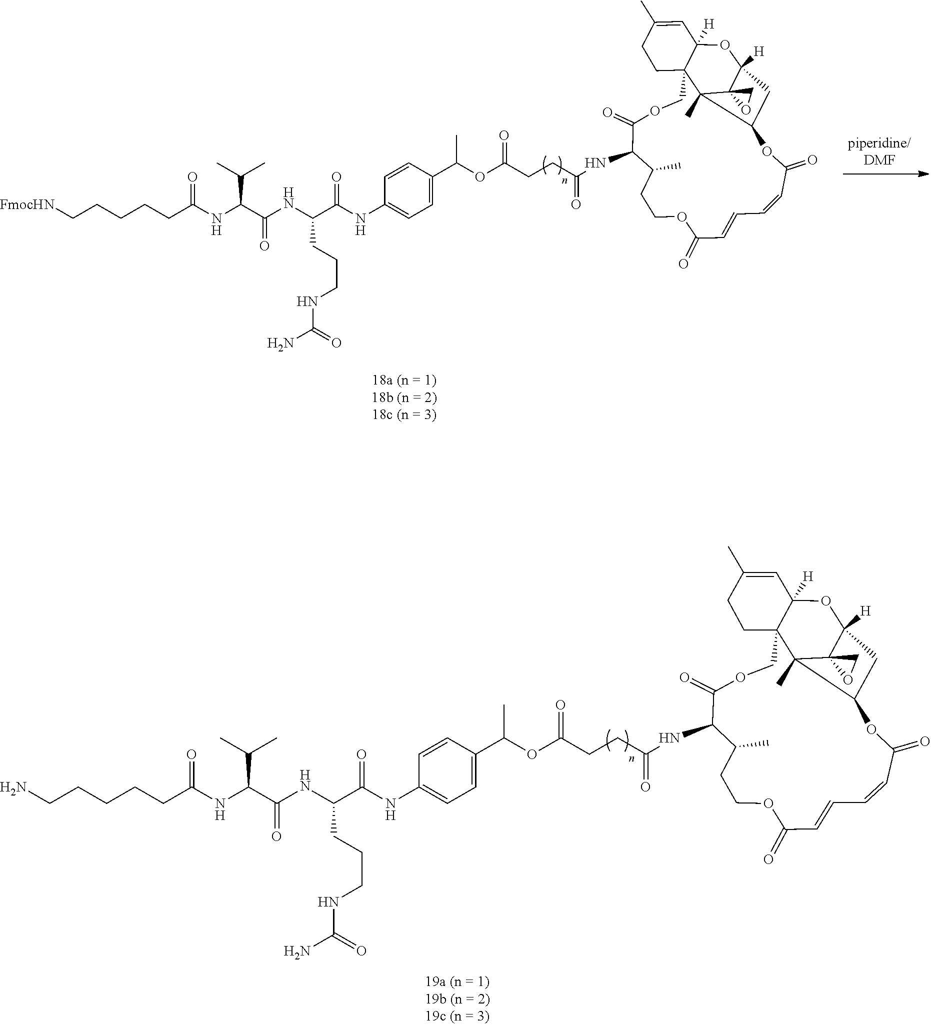

Compound 15: To a solution of Fmoc-Val-Cit-OH (13, 497 mg, 1.0 mmol) and 1-(4-aminophenyl)ethan-1-ol (14, 274 mg, 2.0 mmol) in DCM (4.5 mL) and MeOH (2 mL), was added EEDQ (495 mg, 2.0 mmol) and the reaction was stirred for 1 h at room temperature. The reaction turned into a gum. Additional DCM (4.5 mL) and MeOH (2 mL) were added, and the mixture was stirred overnight. Volatiles were removed under reduced pressure and the residue was washed sequentially with diethyl ether (5 mL), ethyl acetate (5 mL) and diethyl ether (5 mL). The residue was dried under high vacuum to obtain compound 15 (585 mg, 95%) as a light-yellow solid. MS (ESI, pos.): calc'd for C₃₄H₄₁N₅O₆, 615.3; found 616.3 (M+H).

Compound 16: To a solution of compound 15 (150 mg, 0.244 mmol) in DMF (1 mL), was added 5% piperidine solution in DMF (1 mL) and the reaction was stirred for 45 min at room temperature. Purification on a Teledyne ISCO 50 g C18 Aq column using gradient elution 5-95% MeCN/H₂O (both having 0.05% AcOH) afforded compound 16 (103 mg, 94%) as its acetate salt. MS (ESI, pos.): calc'd for C₁₉H₃₁N₅O₄, 393.2; found 394.3 (M+H).

Compound 17: To a solution of compound 16 (30 mg, 0.076 mmol) and Fmoc-N-amido-cap-NHS (34 mg, 0.076 mmol) in DMF, was added DIEA (20 μL, 0.114 mmol) and the reaction was stirred for 1.5 h. Purification on a Teledyne ISCO 50 g C18 Aq column using gradient elution 5-95% MeCN/H₂O (both having 0.05% AcOH) afforded compound 17 (20 mg, 36%). MS (ESI, pos.): calc'd for C₄₀H₅₂N₆O₇, 728.4; found 729.3 (M+H).

Compound 18a: Compound 18a was prepared from acid 5 and alcohol 17 using the same procedure as for compound 9a. Yield=47%, MS (ESI, pos.): calc'd for C₇₁H₈₉N₇O₁₇, 1311.6; found 1312.5 (M+H).

Compound 18b: Compound 18b was prepared from acid 6 and alcohol 17 using the same procedure as for compound 9a. Yield=75%. MS (ESI, pos.): calc'd for C₇₂H₉₁N₁₇O₁₇, 1325.6; found 1326.6 (M+H).

Compound 18c: Compound 18c was prepared from acid 7 and alcohol 17 using the same procedure as for compound 9a. Yield=50%. MS (ESI, pos.): calc'd for C₇₃H₉₃N₇O₁₇, 1339.7; found 1340.5 (M+H).

Compound 19a: Compound 19a was prepared from compound 18a using the same procedure as for compound 10a. Yield=60%. MS (ESI, pos.): calc'd for C₅₆N₇₉N₇O₁₅, 1089.6; found 1090.3 (M+H). ¹H-NMR (500 MHz; DMSO-d₆): δ 9.98 (s, 1H), 8.21 (s, 1H), 8.15-8.11 (m, 1H), 7.97-7.91 (m, 2H), 7.57 (d, J=7.9 Hz, 2H), 7.46 (d, J=7.8 Hz, 1H), 7.27 (t, J=7.0 Hz, 2H), 7.10 (d, J=7.2 Hz, 1H), 6.86 (d, J=11.4 Hz, 1H), 6.61 (s, 1H), 6.24 (t, J=12.3 Hz, 2H), 6.02 (s, 1H), 5.75-5.73 (m, 2H), 5.40 (d, J=6.3 Hz, 2H), 5.27 (t, J=4.4 Hz, 1H), 4.40-4.35 (m, 2H), 4.18-4.11 (m, 2H), 4.03-3.98 (m, 1H), 3.94-3.90 (m, 1H), 3.73-3.71 (m, 1H), 3.68-3.64 (m, 2H), 3.03 (dd, J=5.0, 4.4 Hz, 2H), 3.00-2.93 (m, 3H), 2.74-2.73 (m, 1H), 2.59-2.57 (m, 2H), 2.35 (s, 3H), 2.28 (s, 1H), 2.19-2.15 (m, 3H), 2.06 (s, 1H), 1.95 (td, J=2.2, 0.9 Hz, 2H), 1.86 (s, 2H), 1.66-1.58 (m, 6H), 1.48 (s, 1H), 1.42-1.38 (m, 6H), 1.26-1.23 (m, 4H), 0.90 (d, J=6.2 Hz, 3H), 0.83 (dd, J=11.1, 6.9 Hz, 6H), 0.74 (s, 3H).

Compound 19b: Compound 19b was prepared from compound 18b using the same procedure as for compound 10a. Yield=33%. MS (ESI, pos.): calc'd for C₅₇H₈₁N₇O₁₅, 1103.6; found 1104.3 (M+H). ¹H-NMR (500 MHz; DMSO-d₆): δ 9.98-9.97 (m, 1H), 8.15-8.13 (m, 2H), 7.97-7.91 (m, 1H), 7.56 (d, J=8.3 Hz, 2H), 7.27 (d, J=8.2 Hz, 2H), 6.89-6.84 (m, 1H), 6.51 (s, 1H), 6.26-6.21 (m, 2H), 6.02 (s, 1H), 5.77-5.73 (m, 2H), 5.41-5.39 (m, 2H), 5.28-5.26 (m, 1H), 4.83 (d, J=5.2 Hz, 1H), 4.60 (t, J=5.7 Hz, 1H), 4.40-4.35 (m, 2H), 4.19-4.16 (m, 1H), 4.12-4.09 (m, 1H), 4.02 (dd, J=11.1, 4.1 Hz, 2H), 3.93-3.87 (m, 2H), 3.74-3.71 (m, 1H), 3.67 (ddd, J=7.2, 5.0, 3.1 Hz, 2H), 3.62-3.60 (m, 1H), 3.03-2.91 (m, 4H), 2.73 (d, J=0.4 Hz, 1H), 2.66 (dd, J=1.0, 0.5 Hz, 2H), 2.63 (t, J=1.9 Hz, 1H), 2.36-2.30 (m, 3H), 2.27 (t, J=7.4 Hz, 1H), 2.20-2.15 (m, 4H), 1.98-1.93 (m, 2H), 1.89 (s, 1H), 1.72-1.70 (m, 3H), 1.61-1.59 (m, 4H), 1.49 (d, J=5.7 Hz, 4H), 1.43 (d, J=6.4 Hz, 4H), 0.91-0.90 (m, 3H), 0.83 (m, 8H), 0.73 (d, J=1.5 Hz, 3H).

Compound 19c: Compound 19c was prepared from compound 18c using the same procedure as for compound 10a. Yield=55%. MS (ESI, pos.): calc'd for C₅₈H₈₃N₇O₁₅, 1117.6; found 1118.4 (M+H). ¹H-NMR (500 MHz; DMSO-d₆): δ 9.98 (s, 1H), 8.10 (d, J=6.1 Hz, 1H), 7.97-7.90 (m, 2H), 7.57 (d, J=8.2 Hz, 2H), 7.27 (d, J=7.2 Hz, 2H), 6.87 (t, J=11.3 Hz, 1H), 6.24 (t, J=12.9 Hz, 2H), 5.76-5.73 (m, 2H), 5.40 (ddd, J=6.9, 2.1, 0.9 Hz, 2H), 5.28-5.27 (m, 1H), 4.40-4.34 (m, 2H), 4.16-4.10 (m, 2H), 4.02-3.99 (m, 1H), 3.99-3.90 (m, 1H), 3.76-3.72 (m, 1H), 3.69-3.65 (m, 2H), 3.06-2.91 (m, 6H), 2.73 (t, J=0.6 Hz, 1H), 2.63-2.62 (m, 1H), 2.35-2.32 (m, 1H), 2.33-2.27 (m, 3H), 2.21-2.06 (m, 6H), 1.97-1.93 (m, 2H), 1.83 (s, 2H), 1.66-1.58 (m, 8H), 1.49-1.45 (m, 6H), 1.45-1.40 (m, 4H), 1.39-1.35 (m, 3H), 0.91 (d, J=5.8 Hz, 3H), 0.85-0.82 (m, 6H), 0.74 (d, J=0.3 Hz, 3H).

### Example 6

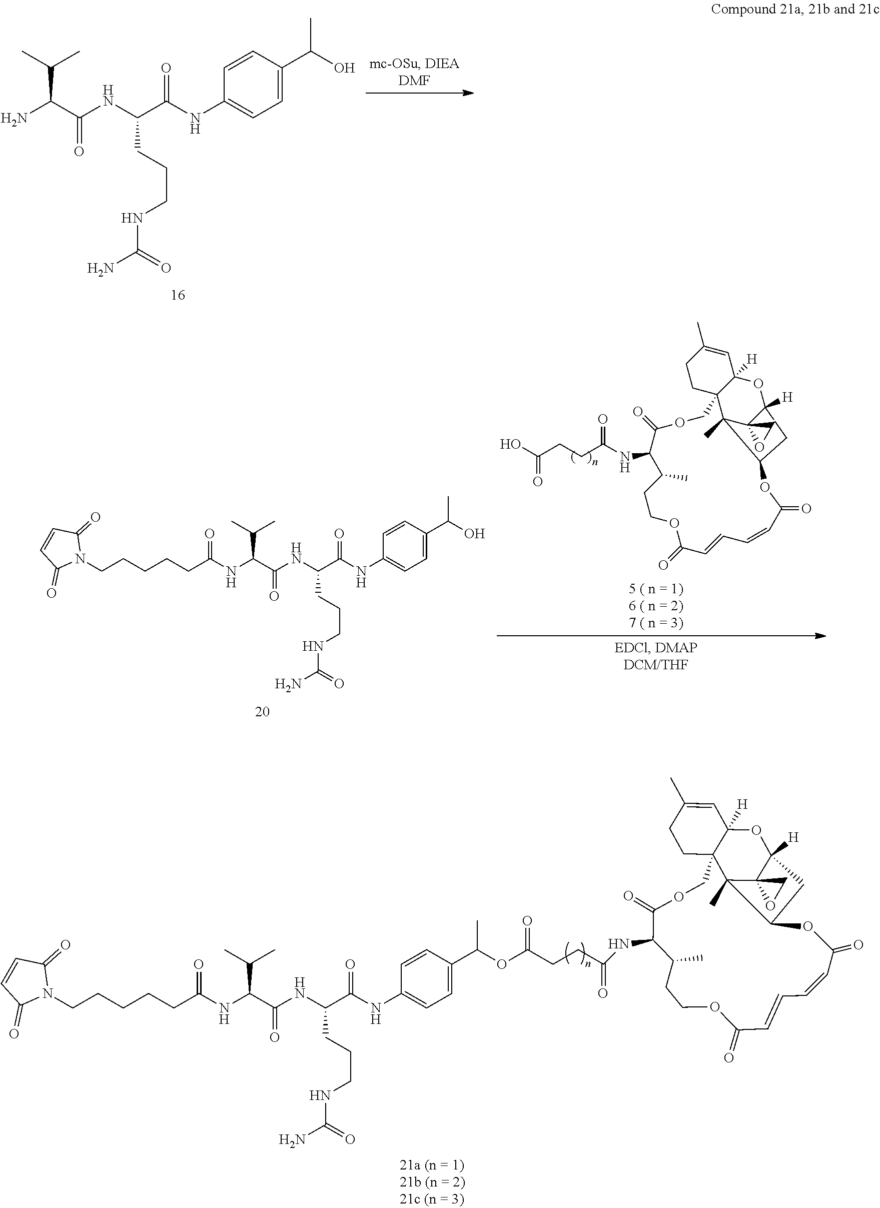

Compound 20: To a solution of compound 16 (30 mg, 0.076 mmol) and mal-cap-NHS (23.5 mg, 0.076 mmol) in DMF (1 mL), was added DIEA (20 μL, 0.114 mmol) and the reaction was stirred for 1 h then purified on a 50 g C18 Aq column using gradient elution 5-95% MeCN/H₂O (both having 0.05% AcOH). Pure fractions were combined and lyophilized to obtain title compound 20 (28 mg, 66%) as a fluffy off-white solid. MS (ESI, pos.): calc'd for C₂₉H₄₂N₆O₇, 586.3; found 587.3 (M+H).

Compound 21a: Compound 21a was prepared from acid 5 and alcohol 20 using the same procedure as for compound 10a. Yield=13%. MS (ESI, pos.): calc'd for C₆₀H₇₉N₇O₁₇, 1169.6; found 1170.3 (M+H). ¹H-NMR (500 MHz; DMSO-d₆): δ 10.00 (s, 1H), 8.21-8.19 (m, 1H), 8.13-8.11 (m, 1H), 7.96-7.90 (m, 1H), 7.83-7.80 (m, 1H), 7.56 (d, J=8.5 Hz, 2H), 7.26 (d, J=8.3 Hz, 2H), 6.99 (s, 2H), 6.87 (t, J=11.5 Hz, 1H), 6.27-6.21 (m, 2H), 6.00-5.97 (m, 1H), 5.77-5.71 (m, 2H), 5.39-5.38 (m, 2H), 5.28-5.26 (m, 1H), 4.41-4.33 (m, 2H), 4.19-4.10 (m, 2H), 4.02-3.99 (m, 1H), 3.95-3.89 (m, 1H), 3.73-3.71 (m, 1H), 3.71-3.65 (m, 3H), 3.04-3.03 (m, 2H), 3.00-2.92 (m, 3H), 2.74-2.73 (m, 1H), 2.43-2.40 (m, 1H), 2.21-2.14 (m, 2H), 2.14-2.06 (m, 2H), 1.98-1.91 (m, 2H), 1.80-1.76 (m, 1H), 1.66-1.57 (m, 9H), 1.48-1.45 (m, 4H), 1.42 (m, 5H), 1.23-1.15 (m, 4H), 0.91-0.90 (m, 3H), 0.84-0.80 (m, 6H), 0.74 (s, 3H).

Compound 21b: Compound 21b was prepared from acid 6 and alcohol 20 using the same procedure as for compound 10a. Yield=15%. MS (ESI, pos.): calc'd for C₆₁H₈₁N₁₇O₁₇, 1183.6; found 1184.4 (M+H). ¹H-NMR (500 MHz; DMSO-d₆): δ 9.99 (s, 1H), 8.15-8.13 (m, 1H), 8.11-8.09 (m, 1H), 7.94 (m, 2H), 7.82-7.79 (m, 1H), 7.57-7.55 (m, 2H), 7.27-7.26 (m, 2H), 6.99 (s, 2H), 6.89-6.84 (m, 1H), 6.26-6.21 (m, 2H), 5.97 (m, 1H), 5.77-5.73 (m, 2H), 5.39 (s, 2H), 5.28-5.26 (m, 1H), 4.41-4.33 (m, 2H), 4.17 (dd, J=8.3, 7.1 Hz, 1H), 4.12-4.09 (m, 1H), 4.03-4.01 (m, 1H), 3.94-3.89 (m, 1H), 3.73 (dd, J=10.0, 6.1 Hz, 1H), 3.69-3.64 (m, 2H), 3.03 (d, J=4.0 Hz, 1H), 3.01-2.99 (m, 1H), 2.93 (t, J=6.3 Hz, 2H), 2.73 (t, J=3.6 Hz, 1H), 2.63 (t, J=1.8 Hz, 1H), 2.53 (t, J=1.9 Hz, 1H), 2.45 (t, J=1.8 Hz, 2H), 2.45-2.39 (m, 2H), 2.35 (t, J=1.8 Hz, 1H), 2.34-2.30 (m, 2H), 2.20-2.16 (m, 3H), 2.13-2.06 (m, 2H), 1.97-1.93 (m, 2H), 1.72-1.69 (m, 3H), 1.66-1.57 (m, 6H), 1.50-1.42 (m, 5H), 1.24-1.23 (m, 2H), 1.20-1.16 (m, 2H), 0.91-0.90 (m, 3H), 0.82 (dd, J=14.7, 6.8 Hz, 6H), 0.73 (d, J=2.0 Hz, 3H).

Compound 21c: Compound 21c was prepared from acid 7 and alcohol 20 using the same procedure as for compound 10a. Yield=11%. MS (ESI, pos.): calc'd for C₆₂H₈₃N₇O₁₇, 1197.6; found 1198.3 (M+H). ¹H-NMR (500 MHz; DMSO-d₆): δ 9.98 (s, 1H), 8.10-8.08 (m, 2H), 7.93-7.90 (m, 1H), 7.58-7.55 (m, 2H), 7.27-7.25 (m, 2H), 6.99 (s, 2H), 6.85 (dd, J=11.1, 0.6 Hz, 1H), 6.24 (t, J=13.0 Hz, 2H), 5.77-5.72 (m, 2H), 5.39-5.38 (m, 2H), 5.28 (td, J=2.2, 1.0 Hz, 1H), 4.39-4.35 (m, 2H), 4.18-4.10 (m, 2H), 4.00 (ddd, J=11.7, 2.0, 0.8 Hz, 1H), 3.92 (s, 1H), 3.76-3.72 (m, 1H), 3.69-3.65 (m, 2H), 3.02-3.00 (m, 2H), 2.98-2.93 (m, 2H), 2.74-2.73 (m, 1H), 2.35-2.29 (m, 3H), 2.20-2.06 (m, 6H), 1.96-1.93 (m, 2H), 1.62-1.59 (m, 6H), 1.49-1.45 (m, 8H), 1.42 (d, J=6.7 Hz, 4H), 0.91 (d, J=6.6 Hz, 3H), 0.82 (dd, J=14.7, 6.7 Hz, 6H), 0.73 (s, 3H).

### Example 7

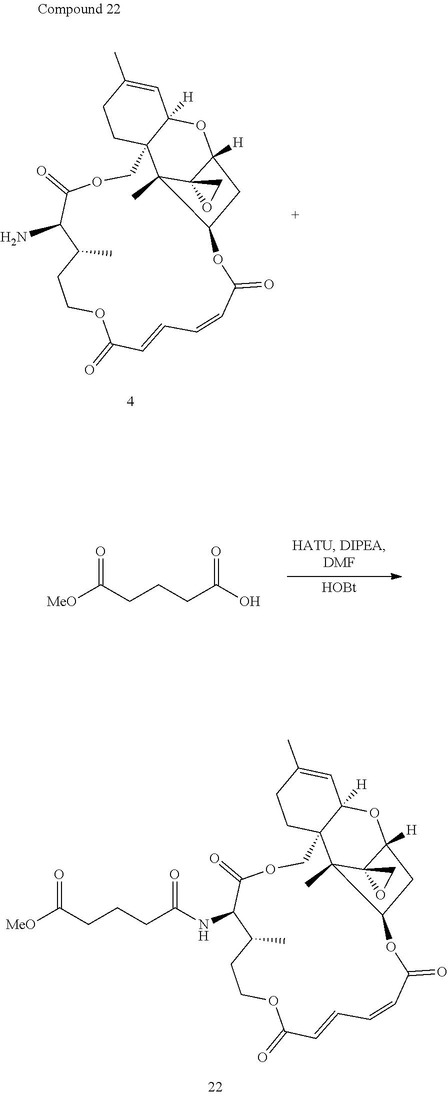 [SMILES: CC1=C[C@H]2O[C@@H]3C[C@H]4OC(=O)/C=C\C=C\C(=O)OCC[C@@H](C)[C@@H](N)C(=O)OC[C@@]2(CC1)[C@]4(C)[C@]31CO1 ; COC(=O)CCCC(=O)O ; COC(=O)CCCC(=O)N[C@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1 ; "HOBt"]

Compound 22: To a solution of methyl glutarate (2.4 mg, 0.016 mmol) in anhydrous DMF (0.8 mL), HATU (6.2 mg, 0.016 mmol), HOBt (2.2 mg, 0.016 mmol) and N,N-diisopropylylethylamine (5.7 μL, 0.033 mmol) were added. The reaction mixture was stirred at room temperature for 5 minutes then cooled to 0° C. (R)-amino-Verrucarin A (4, 8.2 mg, 0.016 mmol) was added and stirring was continued for 20 minutes at 0° C., then 1 hour at room temperature. The product was purified on a 5.5 g C18Aq ISCO column, eluting with 5-100% MeCN in H₂O with 0.05% AcOH in both. Pure fractions were combined and lyophilized to afford compound 22 (7.5 mg, 73%) as a white fluffy solid. MS (ESI, pos.): calc'd for C₃₃H₄₃NO₁₁, 629.28; found 630.3 (M+H). ¹H NMR (500 MHz, DMSO-d₆): δ 8.27 (d, J=6.5 Hz, 1H), 7.98-7.92 (m, 1H), 6.88 (t, J=11.5 Hz, 1H), 6.27-6.23 (m, 2H), 5.78-5.75 (m, 2H), 5.30-5.29 (m, 1H), 4.43-4.39 (m, 1H), 4.12 (d, J=12.0 Hz, 1H), 4.04 (d, J=12.0 Hz, 1H), 3.97-3.90 (m, 1H), 3.75 (dd, J=11.0, 6.5 Hz, 1H), 3.70-3.66 (m, 2H), 3.59 (s, 3H), 3.05 (d, J=4.0 Hz, 1H), 2.76 (d, J=4.0 Hz, 1H), 2.36-2.28 (m, 3H), 2.23-2.17 (m, 3H), 2.12-2.08 (m, 1H), 1.98-1.93 (m, 1H), 1.81-1.68 (m, 2H), 1.67-1.59 (m, 5H), 1.29-1.22 (m, 2H), 0.92 (d, J=6.5 Hz, 3H), 0.75 (s, 3H).

### Example 8

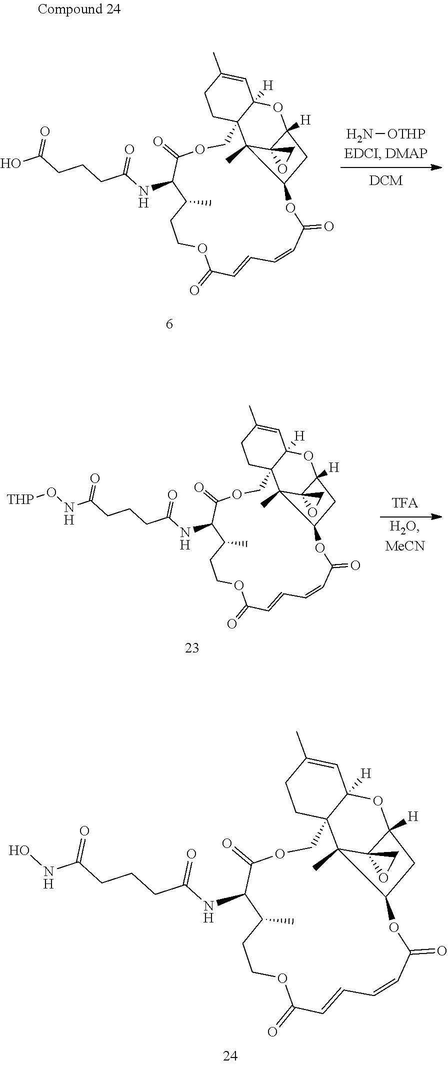

Compound 23: To a solution of compound 6 (15 mg, 0.0244 mmol), O-(tetrahydro-2H-pyran-2-yl)hydroxylamine (3.4 mg, 0.029 mmol) and DMAP (0.3 mg, 0.002 mmol) in anhydrous DCM (0.24 mL), EDCI (7 mg, 0.037 mmol) was added. The reaction mixture was stirred at room temperature for 2 hours. The product was purified on a 5.5 g C18Aq ISCO column, eluting with 5-100% MeCN in H₂O with 0.05% AcOH in both. Pure fractions were combined and lyophilized to afford compound 23 (12 mg, 69%) as a white fluffy solid. MS (ESI, pos.): calc'd for C₃₇H₅₀N₂O₁₂, 714.34; found 715.64 (M+H).

Compound 24: To a solution of compound 23 (6.0 mg, 0.0084 mmol) in 1:1 mixture of MeCN and H₂O (0.16 mL), trifluoroacetic acid (13 μL, 0.1679 mmol) was added. The reaction mixture was stirred at room temperature for 1 hour. The product was purified on a 5.5 g C18Aq ISCO column, eluting with 5-95% MeCN in H₂O with 10 mM NH₄OAc in both. Pure fractions were combined and lyophilized to afford compound 24 (4.0 mg, 76%) as a fluffy white solid. MS (ESI, pos.): calc'd for C₃₂H₄₂N₂O₁₁, 630.69; found 631.44 (M+H). ¹H NMR (500 MHz, DMSO-d₆): δ 10.3 (br, 1H), 8.65 (br, 1H), 8.13 (dd, J=6.0, 0.5 Hz, 1H), 7.95 (dd, J=15.5, 12.5 Hz, 1H), 6.88 (t, J=11.5 Hz, 1H), 6.28-6.23 (m, 2H), 5.78-5.73 (m, 1H), 5.29-5.28 (m, 1H), 4.42-4.39 (m, 1H), 4.12 (d, J=12.0 Hz, 1H), 4.03 (d, J=12.0 Hz, 1H), 3.96-3.91 (m, 1H), 3.75 (dd, J=10.0, 6.5 Hz, 1H), 3.69-3.66 (m, 2H), 3.03 (d, J=1.5 Hz, 1H), 2.81 (d, J=4.0 Hz, 1H), 2.24-2.05 (m, 5H), 1.98-1.93 (m, 3H), 1.81-1.73 (m, 2H), 1.72-1.59 (m, 5H), 1.28-1.23 (m, 2H), 0.92 (d, J=6.5 Hz, 3H), 0.75 (s, 3H).

### Example 9

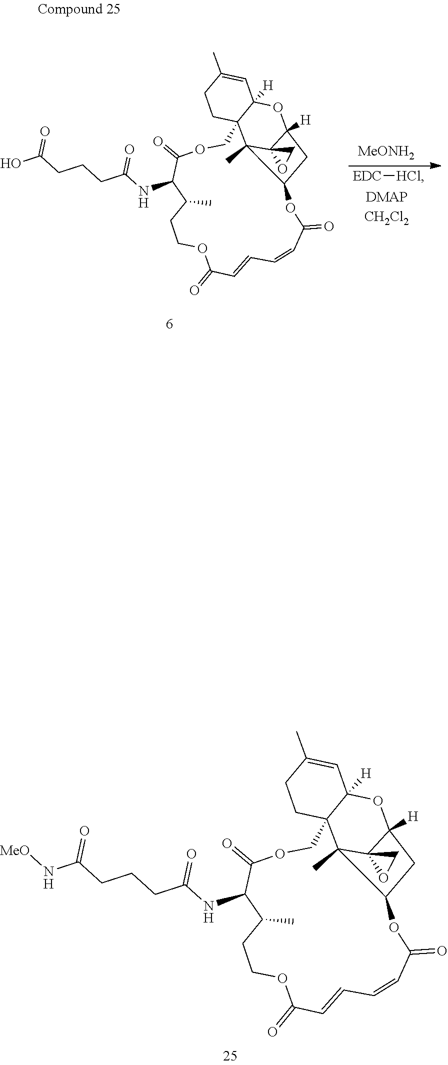

Compound 25: A solution of approximately 0.3 M MeONH₂ in THF was prepared from MeONH₂ HCl salt (42.5 mg, 0.5 mmol) and KOH (28 mg, 0.5 mmol) in THF (1.5 mL). The suspension was filtered prior to use. To a solution of compound 6 (5 mg, 0.00812 mmol) and DMAP (0.1 mg, 0.000818 mmol) in DCM was added 0.3 M MeONH₂ solution in THF (50 mL, 0.015 mmol) and EDC-HCl (3 mg, 0.0156 mmol). The reaction was stirred at room temperature for 6 hours, at which time LCMS indicated the reaction was complete. The reaction was concentrated under vacuum. The product was purified by chromatography on a 5.5 g C18Aq ISCO column, eluting with 5-100% MeCN in H₂O with 0.05% HOAc in both. Pure fractions were combined and lyophilized to afford compound 25 (4.4 mg, 85%) as a white fluffy solid. MS (ESI, pos.): calc'd for C₃₃H₄₄N₂O₁₁, 644.29; found 645.3 (M+H). ¹H-NMR (300 MHz; CDCl₃): δ 8.5 (br s, 1H), 8.11 (dd, J=15.7, 11.6 Hz, 1H), 6.69 (t, J=11.4 Hz, 1H), 6.14 (d, J=11.1 Hz, 1H), 6.10 (d, J=15.8 Hz, 1H) 5.97 (d, J=8.2 Hz, 1H), 5.77 (dd, J=8.1, 4.1 Hz, 1H), 5.41 (br d, J=5.5 Hz, 1H), 4.47 (ddd, J=11.1, 4.7, 3.5 Hz, 1H), 4.38 (d, J=12.2 Hz, 1H), 4.30 (d, J=12.9 Hz, 1H), 4.29 (d, J=8.6 Hz, 1H), 4.07-3.98 (m, 1H), 3.85 (d, J=5.0 Hz, 1H), 3.75 (s, 3H), 3.58 (d, J=5.0 Hz, 1H), 3.12 (d, J=3.8 Hz, 1H), 2.88 (d, J=3.9 Hz, 1H), 2.50 (dd, J=15.5, 8.2 Hz, 1H), 2.31 (t, J=7.0 Hz, 2H), 2.26-2.15 (m, 3H), 2.09-1.85 (m, 6H), 1.73 (s, 3H), 1.68-1.61 (m, 1H), 1.51-1.41 (m, 2H), 1.09 (d, J=6.7 Hz, 3H), 0.86 (s, 3H).

### Example 10

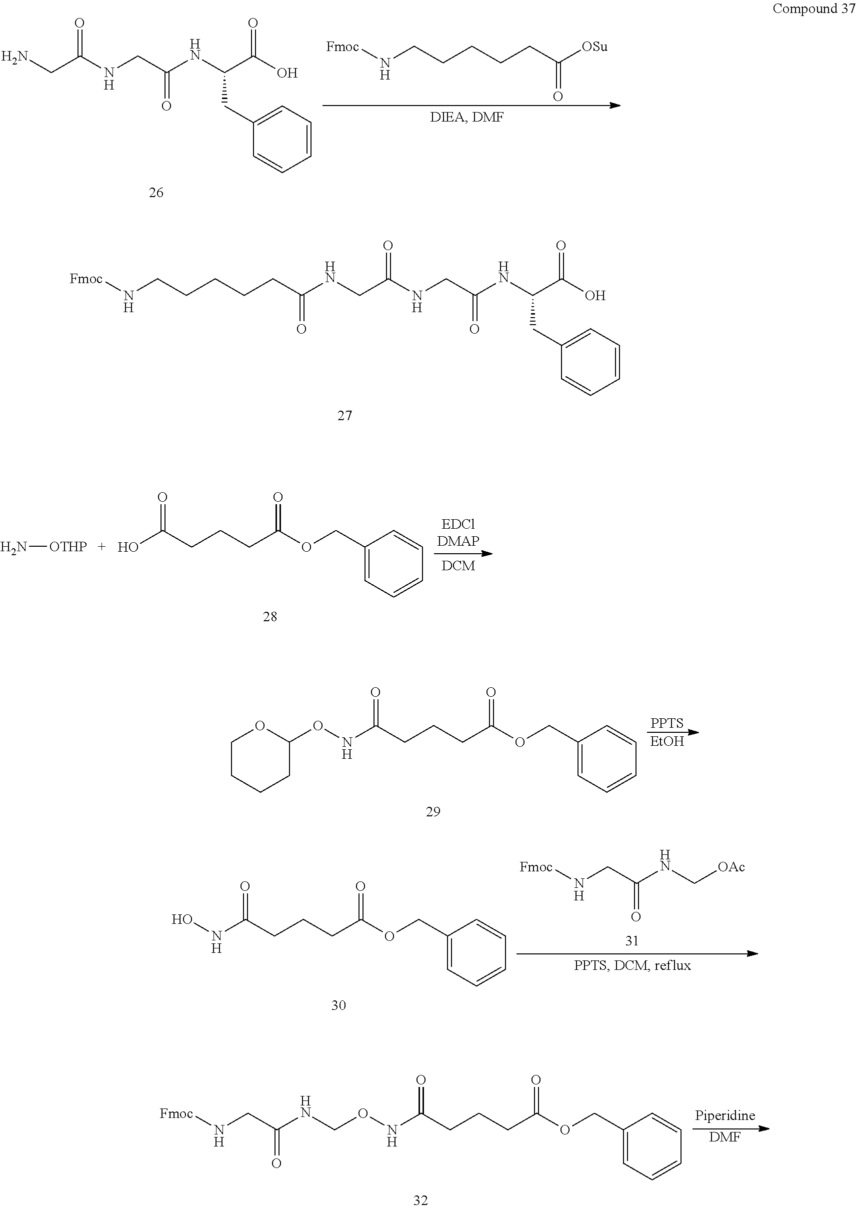

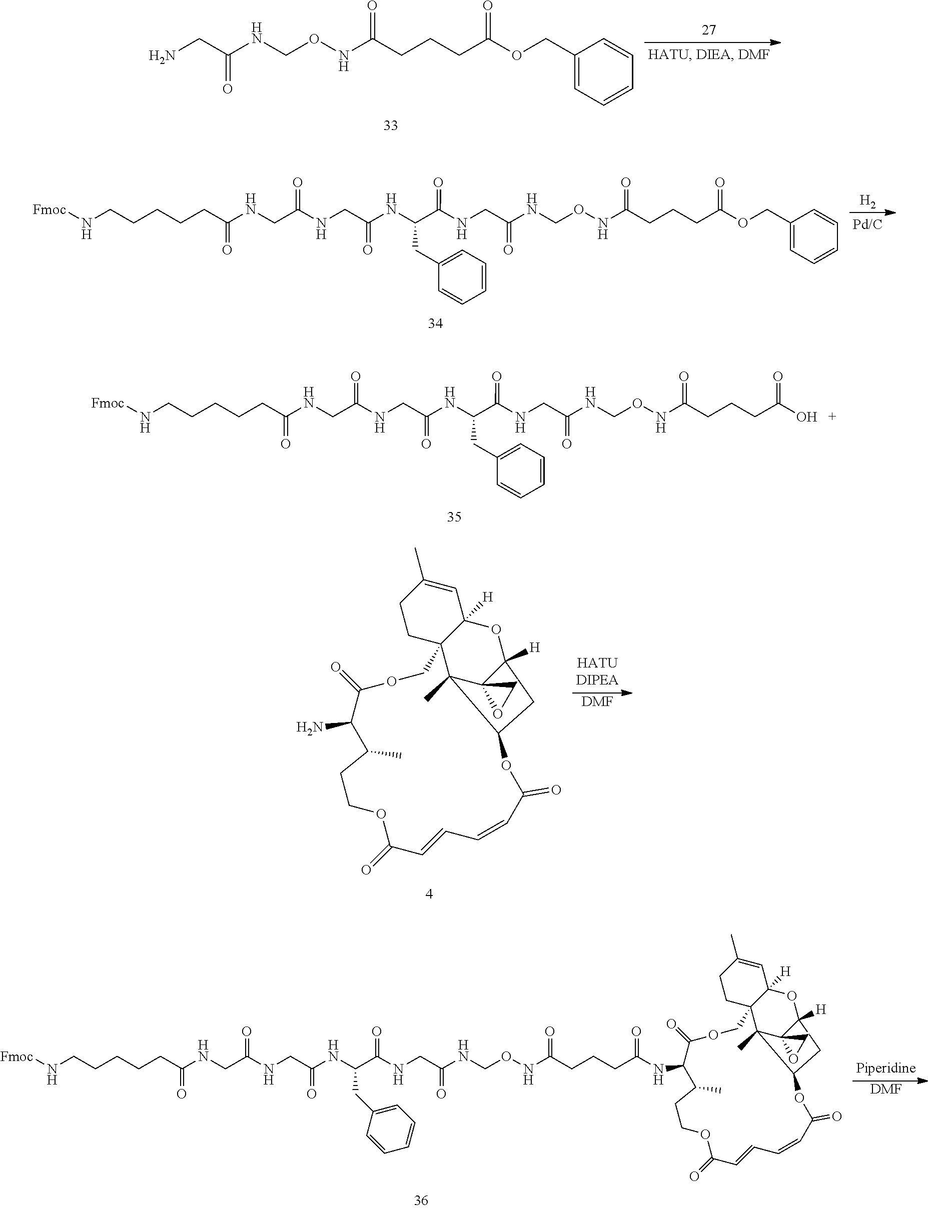

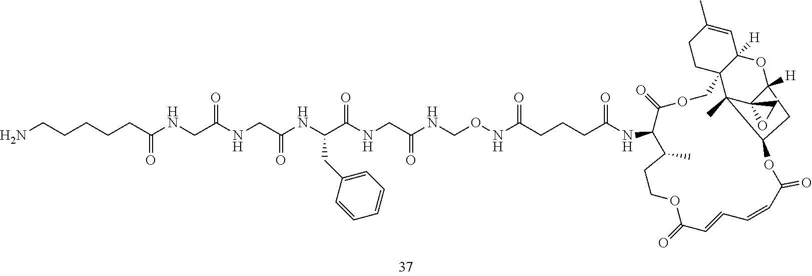 [SMILES: CC1=C[C@H]2O[C@@H]3C[C@H]4OC(=O)/C=C\C=C\C(=O)OCC[C@@H](C)[C@@H](NC(=O)CCCC(=O)NOCNC(=O)CNC(=O)[C@H](Cc5ccccc5)NC(=O)CNC(=O)CNC(=O)CCCCCN)C(=O)OC[C@@]2(CC1)[C@]4(C)[C@]31CO1]

Compound 27: To a solution of compound 26 (100 mg, 0.36 mmol) and Fmoc-6-aminohexanoic acid N-hydroxysuccinimide ester (177.4 mg, 0.39 mmol) in anhydrous DMF (3.5 mL), N,N-diisopropylethylamine (0.19 mL, 1.07 mmol) was added. The reaction was stirred at room temperature for 1 hour. The product was purified by chromatography on a 150 g C18Aq ISCO column, eluting with 0-100% MeCN in H₂O with 0.05% AcOH in both. Pure fractions were combined and lyophilized to afford compound 27 (200 mg, 91%) as a white fluffy solid. MS (ESI, pos.): calc'd for C₃₄H₃₈N₄O₇, 614.6; found 615.6 (M+H).

Compound 29: To a solution of compound 28 (2.00 g, 9.00 mmol), O-(tetrahydro-2H-pyran-2-yl)hydroxylamine (1.27 g, 10.80 mmol) and DMAP (0.11 g, 0.90 mmol) in anhydrous DCM (90 mL), EDCI (2.59 g, 13.50 mmol) was added. The reaction mixture was stirred at room temperature for 2 hours. The product was purified on an 80 g SiO₂ column, eluting with 50-100% EtOAc in hexanes. Pure fractions were combined and concentrated to afford compound 29 (2.8 g, 97%) as a colorless gel. MS (ESI, pos.): calc'd for C₁₇H₂₃NO₅, 321.37; found 344.39 (M+Na).

Compound 32: To a solution of compound 29 (576 mg, 1.79 mmol) in ethanol (6.0 mL), PPTS (225 mg, 0.90 mmol) was added. The reaction mixture was stirred at 60° C. for 2 days until compound 30 was obtained as the major product. The reaction was concentrated in vacuo and the residue was dissolved in DCM (6.0 mL). Compound 31 (660 mg, 1.79 mmol) and PPTS (225 mg, 0.90 mmol) were added. The reaction mixture was stirred at 40° C. for 1 day. The reaction was diluted with EtOAc (20 mL) and washed with sat. NaHCO₃ (20 mL), water (20 mL), then brine (20 mL). The product was purified on a 40 g SiO₂ column (pre-treated with 0.1% Et₃N), eluting with 0-100% EtOAc in DCM. Pure fractions were combined and concentrated to afford compound 32 (293 mg, 30%) as a colorless gel. MS (ESI, pos.): calc'd for C₃₀H₃₁N₃O₇, 545.59; found 546.55 (M+H).

Compound 33: To a solution of compound 32 (200 mg, 0.367 mmol) in DMF (1 mL), was added 5% piperidine solution in DMF (1 mL) and the reaction was stirred for 2 hours at room temperature. The product was purified on a 50 g C18Aq ISCO column, eluting with 0-40% MeCN in H₂O with 10 mM NH₄OAc in both. Pure fractions were combined and lyophilized to afford compound 33 (80.0 mg, 67%) as a colorless gel. MS (ESI, pos.): calc'd for C₁₅H₂₁N₃O₅, 323.35; found 324.34 (M+H).

Compound 34: To a solution of compound 27 (50.0 mg, 0.081 mmol), compound 33 (34.2 mg, 0.106 mmol) and HATU (30.9 mg, 0.081 mmol) in anhydrous DMF (1 mL), N,N-diisopropylethylamine (28.3 μL, 0.163 mmol) was added. The reaction mixture was stirred at room temperature overnight. The product was purified on a 15.5 g C18Aq ISCO column, eluting with 0-80% MeCN in H₂O with 10 mM NH₄OAc in both. Pure fractions were combined and lyophilized to afford compound 34 (41 mg, 55%) as a white fluffy solid. MS (ESI, pos.): calc'd for C₄₉H₅₇N₇O₁₁, 920.03; found 921.13 (M+H).

Compound 35: A solution of compound 34 (35.0 mg, 0.038 mmol) and Pd/C (7 mg, 20% by weight) in absolute ethanol (760 μL) was prepared in an oven-dried round bottom flask. The flask was evacuated and backfilled with H₂ three times. The reaction was stirred at room temperature overnight under a balloon-pressure of H₂. The mixture was filtered through a 0.45 μm syringe filter and the filtrate was concentrated in vacuo. Compound 35 (16 mg, 51%) was obtained as a colorless gel and was used without purification. MS (ESI, pos.): calc'd for C₄₂H₅₁N₇O₁₁, 829.91; found 830.93 (M+H).

Compound 36: To a solution of (R)-amino-verrucarin A (4, 9.0 mg, 0.018 mmol), compound 35 (16.0 mg, 0.019 mmol) and HATU (10.2 mg, 0.027 mmol) in anhydrous DMF (0.4 mL), N,N-diisopropylethylamine (6.3 μL, 0.036 mmol) was added. The reaction mixture was stirred at room temperature overnight. The product was purified on a 5.5 g C18Aq ISCO column, eluting with 0-80% MeCN in H₂O with 10 mM NH₄OAc in both. Pure fractions were combined and lyophilized to afford compound 36 (9 mg, 38%) as a white solid. MS (ESI, pos.): calc'd for C₆₉H₈₄N₈O₁₈, 1313.47; found 1314.50 (M+H).

Compound 37: To a solution of compound 36 (9 mg, 0.0069 mmol) in DMF (0.1 mL), was added 5% piperidine solution in DMF (0.3 mL) and the reaction was stirred for 1 hour at room temperature. The product was purified on a 5.5 g C18Aq ISCO column, eluting with 0-80% MeCN in H₂O with 10 mM NH₄OAc in both. Pure fractions were combined and lyophilized to afford compound 37 (3.1 mg, 41%) as a white fluffy solid. MS (ESI, pos.): calc'd for C₅₄H₇₄N₈O₁₈, 1091.23; found 1092.4 (M+H). ¹H NMR (500 MHz, DMSO-d₆): δ 8.20-8.12 (m, 5H), 8.08-8.04 (m, 1H), 7.95 (dd, J=15.5, 12.5 Hz, 1H), 7.25-7.24 (m, 5H), 7.19-7.17 (m, 1H), 6.87 (t, J=11.5 Hz, 1H), 6.27-6.22 (m, 2H), 5.78-5.75 (m, 1H), 5.29-5.28 (m, 1H), 4.76-4.75 (m, 2H), 4.53-4.49 (m, 1H), 4.42-4.38 (m, 1H), 4.12 (d, J=12.0 Hz, 1H), 4.03 (d, J=12.0 Hz, 1H), 3.96-3.90 (m, 1H), 3.76-3.71 (m, 4H), 3.69-3.66 (m, 5H), 3.60-3.55 (m, 1H), 3.07-3.02 (m, 3H), 2.81-2.80 (m, 1H), 2.59-2.56 (m, 2H), 2.46-2.41 (m, 1H), 2.18-2.11 (m, 5H), 1.99-1.93 (m, 3H), 1.82-1.58 (m, 12H), 1.50-1.46 (m, 2H), 1.40-1.35 (m, 2H), 1.29-1.22 (m, 4H), 0.92 (d, J=7.0 Hz, 3H), 0.75 (s, 3H).

### Example 11

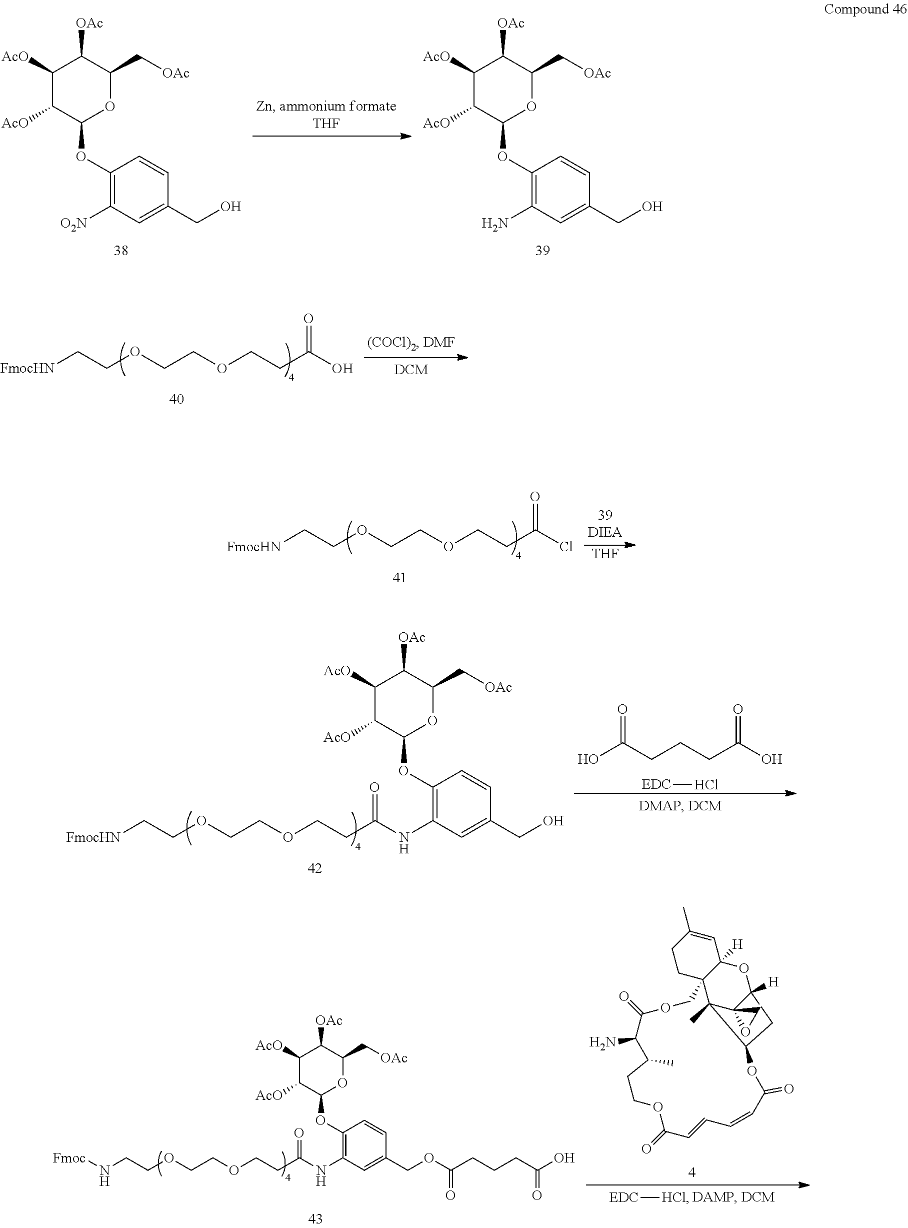

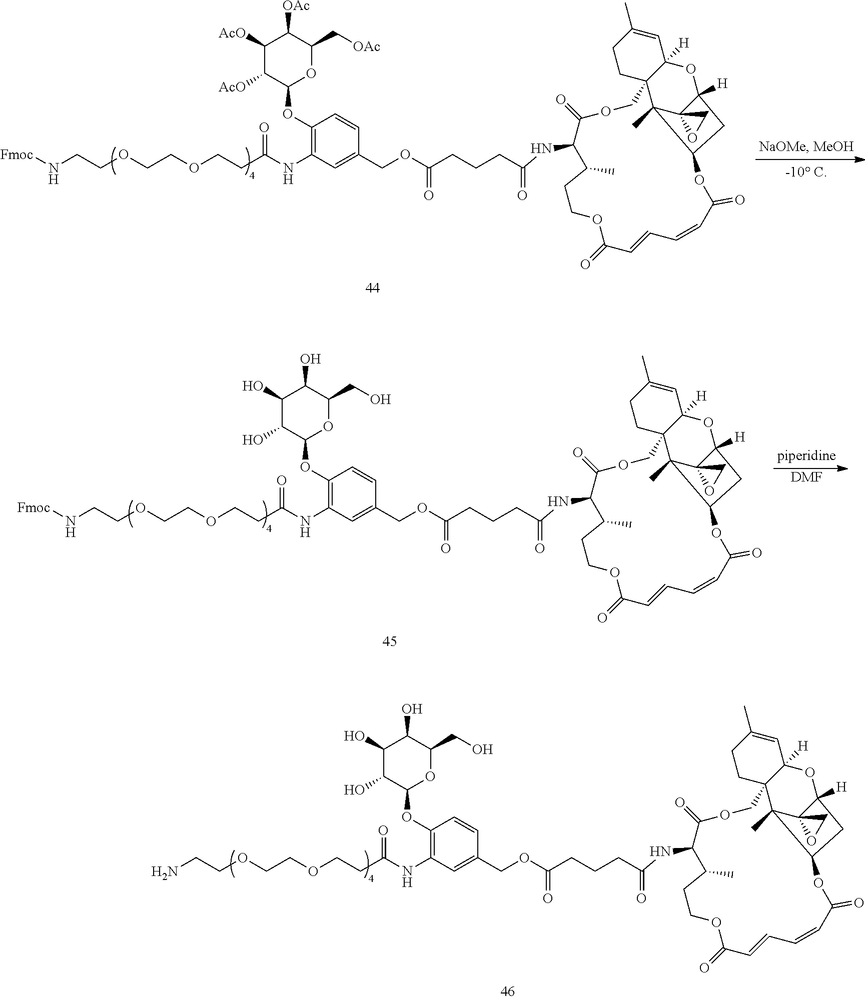

Compound 38 was prepared using the literature procedure in Bioconjugate Chemistry (2016), 27(10), 2549-2557.

Compound 39: Argon was bubbled through a THF (10 mL) solution of compound 38 (150 mg, 0.30 mmol) for 10 minutes. Zinc powder (505 mg, 7.72 mmol) and ammonium formate (60 mg, 0.92 mmol) were added, and the reaction was heated to 65° C. for 16 hours. The reaction was filtered through a pad of celite, and the filtrate was concentrated to afford compound 39 (141 mg, 99%), which was used without purification. MS (ESI, pos.): calc'd for C₂₁H₂₇NO₁₁, 469.1; found 470.2 (M+H).

Compound 42: To a solution of Fmoc-PEG8-amido-COOH (40, 240 mg, 0.36 mmol) in anhydrous DCM (10 mL), oxalyl chloride (62 μL, 0.72 mmol) and DMF (2 μL) were added. After stirring for 30 minutes, volatiles were removed in vacuo to afford Fmoc-PEG8-amido-COCl, 41. In a separate vial, compound 39 (141 mg, 0.30 mmol) was dissolved in anhydrous THF (5 mL) and N,N-diisopropylylethylamine (125 μL, 0.72 mmol) was added followed by the acid chloride (41) in THF (5 mL). After 1 hour, solvents were removed under reduced pressure and the residue was purified on an ISCO 100 g C18Aq column, eluting with 5-95% MeCN/H₂O (both having 0.05% AcOH). Fractions containing the desired product were combined and lyophilized to obtain compound 42 (205 mg, 61%) as a fluffy off-white solid. MS (ESI, pos.): calc'd for C₅₅H₇₄N₂O₂₂, 1114.5; found 1115.4 (M+H).

Compound 43: To a solution of compound 42 (45 mg, 0.0404 mmol), glutaric acid (10 mg, 0.0757 mmol), and DMAP (9.0 mg, 0.0737 mmol) in DCM (0.7 mL) was added EDC-HCl (10 mg, 0.0522 mmol). The yellow solution was stirred at room temperature for 18 hours. The reaction was shown to be complete by LCMS and was concentrated under vacuum. The product was purified by chromatography on a 50 g C18Aq ISCO column, eluting with 25-80% MeCN in H₂O (both containing 0.05% HOAc). Clean fractions were combined and lyophilized to afford compound 43 (40 mg, 81%) as a colorless viscous oil. MS (ESI, pos.): calc'd for C₆₀H₈₀N₂O₂₅, 1228.5; found, 1229.7 (M+H).

Compound 44: To a solution of compound 43 (32 mg, 0.0260 mmol), (R)-amino-Verrucarin (4, 12 mg, 0.0239 mmol), and DMAP (3.2 mg, 0.0263 mmol) in DCM (1 mL) was added EDC-HCl (5.0 mg, 0.0263 mmol). The resulting solution was stirred at room temperature for 2.5 hours, then was diluted with DCM (4 mL) and washed with 0.5 N HCl(aq) (1 mL). The layers were separated, and the aqueous layer was extracted with DCM (2×3 mL). The combined organic layers were washed with sat′d NaHCO₃(aq) then with sat′d brine, then were dried over Na₂SO₄, filtered, and concentrated in vacuo to give a viscous oil. The product was purified by chromatography on a 12 g Silica ISCO column, eluting with 0-30% methanol in DCM to afford compound 44 (29 mg, 71%) as a colorless oil. MS (ESI, pos.): calc'd for C₈₇H₁₁₃N₃O₃₂, 1711.73; found 1713.27 (M+H).

Compound 45: To a −10° C. solution of compound 44 (10 mg, 0.00584 mmol) in methanol (0.9 mL) was added 0.1 M sodium methoxide solution in MeOH (117 μL, 0.0117 mmol) dropwise from a syringe. The reaction was stirred in the cold bath for 1.25 hours then was quenched by the addition of Dowex 50×8 resin (hydrogen form, 200-400 mesh, 30 mg). The mixture was stirred at room temperature 2 minutes, then the solids were removed by filtration through a cotton plug, rinsing with additional methanol. The filtrate was concentrated in vacuo. The product was purified by chromatography on a 5.5 g ISCO C18Aq column, eluting with 10-100% MeCN in H₂O (both containing 0.05% HOAc.) Pure fractions were combined and lyophilized to afford compound 45 (3.2 mg, 35%) as a white fluffy solid. MS (ESI, pos.): calc'd for C₇₉H₁₀₅N₃O₂₈, 1543.7; found 1545.2 (M+H).

Compound 46: To a solution of compound 45 (5.8 mg, 0.0038 mmol) in DMF (360 μL) was added 3.8 μL of a 10% solution of piperidine in DMF (0.38 μL, 0.0038 mmol). The reaction was stirred at room temperature for 6 h, at which time LCMS indicated the reaction was complete. The reaction solution was loaded onto an ISCO 5.5 g C18Aq column and eluted with 0-100% MeCN in H₂O (both containing 0.05% HOAc). Pure fractions were combined and lyophilized to afford compound 46 (2.4 mg, 48%) as a white fluffy solid. MS (ESI, pos.): calc'd for C₆₄H₉₅N₃O₂₆, 1321.6; found 1323.1 (M+H). ¹H NMR (300 MHz; CD₃OD) δ 8.27-8.17 (m, 2H), 7.29 (d, J=8.4 Hz, 1H), 7.12-7.08 (m, 1H), 6.85 (t, J=11.3 Hz, 1H), 6.22 (d, J=11.1 Hz, 1H), 6.14 (d, J=15.6 Hz, 1H), 5.87-5.83 (m, 1H), 5.41-5.39 (m, 1H), 5.16-5.06 (m, 2H), 4.79 (d, J=7.8 Hz, 1H), 4.54-4.48 (m, 1H), 4.37-4.24 (m, 2H), 4.10-4.01 (m, 2H), 3.95-3.75 (m, 10H), 3.69-3.58 (m, 36H), 3.04 (d, J=4.1 Hz, 1H), 2.96 (dd, J=6.5, 3.9 Hz, 2H), 2.81 (d, J=3.8 Hz, 1H), 2.72 (t, J=5.9 Hz, 2H), 2.56-2.43 (m, 3H), 2.43-2.26 (m, 4H), 2.19-2.11 (m, 1H), 1.99-1.89 (m, 4H), 1.85-1.66 (m, 7H), 1.39-1.28 (m, 4H), 1.07 (d, J=6.5 Hz, 3H), 0.89 (s, 3H).

### Example 12

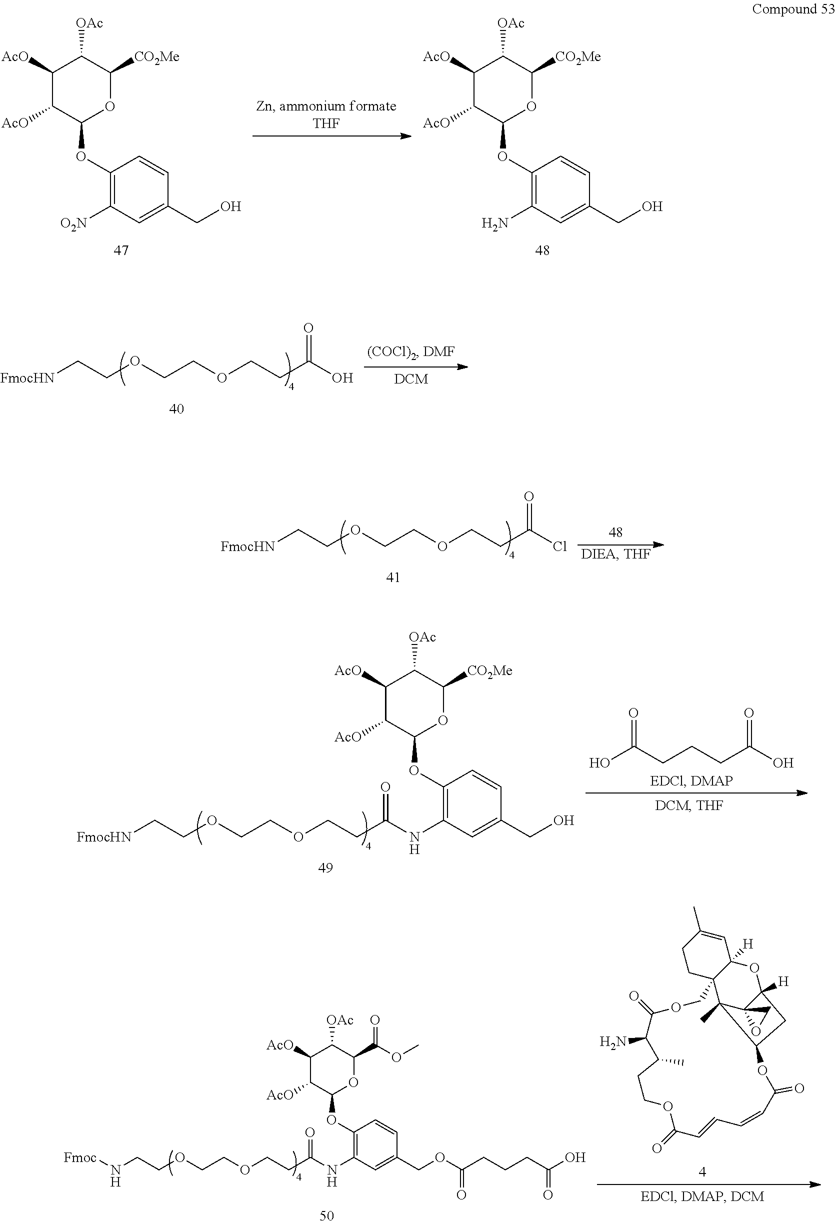

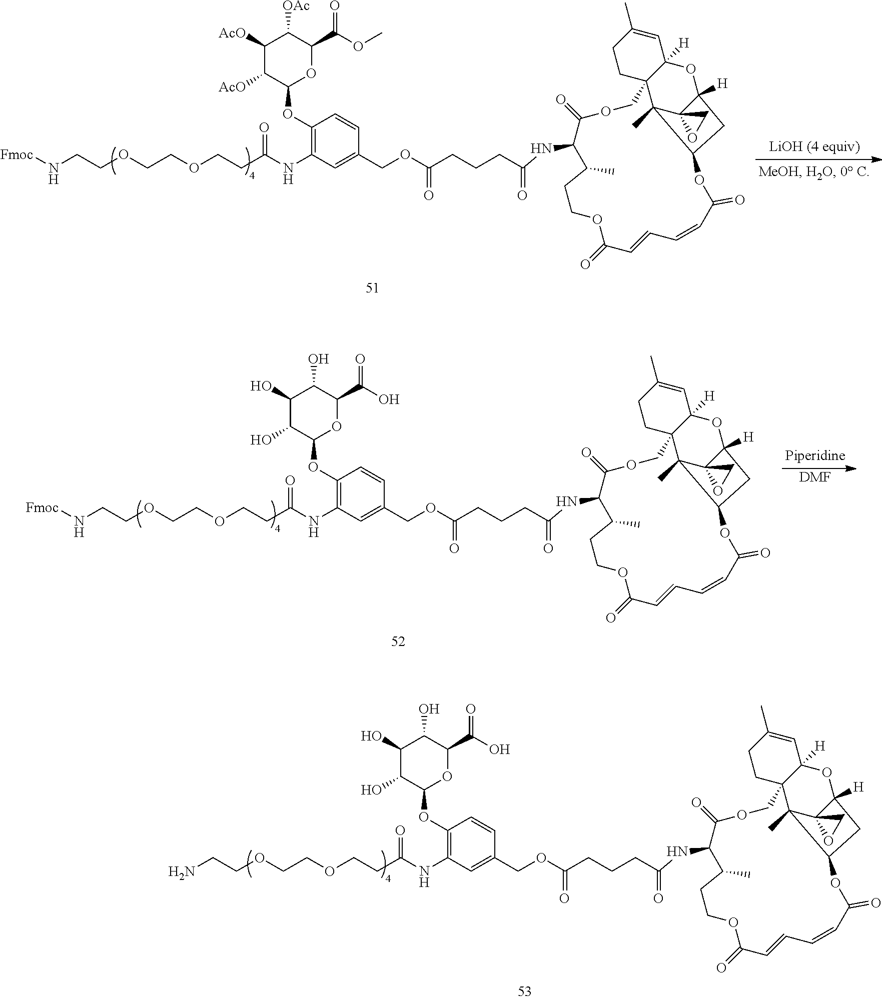

Compound 47 was prepared following the literature procedure from Bioconjugate Chemistry (2016), 27(10), 2549-2557.

Compound 48: Argon was bubbled through a THF (10 mL) solution of compound 47 (80 mg, 0.165 mmol) for 10 mins. Then, zinc powder (268 mg, 4.12 mmol) and ammonium formate (32 mg, 0.495 mmol) were added, and the reaction was heated to 65° C. for 16 hours. The reaction was filtered through a pad of celite, and the filtrate was concentrated to afford compound 48 (75 mg, 99%), which was used without purification. MS (ESI, pos.): calc'd for C₂₀H₂₅NO₁₁, 455.1; found 456.1 (M+H).

Compound 49: To a solution of Fmoc-PEG8-amido-COOH (40, 142 mg, 0.215 mmol) in anhydrous DCM (10 mL), oxalyl chloride (37 μL, 0.430 mmol) and DMF (2 μL) were added. After stirring for 30 minutes, volatiles were removed in vacuo to obtain Fmoc-PEG8-amido-COCl (41). In a separate vial, compound 48 (75 mg, 0.165 mmol) was dissolved in anhydrous THF (5 mL) and N,N-diisopropylylethylamine (73 μL, 0.430 mmol) was added, followed by compound 41 in THF (5 mL). After 1 hour, volatiles were removed under reduced pressure and the residue was purified on an ISCO 100 g C18Aq column, eluting with 5-95% MeCN/H₂O (both having 0.05% AcOH). Fractions containing the desired product were combined and lyophilized to afford compound 49 (47.1 mg, 25%) as a fluffy white solid. MS (ESI, pos.): calc'd for C₅₄H₇₂N₂O₂₂, 1100.5; found 1101.4 (M+H).

Compound 50: To a solution of compound 48 (56 mg, 0.051 mmol), glutaric acid (67 mg, 0.509 mmol) and DMAP (9.3 g, 0.076 mmol) in 1:1 mixture of anhydrous DCM and THF (5 mL), EDCI (58.5 mg, 0.305 mmol) was added. The reaction mixture was stirred at room temperature overnight. The product was purified on a 15.5 g C18Aq ISCO column, eluting with 0-100% MeCN in H₂O with 0.05% AcOH in both. Pure fractions were combined and lyophilized to afford compound 50 (37.5 mg, 61%) as a white solid. MS (ESI, pos.): calc'd for C₅₉H₇₈N₂O₂₅, 1215.26; found 1216.45 (M+H).

Compound 51: To a solution of compound 50 (17 mg, 0.014 mmol), (R)-amino-verrucarin A (4, 7.0 mg, 0.014 mmol) and DMAP (0.9 mg, 0.007 mmol) in anhydrous DCM (2.3 mL), EDCI (8.1 mg, 0.042 mmol) was added. The reaction mixture was stirred at room temperature for 1 hour. The product was purified on a 5.5 g C18Aq ISCO column, eluting with 0-100% MeCN in H₂O with 0.05% AcOH in both. Pure fractions were combined and lyophilized to afford compound 51 (20.5 mg, 86%) as a white solid. MS (ESI, pos.): calc'd for C₈₆H₁₁₁N₃O₃₂, 1698.82; found 1699.81 (M+H).

Compound 52: A solution of compound 51 (20.5 mg, 0.012 mmol) in 2:1 mixture of MeOH and H₂O (4.5 mL) was cooled to 0° C. in an ice water bath. 0.1 M LiOH solution in H₂O (483 μL) was added dropwise and the reaction mixture was stirred for 30 minutes while maintaining the temperature at 0° C. A drop of AcOH was added to quench the reaction. The product was purified on a 15.5 g C18Aq ISCO column, eluting with 0-100% MeCN in H₂O with 0.05% AcOH in both. Pure fractions were combined and lyophilized to afford compound 52 (6.8 mg, 30%) as a white solid. MS (ESI, pos.): calc'd for C₇₉H₁₀₃N₃O₂₉, 1558.69; found 1559.63 (M+H).

Compound 53: To a solution of compound 52 (6.8 mg, 0.004 mmol) in DMF (121 μL), was added 5% piperidine solution in DMF (24 μL) and the reaction was stirred for 1 hour at room temperature. The product was purified by Teledyne ISCO EZ prep on a 30×150 mm Gemini column, eluting with 0-60% MeCN in H₂O with 0.05% AcOH in both. Pure fractions were combined and lyophilized to afford compound 53 (3.1 mg, 53%) as a white solid. MS (ESI, pos.): calc'd for C₆₄H₉₃N₃O₂₇, 1336.44; found 1337.4 (M+H). ¹H NMR (500 MHz, MeOD): δ 8.22-8.17 (m, 2H), 7.35 (d, J=8.5 Hz, 1H), 7.08 (dd, J=8.5, 1.5 Hz, 1H), 6.82 (t, J=11.0 Hz, 1H), 6.18 (d, J=11.5 Hz, 1H), 6.11 (d, J=15.5 Hz, 1H), 5.82 (dd, J=8.0, 3.5 Hz, 1H), 5.38-5.37 (m, 1H), 5.08 (br, 2H), 4.85 (br, 1H), 4.78 (d, J=7.0 Hz, 1H), 4.49-4.46 (m, 1H), 4.32 (d, J=12.5 Hz, 1H), 4.23 (d, J=12.5 Hz, 1H), 4.03 (td, J=11.5, 3.0 Hz, 1H), 3.89-3.83 (m, 3H), 3.76-3.70 (m, 5H), 3.67-3.59 (m, 34H), 3.53-3.51 (m, 3H), 3.10 (t, J=10.5 Hz, 2H), 3.02 (d, J=4.0 Hz, 1H), 2.81 (d, J=3.5 Hz, 1H), 2.72 (t, J=5.0 Hz, 2H), 2.52-2.23 (m, 8H), 2.12 (dt, J=15.5, 5.0 Hz, 1H), 1.94-1.89 (m, 3H), 1.81-1.73 (m, 3H), 1.67 (s, 3H), 1.35-1.29 (m, 1H), 1.05 (d, J=6.5 Hz, 3H), 0.88 (s, 3H).

### Example 13

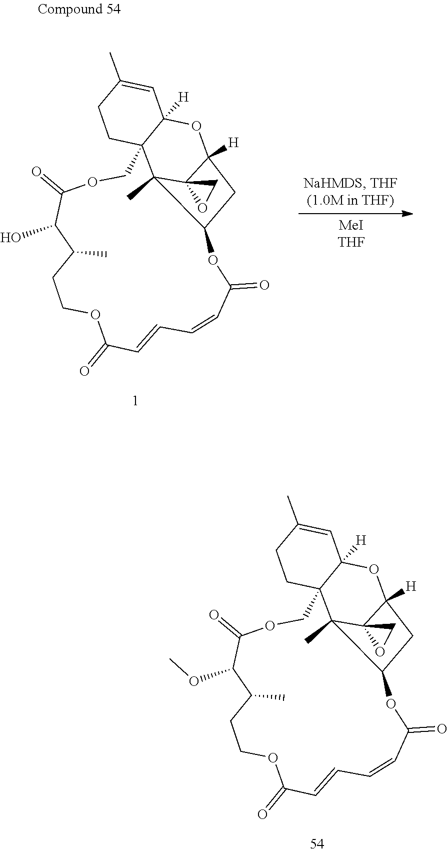 [SMILES: "MeITHF" ; CC1=C[C@H]2O[C@@H]3C[C@H]4OC(=O)/C=C\C=C\C(=O)OCC[C@@H](C)[C@H](O)C(=O)OC[C@@]2(CC1)[C@]4(C)[C@]31CO1 ; "NaHMDS, THF(1.0M in THF)" ; CO[C@@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1]

Compound 54: To a 0° C. solution of Verrucarin A (1, 5 mg, 0.01 mmol) in anhydrous THF (1 mL) was added NaHMDS (11 μL, 1.0 M in THF, 0.011 mmol). The yellow solution was stirred for 5 minutes, then methyl iodide (6.1 μL, 0.1 mmol) was added. The reaction was stirred at 0° C. for 30 minutes, at which time LC/MS indicated the reaction was complete. The reaction was diluted with saturated aqueous ammonium chloride solution (1 mL) and water (1 mL). The mixture was extracted with dichloromethane (3×3 mL). The combined organics were washed with brine, then dried over anhydrous sodium sulfate, filtered, and concentrated. The residue was purified by chromatography on an ISCO 4 g silica gold column using 35% ethyl acetate/hexanes to afford compound 54 (5 mg, 97%). MS (ESI, pos.): calc'd for C₂₈H₃₆O₉, 516.2; found 517.3 (M+H), 539.2 (M+Na). ¹H-NMR (500 MHz; CDCl₃): δ 8.02 (dd, J=15.6, 11.7 Hz, 1H), 6.67 (t, J=11.3 Hz, 1H), 6.16 (d, J=11.0 Hz, 1H), 6.05 (d, J=15.7 Hz, 1H), 5.80 (dd, J=7.9, 3.8 Hz, 1H), 5.45-5.44 (m, 1H), 4.72 (d, J=12.2 Hz, 1H), 4.48-4.46 (m, 1H), 4.20 (d, J=12.2 Hz, 1H), 4.02 (td, J=11.4, 2.9 Hz, 1H), 3.87 (d, J=5.0 Hz, 1H), 3.69 (s, 1H), 3.59 (d, J=5.0 Hz, 1H), 3.38 (s, 3H), 3.13 (d, J=3.8 Hz, 1H), 2.82 (d, J=3.8 Hz, 1H), 2.50 (dd, J=15.4, 8.2 Hz, 1H), 2.35-2.33 (m, 1H), 2.23 (dt, J=15.4, 4.5 Hz, 1H), 2.02-1.83 (m, 3H), 1.77 (s, 3H), 1.56 (s, 3H), 0.98 (d, J=6.9 Hz, 3H), 0.87 (s, 3H).

### Example 14

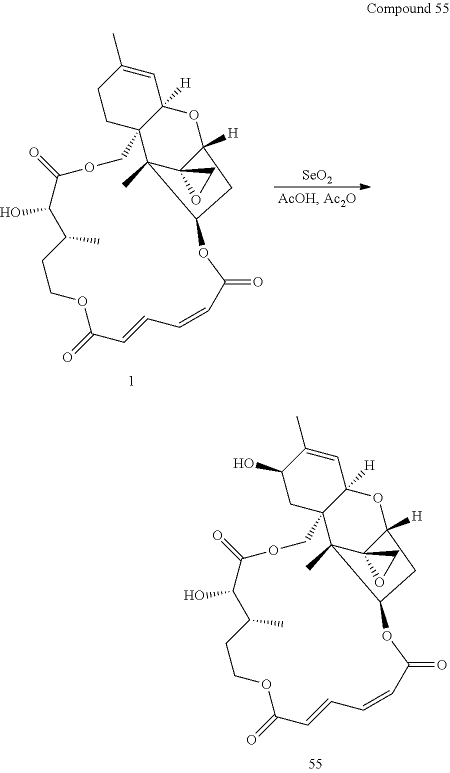

Compound 55: A solution of Verrucarin A (1, 5 mg, 0.01 mmol) and SeO₂ in 1:1 v/v AcOH—Ac₂O (1 mL) was refluxed for 1 hour. The reaction was cooled to ambient temperature and concentrated to dryness. The residue was purified by prep HPLC on a 30×150 mm Gemini column using 5-95% MeCN/H₂O (both having 0.05% AcOH). Pure fractions were combined and lyophilized to obtain compound 55 (3 mg, 58%) as a fluffy off-white solid. MS (ESI, pos.): calc'd for C₂₇H₃₄O₁₀, 518.2; found 519.2 M+H). ¹H-NMR (500 MHz; CDCl₃): δ 8.05 (dd, J=15.2, 12.0 Hz, 1H), 6.69 (t, J=11.3 Hz, 1H), 6.16 (d, J=11.1 Hz, 1H), 6.06 (d, J=15.5 Hz, 1H), 5.77 (dd, J=8.1, 4.0 Hz, 1H), 5.53 (d, J=4.9 Hz, 1H), 4.73 (d, J=12.2 Hz, 1H), 4.54-4.51 (m, 1H), 4.29 (d, J=12.0 Hz, 1H), 4.18 (d, J=1.1 Hz, 1H), 4.04-3.97 (m, 2H), 3.90 (d, J=5.1 Hz, 1H), 3.59 (d, J=4.8 Hz, 1H), 3.15 (d, J=3.7 Hz, 1H), 2.85 (d, J=3.7 Hz, 1H), 2.48 (dd, J=15.3, 7.8 Hz, 1H), 2.39-2.35 (m, 1H), 2.26 (m, 1H), 2.18 (dd, J=8.7, 3.2 Hz, 1H), 2.11 (dd, J=12.5, 6.4 Hz, 1H), 1.99-1.93 (m, 1H), 1.89-1.86 (m, 4H), 1.85-1.77 (m, 2H), 0.89 (d, J=6.7 Hz, 3H), 0.87 (s, 3H).

### Example 15

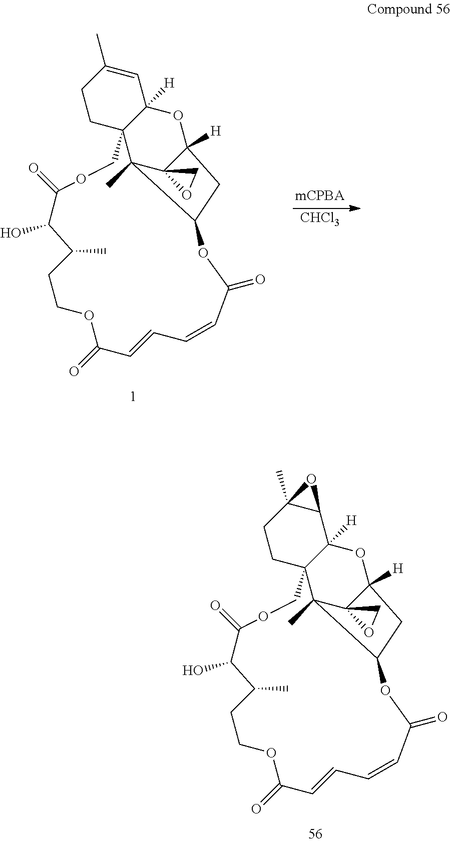 [SMILES: CC1=C[C@H]2O[C@@H]3C[C@H]4OC(=O)/C=C\C=C\C(=O)OCC[C@@H](C)[C@H](O)C(=O)OC[C@@]2(CC1)[C@]4(C)[C@]31CO1 ; "CHCl3" ; C[C@@H]1CCOC(=O)/C=C/C=C\C(=O)O[C@@H]2C[C@H]3O[C@@H]4[C@@H]5O[C@]5(C)CC[C@]4(COC(=O)[C@H]1O)[C@]2(C)[C@]31CO1]

Compound 56: To a solution of Verrucarin A (1, 5 mg, 0.01 mmol) in anhydrous chloroform (0.5 mL), m-CPBA (1.9 mg, 0.011 mmol) was added. The reaction was stirred for 24 hours then volatiles were removed under reduced pressure. The residue was purified by prep HPLC on a 30×150 mm Gemini column using 5-95% MeCN/H₂O (both having 0.05% AcOH). Pure fractions were combined and lyophilized to obtain compound 56 (3.5 mg, 68%) as a fluffy off-white solid. MS (ESI, pos.): calc'd for C₂₇H₃₄O₁₀, 518.2; found 519.2 M+H). ¹H-NMR (500 MHz; CDCl₃): δ 8.06 (dd, J=15.6, 11.8 Hz, 1H), 6.69 (t, J=11.3 Hz, 1H), 6.15 (d, J=11.1 Hz, 1H), 6.05 (d, J=15.7 Hz, 1H), 5.78 (dd, J=7.9, 4.0 Hz, 1H), 4.67 (d, J=12.1 Hz, 1H), 4.54-4.51 (m, 1H), 4.28 (d, J=12.2 Hz, 1H), 4.16 (s, 1H), 4.00-3.96 (m, 1H), 3.96 (d, J=4.8 Hz, 1H), 3.55 (d, J=4.2 Hz, 1H), 3.17 (d, J=3.6 Hz, 1H), 3.12 (d, J=5.2 Hz, 1H), 2.76 (d, J=3.5 Hz, 1H), 2.67-2.64 (m, 1H), 2.46 (dd, J=15.4, 8.3 Hz, 1H), 2.36 (t, J=5.8 Hz, 1H), 2.24 (dt, J=15.3, 4.4 Hz, 1H), 2.19 (s, 1H), 1.98-1.91 (m, 2H), 1.83-1.70 (m, 2H), 1.59-1.52 (m, 1H), 1.41 (s, 3H), 0.91 (d, J=6.7 Hz, 3H), 0.80 (s, 3H).

### Example 16

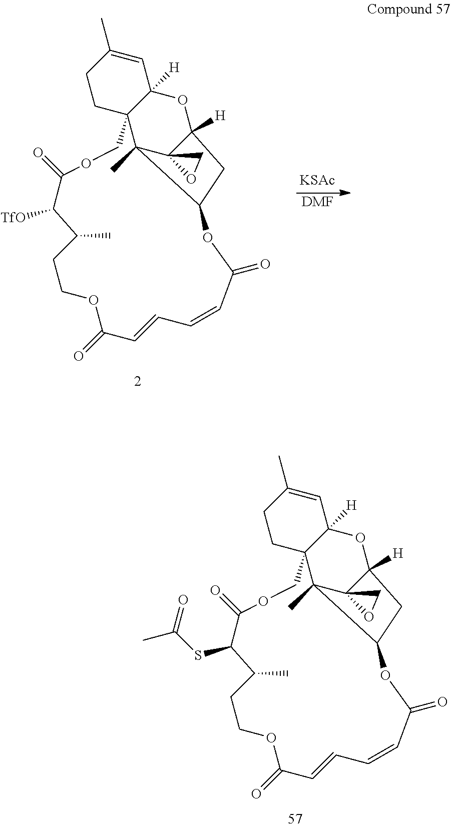

Compound 57: To a solution of the Verrucarin A triflate (2, 5.0 mg, 0.0079 mmol) in DMF (0.1 mL) under argon, was added potassium thioacetate (2.7 mg, 0.0236 mmol). The reaction was stirred at room temperature for 20 minutes, at which time LCMS indicated complete consumption of starting material. The reaction was diluted with EtOAc (1 mL) and washed with 1:1 brine/H₂O (1 mL). The aqueous layer was extracted with EtOAc (2×1 mL). The combined organic layers were washed with brine (1 mL), then were dried over Na₂SO₄, filtered, and concentrated in vacuo. Chromatography on an ISCO 4 g Silica Gold column, eluting with EtOAc/hexanes (0-100%) provided compound 57 (4.4 mg, quant.) as a white solid. MS (ESI, pos.): calc'd for C₂₉H₃₆O₉S, 560.21; found 561.2 (M+H). ¹H-NMR (500 MHz; CDCl₃): δ 8.05-8.00 (m, 1H), 6.69-6.64 (m, 1H), 6.15-6.13 (m, 1H), 6.03 (d, J=15.5 Hz, 1H), 5.78-5.76 (m, 1H), 5.41 (d, J=1.3 Hz, 1H), 4.55 (d, J=11.8 Hz, 1H), 4.49-4.46 (m, 1H), 4.20 (d, J=12.0 Hz, 1H), 3.98-3.95 (m, 2H), 3.85 (s, 1H), 3.56 (t, J=0.6 Hz, 1H), 3.13 (s, 1H), 2.85 (s, 1H), 2.52-2.47 (m, 1H), 2.37 (s, 3H), 2.31-2.26 (m, 1H), 2.22-2.16 (m, 2H), 1.98-1.83 (m, 3H), 1.80-1.74 (m, 4H), 1.33-1.25 (m, 1H), 1.13 (d, J=5.4 Hz, 3H), 0.89-0.86 (m, 3H).

### Example 17

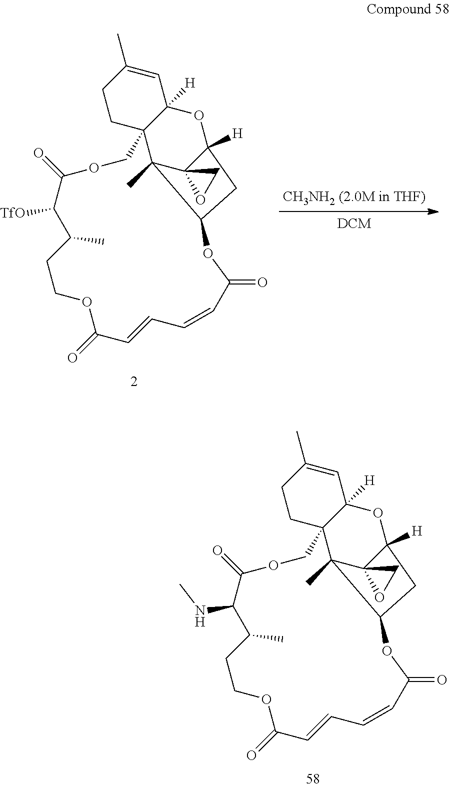 [SMILES: CC1=C[C@H]2O[C@@H]3C[C@H]4OC(=O)/C=C\C=C\C(=O)OCC[C@@H](C)[C@H](OS(=O)(=O)C(F)(F)F)C(=O)OC[C@@]2(CC1)[C@]4(C)[C@]31CO1 ; CN[C@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1]

Compound 58: To a solution of Verrucarin A triflate (2, 8 mg, 0.012 mmol) in DCM (2 mL) at room temperature was added methylamine (2 M in THF, 2 mL, 4 mmol). The reaction was stirred for 30 minutes, at which time LCMS analysis showed -40% conversion to the desired product. The reaction was concentrated in vacuo, re-dissolved in DCM (2 mL) and additional methylamine solution (2 mL) was added. The reaction was stirred for 2 hours, then the process was repeated. At that time LCMS indicated the reaction had reached completion. Volatiles were removed in vacuo. The residue was dissolved in DMF (0.5 mL), injected onto an ISCO 5.5 g C18Aq column, and eluted with 5-95% MeCN/H₂O (both having 0.05% AcOH). Pure fractions were combined and lyophilized and to obtain compound 58 (3.2 mg, 49%) as a white fluffy solid. MS (ESI, pos.): calc'd for C₂₈H₃₇NO₈, 515.3; found 516.3 (M+H). ¹H-NMR (500 MHz; CDCl₃): δ 8.11 (dd, J=15.5, 11.6 Hz, 1H), 6.70 (t, J=11.3 Hz, 1H), 6.15 (d, J=11.1 Hz, 1H), 6.06 (d, J=15.6 Hz, 1H), 5.84-5.82 (m, 1H), 5.46 (dd, J=3.1, 1.0 Hz, 1H), 4.65 (d, J=12.2 Hz, 1H), 4.55-4.52 (m, 1H), 4.17 (d, J=12.3 Hz, 1H), 4.04-3.99 (m, 1H), 3.89 (d, J=5.1 Hz, 1H), 3.61 (d, J=4.6 Hz, 1H), 3.15 (d, J=3.8 Hz, 1H), 2.88-2.84 (m, 2H), 2.54-2.49 (m, 3H), 2.42 (s, 3H), 2.27-2.22 (m, 1H), 2.00-1.95 (m, 4H), 1.77-1.75 (m, 1H), 1.46 (s, 3H), 1.28-1.24 (m, 1H), 1.03 (d, J=6.6 Hz, 3H), 0.90 (s, 3H).

### Example 18

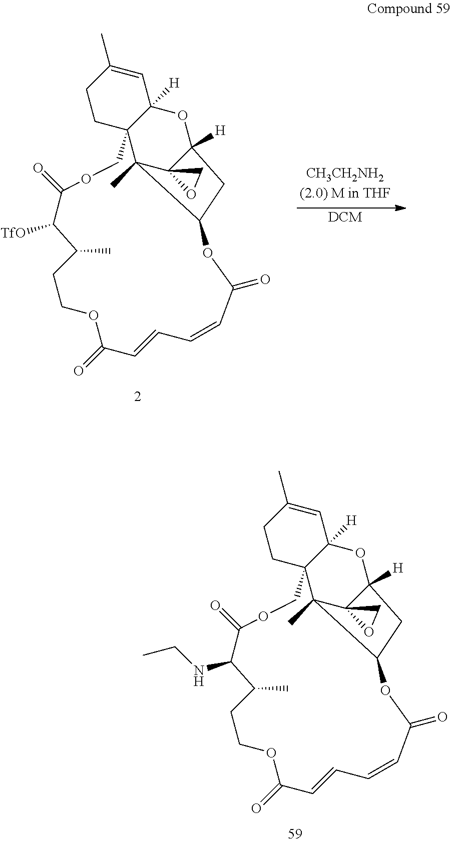

Compound 59: To a solution of Verrucarin A triflate (2, 6.3 mg, 0.01 mmol) in THF (1 mL) at room temperature, ethylamine (2 M in THF, 1 mL) was added, and the reaction was stirred for 18 hours, at which time LCMS indicated complete consumption of the triflate. Volatiles were removed in vacuo. The residue was dissolved in DMF (0.5 mL) and injected onto an ISCO 5.5 g C18Aq column and eluted with 5-95% MeCN/H₂O (both having 0.05% AcOH). Pure fractions were combined and lyophilized and to obtain compound 59 (3.4 mg, 64%) as a white fluffy solid. MS (ESI, pos.): calc'd for C29H39N08, 529.3; found 530.3 (M+H). ¹H-NMR (500 MHz; CDCl₃): δ 8.11 (dd, J=15.7, 11.8 Hz, 1H), 6.70 (t, J=11.3 Hz, 1H), 6.15 (d, J=11.0 Hz, 1H), 6.06 (d, J=15.7 Hz, 1H), 5.83 (dd, J=7.9, 3.8 Hz, 1H), 5.46 (d, J=4.1 Hz, 1H), 4.62 (d, J=12.2 Hz, 1H), 4.55-4.52 (m, 1H), 4.17 (d, J=12.3 Hz, 1H), 4.02 (d, J=2.1 Hz, 1H), 3.89 (d, J=5.0 Hz, 1H), 3.61 (d, J=4.7 Hz, 1H), 3.15 (d, J=3.9 Hz, 1H), 2.95 (d, J=10.0 Hz, 1H), 2.84 (d, J=3.9 Hz, 1H), 2.63-2.49 (m, 4H), 2.25-2.23 (m, 1H), 2.02-1.95 (m, 4H), 1.77 (s, 3H), 1.69 (d, J=8.1 Hz, 1H), 1.26-1.23 (m, 1H), 1.12 (t, J=7.1 Hz, 3H), 1.02 (d, J=6.6 Hz, 3H), 0.90 (s, 3H).

### Example 19

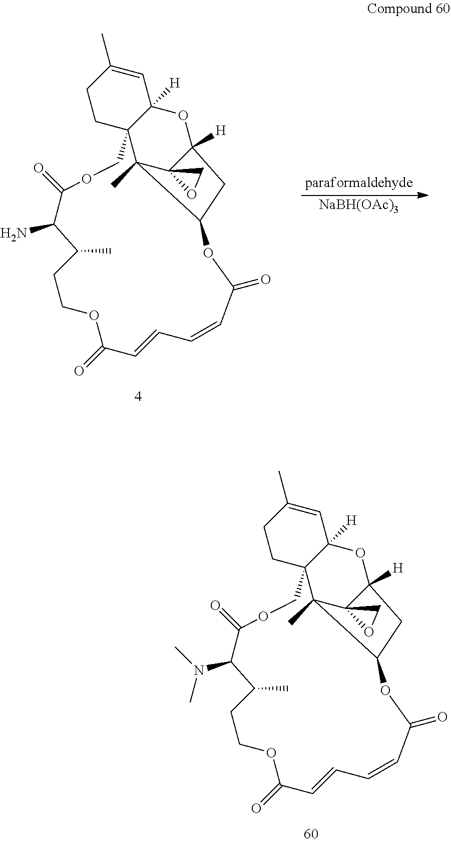 [SMILES: CC1=C[C@H]2O[C@@H]3C[C@H]4OC(=O)/C=C\C=C\C(=O)OCC[C@@H](C)[C@@H](N)C(=O)OC[C@@]2(CC1)[C@]4(C)[C@]31CO1 ; CC1=C[C@H]2O[C@@H]3C[C@H]4OC(=O)/C=C\C=C\C(=O)OCC[C@@H](C)[C@@H](N(C)C)C(=O)OC[C@@]2(CC1)[C@]4(C)[C@]31CO1 ; "NaBH(OAc)3"]

Compound 60: To a solution of (R)-amino-verrucarin A acetic acid salt (4, 7 mg, 0.012 mmol) in DCE (1 mL), were added paraformaldehyde (3.6 mg, 0.12 mmol) and sodium triacetoxyborohydride (25 mg, 0.12 mmol). The reaction was heated to 60° C. for 5 hours, at which time LCMS indicated the reaction was complete. Volatiles were removed in vacuo. The residue was dissolved in DMF (0.5 mL) and injected onto an ISCO 5.5 g C18Aq column and eluted with 5-95% MeCN/H₂O (both having 0.05% AcOH). Pure fractions were combined and lyophilized to obtain compound 60 (2.0 mg, 29%) as a white fluffy solid. MS (ESI, pos.): calc'd for C₂₉H₃₉NO₈, 529.3; found 530.3 (M+H). ¹H-NMR (500 MHz; CDCl₃): δ 8.08 (ddd, J=15.7, 11.6, 1.0 Hz, 1H), 6.68 (t, J=11.3 Hz, 1H), 6.15 (d, J=11.1 Hz, 1H), 6.05 (d, J=15.7 Hz, 1H), 5.83 (dd, J=8.1, 3.8 Hz, 1H), 5.44 (dd, J=5.3, 1.2 Hz, 1H), 4.76 (d, J=12.3 Hz, 1H), 4.52 (ddd, J=11.1, 6.1, 1.9 Hz, 1H), 4.09 (d, J=12.3 Hz, 1H), 4.03 (td, J=11.6, 3.9 Hz, 1H), 3.87 (d, J=5.2 Hz, 1H), 3.58 (d, J=5.3 Hz, 1H), 3.12 (d, J=4.0 Hz, 1H), 2.99 (d, J=11.0 Hz, 1H), 2.82 (d, J=4.0 Hz, 1H), 2.48 (dd, J=15.5, 8.2 Hz, 1H), 2.34 (s, 6H), 2.31-2.21 (m, 3H), 2.01-1.95 (m, 3H), 1.83-1.81 (m, 1H), 1.77 (s, 3H), 1.26-1.19 (m, 1H), 1.02 (d, J=6.5 Hz, 3H), 0.87 (s, 3H).

### Example 20

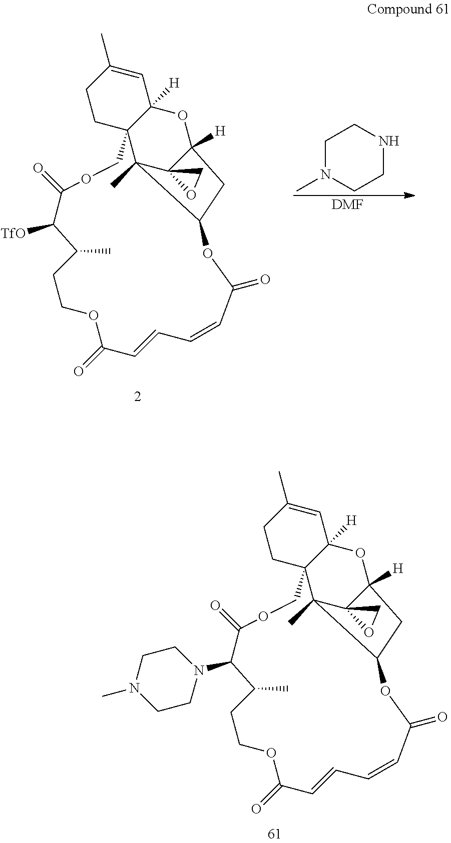

Compound 61: To a solution of Verrucarin A triflate (2, 10 mg, 0.0158 mmol) in DMF (100 mL) was added N-methylpiperazine (9 mL, 0.0811 mmol). The resulting solution was stirred for 3.5 days, at which time LCMS indicated the reaction was complete. The reaction solution was loaded onto an ISCO 5.5 g C18 column and eluted with 0-100% MeCN in H₂O (both containing 0.05% HOAc). Fractions that were one peak by LCMS were combined and lyophilized. ¹H NMR analysis indicated the product was not pure. The impure product was chromatographed on an ISCO 4 g Silica column, eluting with a gradient of MeOH/DCM (0-10%). Fractions containing pure product were combined and concentrated to afford compound 61 as a white solid (1.8 mg, 20%). MS (ESI, pos.): calc'd for C₃₂H₄₄N₂O₈, 584.3; found 585.3 (M+H). ¹H NMR (300 MHz; CDCl₃) δ 8.09 (dd, J=15.7, 11.6 Hz, 1H), 6.67 (t, J=11.5 Hz, 1H), 6.14 (d, J=11.1 Hz, 1H), 6.04 (d, J=15.7 Hz, 1H), 5.82 (dd, J=8.1, 3.8 Hz, 1H), 5.42-5.41 (m, 1H), 4.80 (d, J=12.3 Hz, 1H), 4.53-4.48 (m, 1H), 4.08-3.96 (m, 2H), 3.85 (d, J=5.0 Hz, 1H), 3.65-3.51 (m, 2H), 3.10 (d, J=3.9 Hz, 1H), 2.99 (d, J=11.2 Hz, 1H), 2.83 (d, J=3.8 Hz, 1H), 2.66-2.52 (m, 4H), 2.52-2.29 (m, 6H), 2.28-2.22 (m, 4H), 2.22-2.11 (m, 2H), 2.05-1.77 (m, 6H), 1.75 (s, 3H), 0.99 (d, J=6.4 Hz, 3H), 0.85 (s, 3H).

### Example 21

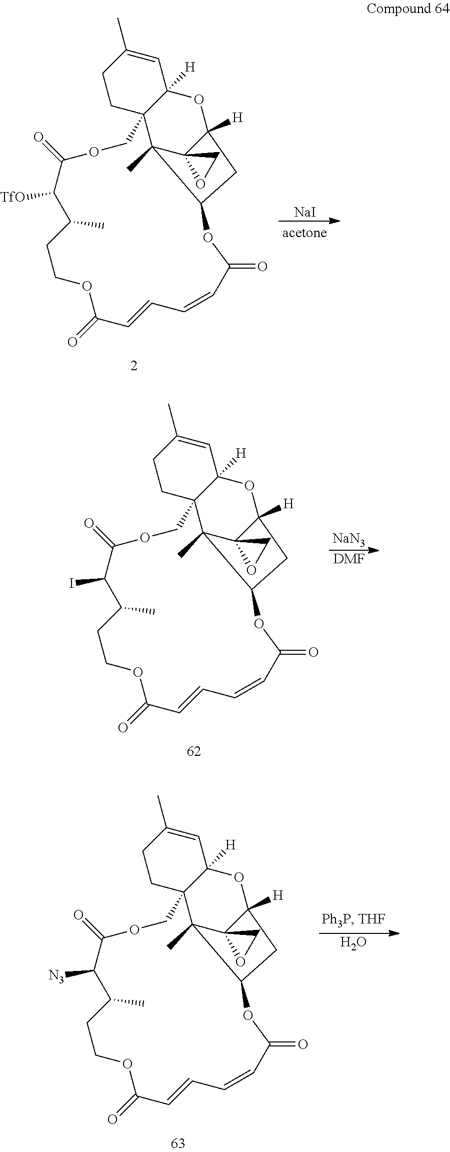

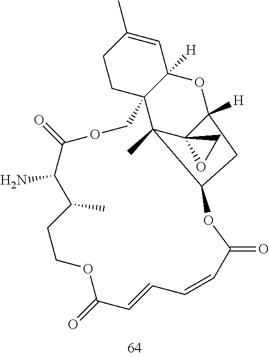 [SMILES: CC1=C[C@H]2O[C@@H]3C[C@H]4OC(=O)/C=C\C=C\C(=O)OCC[C@@H](C)[C@H](N)C(=O)OC[C@@]2(CC1)[C@]4(C)[C@]31CO1]

Compound 62: To a solution of the Verrucarin A triflate (2, 24 mg, 0.0378 mmol) in acetone (0.4 mL) was added sodium iodide (9 mg, 0.0600 mmol). The reaction was stirred at room temperature for 12.5 hours, at which time LCMS indicated the reaction was complete. The reaction was diluted with EtOAc (2 mL) and washed with H₂O (1 mL). The aqueous layer was extracted with EtOAc (3×1 mL). The combined organic layers were washed with brine (2 mL), then were dried over Na₂SO₄, filtered, and concentrated in vacuo. Chromatography on an ISCO 5.5 g C18Aq column, eluting with 20-80% MeCN in H₂O (both containing 0.05% HOAc), afforded compound 62 (11 mg, 43%). MS (ESI, pos.): calc'd for C₂₇H₃₃IO₈, 612.12; found 613.1 (M+H), 635.1 (M+Na).

Compound 63: To a solution of compound 62 (11 mg, 0.0178 mmol) in DMF (0.175 mL) was added sodium azide (3.5 mg, 0.0538 mmol). The reaction was stirred at room temperature for 4 hours, then was diluted with EtOAc (1 mL) and washed with H₂O (1 mL). The aqueous layer was extracted with EtOAc (3×1 mL). The combined organic layers were washed with brine (1 mL) then were dried over Na₂SO₄, filtered, and concentrated in vacuo. Chromatography on an ISCO 4 g Silica column, eluting with EtOAc/hexanes, afforded compound 63 (6 mg, 64%). MS (ESI, pos.): calc'd for C₂₇H₃₃N₃O₈, 527.23; found 528.3 (M+H).

Compound 64: To a solution of compound 63 (6 mg, 0.011 mmol) in THF (1 mL) was added triphenylphosphine (6 mg, 0.023 mmol). The reaction was stirred at room temperature for 18 hours. To the reaction was added DI water (0.2 mL) and the mixture was heated in a 45° C. aluminum block for 4.5 hours. After cooling to room temperature, the reaction was concentrated in vacuo. The product was purified by chromatography on an ISCO 5.5 g C18Aq column, eluting with 5-100% MeCN in H₂O (both containing 0.05% HOAc). Fractions containing pure product were combined and lyophilized to afford (S)-amino-verrucarin A (64, 2 mg, 36%) as a fluffy white solid. MS (ESI, pos.): calc'd for C₂₇H₃₅NO₆, 501.24; found 502.2 (M+H). ¹H NMR (300 MHz; CDCl₃) δ 8.01 (dd, J=15.7, 11.6 Hz, 1H), 6.66 (t, J=11.3 Hz, 1H), 6.16 (d, J=10.6 Hz, 1H), 6.04 (d, J=15.7 Hz, 1H), 5.79 (dd, J=8.0, 3.9 Hz, 1H), 5.44-5.42 (m, 1H), 4.66 (d, J=12.1 Hz, 1H), 4.51-4.44 (m, 1H), 4.17 (d, J=12.1 Hz, 1H), 4.04-3.95 (m, 1H), 3.86 (d, J=5.0 Hz, 1H), 3.65 (d, J=4.3 Hz, 1H), 3.57 (d, J=5.4 Hz, 1H), 3.44 (d, J=2.1 Hz, 1H), 3.12 (d, J=3.9 Hz, 1H), 2.81 (d, J=4.0 Hz, 1H), 2.49 (dd, J=15.4, 8.2 Hz, 1H), 2.41-2.36 (m, 1H), 2.22 (dt, J=15.6, 4.6 Hz, 1H), 1.99-1.74 (m, 7H), 1.74 (s, 3H), 0.88 (d, J=6.8 Hz, 3H) overlapping 0.87 (s, 3H).

### Example 22

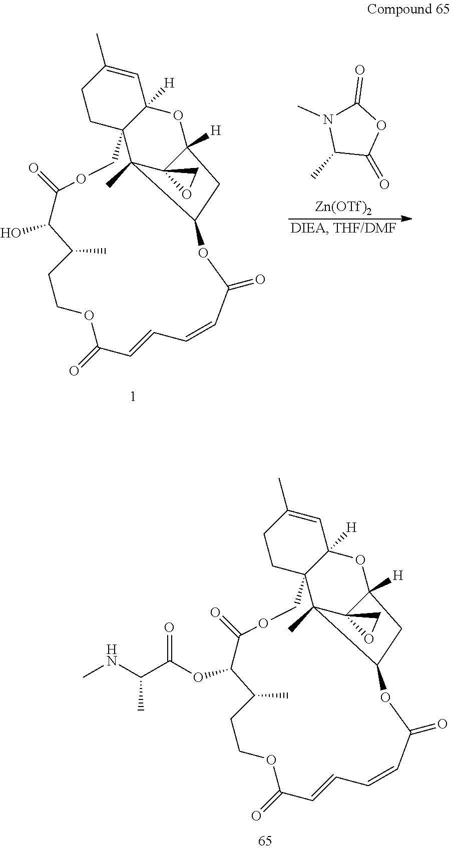

Compound 65: Verrucarin A (1, 5 mg, 0.01 mmol) in a small vial was dried azeotropically with toluene (2×3 mL). To the vial containing Verrucarin A in a glove box was added (S)-3,4-dimethyloxazolidine-2,5-dione (9 mg, 0.07 mmol), THF (0.6 mL) and DMF (0.2 mL). N,N-diisopropylethylamine (11 μL, 0.06 mmol) was added followed by zinc triflate (11 mg, 0.03 mmol). The reaction vial was taken out of the glove box and stirred under argon for 22 hours, at which time the reaction was complete by LCMS. The reaction was quenched with water (0.3 mL) and stirred for 5 minutes then injected onto an ISCO 15.5 g C18Aq column and eluted using 5-40% MeCN/H₂O (both having 0.05% AcOH). Pure fractions were combined and lyophilized to obtain compound 65 (4.9 mg, 85%) as a fluffy off-white solid. MS (ESI, pos.): calc'd for C₃₁H₄₁NO₁₀, 587.3; found 588.2 (M+H). ¹H-NMR (500 MHz; CDCl₃): δ 8.04 (dd, J=15.4, 11.9 Hz, 1H), 6.69 (t, J=11.3 Hz, 1H), 6.19 (d, J=11.1 Hz, 1H), 6.07 (d, J=15.6 Hz, 1H), 5.82 (dd, J=7.8, 3.3 Hz, 1H), 5.42 (d, J=3.9 Hz, 1H), 4.88 (s, 1H), 4.69 (d, J=12.0 Hz, 1H), 4.49 (d, J=10.9 Hz, 1H), 4.23-4.20 (m, 1H), 4.03 (t, J=11.1 Hz, 1H), 3.88 (d, J=5.0 Hz, 1H), 3.58 (d, J=4.9 Hz, 1H), 3.41-3.36 (m, 1H), 3.15-3.14 (m, 1H), 2.84-2.83 (m, 1H), 2.56-2.47 (m, 2H), 2.44 (s, 3H), 2.25 (dt, J=15.2, 4.6 Hz, 1H), 2.05-2.02 (m, 1H), 1.96-1.84 (m, 4H), 1.75 (d, J=6.2 Hz, 3H), 1.73-1.68 (m, 1H), 1.40 (d, J=7.0 Hz, 3H), 1.07 (d, J=6.8 Hz, 3H), 0.89 (s, 3H).

### Example 23

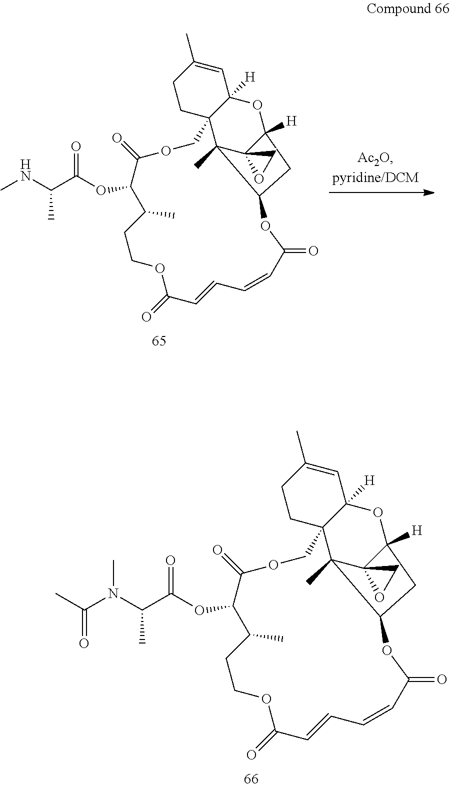 [SMILES: CN[C@@H](C)C(=O)O[C@@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1 ; CC(=O)N(C)[C@@H](C)C(=O)O[C@@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1]

Compound 66: To the solution of compound 65 (3.8 mg, 0.0065 mmol) in DCM (1 mL) at 0° C., pyridine (1 mL) and acetic anhydride (6.6 μL, 0.065 mmol) were added. The reaction was stirred for 10 minutes at 0° C., at which time LCMS indicated the reaction was complete. Volatiles were removed in vacuo. The residue was dissolved in DMF (0.5 mL) and purified on an ISCO 5.5 g C18Aq column using 5-95% MeCN/H₂O (both having 0.05% AcOH). Pure fractions were combined and lyophilized to obtain compound 66 (2.6 mg, 65%) as a fluffy off-white solid. MS (ESI, pos.): calc'd for C₃₃H₄₃NO₁₁, 629.3; found 630.3 (M+H), 652.3 (M+Na). ¹H-NMR (500 MHz; CDCl₃): δ 8.01 (dd, J=15.6, 11.7 Hz, 1H), 6.68 (t, J=11.4 Hz, 1H), 6.18 (d, J=11.1 Hz, 1H), 6.06 (d, J=15.8 Hz, 1H), 5.81 (dd, J=7.1, 3.1 Hz, 1H), 5.43 (d, J=3.0 Hz, 1H), 5.20 (q, J=7.3 Hz, 1H), 4.82 (s, 1H), 4.69 (d, J=12.2 Hz, 1H), 4.47 (dd, J=11.4, 1.0 Hz, 1H), 4.22 (d, J=11.9 Hz, 1H), 4.05-4.00 (m, 1H), 3.87 (d, J=4.8 Hz, 1H), 3.58 (d, J=4.9 Hz, 1H), 3.14 (d, J=3.5 Hz, 1H), 3.01 (s, 3H), 2.84 (s, 2H), 2.63 (s, 1H), 2.49 (dd, J=15.3, 8.2 Hz, 2H), 2.26-2.22 (m, 1H), 2.14 (s, 3H), 2.05-2.01 (m, 1H), 1.95-1.83 (m, 2H), 1.75 (s, 3H), 1.70-1.63 (m, 1H), 1.51 (d, J=7.3 Hz, 3H), 1.04 (d, J=6.9 Hz, 3H), 0.88 (s, 3H).

### Example 24

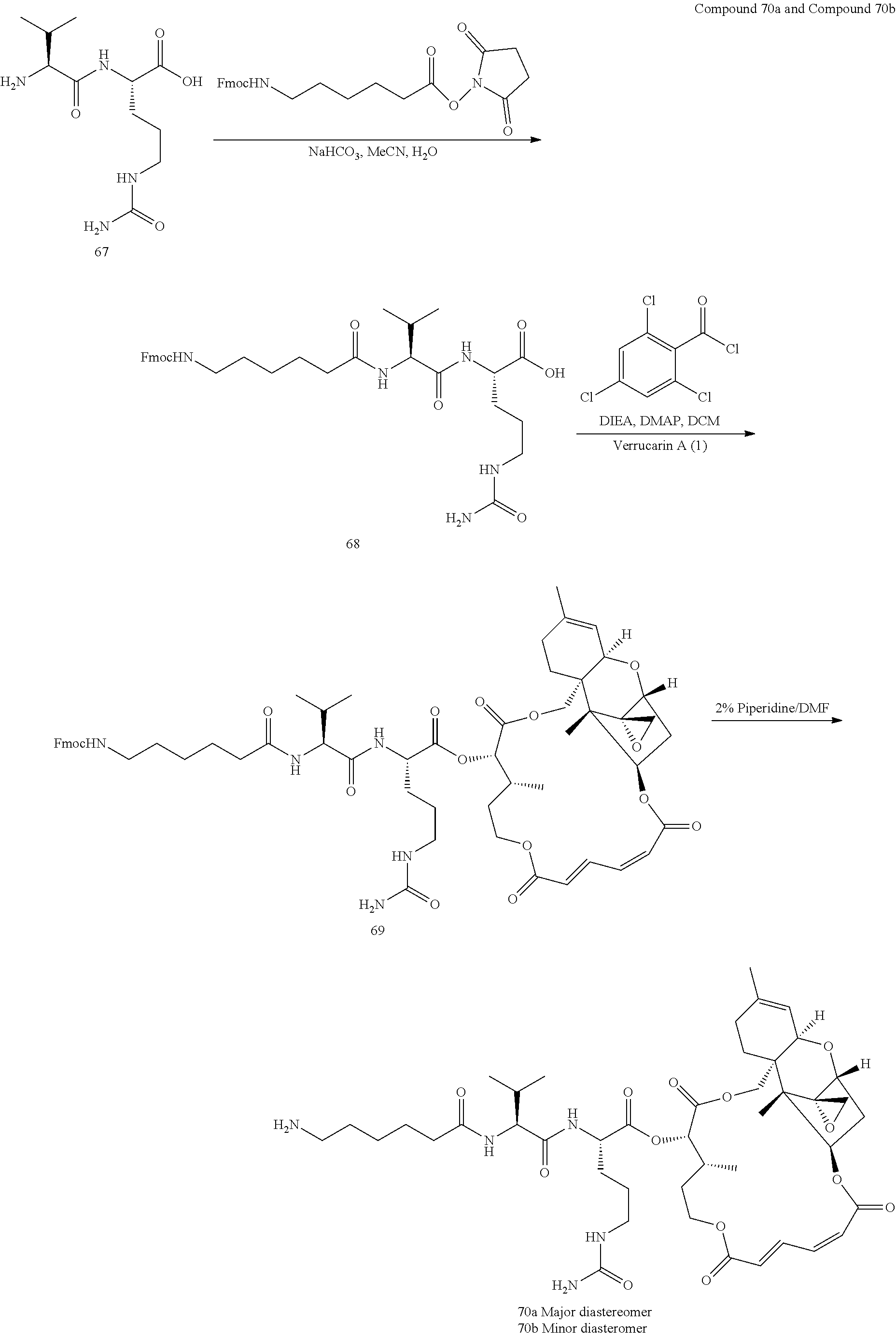

Compound 68: To the solution of Fmoc-6-aminohexanoic acid N-hydroxysuccinimide ester (150 mg, 0.33 mmol) and Val-Cit-OH TFA salt (67,127 mg, 0.33 mmol) in acetonitrile (3 mL) and water (2 mL), saturated aqueous NaHCO₃ solution (1 mL) was added at room temperature. After stirring for 18 hours, the pH was adjusted to ˜6 by adding acetic acid. The reaction was purified on an ISCO 50 g C18Aq column using 5-95% MeCN/H₂O (both having 0.05% AcOH). Pure fractions were combined and lyophilized to obtain compound 68 (133 mg, 66%) as a fluffy white solid. MS (ESI, pos.): calc'd for C₃₂H₄₃N₅O₇, 609.3; found 610.3 (M+H).

Compound 69: To a solution of compound 68 (39.6 mg, 0.065 mmol) and N,N-diisopropylethylamine (15.6 μL, 0.09 mmol) in DCM (1 mL) at 0° C., 2,4,6-trichlorobenzoyl chloride (11 μL, 0.07 mmol) was added dropwise and the solution was stirred for 1 hour at 0° C. To the resulting solution was added a DCM (1 mL) solution of DMAP (9.2 mg, 0.075 mmol) then a DCM (1 mL) solution of Verrucarin A (1, 25 mg, 0.05 mmol) at 0° C. The reaction was slowly warmed to room temperature and stirred for 2 hours. Volatiles were removed in vacuo. The residue was dissolved in DMF (1 mL) and purified on an ISCO 50 g C18Aq column using 5-95% MeCN/H₂O (both having 0.05% AcOH). Fractions containing unreacted 1 were combined and lyophilized to recover 14 mg (56%) of Verrucarin A. Fractions containing pure product were combined and lyophilized to obtain compound 69 (10 mg, 42% BRSM) as a fluffy off-white solid. MS (ESI, pos.): calc'd for C₅₉H₇₅N₅O₁₅, 1093.5; found 1094.2 (M+H), 1116.0 (M+Na).

Compound 70a: To a solution of compound 69 (25 mg, 0.023 mmol) in DMF (1.5 mL) at room temperature, 5% piperidine in DMF solution (0.5 mL) was added and the reaction was stirred for 30 minutes. Two isomers (29:71 at 254 nm) were observed in the LC/MS. The reaction was directly injected onto an ISCO 15 g C18Aq column and eluted with 5-95% MeCN/H₂O (both having 0.05% AcOH). Pure fractions containing the major isomer were combined and lyophilized to obtain compound 70a (10.1 mg, 51%) as a fluffy off-white solid. MS (ESI, pos.): calc'd for C₄₄H₆₅N₅O₁₃, 871.5; found 872.3 (M+H). 1H-NMR (500 MHz; CDCl₃): δ 8.02 (dd, J=15.3, 11.5 Hz, 1H), 7.83-7.79 (m, 1H), 7.19-7.14 (m, 1H), 6.71-6.66 (m, 1H), 6.18 (d, J=11.0 Hz, 1H), 6.07 (s, 1H), 5.99-5.96 (m, 1H), 5.81-5.78 (m, 1H), 5.42-5.41 (m, 2H), 4.87 (s, 1H), 4.66-4.59 (m, 2H), 4.49-4.45 (m, 1H), 4.27 (t, J=8.0 Hz, 1H), 4.22 (d, J=12.8 Hz, 1H), 4.01 (t, J=11.2 Hz, 1H), 3.87 (d, J=4.6 Hz, 1H), 3.57 (d, J=3.9 Hz, 1H), 3.18-3.12 (m, 7H), 2.83 (d, J=3.2 Hz, 3H), 2.50 (td, J=14.7, 8.1 Hz, 2H), 2.23 (dt, J=10.1, 4.9 Hz, 3H), 2.09-1.81 (m, 9H), 1.76 (s, 3H), 1.64-1.59 (m, 5H), 1.37 (dd, J=12.9, 6.6 Hz, 2H), 1.04 (d, J=6.6 Hz, 3H), 0.98 (t, J=7.2 Hz, 6H), 0.87 (s, 3H).

Compound 70b: The mixed fractions from the RP purification were lyophilized then re-purified on an ISCO 15 g C18Aq column, eluting with 5-95% MeCN/H₂O (both having 0.05% AcOH). Pure fractions containing the minor diastereomer were combined and lyophilized to obtain compound 70b (6.1 mg, 31%). MS (ESI, pos.): calc'd for C₄₄H₆₅N₅O₁₃, 871.5; found 872.3 (M+H). ¹H-NMR (500 MHz; CDCl₃): δ 8.00 (dd, J=14.0, 12.6 Hz, 1H), 7.84-7.81 (m, 1H), 6.99-6.93 (m, 1H), 6.68 (t, J=11.3 Hz, 1H), 6.17 (dd, J=11.1, 0.8 Hz, 1H), 6.06-6.03 (m, 1H), 5.94-5.86 (m, 1H), 5.82-5.77 (m, 1H), 5.41 (td, J=1.9, 0.8 Hz, 1H), 5.21-5.09 (m, 1H), 4.85 (t, J=1.7 Hz, 1H), 4.66-4.62 (m, 1H), 4.59-4.56 (m, 1H), 4.46-4.42 (m, 1H), 4.29-4.19 (m, 2H), 4.00-3.98 (m, 1H), 3.86 (td, J=1.7, 0.8 Hz, 1H), 3.56 (ddd, J=4.0, 1.5, 0.8 Hz, 1H), 3.24-3.22 (m, 1H), 3.15-3.11 (m, 2H), 2.82 (m, 4H), 2.51-2.47 (m, 6H), 2.33-2.14 (m, 6H), 1.94-1.85 (m, 8H), 1.74 (m, 4H), 1.42-1.38 (m, 3H), 1.27 (s, 2H), 1.03-0.96 (m, 6H), 0.86 (d, J=0.4 Hz, 3H).

### Example 25

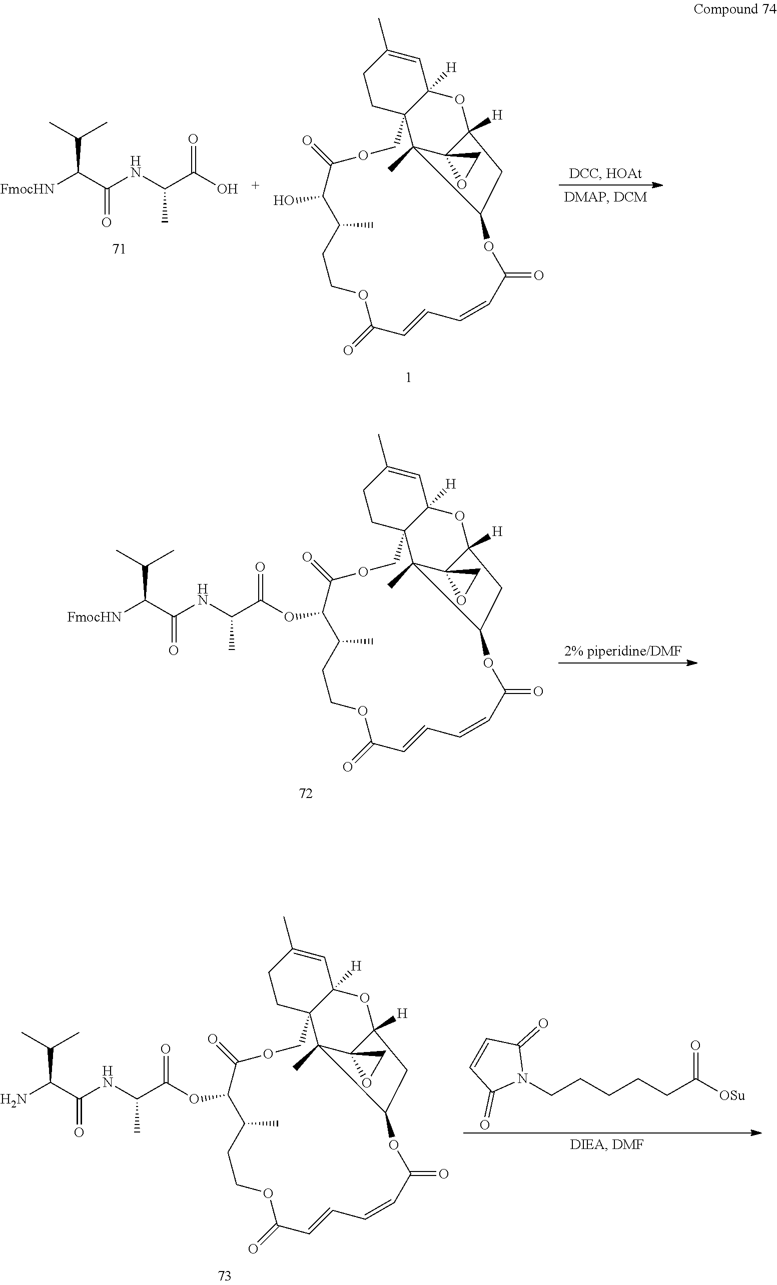

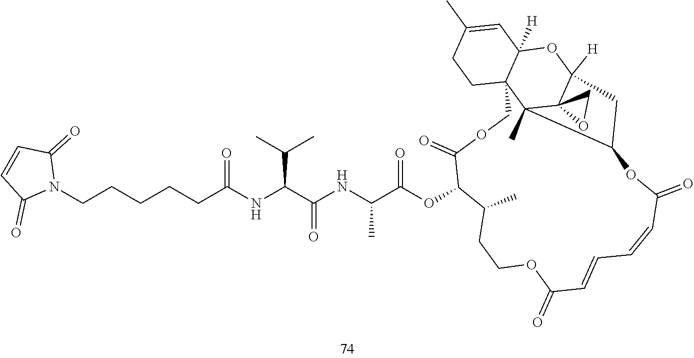 [SMILES: CC1=C[C@H]2O[C@@H]3C[C@H]4OC(=O)/C=C\C=C\C(=O)OCC[C@@H](C)[C@H](OC(=O)[C@H](C)NC(=O)[C@@H](NC(=O)CCCCCN5C(=O)C=CC5=O)C(C)C)C(=O)OC[C@@]2(CC1)[C@]4(C)[C@]31CO1]

Compound 72: To a mixture of Fmoc-Val-Ala-OH (71, 10.3 mg, 0.025 mmol), Verrucarin A, (1, 12.5 mg, 0.025 mmol), N,N-dicyclohexylcarbodiimide (10.3 mg, 0.05 mmol), 1-hydroxy-7-azabenzotriazole (3.5 mg, 0.025 mmol) and DMAP (3.1 mg, 0.025 mmol) was added anhydrous dichloromethane (1 mL). The reaction was stirred for 18 h then concentrated in vacuo and purified on an ISCO 15.5 g C18Aq column using 5-100% MeCN/H₂O (both containing 0.05% AcOH). Pure fractions were combined and lyophilized to obtain compound 72 (15 mg, 48%) as a fluffy off-white solid. MS (ESI, pos.): calc'd for C₅₀H₅₈N₂O₁₃, 894.4; found 895.2 (M+H), 917.3 (M+Na).

Compound 73: To a solution of compound 72 (14 mg, 0.015 mmol) in DMF (0.5 mL), a 5% piperidine solution in DMF (0.3 mL) was added and the reaction was stirred for 30 minutes. The reaction solution was directly injected onto an ISCO 15.5 g C18Aq column and eluted with 5-95% MeCN/H₂O (both containing 0.05% AcOH). Pure fractions were combined and lyophilized to obtain compound 73 (9 mg, 76%) as a fluffy off-white solid. MS (ESI, pos.): calc'd for C₃₅H₄₈N₂O₁₁, 672.3; found 873.2 (M+H).

Compound 74: To a solution of compound 73 (9 mg, 0.012 mmol) and 6-maleimidocaproic acid N-hydroxysuccinimide ester (8.7 mg, 0.027 mmol) in anhydrous DMF at 0° C., was added N,N-diisopropylethylamine. The reaction was warmed to room temperature and stirred for 2 hours. The reaction was directly injected onto an ISCO 15.5 g C18Aq column and eluted with 5-95% MeCN/H₂O (both containing 0.05% AcOH). Pure fractions were combined and lyophilized to obtain compound 74 (9 mg, 75%) as a fluffy off-white solid. MS (ESI, pos.): calc'd for C₄₅H₅₉N₃O₁₄, 865.4; found 866.3 (M+H). ¹H-NMR (500 MHz; CDCl₃): δ 8.03 (dd, J=15.7, 11.6 Hz, 1H), 6.69 (q, J=7.1 Hz, 3H), 6.41 (d, J=6.9 Hz, 1H), 6.18 (d, J=11.0 Hz, 1H), 6.07-6.03 (m, 2H), 5.81 (dd, J=7.8, 3.9 Hz, 1H), 5.42 (d, J=4.9 Hz, 1H), 4.89 (d, J=1.6 Hz, 1H), 4.75-4.66 (m, 2H), 4.49-4.46 (m, 1H), 4.31 (dd, J=8.5, 6.7 Hz, 1H), 4.22 (dd, J=12.3, 4.6 Hz, 1H), 4.03-3.98 (m, 1H), 3.88 (d, J=5.1 Hz, 1H), 3.58 (d, J=4.7 Hz, 1H), 3.54 (d, J=7.1 Hz, 2H), 3.14 (d, J=3.8 Hz, 1H), 2.83 (t, J=4.1 Hz, 1H), 2.51 (td, J=16.2, 7.5 Hz, 2H), 2.27-2.21 (m, 3H), 2.10 (dt, J=13.0, 6.4 Hz, 1H), 2.06-2.01 (m, 1H), 1.93 (t, J=10.7 Hz, 2H), 1.82 (d, J=19.5 Hz, 1H), 1.77 (s, 3H), 1.69 (dt, J=14.3, 7.3 Hz, 3H), 1.62 (t, J=7.5 Hz, 4H), 1.49 (d, J=7.1 Hz, 2H), 1.35 (q, J=7.7 Hz, 2H), 1.05 (t, J=5.6 Hz, 3H), 0.97 (q, J=7.1 Hz, 6H), 0.87 (s, 3H).

### Example 26

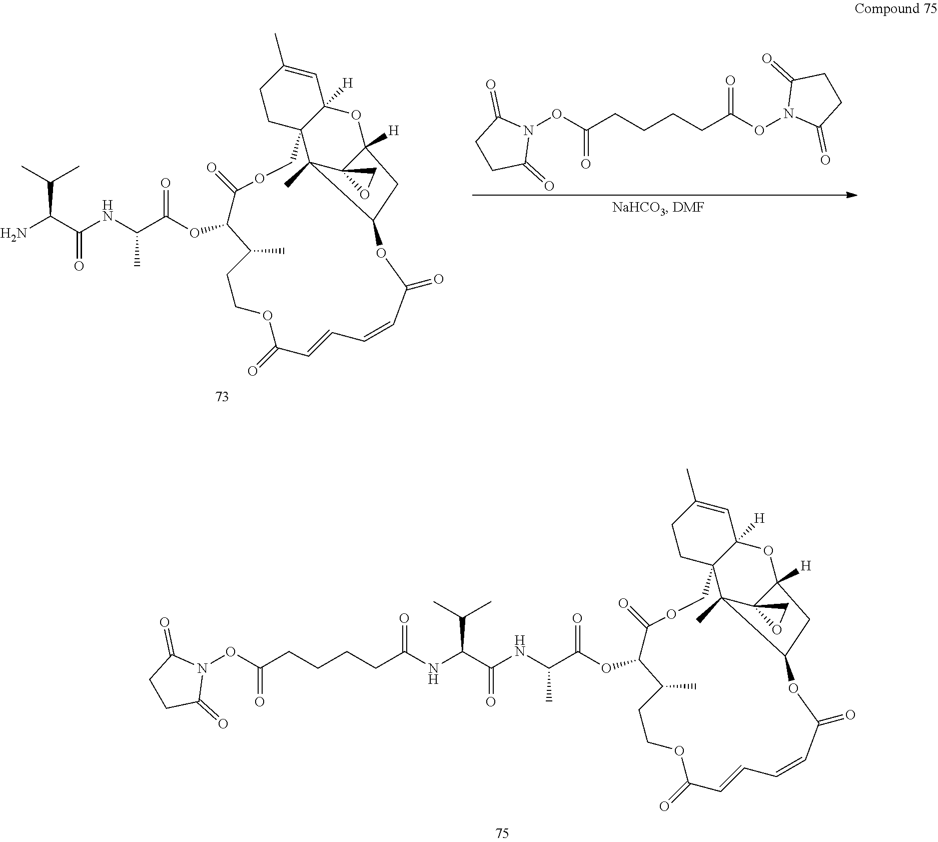 [SMILES: CC1=C[C@H]2O[C@@H]3C[C@H]4OC(=O)/C=C\C=C\C(=O)OCC[C@@H](C)[C@H](OC(=O)[C@H](C)NC(=O)[C@@H](N)C(C)C)C(=O)OC[C@@]2(CC1)[C@]4(C)[C@]31CO1 ; O=C(CCCCC(=O)ON1C(=O)CCC1=O)ON1C(=O)CCC1=O ; CC1=C[C@H]2O[C@@H]3C[C@H]4OC(=O)/C=C\C=C\C(=O)OCC[C@@H](C)[C@H](OC(=O)[C@H](C)NC(=O)[C@@H](NC(=O)CCCCC(=O)ON5C(=O)CCC5=O)C(C)C)C(=O)OC[C@@]2(CC1)[C@]4(C)[C@]31CO1]

Compound 75: A mixture of compound 73 (9 mg, 0.012 mmol), NaHCO₃ (3 mg, 0.036 mmol) and bis(2,5-dioxopyrrolidin-1-yl) adipate (77.4 mg, 0.24 mmol) in anhydrous DMF (1 mL) was stirred at room temperature for 15 minutes. LCMS indicated the reaction was complete. The reaction was injected onto an ISCO 15.5 g C18Aq column and eluted with 5-95% MeCN/H₂O (both containing 0.05% AcOH). Pure fractions were combined and lyophilized to obtain compound 75 (9.7 mg, 95%) as a fluffy off-white solid. MS (ESI, pos.): calc'd for C₄₅H₅₉N₃O₁₆, 897.4; found 898.3 (M+H). ¹H-NMR (500 MHz; CDCl₃): δ 8.03 (dd, J=15.7, 11.6 Hz, 1H), 6.68 (t, J=11.4 Hz, 1H), 6.48 (d, J=7.0 Hz, 1H), 6.17 (t, J=9.4 Hz, 2H), 6.06 (d, J=15.7 Hz, 1H), 5.81 (dd, J=8.1, 4.0 Hz, 1H), 5.43-5.42 (m, 1H), 4.89 (d, J=1.9 Hz, 1H), 4.74 (t, J=7.1 Hz, 1H), 4.68 (dd, J=11.8, 3.7 Hz, 1H), 4.49-4.45 (m, 1H), 4.31 (dd, J=8.5, 6.5 Hz, 1H), 4.25-4.20 (m, 1H), 4.01 (td, J=11.7, 3.0 Hz, 1H), 3.88 (d, J=5.1 Hz, 1H), 3.58 (d, J=4.9 Hz, 1H), 3.14 (d, J=3.9 Hz, 1H), 2.86-2.83 (m, 6H), 2.68-2.64 (m, 2H), 2.54-2.47 (m, 2H), 2.33-2.30 (m, 2H), 2.27-2.22 (m, 1H), 2.19-2.11 (m, 1H), 2.05-2.00 (m, 1H), 1.92 (d, J=10.1 Hz, 2H), 1.84-1.81 (m, 6H), 1.77 (s, 3H), 1.48 (d, J=7.1 Hz, 3H), 1.04 (d, J=6.8 Hz, 3H), 0.96 (dd, J=8.7, 6.9 Hz, 6H), 0.87 (s, 3H).

### Example 27

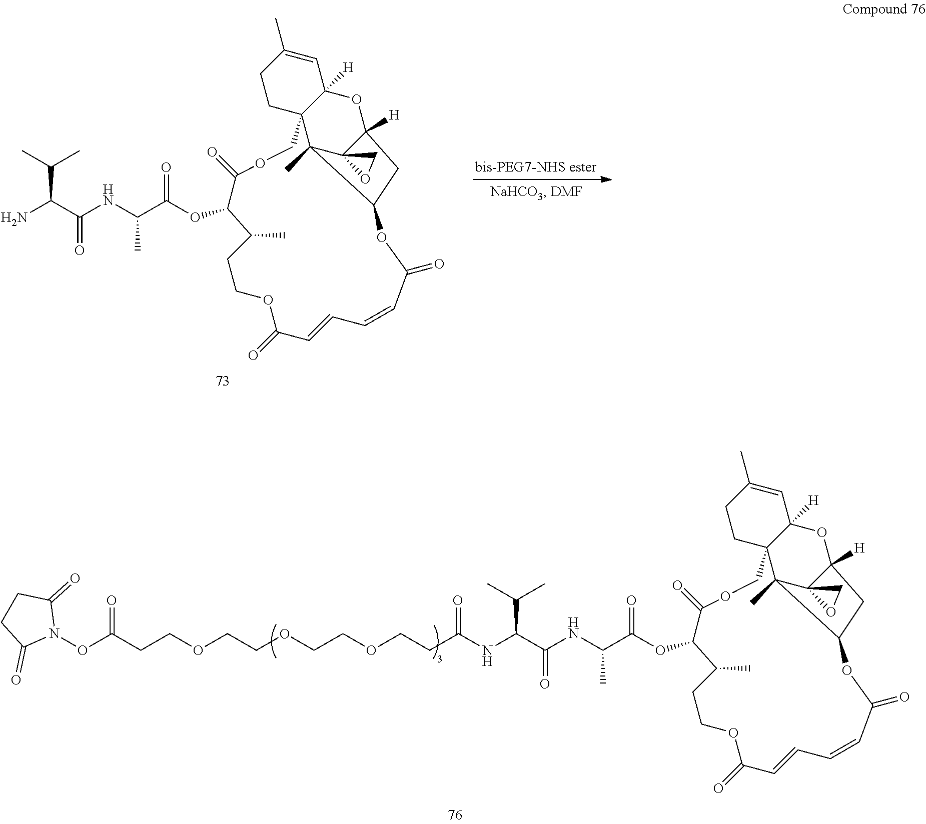

Compound 76: A mixture of compound 73 (7.4 mg, 0.0094 mmol), Bis-PEG-7-NHS ester (87.4 mg, 0.14 mmol) and NaHCO₃ (2.3 mg, 0.028 mmol) in anhydrous DMF (2 mL) was stirred for 30 minutes at room temperature. The crude reaction mixture was injected onto an ISCO 15.5 g C18Aq column and eluted with 5-95% MeCN/H₂O (both containing 0.05% AcOH). Pure fractions were combined and lyophilized to obtain compound 76 (5 mg, 45%) as a fluffy off-white solid. MS (ESI, pos.): calc'd for C57H₈₃N₃O₂₃, 1177.5; found 1178.5 (M+H). ¹H-NMR (300 MHz; CDCl₃): δ 8.05 (dd, J=15.3, 12.1 Hz, 1H), 6.92-6.89 (m, 1H), 6.70 (t, J=11.3 Hz, 2H), 6.20 (d, J=11.1 Hz, 1H), 6.08 (d, J=15.4 Hz, 1H), 5.84-5.80 (m, 1H), 5.45-5.43 (m, 1H), 4.91-4.88 (m, 1H), 4.76-4.66 (m, 2H), 4.52-4.45 (m, 1H), 4.34-4.21 (m, 2H), 4.07-3.98 (m, 1H), 3.88 (t, J=6.2 Hz, 3H), 3.79 (t, J=5.6 Hz, 3H), 3.74 (S, 24H), 3.59 (d, J=5.1 Hz, 1H), 3.16 (d, J=4.0 Hz, 1H), 2.96-2.85 (m, 5H), 2.58-2.47 (m, 4H), 2.30 (d, J=1.1 Hz, 2H), 2.30-2.20 (m, 3H), 2.09-1.81 (m, 4H), 1.81-1.76 (m, 3H), 1.55-1.48 (m, 3H), 1.06 (d, J=6.8 Hz, 3H), 0.99 (t, J=6.3 Hz, 6H), 0.89 (s, 3H).

### Example 28

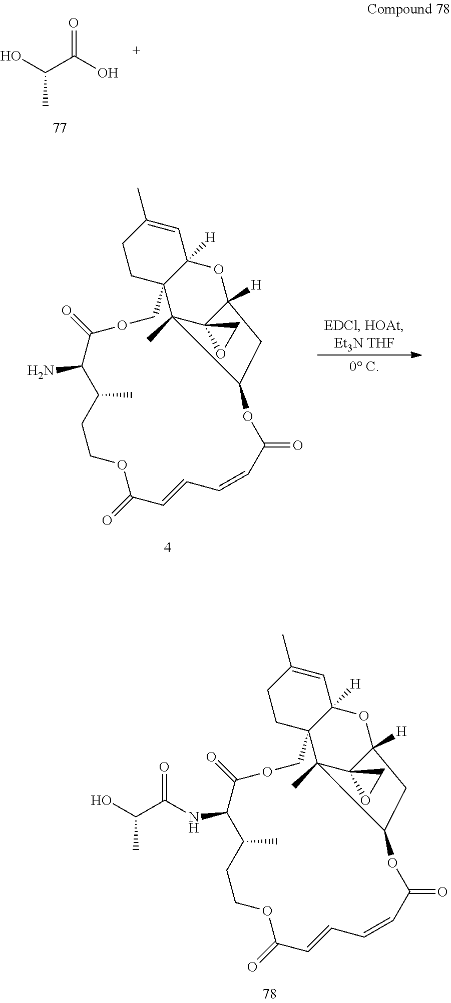

Compound 78: To a solution of compound 4 (5 mg, 0.01 mmol) and L-lactic acid (77, 0.9 mg, 0.01 mmol) in anhydrous THF (0.5 mL) at 0° C., were added EDCI (2.9 mg, 0.015 mmol), HOAt (2.0 mg, 0.015 mmol) and triethylamine (4.2 μL, 0.03 mmol). The reaction was stirred for 2 hours, then volatiles were removed in vacuo. The residue was purified on by prep HPLC on a 30×150 mm Gemini column using 5-95% MeCN/H₂O (both having 0.05% acetic acid). Pure fractions were combined and lyophilized to obtain compound 78 as a fluffy white solid (4.9 mg, 86%). MS (ESI, pos.): calc'd for C₃₀H₃₉NO₁₀, 573.3; found 574.2 (M+H)¹H-NMR (500 MHz; CDCl₃): δ 8.11-8.06 (m, 1H), 6.93 (d, J=8.9 Hz, 1H), 6.69 (t, J=10.9 Hz, 1H), 6.15 (d, J=11.1 Hz, 1H), 6.05 (d, J=15.7 Hz, 1H), 5.77 (dd, J=7.8, 3.4 Hz, 1H), 5.43 (d, J=4.2 Hz, 1H), 4.49-4.45 (m, 1H), 4.39 (t, J=10.4 Hz, 1H), 4.33 (m, 3H), 4.03 (td, J=11.5, 2.5 Hz, 1H), 3.87 (d, J=5.2 Hz, 1H), 3.59 (d, J=5.1 Hz, 1H), 3.13 (dd, J=4.0, 1.3 Hz, 1H), 2.82 (d, J=3.3 Hz, 1H), 2.51 (dd, J=15.1, 8.1 Hz, 1H), 2.22 (td, J=9.6, 4.4 Hz, 3H), 2.08-1.88 (m, 4H), 1.77-1.74 (m, 4H), 1.45 (m, 4H), 1.12 (d, J=5.8 Hz, 3H), 0.89 (s, 3H).

### Example 29

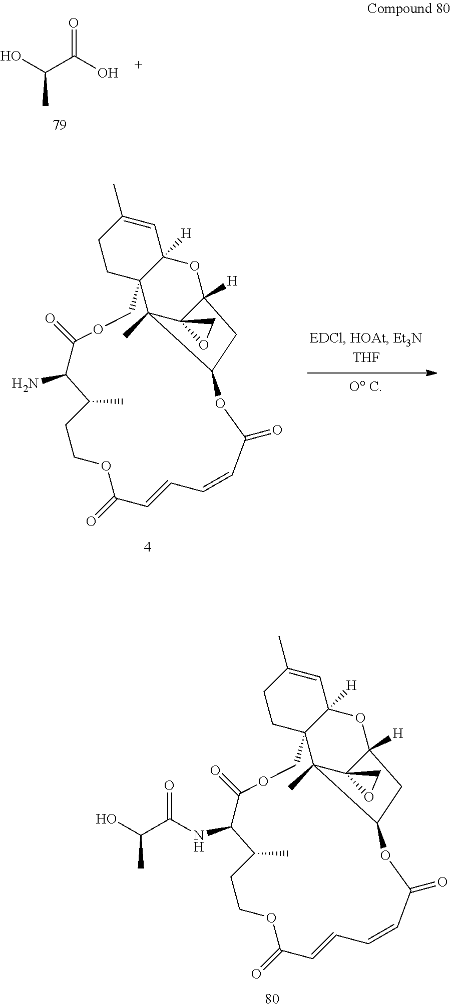

Compound 80 (4.6 mg, 80%) was prepared using the above procedure on the same scale but starting with D-Lactic acid (79). MS (ESI, pos.): calc'd for C₃₀H₃₉NO₁₀, 573.3; found 574.2 (M+H)¹H-NMR (500 MHz; CDCl₃): δ 8.09 (dd, J=15.7, 11.6 Hz, 1H), 6.97 (d, J=8.8 Hz, 1H), 6.69 (t, J=12.0 Hz, 1H), 6.15 (d, J=11.0 Hz, 1H), 6.05 (d, J=15.7 Hz, 1H), 5.78 (dd, J=7.9, 4.0 Hz, 1H), 5.42 (d, J=4.9 Hz, 1H), 4.48-4.45 (m, 1H), 4.40-4.32 (m, 3H), 4.28 (ddd, J=6.6, 4.3, 2.1 Hz, 1H), 4.06-4.00 (m, 1H), 3.86 (d, J=5.0 Hz, 1H), 3.59 (d, J=5.4 Hz, 1H), 3.13 (d, J=3.9 Hz, 1H), 2.81 (d, J=4.0 Hz, 1H), 2.51 (dd, J=15.5, 8.3 Hz, 1H), 2.24-2.19 (m, 2H), 2.01-1.86 (m, 5H), 1.74 (s, 3H), 1.49 (d, J=6.8 Hz, 3H), 1.48-1.44 (m, 1H), 1.11 (d, J=6.7 Hz, 3H), 0.88 (s, 3H).

### Example 30

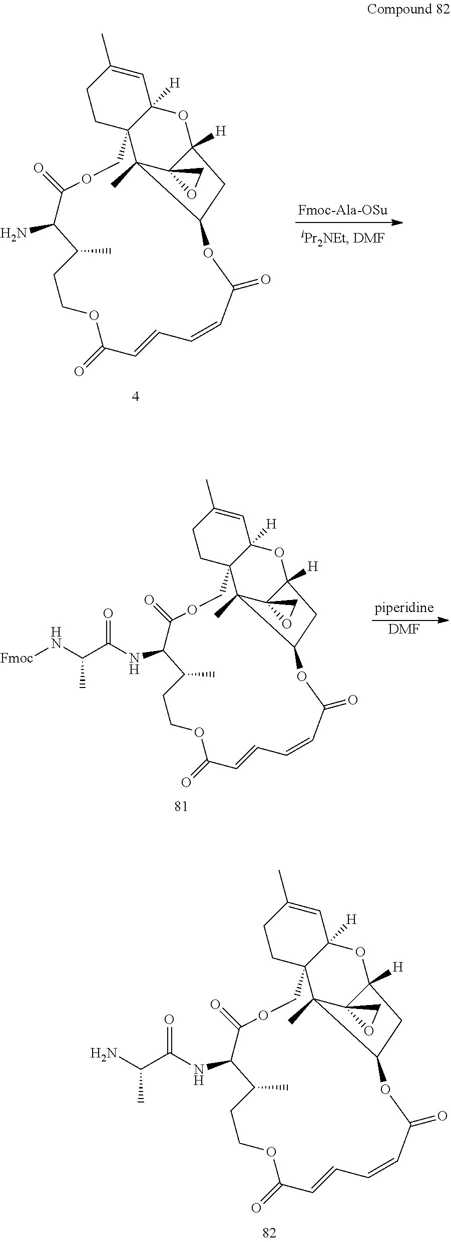

Compound 81: To a solution of (R)-amino-verrucarin A (4, 8 mg, 0.0142 mmol) and Fmoc-Ala-OSu (13 mg, 0.0318 mmol) in anhydrous DMF (280 μL) was added N,N-diisopropylethylamine (10 μL, 0.0574 mmol). The reaction was stirred at room temperature for 4 hours, at which time LCMS indicated reaction was complete. The reaction solution was loaded onto an ISCO 5.5 g C18 column and eluted with 10-80% MeCN in H₂O (both containing 0.05% HOAc). Fractions containing pure product were combined and lyophilized to afford compound 81 (8 mg, 73%) as a white solid. MS MS (ESI, pos.): calc'd for C₄₅H₅₀N₂O₁₁, 794.3; found 817.2 (M+Na).

Compound 82: Compound 81 (8.5 mg, 0.011 mmol) was treated with a 10% solution of piperidine in DMF (100 μL). The solution was stirred at room temperature for 40 minutes, at which time LCMS indicated the reaction was complete. The reaction solution was loaded onto an ISCO 5.5 g C18 column and eluted with 5-30% MeCN in H₂O (both containing 0.05% HOAc). Fractions with pure product were combined and lyophilized to afford compound 82 (6 mg, 98%) as a fluffy white solid. MS (ESI, pos.): calc'd for CH₃₀H₄₀N₂O₉, 572.3; found 573.3 (M+H). ¹H NMR (300 MHz; CDCl₃) δ 8.07 (dd, J=15.6, 11.5 Hz, 1H), 7.88-7.85 (m, 1H), 6.68 (t, J=11.4 Hz, 1H), 6.14 (d, J=11.1 Hz, 1H), 6.04 (d, J=15.6 Hz, 1H), 5.78-5.74 (m, 1H), 5.43-5.41 (m, 1H), 4.47-4.40 (m, 1H), 4.33 (dd, J=10.4, 7.2 Hz, 3H), 4.05-4.01 (m, 1H), 3.86 (d, J=5.1 Hz, 1H), 3.57 (q, J=5.7 Hz, 2H), 3.13 (d, J=4.0 Hz, 1H), 2.80 (d, J=4.0 Hz, 1H), 2.51 (dd, J=15.5, 8.1 Hz, 1H), 2.25-2.17 (m, 2H), 2.06-1.76 (m, 6H), 1.76-1.70 (m, 3H), 1.49-1.39 (m, 2H), 1.34 (d, J=7.0 Hz, 3H), 1.10 (d, J=6.7 Hz, 3H), 0.88 (s, 3H).

### Example 31

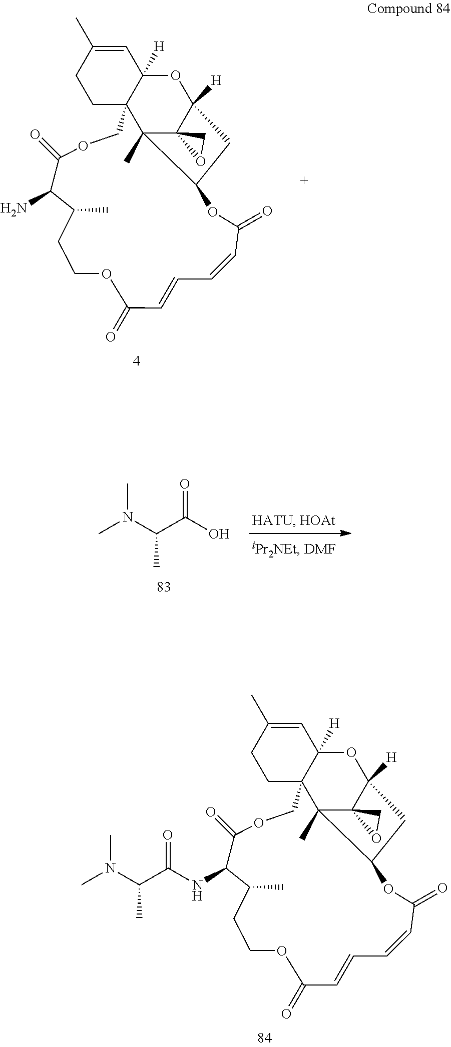

Compound 84: To a solution of N,N-dimethylalanine (83, 2.5 mg, 0.021 mmol), HOAt (2.7 mg, 0.020 mmol), and HATU (7.6 mg (0.020 mmol) in anhydrous DMF (75 mL) was added N,N-diisopropylethylamine (3.5 mL, 0.020 mmol). The resulting yellow solution was stirred for 5 minutes, then a solution of (R)-amino-verrucarin A (4, 5 mg, 0.01 mmol) in DMF (175 mL) was added. The reaction was stirred at room temperature for 1 hour, at which time LCMS indicated the reaction was complete. The reaction solution was loaded onto an ISCO 5.5 g C18 column and eluted with 5-100% MeCN in H₂O (both containing 0.05% HOAc). Product-containing fractions were lyophilized to afford compound 84 (4.2 mg, 50%) as a white fluffy solid. MS (ESI, pos.): calc'd for C₃₂H₄₄N₂O₉, 600.3; found 601.3 (M+H). ¹H NMR (300 MHz; CDCl₃) δ 8.09 (dd, J=15, 7, 11.7 Hz, 1H), 7.64 (d, J=9.2 Hz, 1H), 6.69 (t, J=11.3 Hz, 1H), 6.14 (d, J=10.8 Hz, 1H), 6.04 (d, J=15.6 Hz, 1H), 5.76 (dd, J=8.0, 4.0 Hz, 1H), 5.44-5.42 (m, 1H), 4.47-4.41 (m, 2H), 4.38-4.29 (m, 2H), 4.05-3.96 (m, 1H), 3.87 (d, J=5.1 Hz, 1H), 3.59 (d, J=5.0 Hz, 1H), 3.15 (d, J=3.9 Hz, 1H), 3.03 (q, J=7.0 Hz, 1H), 2.78 (d, J=4.1 Hz, 1H), 2.51 (dd, J=15.6, 8.1 Hz, 1H), 2.25 (s, 6H), 2.25-2.18 (m, 1H), 2.00-1.76 (m, 6H), 1.73 (s, 3H), 1.46-1.37 (m, 1H), 1.20 (d, J=7.0 Hz, 3H), 1.09 (d, J=6.6 Hz, 3H), 0.91 (s, 3H).

### Example 32

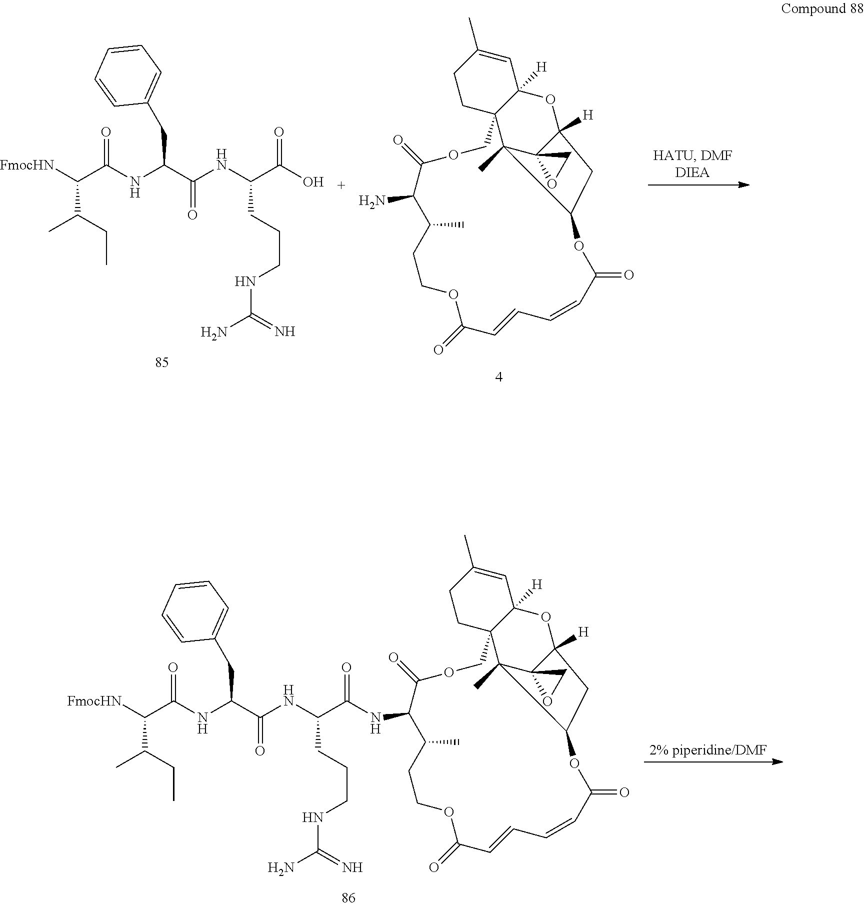

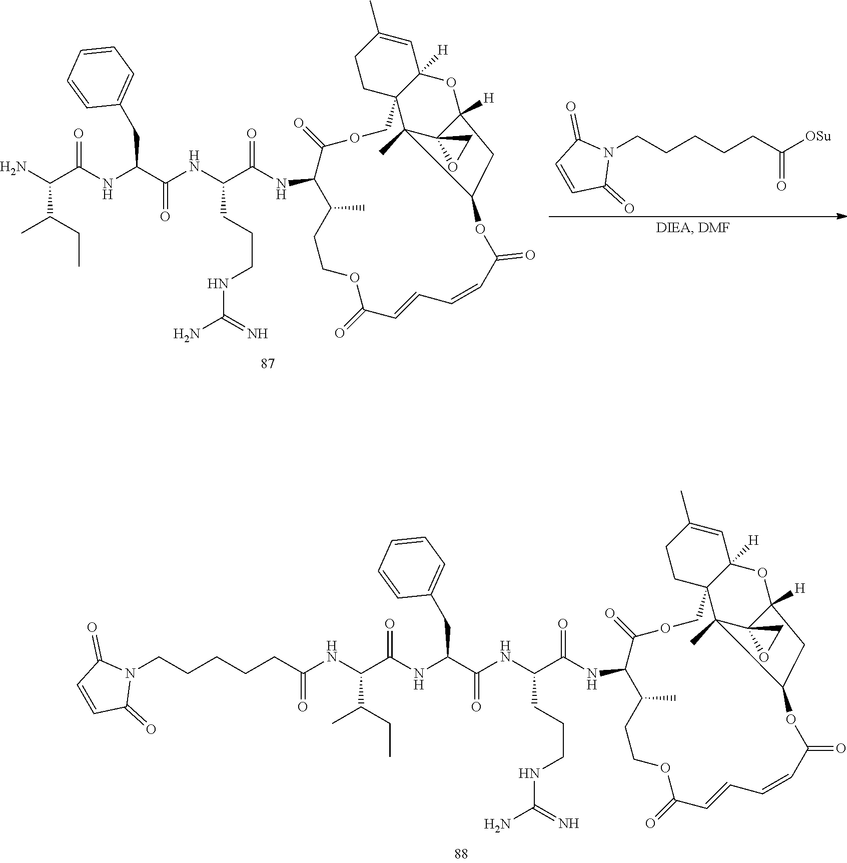

Compound 85 (Fmoc-Ile-Phe-Arg-OH) was prepared using a peptide synthesizer.

Compound 86: To a solution of compound 85 (21 mg, 0.032 mmol) in DMF (1.3 mL) at room temperature, HATU (15.2 mg, 0.04 mmol) and N,N-diisopropylethylamine (14 μL, 0.08 mmol) were added, followed by (R)-amino-verrucarin A (4, 9 mg, 0.016 mmol). The reaction was stirred for 6 hours then was injected onto an ISCO 30 g C18Aq column and eluted with 5-95% MeCN/H₂O (both having 0.05% AcOH). Pure fractions were combined and lyophilized to obtain compound 86 (12 mg, 59% yield) as a white fluffy solid. MS (ESI, pos.): calc'd for C₆₃H₇₇N₇O₁₃, 1139.6; found 1140.5 (M+H).

Compound 87: To a solution of compound 86 (12 mg, 0.01 mmol) in DMF (0.8 mL) at room temperature, a 5% solution of piperidine in DMF (0.4 mL) was added and the reaction was stirred for 20 minutes. The reaction solution was injected onto an ISCO 30 g C18Aq column and eluted with 5-95% MeCN/H₂O (both having 0.05% AcOH). Pure fractions were combined and lyophilized and to obtain compound 87 (7.9 mg, 82%) as a fluffy white solid. MS (ESI, pos.): calc'd for C₄₈H₆₇N₇O₁₁, 917.5; found 918.2 (M+H).

Compound 88: To a solution of compound 87 (4.3 mg, 0.0046 mmol) in DMF at room temperature, N-succinimidyl 6-maleimidohexanoate (2.1 mg, 0.0069 mmol) was added followed by diisopropylethylamine (2.4 μL, 0.013 mmol). The reaction was stirred for 1 hour then was injected onto an ISCO 15.5 g C18Aq column and eluted with 5-95% MeCN/H₂O (both having 0.05% AcOH). Pure fractions were combined and lyophilized and to obtain compound 88 (2 mg, 38%) as a fluffy white solid. MS (ESI, pos.): calc'd for C₅₈H₇₈N₈O₁₄, 1110.6; found 1111.3 (M+H). ¹H-NMR (500 MHz; DMSO-d₆): δ 9.04-9.00 (m, 1H), 8.22-8.20 (m, 1H), 7.98-7.86 (m, 3H), 7.79-7.75 (m, 1H), 7.21-7.15 (m, 5H), 6.99 (s, 2H), 6.88-6.83 (m, 1H), 6.26-6.21 (m, 3H), 5.75-5.73 (m, 1H), 5.27-5.23 (m, 2H), 4.54-4.48 (m, 2H), 4.47-4.40 (m, 1H), 4.14-4.03 (m, 3H), 3.93-3.87 (m, 1H), 3.68-3.63 (m, 4H), 3.02-3.00 (m, 3H), 2.85-2.78 (m, 2H), 2.43-2.36 (m, 3H), 2.24-2.19 (m, 3H), 2.15-2.08 (m, 3H), 2.05-2.00 (m, 2H), 1.95-1.91 (m, 2H), 1.74-1.69 (m, 2H), 1.70-1.57 (m, 5H), 1.46-1.42 (m, 3H), 1.25-1.13 (m, 6H), 1.01-0.94 (m, 1H), 0.91 (d, J=7.7 Hz, 3H), 0.77-0.68 (m, 9H).

### Example 33

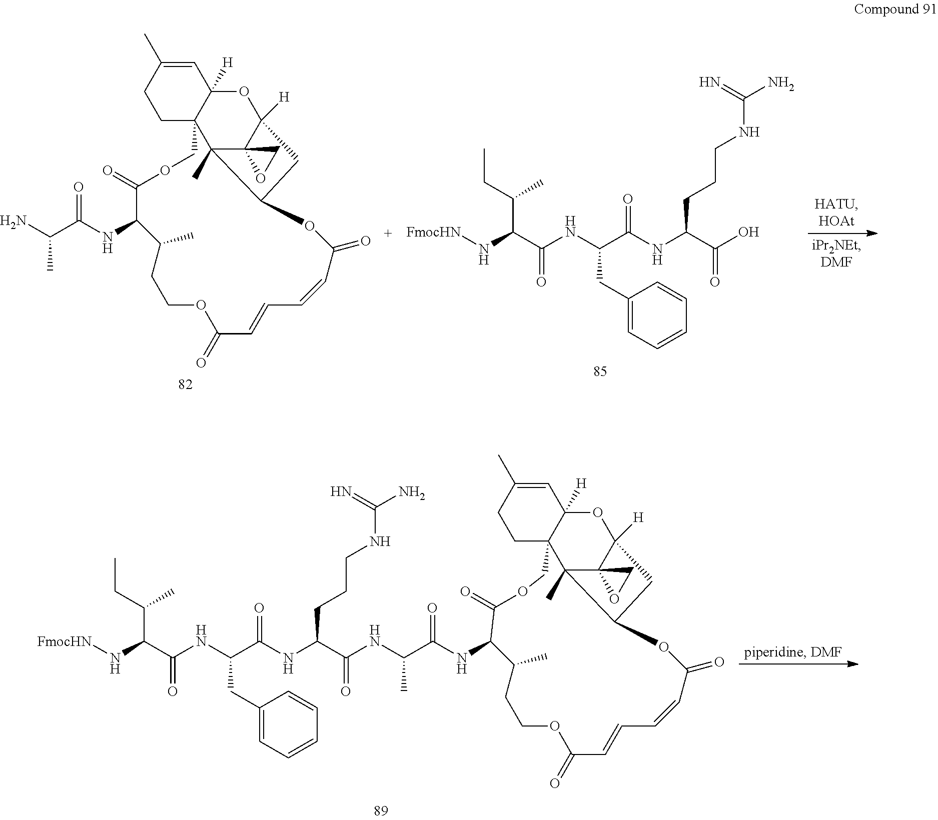

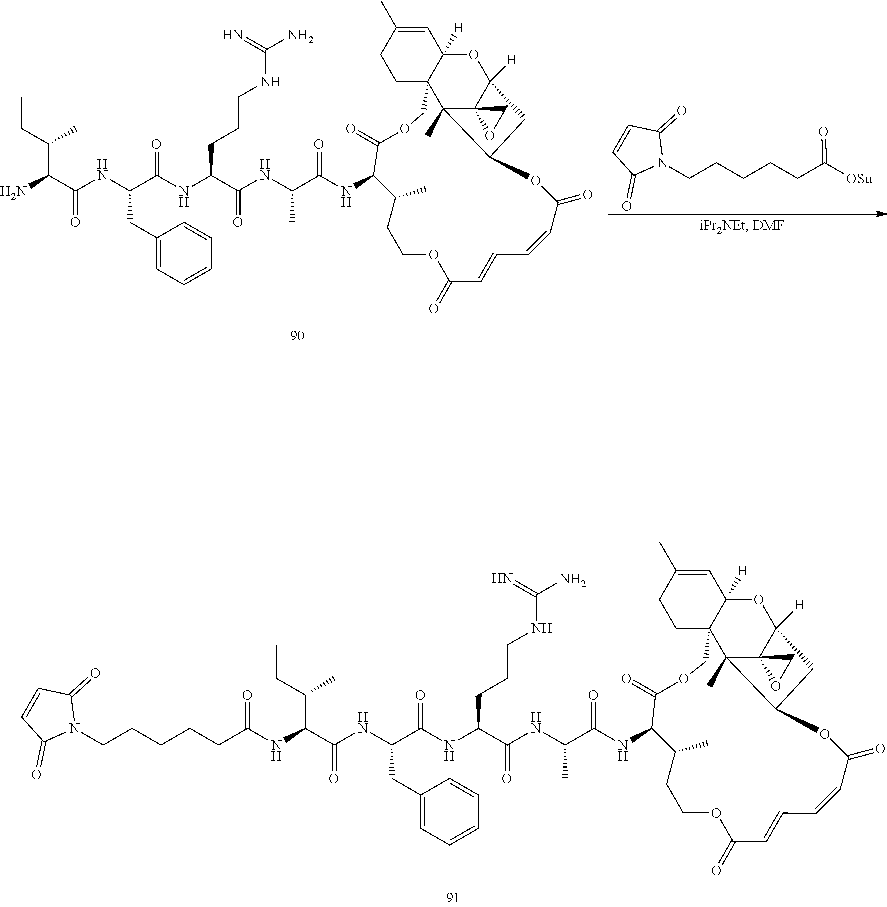

Compound 89: To a solution of compound 82 (14 mg, 0.0213 mmol), HATU (10 mg, 0.0262 mmol) and HOAt (3 mg, 0.0220 mmol) in anhydrous DMF (250 μL) was added compound 85 (6 mg, 0.0105 mmol) in DMF (250 μL). N,N-diisopropylethylamine 0.0230 mmol) was added and the reaction was stirred at room temperature for 1 hour, at which time LCMS indicated the reaction was complete. The solution was loaded onto an ISCO 15.5 g C18Aq column and eluted with 0-100% MeCN in H₂O (both containing 0.05% HOAc). Fractions containing pure product were combined and lyophilized to afford compound 89 (5 mg, 38%) as a white fluffy solid. MS (ESI, pos.): calc'd for C₆₆H₈₂N₈O₁₄, 1210.6; found, 1210.5 (M+).

Compound 90: Compound 89 (4.5 mg, 0.0037 mmol) was treated with 10% piperidine in DMF (150 μL). After 40 min LCMS indicated the reaction was complete. The reaction was loaded onto an ISCO 5.5 g C18 column and eluted with 5-40% MeCN in H₂O (both containing 0.05% HOAc). Fractions containing product of >95% purity were combined and lyophilized to afford compound 90 (2.3 mg, 62%) as a white fluffy solid. MS (ESI, pos.): calc'd for C₅₁H₇₂N₈O₁₂, 988.5; found 989.5 (M+H).

Compound 91: To a solution of compound 90 (4 mg, 0.00404 mmol) and N-succinimidyl 6-maleimidohexanoate (2 mg, 0.00649 mmol) in anhydrous DMF (100 μL) was added N,N-diisopropylethylamine (2 μL, 0.0115 mmol). After stirring at room temperature for 2.75 hours, LCMS showed the presence of unreacted amine, but the NHS ester had been consumed. Additional N-succinimidyl 6-maleimidohexanoate (1 mg, 0.00324 mmol) was added, and the reaction was stirred for 2 hours longer. At that time LCMS indicated the reaction was complete. The reaction was loaded onto an ISCO 5.5 g C18Aq column and eluted with MeCN in H₂O (both containing 0.05% HOAc). Clean fractions were combined and lyophilized to obtain compound 91 (1 mg, 17%) as a white fluffy solid. MS (ESI, pos.): calc'd for C₆₁H₈₃N₉O₁₅, 1181.6; found, 1182.5 (M+H). ¹H NMR (300 MHz; CDCl₃) δ 8.41 (br s, 1H), 8.10 (dd, J=12.3, 16.6 Hz, 1H), 7.69 (br s, 1H), 7.49 (br s, 1H), 7.18-7.15 (m, 5H), 7.03-7.00 (m, 1H), 6.78-6.73 (m, 2H), 6.55 (dd, J=11.6, 10.8 Hz, 1H), 6.03 (d, J=11.9 Hz, 1H), 5.95 (d, J=15.5 Hz, 1H), 5.78-5.74 (m, 1H), 5.43-5.41 (m, 1H), 4.67-4.57 (m, 1H), 4.47-3.91 (m, 6H), 3.90-3.85 (m, 1H), 3.60-3.49 (m, 4H), 3.24-3.20 (m, 2H), 3.08-2.99 (m, 2H), 2.84-2.83 (m, 1H), 2.65-2.54 (m, 2H), 2.37-2.16 (m, 4H), 2.05-2.04 (m, 3H), 1.46-1.27 (m, 9H), 1.10 (d, J=6.5 Hz, 3H), 0.99 (s, 3H), 0.82 (t, J=7.8 Hz, 3H), 0.72 (d, J=6.6 Hz, 3H).

### Example 34

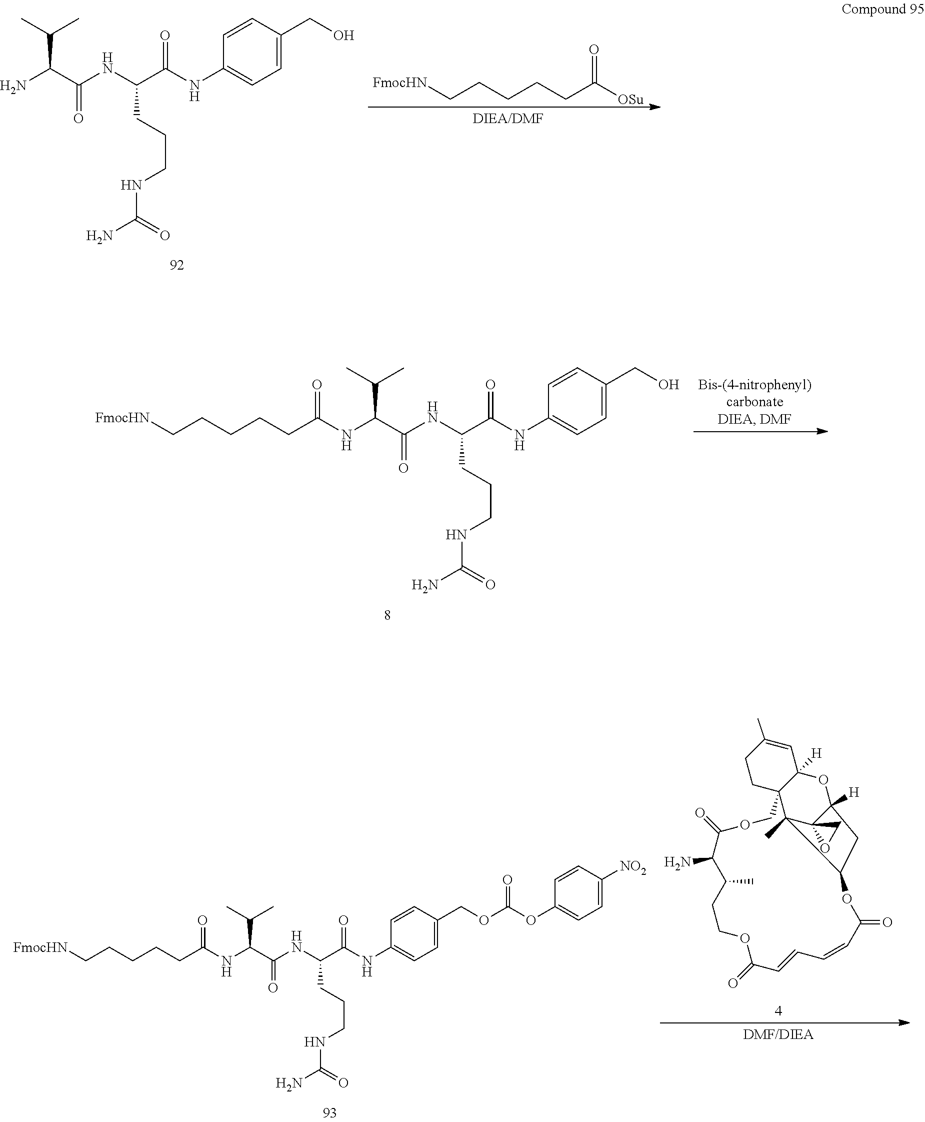

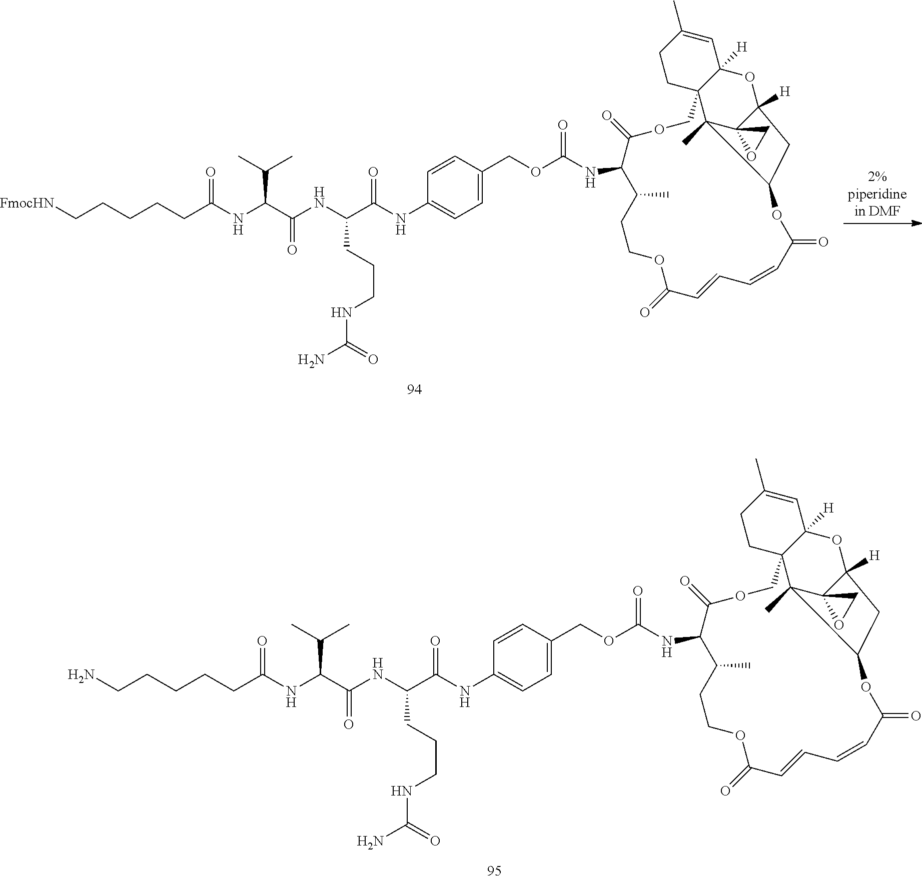

Compound 8: To a solution of Fmoc-6-aminohexanoic acid n-hydroxysuccinimide ester (50 mg, 0.11 mmol) and Val-Cit-PAB-OH TFA salt (92, 50 mg, 0.1 mmol) in DMF (1.3 mL) at room temperature, N,N-diisopropylethylamine (52 μL, 0.3 mmol) was added. The reaction was stirred for 20 minutes then was injected onto an ISCO 30 g C18Aq column and eluted with 5-95% MeCN/H₂O (both containing 0.05% AcOH). Pure fractions were combined and lyophilized to obtain compound 8 (58 mg, 81%) as a fluffy white solid. MS (ESI, pos.): calc'd for C₃₉H₅₀N₆O₇, 714.4; found 715.3 (M+H).

Compound 93: To a solution of compound 8 (40 mg, 0.056 mmol) in DMF (1.5 mL) at room temperature, bis-(4-nitrophenyl) carbonate (20.4 mg, 0.067 mmol) and N,N-diisopropylethylamine (30 μL, 0.168 mmol) were added. The reaction was stirred for 6 hours then was injected onto an ISCO 50 g C18Aq column and eluted with 5-95% MeCN/H₂O (both containing 0.05% AcOH). Pure fractions were combined and lyophilized to obtain compound 93 (29 mg, 57%) as a fluffy white solid. MS (ESI, pos.): calc'd for C₄₆H₅₃N₆O₁₁, 879.4; found 880.3 (M+H).

Compound 94: To a solution of compound 93 (30 mg, 0.034 mmol) and (R)-amino-verrucarin A (4, 17.1 mg, 0.031 mmol) in anhydrous DMF (1.3 mL) at room temperature, diisopropylethylamine (16 μL, 0.093 mmol) was added. The reaction was stirred for 36 hours then was injected onto an ISCO EZ-Prep HPLC (30×150 mm Gemini column) and eluted with 5-95% MeCN/H₂O (both containing 0.05% AcOH). Fractions containing unreacted starting amine were combined and lyophilized to recover (R)-amino verrucarin A (4, 4 mg, 23% recovery). Fractions containing the desired product were combined and lyophilized to obtain compound 94 (10 mg, 32% yield BRSM) as a fluffy white solid. MS (ESI, pos.): calc'd for C₆₇H₈₃N₇O₁₆, 1241.6; found 1242.3 (M+H).

Compound 95: To a solution of compound 94 (10 mg, 0.008 mmol) in DMF (0.5 mL) at room temperature, was added a 5% solution of piperidine in DMF (0.5 mL), and the reaction was stirred for 20 minutes. The product was purified by prep HPLC chromatography on a 30×150 mm Gemini column, eluting with 5-95% MeCN/H₂O (both containing 0.05% AcOH). Pure fractions were combined and lyophilized to obtain compound 95 (6.4 mg, 78%) as a fluffy white solid. MS (ESI, pos.): calc'd for C₅₂H₇₃N₇O₁₄, 1019.5; found 1020.2 (M+H). ¹H-NMR (500 MHz; DMSO-d₆): δ 10.02 (s, 1H), 8.14-8.11 (m, 1H), 7.92 (ddd, J=15.5, 11.7, 0.9 Hz, 1H), 7.89-7.82 (m, 1H), 7.75 (d, J=6.5 Hz, 1H), 7.61 (d, J=8.4 Hz, 2H), 7.28 (d, J=8.3 Hz, 2H), 6.85 (t, J=11.4 Hz, 1H), 6.23-6.18 (m, 2H), 6.01-5.98 (m, 1H), 5.75-5.73 (m, 1H), 5.40 (s, 2H), 5.26 (d, J=4.0 Hz, 1H), 5.03 (d, J=12.4 Hz, 1H), 4.86 (d, J=12.3 Hz, 1H), 4.45-4.41 (m, 1H), 4.39-4.35 (m, 1H), 4.17 (t, J=7.8 Hz, 1H), 4.12-4.03 (m, 2H), 3.90-3.85 (m, 1H), 3.66-3.61 (m, 3H), 3.00-2.91 (m, 4H), 2.41 (dd, J=15.2, 8.2 Hz, 1H), 2.19-2.08 (m, 4H), 1.98-1.89 (m, 2H), 1.81 (s, 1H), 1.70-1.66 (m, 3H), 1.62 (s, 3H), 1.50-1.41 (m, 6H), 1.37-1.32 (m, 3H), 1.26-1.22 (m, 6H), 0.91 (d, J=6.6 Hz, 3H), 0.83 (dd, J=12.4, 6.7 Hz, 6H), 0.65 (s, 3H).

### Example 35

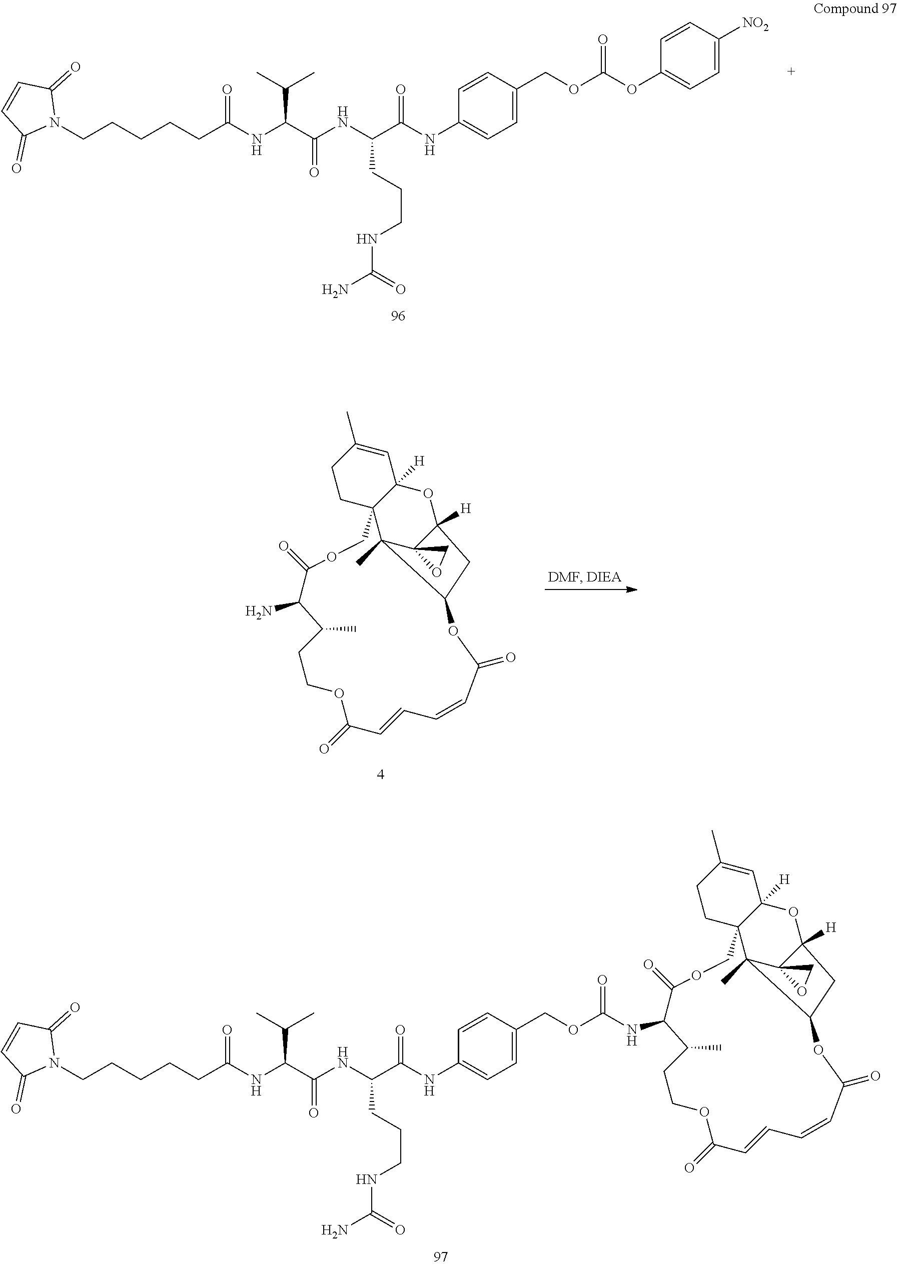 [SMILES: CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1 ; CC1=C[C@H]2O[C@@H]3C[C@H]4OC(=O)/C=C\C=C\C(=O)OCC[C@@H](C)[C@@H](N)C(=O)OC[C@@]2(CC1)[C@]4(C)[C@]31CO1 ; CC1=C[C@H]2O[C@@H]3C[C@H]4OC(=O)/C=C\C=C\C(=O)OCC[C@@H](C)[C@@H](NC(=O)OCc5ccc(NC(=O)[C@H](CCCNC(N)=O)NC(=O)[C@@H](NC(=O)CCCCCN6C(=O)C=CC6=O)C(C)C)cc5)C(=O)OC[C@@]2(CC1)[C@]4(C)[C@]31CO1]

Compound 96 was prepared following the same procedures as for compound 93, except starting with N-succinimidyl 6-maleimidohexanoate instead of Fmoc-6-aminohexanoic acid N-hydroxysuccinimide ester

Compound 97: To a DMF (1 mL) solution (R)-amino-verrucarin A (4, 6.9 mg, 0.014 mmol) at room temperature, compound 96 (11.4 mg, 0.015 mmol) was added followed by N,N-diisopropylethylamine (7 μL, 0.041 mmol). The resulting mixture was stirred for 18 hours, at which time the reaction was judged complete by LCMS analysis. The reaction mixture was injected onto an ISCO 15.5 g C18Aq column and eluted with 5-95% MeCN/H₂O (both containing 0.05% AcOH). Pure fractions were combined and lyophilized to obtain compound 97 (9 mg, 60% yield) as white fluffy solid. MS (ESI, pos.): calc'd for C₅₆H₇₃N₇O₁₆, 1099.5; found 1100.5 (M+H). ¹H-NMR (500 MHz; DMSO-d₆): δ 10.06 (s, 1H), 8.17 (s, 1H), 7.93 (dd, J=15.1, 12.1 Hz, 1H), 7.84 (d, J=7.8 Hz, 1H), 7.75 (d, J=6.5 Hz, 1H), 7.62 (d, J=8.3 Hz, 2H), 7.29 (d, J=8.3 Hz, 2H), 7.00 (s, 2H), 6.86 (t, J=11.4 Hz, 1H), 6.23 (m, 2H), 6.02 (s, 1H), 5.75 (dd, J=7.0, 2.9 Hz, 1H), 5.41 (s, 2H), 5.27 (s, 1H), 5.04 (d, J=12.3 Hz, 1H), 4.87 (d, J=12.3 Hz, 1H), 4.46-4.43 (m, 1H), 4.38 (q, J=6.5 Hz, 1H), 4.18 (t, J=7.7 Hz, 1H), 4.08 (q, J=14.7 Hz, 2H), 3.89 (td, J=9.5, 3.3 Hz, 1H), 3.67-3.62 (m, 4H), 3.02-2.95 (m, 4H), 2.44-2.39 (m, 1H), 2.21-2.13 (m, 3H), 2.13-2.07 (m, 3H), 1.98-1.90 (m, 2H), 1.70-1.63 (m, 5H), 1.50-1.43 (m, 6H), 1.26-1.16 (m, 3H), 0.92 (d, J=6.5 Hz, 3H), 0.83 (dd, J=15.1, 6.7 Hz, 9H), 0.66 (s, 3H).

### Example 36

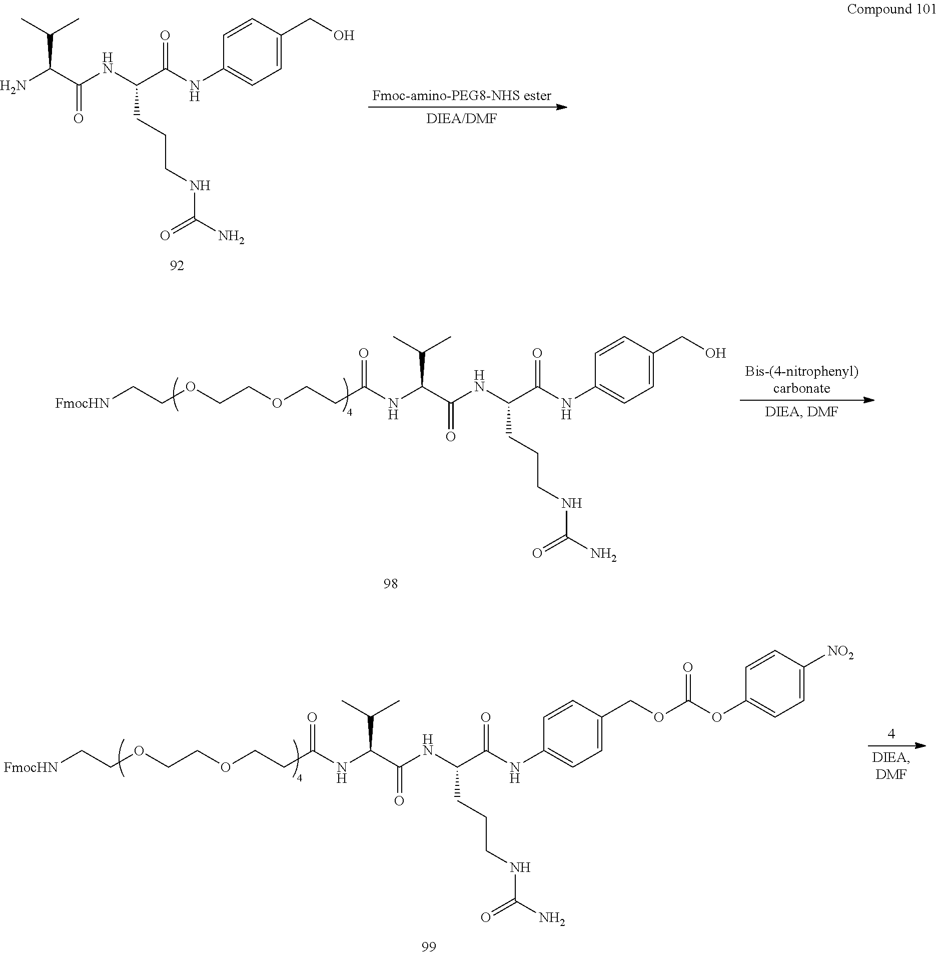

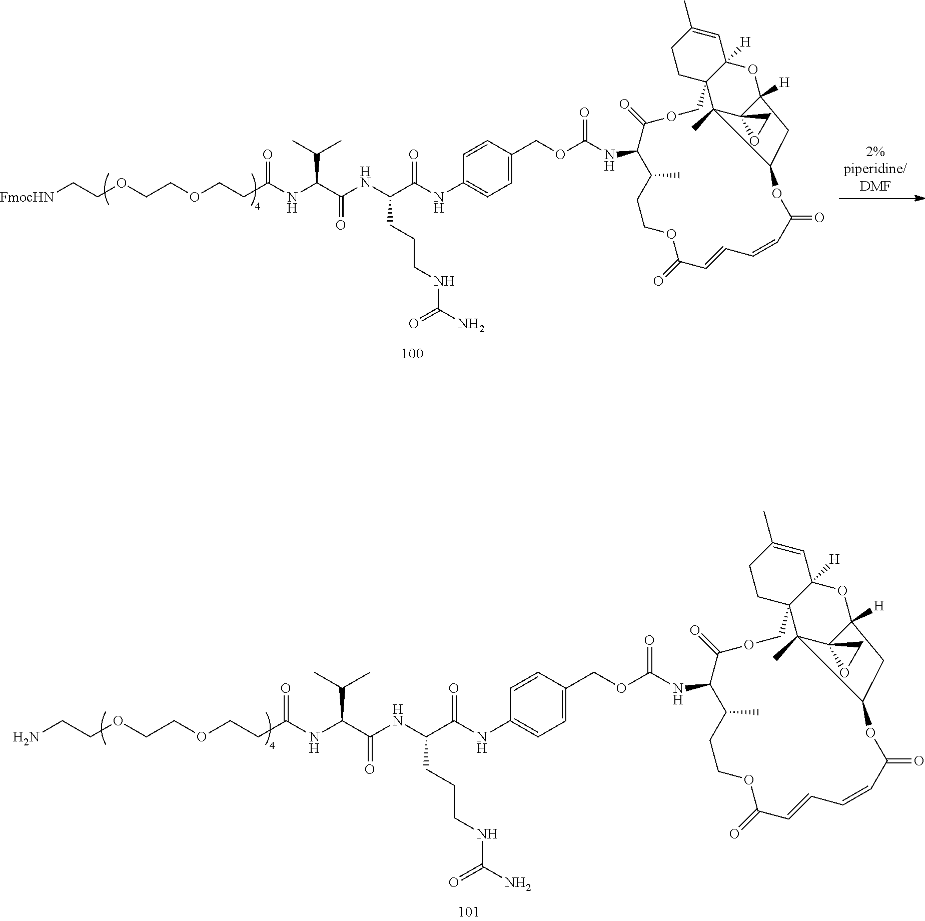

Compound 98: To a solution of Valine-Citrulline-PAB-OH TFA salt, (92, 100 mg, 0.2 mmol) and Fmoc-amino-PEG8-NHS ester (167 mg, 0.22 mmol) in anhydrous DMF (2 mL), N,N-diisopropylethylamine (40 μL, 0.22 mmol) was added and the reaction was stirred for 45 min, at which time LC/MS indicated the reaction was complete. The product was purified by chromatography on an ISCO 100 g C18Aq column, eluting with 5-95% MeCN/H₂O (both having 0.05% AcOH). Pure fractions were combined and lyophilized to obtain compound 98 (145 mg, 71%) as an off-white fluffy solid. MS (ESI, pos.): calc'd for C₅₂H₇₆N₆O₁₅, 1024.54; found 1025.5 (M+H).

Compound 99: To a solution of compound 98 (50 mg, 0.0488 mmol) and bis-(4-nitrophenyl) carbonate (18 mg, 0.0585 mmol) in anhydrous DMF (1 mL) at room temperature, N,N-diisopropylethylamine (19 μL, 0.146 mmol) was added and the reaction was stirred for 18 hours. Additional bis-(4-nitrophenyl) carbonate (18 mg, 0.0585 mmol) was added and stirring was continued for 3 hours, at which time LCMS indicated the reaction was complete. The reaction solution was injected onto an ISCO 50 g C18Aq column and eluted with 5-95% MeCN/H₂O (both having 0.05% AcOH). Pure fractions were combined and lyophilized to obtain compound 99 (37 mg, 64%) as an off-white fluffy solid. MS (ESI, pos.): calc'd for C₅₉H₇₉N₇O₁₉, 1189.5; found 1190.5 (M+H).

Compound 100: To a solution of compound 99 (30 mg, 0.025 mmol) and (R)-amino-verrucarin A (4, 14 mg, 0.025 mmol) in anhydrous DMF at room temperature, N,N-diisopropylethylamine (12 μL, 0.075 mmol) was added and the reaction was stirred for 24 hours. The reaction mixture was injected onto an ISCO 30 g C18Aq column and eluted with 5-95% MeCN/H₂O (both having 0.05% AcOH). Fractions containing the desired product were combined and lyophilized. The lyophilized solids were dissolved in DMF (1 mL) and further purified by prep HPLC on a Gemini 30×150 mm column, eluting with 5-95% MeCN/H₂O (both having 0.05% AcOH). Pure fractions were combined and lyophilized to obtain compound 100 (8 mg, 21%) as an off-white fluffy solid. MS (ESI, pos.): calc'd for C₈₀H₁₀₉N₇O₂₄, 1551.8; found 1552.7 (M+H).

Compound 101: To a solution of compound 100 (8 mg, 0.005 mmol) in DMF (0.6 mL) at room temperature, a 5% solution of piperidine in DMF (0.3 mL) was added and the reaction was stirred for 45 minutes. The reaction solution was injected onto an ISCO 30 g C18Aq column and eluted with 5-95% MeCN/H₂O (both having 0.05% AcOH). Pure fractions were combined and lyophilized to obtain compound 101 (3.0 mg, 45%) as an off-white fluffy solid. MS (ESI, pos.): calc'd for C₆₅H₉₉N₇O₂₂, 1329.7; found 1330.6 (M+H). ¹H-NMR (500 MHz; DMSO-d₆): δ 10.00 (s, 1H), 8.11 (d, J=7.5 Hz, 1H), 7.92 (dd, J=15.5, 11.7 Hz, 1H), 7.85 (d, J=8.7 Hz, 1H), 7.74 (d, J=6.6 Hz, 1H), 7.60 (d, J=8.4 Hz, 2H), 7.28 (d, J=8.4 Hz, 2H), 6.85 (t, J=11.4 Hz, 1H), 6.21 (dd, J=13.2, 10.3 Hz, 2H), 5.96 (t, J=5.6 Hz, 1H), 5.74 (dd, J=8.1, 4.1 Hz, 1H), 5.39 (s, 2H), 5.27-5.26 (m, 1H), 5.04 (d, J=12.4 Hz, 1H), 4.86 (d, J=12.3 Hz, 1H), 4.45-4.41 (m, 1H), 4.39-4.36 (m, 1H), 4.22 (dd, J=8.6, 6.9 Hz, 1H), 4.10 (d, J=12.1 Hz, 1H), 4.05 (d, J=12.1 Hz, 1H), 3.90-3.85 (m, 1H), 3.66-3.56 (m, 5H), 3.46 (d, J=16.6 Hz, 28H), 3.03-2.91 (m, 4H), 2.77 (t, J=5.5 Hz, 2H), 2.41-2.35 (m, 3H), 2.18-2.16 (m, 1H), 2.09-2.06 (m, 1H), 1.94 (dt, J=13.5, 5.6 Hz, 2H), 1.90 (s, 1H), 1.70-1.68 (m, 3H), 1.62 (s, 3H), 1.51-1.43 (m, 6H), 1.22 (s, 3H), 0.91 (d, J=6.6 Hz, 3H), 0.83 (dd, J=15.6, 6.7 Hz, 6H), 0.65 (s, 3H).

### Example 37

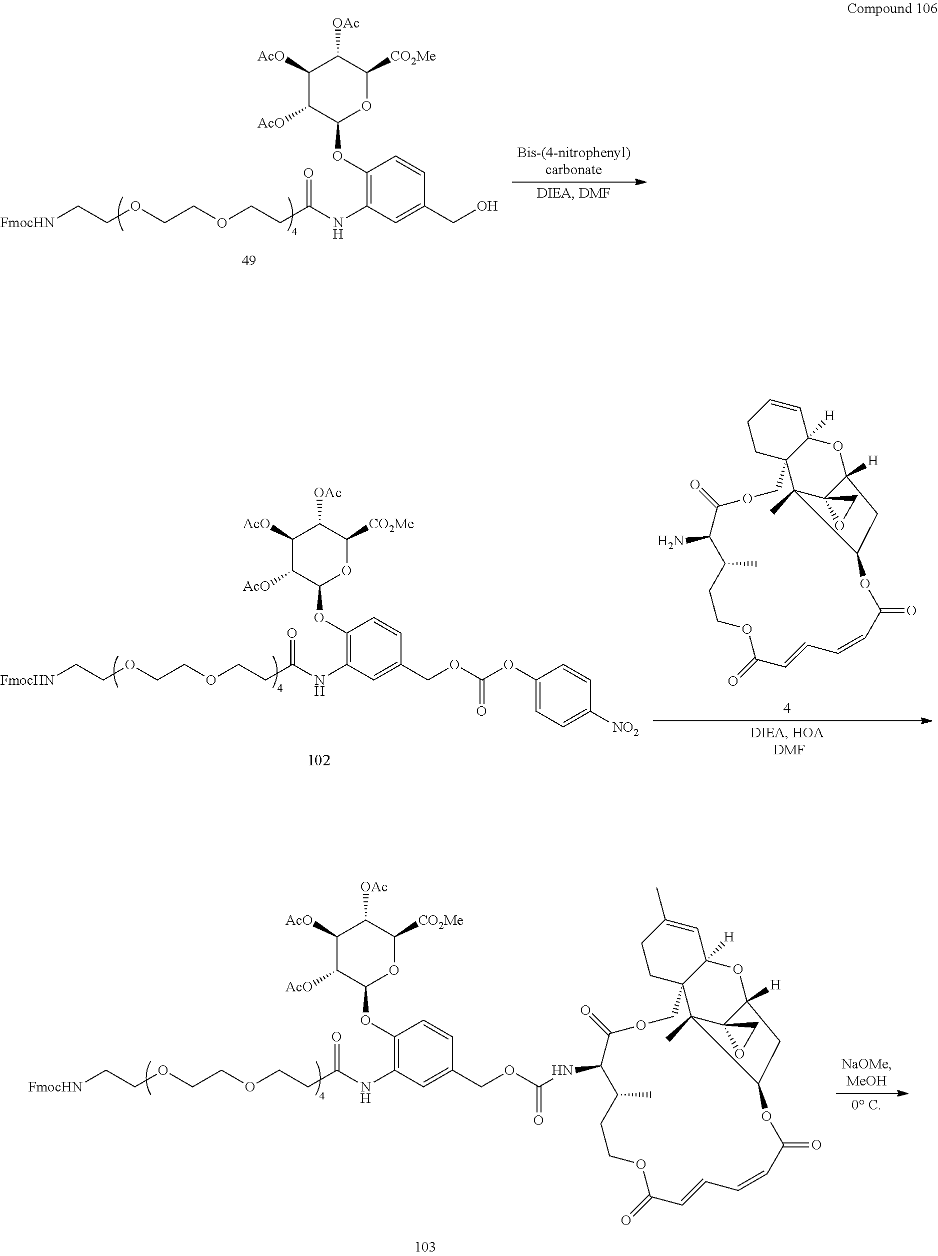

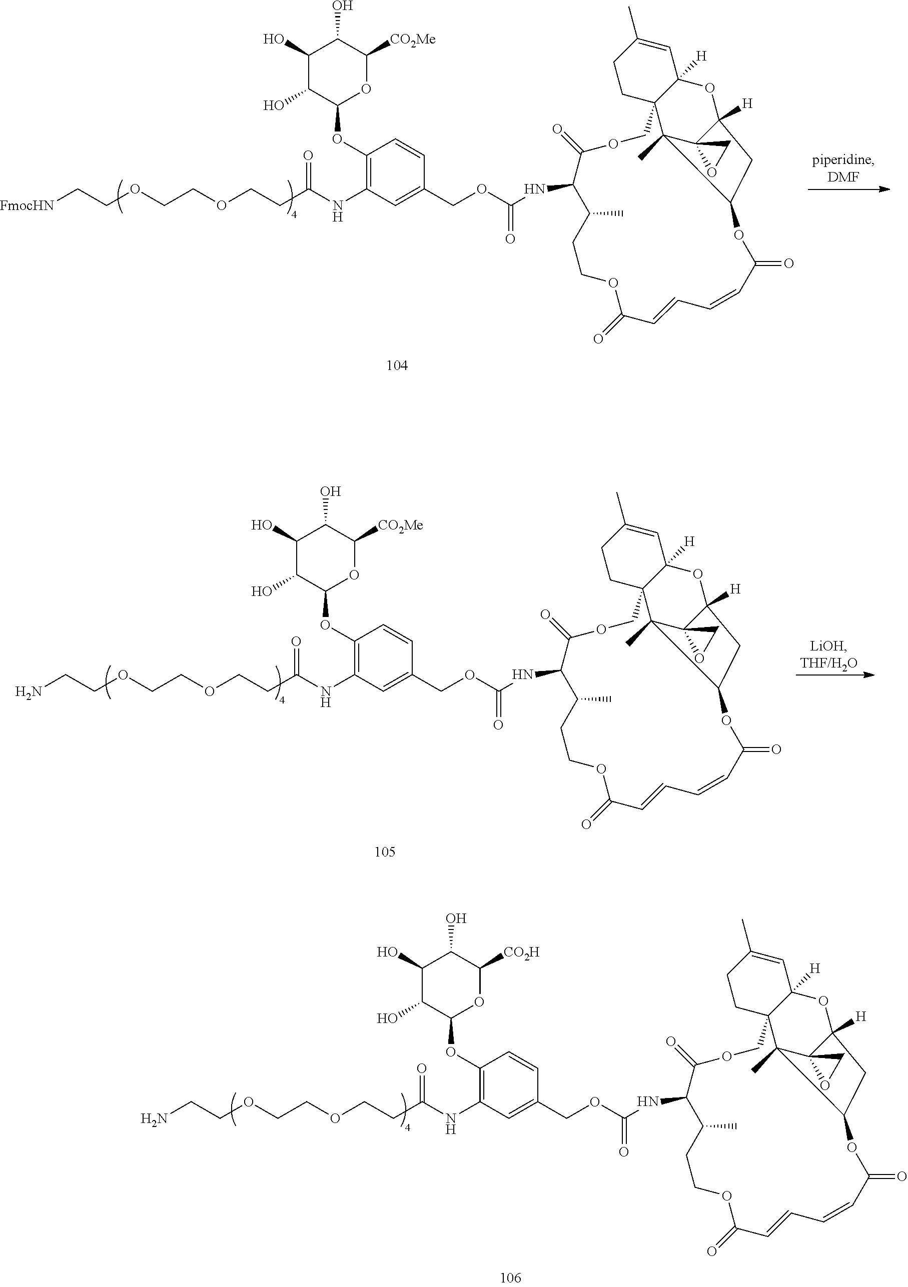

Compound 102: To a solution of compound 49 (47.1 mg, 0.0428 mmol) in anhydrous DMF (1 mL) were added bis-(4-nitrophenyl) carbonate (20 mg, 0.064 mmol) and N,N-diisopropylethylamine (22 μL, 0.128 mmol). After stirring for 5 hours, the reaction was purified on an ISCO 50 g C18Aq column, eluting with 5-95% MeCN/H₂O (both having 0.05% AcOH). Fractions containing pure product were combined and lyophilized to afford compound 102 (36 mg, 66%) as a yellowish fluffy solid. MS (ESI, pos.): calc'd for C₆₁H₇₅N₃O₂₆, 1265.5; found 1266.8 (M+H).

Compound 103: To a solution of compound 102 (18 mg, 0.014 mmol) and compound 4 (7 mg, 0.014 mmol) in anhydrous DMF (1 mL) were added N,N-diisopropylethylamine (7.3 μL, 0.042 mmol) and HOAt (1.0 mg, 0.007 mmol). After stirring for 24 hours, the reaction was purified on an ISCO 30 g C18Aq column, eluting with 5-95% MeCN/H₂O (both having 0.05% AcOH). Pure fractions were combined and lyophilized to obtain compound 103 (12 mg, 53%) as a fluffy white solid. MS (ESI, pos.): calc'd for C₈₂H₁₀₅N₃O₃₁, 1627.7; found 1628.6 (M+H).

Compound 105: To a solution of compound 103 (20 mg, 0.012 mmol) in anhydrous MeOH (1.5 mL) at 0° C. was added 0.05 M NaOMe in MeOH (0.48 mL, 0.024 mmol). After stirring for 10 minutes at 0° C., the reaction was neutralized with Dowex® Resin (ca 10 mg). Solids were filtered off and the filtrate was concentrated in vacuo to afford crude compound 104, which was then dissolved in DMF (0.6 mL), and 5% piperidine in DMF (0.3 mL) was added. After stirring for 20 minutes, the reaction was purified on an ISCO 30 g C18Aq column, eluting with 5-95% MeCN/H₂O (both having 0.05% AcOH). Pure fractions were combined and lyophilized to obtain compound 105 (4 mg, 26% over two steps) as a fluffy off-white solid. MS (ESI, pos.): calc'd for C₆₁H₈₉N₃O₂₆, 1279.6; found 1280.5 (M+H).

Compound 106: To a solution of compound 105 (4 mg, 0.003 mmol) in THF (0.5 mL) and water (375 μL), 0.025M aqueous LiOH solution (250 μL, 0.006 mmol) was added and the reaction was stirred for 2 hours. Volatiles were removed under reduced pressure and the residue was purified by prep HPLC on a 30×150 mm Gemini column using 5-95% MeCN/H₂O (both having 0.05% AcOH). Pure fractions were combined and lyophilized to obtain compound 106 (2.5 mg, 66%) as a fluffy off-white solid. MS (ESI, pos.): calc'd for C₆₀H₈₇N₃O₂₆, 1265.6; found 1266.5 (M+H). ¹H-NMR (500 MHz; DMSO-d₆): δ 9.31 (s, 1H), 8.17 (dd, J=1.3, 0.8 Hz, 1H), 7.90 (dd, J=15.2, 11.1 Hz, 1H), 7.77 (d, J=6.5 Hz, 1H), 7.16 (d, J=8.2 Hz, 1H), 7.03 (d, J=7.3 Hz, 1H), 6.84 (t, J=11.4 Hz, 1H), 6.21 (dd, J=17.1, 13.2 Hz, 2H), 5.75-5.72 (m, 1H), 5.69-5.68 (m, 1H), 5.26 (d, J=4.5 Hz, 1H), 5.07-5.05 (m, 1H), 4.90 (q, J=14.7 Hz, 2H), 4.56 (d, J=6.5 Hz, 1H), 4.44-4.41 (m, 1H), 4.11 (d, J=10.8 Hz, 1H), 4.02 (d, J=11.7 Hz, 1H), 3.89-3.85 (m, 1H), 3.73-3.61 (m, 5H), 3.52-3.48 (m, 24H), 3.09 (d, J=5.4 Hz, 1H), 2.89 (t, J=5.1 Hz, 4H), 2.58 (d, J=4.6 Hz, 1H), 2.19-2.16 (m, 2H), 2.08 (d, J=2.9 Hz, 2H), 1.93-1.90 (m, 2H), 1.62 (m, 4H), 1.47 (s, 3H), 1.25 (dd, J=12.2, 10.5 Hz, 2H), 0.91 (d, J=6.8 Hz, 3H), 0.67 (s, 3H).

### Example 38

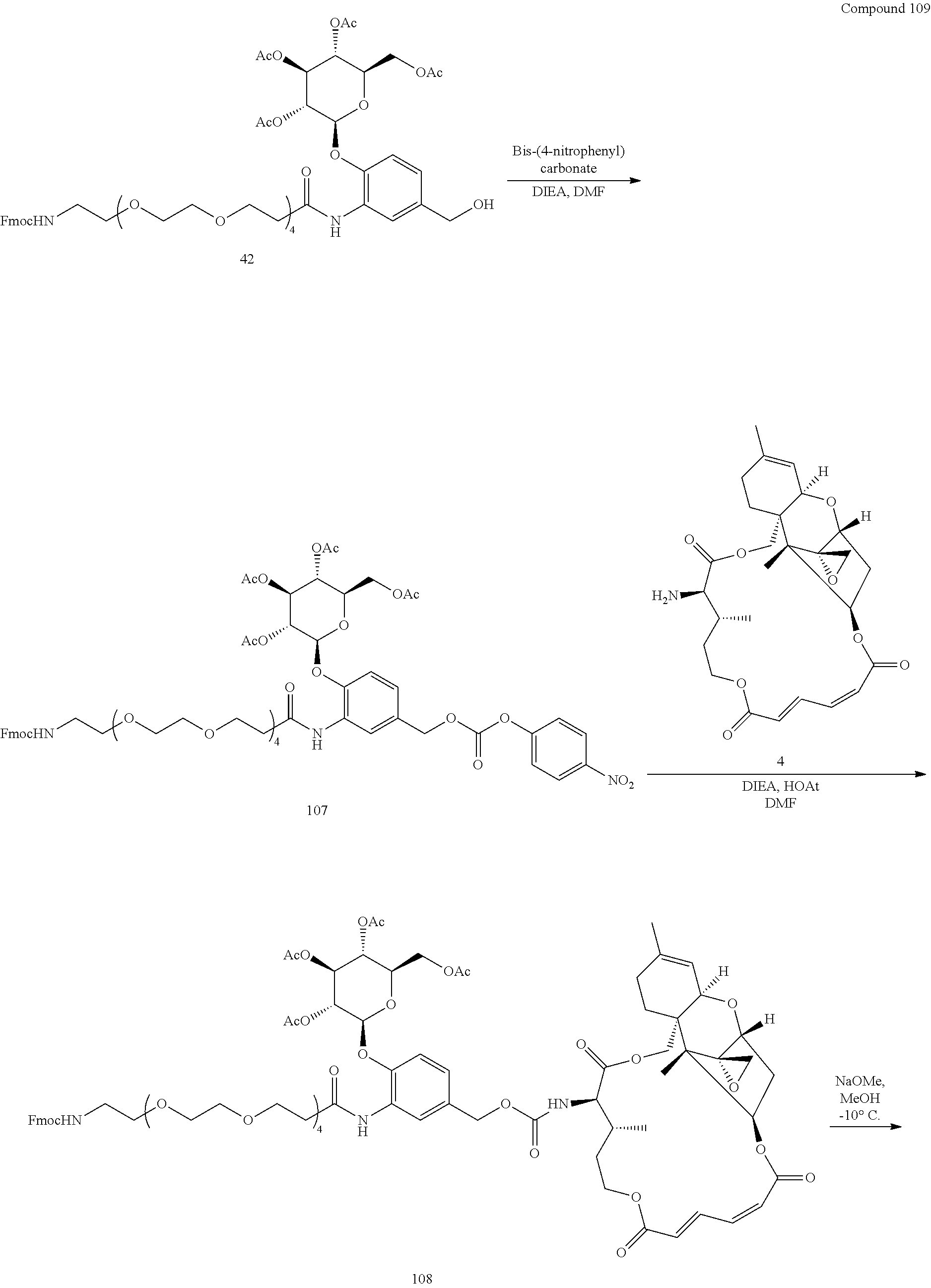

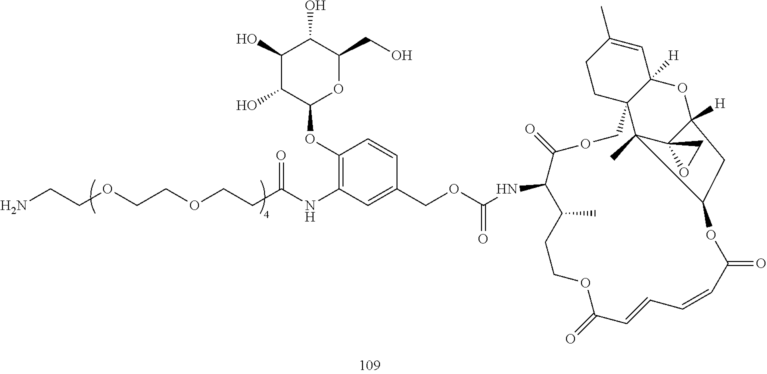

Compound 107: To a solution of compound 42 (190 mg, 0.17 mmol) in anhydrous DMF (2 mL) were added bis-(4-nitrophenyl) carbonate (104 mg, 0.34 mmol) and N,N-diisopropylethylamine (89 μL, 0.51 mmol). After stirring for 22 hours, the reaction was loaded onto an ISCO 100 g C18Aq column and eluted with 5-95% MeCN/H₂O (both having 0.05% AcOH). Fractions containing pure product were combined and lyophilized to afford compound 107 (130 mg, 60%) as a fluffy solid. MS (ESI, pos.): calc'd for C₆₂H₇₇N₃O₂₆, 1279.5; found 1280.4 (M+H).

Compound 108: To a solution of compound 107 (45 mg, 0.035 mmol) and compound 4 (17.1 mg, 0.035 mmol) in anhydrous DMF (1.5 mL) were added N,N-diisopropylethylamine (18 μL, 0.105 mmol) and HOAt (2.5 mg, 0.018 mmol). After stirring for 9 hours, the reaction was purified on an ISCO 100 g C18Aq column, eluting with 5-95% MeCN/H₂O (both having 0.05% AcOH). Pure fractions were combined and lyophilized to obtain compound 108 (44 mg, 76%) as a fluffy off-white solid. MS (ESI, pos.): calc'd for C₈₃H₁₀₅N₃O₃₁, 1641.7; found 1642.7 (M+H).

Compound 109: To a solution of compound 108 (30 mg, 0.018 mmol) in anhydrous MeOH (1.5 mL) at −10° C. was added 0.1 M NaOMe in MeOH (0.36 mL, 0.036 mmol). After stirring for 90 minutes at −10° C., the reaction was neutralized with Dowex® Resin (ca. 100 mg). Solids were filtered off and the filtrate was concentrated in vacuo. The residue was purified by prep HPLC on a 50×250 mm Luna column, eluting with 5-95% MeCN/H₂O (both having 0.05% AcOH). Pure fractions were combined and lyophilized to obtain compound 109 (5 mg, 18% over two steps) as a fluffy off-white solid. MS (ESI, pos.): calc'd for C₆₀H₈₉N₃O₂₆, 1251.6; found 1252.5 (M+H). ¹H-NMR (500 MHz; DMSO-d₆): δ 9.16 (s, 1H), 8.23 (d, J=1.4 Hz, 1H), 7.91 (dd, J=15.7, 11.8 Hz, 1H), 7.77 (dd, J=6.3, 4.2 Hz, 1H), 7.15 (dd, J=8.3, 2.4 Hz, 1H), 7.02-6.99 (m, 1H), 6.84 (t, J=11.5 Hz, 1H), 6.20 (t, J=13.3 Hz, 2H), 5.74 (dd, J=8.1, 3.7 Hz, 1H), 5.26 (dt, J=4.1, 1.1 Hz, 1H), 4.98-4.85 (m, 3H), 4.56 (d, J=7.7 Hz, 1H), 4.45-4.41 (m, 1H), 4.19-3.98 (m, 3H), 3.90-3.84 (m, 2H), 3.71-3.63 (m, 8H), 3.52-3.47 (m, 26H), 3.12-3.00 (m, 3H), 2.67-2.62 (m, 7H), 2.35 (t, J=1.9 Hz, 1H), 2.20-2.06 (m, 2H), 1.94-1.90 (m, 1H), 1.88 (d, J=3.6 Hz, 2H), 1.67-1.62 (m, 5H), 1.55-1.46 (m, 2H), 1.27-1.22 (m, 1H), 0.91 (d, J=6.7 Hz, 3H), 0.66 (s, 3H).

### Example 39

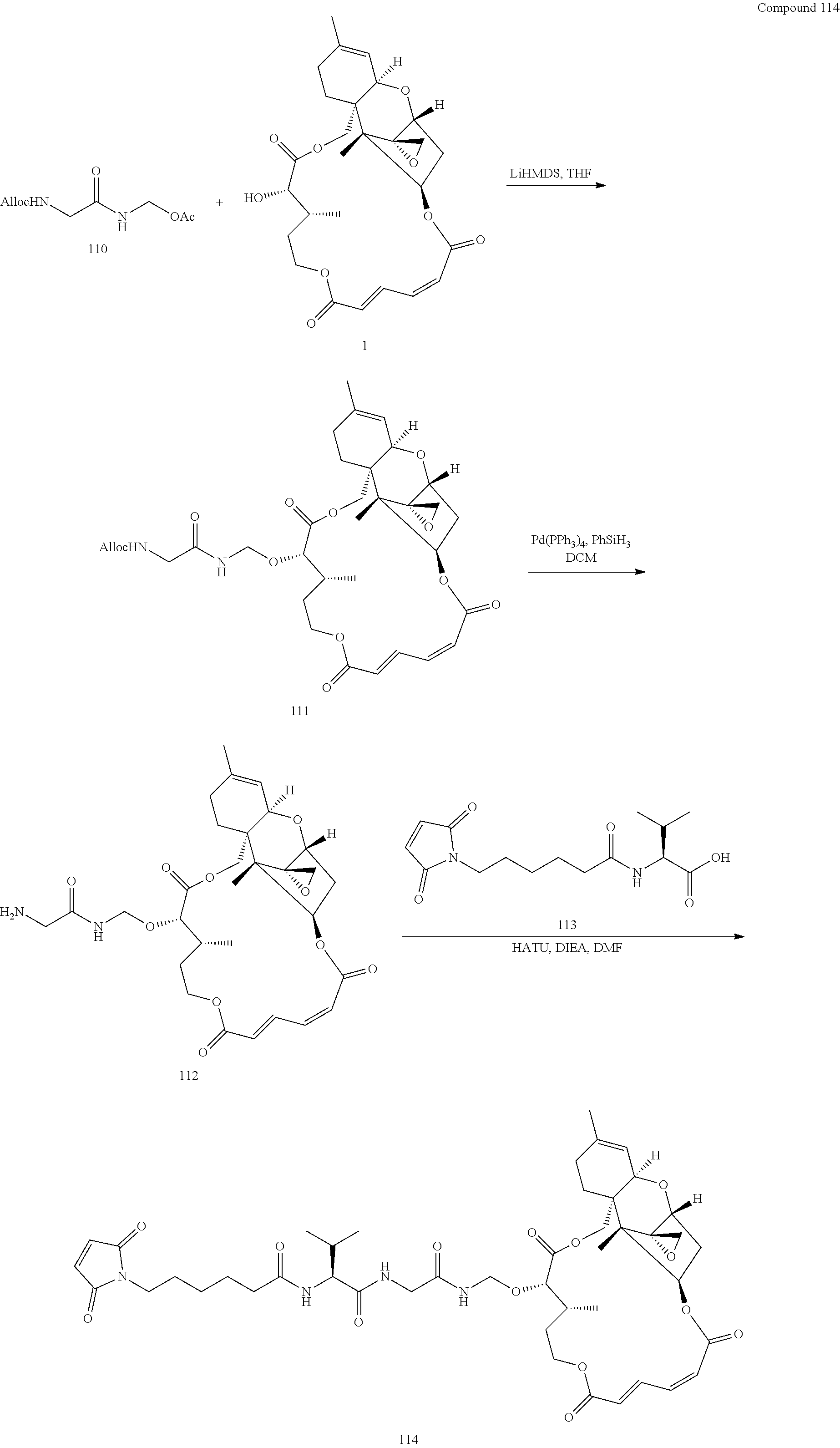

Compound 110: (2-(((allyloxy)carbonyl)amino)acetamido)methyl acetate (110) was prepared using the procedure from Tetrahedron, 2018, 74(15), 1951-1956.

Compound 111: To the solution of compound 110 (27.6 mg, 0.12 mmol) and Verrucarin A (1, 30 mg, 0.06 mmol) in anhydrous THF (2.5 mL) at 0° C., LiHMDS (72 μL, 1.0 M solution in hexanes, 0.072 mmol) was added. After 30 minutes LCMS showed 26% desired product and 60% unreacted 1. Additional 110 (14 mg, 0.06 mmol) was added followed by LiHMDS (60 μL, 0.06 mmol) and the reaction was stirred for another 30 minutes. The reaction was quenched with saturated aqueous ammonium chloride (5 mL) and extracted with ethyl acetate (3×10 mL). The combined organics were dried over anhydrous sodium sulfate, filtered and concentrated. The product was chromatographed on an ISCO 4 g Silica Gold column, eluting with 50% ethyl acetate/hexanes to recover unreacted verrucarin A (1, 10 mg, 29%) and then 80% ethyl acetate/hexanes to elute compound 111 (20.5 mg, 45%). MS (ESI, pos.): calc'd for C₃₄H₄₄N₂O₁₂, 672.3; found 673.3 (M+H).

Compound 112: To a solution of compound 111 (15 mg, 0.022 mmol) in dichloromethane (2.5 mL), were added Pd(PPh₃)₄ (2.5 mg, 0.0022 mmol) and phenylsilane (4 μL, 0.033 mmol). The reaction was stirred for 1 hour, at which time LCMS indicated the reaction was complete. The reaction was filtered through a pad of celite and washed with dichloromethane (3 mL). The filtrate was concentrated to obtain compound 112, which was used in the next step without purification. MS (ESI, pos.): calc'd for C₃₀H₄₀N₂O₀, 588.3; found 589.3 (M+H).

Compound 114: To a solution of compound 112 (0.022 mmol) in anhydrous DMF (1 mL) at room temperature, Mal-cap-Val-OH (113, 3.7 mg, 0.022 mmol) and HATU (12.6 mg, 0.033 mmol) were added followed by N,N-diisopropylethylamine (12 μL, 0.066 mmol). The reaction was complete in 30 minutes by LCMS. The product was purified by prep HPLC on a 30×150 mm Gemini column, eluting with 5-95% MeCN/H₂O (both having 0.05% AcOH). Pure fractions were combined and lyophilized to obtain compound 114 (5 mg, 25% over two steps). MS (ESI, pos.): calc'd for C₄₅H₆₀N₄O₁₄, 880.4; found 881.3 (M+H). ¹H-NMR (500 MHz; CDCl₃): δ 8.01 (dd, J=15.0, 11.6 Hz, 1H), 6.71 (s, 2H), 6.67 (t, J=11.4 Hz, 1H), 6.46 (t, J=5.2 Hz, 1H), 6.17 (d, J=11.1 Hz, 1H), 6.04 (d, J=15.6 Hz, 1H), 5.99 (d, J=6.9 Hz, 1H), 5.82 (dd, J=7.9, 3.7 Hz, 1H), 5.44 (d, J=3.3 Hz, 1H), 4.91 (dd, J=10.8, 8.5 Hz, 1H), 4.77 (d, J=12.0 Hz, 1H), 4.63 (dd, J=10.9, 5.5 Hz, 1H), 4.47-4.44 (m, 1H), 4.14-4.08 (m, 3H), 4.01-3.96 (m, 2H), 3.87 (d, J=5.0 Hz, 1H), 3.79 (dd, J=16.9, 5.0 Hz, 1H), 3.58 (d, J=5.0 Hz, 1H), 3.53 (t, J=7.1 Hz, 2H), 3.14 (d, J=3.9 Hz, 1H), 2.85 (d, J=3.9 Hz, 1H), 2.49 (t, J=7.5 Hz, 1H), 2.38-2.35 (m, 1H), 2.25-2.20 (m, 3H), 2.12-2.04 (m, 3H), 1.91-1.87 (m, 2H), 1.77 (s, 3H), 1.69-1.62 (m, 3H), 1.36-1.27 (m, 6H), 0.99 (dd, J=12.7, 6.7 Hz, 6H), 0.93 (d, J=6.8 Hz, 3H), 0.86 (s, 3H).

### Example 40

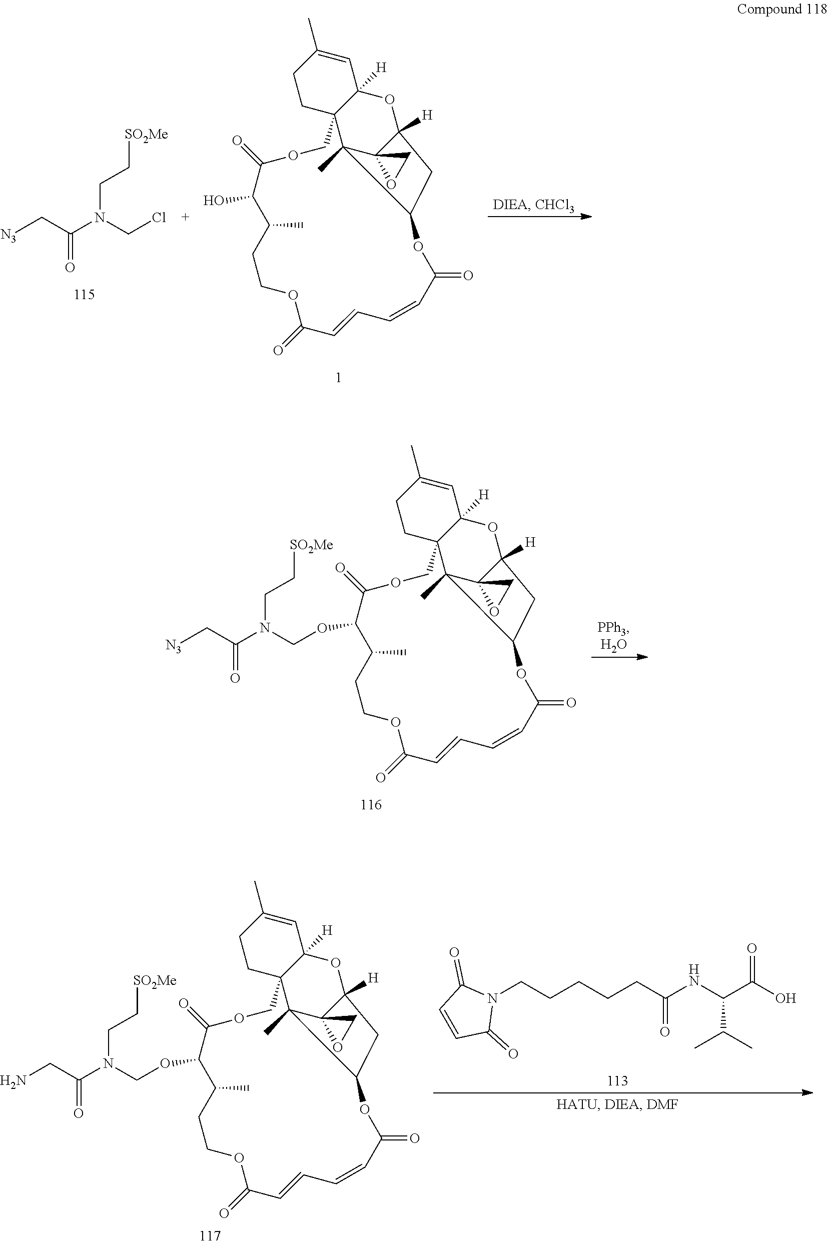 [SMILES: CS(=O)(=O)CCN(CCl)C(=O)CN=[N+]=[N-] ; CC1=C[C@H]2O[C@@H]3C[C@H]4OC(=O)/C=C\C=C\C(=O)OCC[C@@H](C)[C@H](O)C(=O)OC[C@@]2(CC1)[C@]4(C)[C@]31CO1 ; CC1=C[C@H]2O[C@@H]3C[C@H]4OC(=O)/C=C\C=C\C(=O)OCC[C@@H](C)[C@H](OCN(CCS(C)(=O)=O)C(=O)CN=[N+]=[N-])C(=O)OC[C@@]2(CC1)[C@]4(C)[C@]31CO1 ; CC1=C[C@H]2O[C@@H]3C[C@H]4OC(=O)/C=C\C=C\C(=O)OCC[C@@H](C)[C@H](OCN(CCS(C)(=O)=O)C(=O)CN)C(=O)OC[C@@]2(CC1)[C@]4(C)[C@]31CO1 ; CC(C)[C@H](NC(=O)CCCCCN1C(=O)C=CC1=O)C(=O)O]

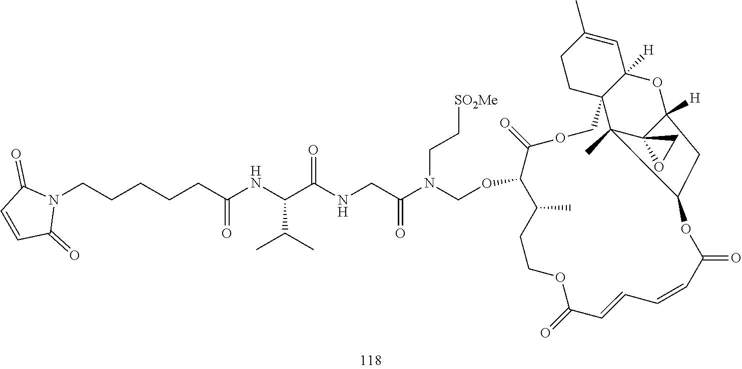 [SMILES: CC1=C[C@H]2O[C@@H]3C[C@H]4OC(=O)/C=C\C=C\C(=O)OCC[C@@H](C)[C@H](OCN(CCS(C)(=O)=O)C(=O)CNC(=O)[C@@H](NC(=O)CCCCCN5C(=O)C=CC5=O)C(C)C)C(=O)OC[C@@]2(CC1)[C@]4(C)[C@]31CO1]

Compound 116: To a solution of Verrucarin A (1, 25 mg, 0.05 mmol) in anhydrous chloroform (0.25 mL), compound 115 (38 mg, 0.15 mmol) and N,N-diisopropylethylamine (35 μL, 0.2 mmol) were added and the reaction was heated to 40° C. for 22 hours. Volatiles were removed under reduced pressure and the residue was purified by prep HPLC on a 30×150 mm Gemini column using 5-95% MeCN/H₂O (both having 0.05% AcOH). Pure fractions were combined and lyophilized to obtain compound 116 (15 mg, 43%) as a fluffy white solid. MS (ESI, pos.): calc'd for C₃₃H₄₄N₄O₁₂S, 720.3; found 721.3 M+H) and 743.3 (M+Na).

Compound 117: To a solution of compound 116 (15 mg, 0.02 mmol) in 10:1 THF/H₂O (0.4 mL), triphenylphosphine (13.6 mg, 0.05 mmol) was added. After stirring for 24 hours at room temperature, the reaction was concentrated to dryness and used for the next step without purification. MS (ESI, pos.): calc'd for C₃₃H₄₆N₂O₁₂S, 694.3; found 695.3 M+H).

Compound 118: To a solution of compound 117 (0.02 mmol) and Mal-cap-Val-OH (113, 18.6 mg, 0.06 mmol) in anhydrous DMF (0.4 mL), HATU (19 mg, 0.05 mmol) and N,N-diisopropylethylamine (10.3 μL, 0.06 mmol) were added. After stirring for 1 hour, the reaction was chromatographed on an ISCO 30 g C18Aq column using 5-95% MeCN/H₂O (both having 0.05% AcOH). Pure fractions were combined and lyophilized to obtain compound 118 (5.2 mg, 27%) as a fluffy off-white solid. MS (ESI, pos.): calc'd for C₄₈H₆₆N₄O₁₆S, 986.4; found 987.4 M+H). ¹H-NMR (500 MHz; acetone-d₆): δ 8.11-8.05 (t, J=14.2 Hz, 1H), 7.39-7.37 (m, 1H), 7.10-7.08 (m, 1H), 6.87 (d, J=9.8 Hz, 2H), 6.26 (d, J=11.2 Hz, 1H), 6.15 (d, J=15.8 Hz, 1H), 5.90-5.89 (m, 1H), 5.40-5.39 (m, 1H), 4.99 (d, J=1.8 Hz, 2H), 4.73-4.71 (m, 1H), 4.46-4.43 (m, 1H), 4.37-4.34 (m, 1H), 4.31 (s, 1H), 4.27-4.23 (m, 2H), 4.18-4.14 (m, 1H), 4.03-3.92 (m, 2H), 3.85-3.84 (m, 1H), 3.73 (t, J=6.5 Hz, 2H), 3.49-3.43 (m, 3H), 3.40-3.38 (m, 1H), 3.06-3.03 (m, 2H), 3.00 (s, 3H), 2.54-2.48 (m, 2H), 2.30-2.27 (m, 1H), 2.19-2.16 (m, 1H), 1.99-1.94 (m, 3H), 1.90-1.79 (m, 4H), 1.71 (s, 3H), 1.67-1.64 (m, 2H), 1.60-1.56 (m, 2H), 1.33 (t, J=7.6 Hz, 3H), 0.97-0.91 (m, 9H), 0.89 (s, 3H).

### Example 41

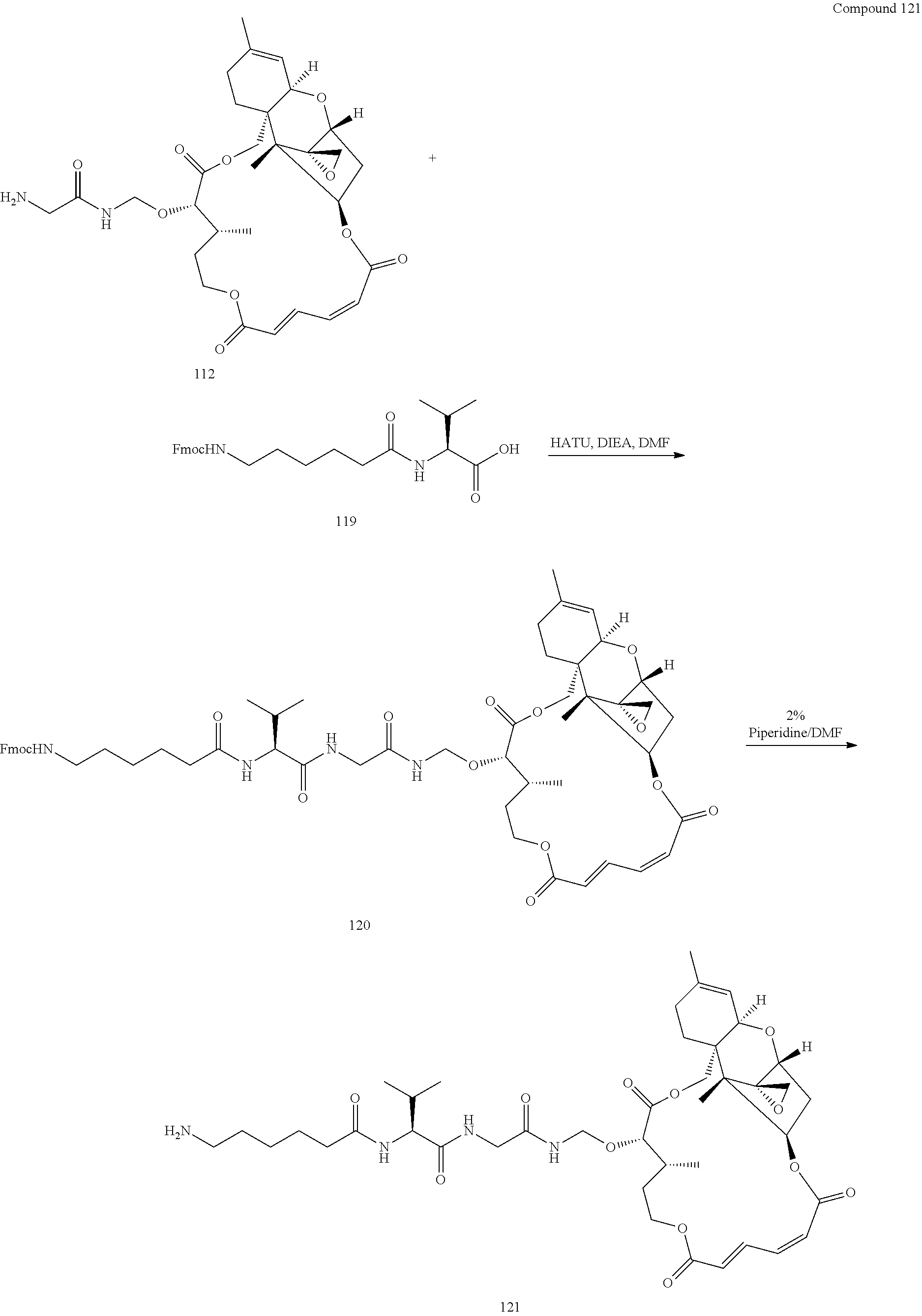

Compound 120: To a solution of crude 112 (20 mg, 0.03 mmol), in DMF (2 mL) at room temperature, HATU (17.2 mg, 0.045 mmol) and Fmoc-amino-cap-Val-OH (119, 15 mg, 0.033 mmol) were added followed by N,N-diisopropylethylamine (16 μL, 0.09 mmol). The reaction was complete after 30 minutes by LCMS. The reaction solution was loaded onto an ISCO with 15.5 g C18Aq column and eluted with 5-95% MeCN/H₂O (both containing 0.05% AcOH). Pure fractions were combined and lyophilized to obtain compound 120 (18.2 mg, 50% over 2 steps). MS (ESI, pos.): calc'd for C₅₆H₇₀N₄O₁₄, 1022.5; found 1023.4 (M+H), 1045.4 (M+Na).

Compound 121: To a solution of compound 120 (18.2 mg, 0.0178 mmol) in DMF (1 mL) at room temperature, 5% piperidine in DMF solution (0.4 mL) was added. The reaction was complete after 30 minutes by LCMS. The reaction was purified by prep HPLC on a Gemini 30×150 mm column, eluting with 5-95% MeCN/H₂O (both containing 0.05% AcOH). Pure fractions were combined and lyophilized to obtain compound 121 (12 mg, 83%) as a fluffy off-white solid. MS (ESI, pos.): calc'd for C₄₁H₆₀N₄O₁₂, 800.4; found 801.4 (M+H) ¹H-NMR (300 MHz; CDCl₃): δ 8.02 (m, 2H), 7.09 (d, J=6.7 Hz, 1H), 7.01-6.97 (m, 1H), 6.68 (t, J=11.3 Hz, 1H), 6.19 (d, J=11.2 Hz, 1H), 6.05 (d, J=15.7 Hz, 1H), 5.85-5.81 (m, 1H), 5.46 (dt, J=2.6, 1.2 Hz, 1H), 4.79-4.72 (m, 3H), 4.48-4.44 (m, 1H), 4.20-4.12 (m, 3H), 4.06-4.00 (m, 2H), 3.89 (d, J=5.0 Hz, 1H), 3.76-3.69 (m, 1H), 3.61 (d, J=5.3 Hz, 1H), 3.15 (d, J=4.0 Hz, 1H), 2.86 (m, 1H), 2.56-2.48 (m, 2H), 2.37-2.23 (m, 7H), 2.04 (d, J=5.3 Hz, 5H), 1.92-1.88 (m, 2H), 1.78 (s, 3H), 1.75-1.61 (m, 4H), 1.47-1.33 (m, 2H), 1.01 (dd, J=11.9, 6.7 Hz, 6H), 0.93 (d, J=6.8 Hz, 3H), 0.88 (s, 3H).

### Example 42

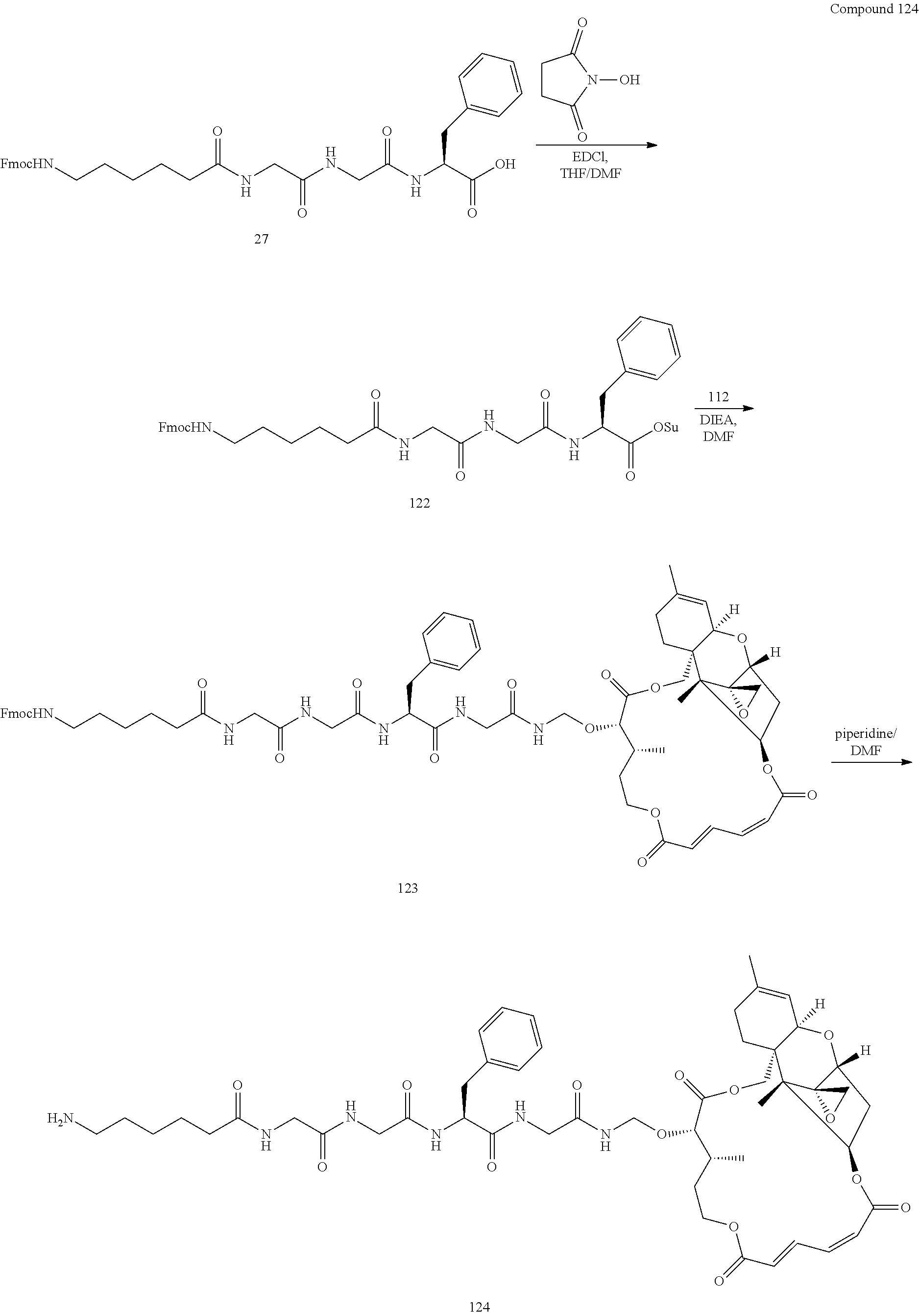

Compound 122: To a solution of compound 27 (80 mg, 0.130 mmol) and N-hydroxysuccinimide (18 mg, 0.156 mmol) in 1:1 THF/DMF (0.8 mL), EDCI (37.4 mg, 0.195 mmol) was added, and the reaction was stirred for 2 hours. Volatiles were removed under reduced pressure and the product was purified by prep HPLC on a 30×150 mm Gemini column using 30-100% MeCN/H₂O (both containing 0.05% AcOH). Pure fractions were combined and lyophilized to obtain compound 122 (66.2 mg, 72%) as a fluffy off-white solid. MS (ESI, pos.): calc'd for C₃₈H₄₁N₅O₉, 711.3; found 712.3 (M+H).

Compound 123: To a solution of compound 112 (12.3 mg, 0.021 mmol) and compound 122 (16.5 mg, 0.023 mmol) in anhydrous DMF (1.5 mL), N,N-diisopropylethylamine was added. After stirring for 30 minutes, the reaction was loaded onto an ISCO 15.5 g C18Aq column and eluted with 5-95% MeCN/H₂O (both containing 0.05% AcOH). Pure fractions were combined and lyophilized to obtain compound 123 (14.2, 57%) as a fluffy off-white solid. MS (ESI, pos.): calc'd for C₆₄H₇₆N₆O₁₆, 1184.5; found 1185.5 (M+H).

Compound 124: To a solution of compound 123 (14.2 mg, 0.012 mmol) in DMF (1.0 mL), 5% piperidine solution in DMF (0.7 mL) was added. After stirring for 40 minutes, the reaction was purified by prep HPLC on a 30×150 mm Gemini column using 5-95% MeCN/H₂O (both having 0.05% AcOH). Pure fractions were combined and lyophilized to obtain compound 124 (9.2, 80%) as a fluffy off-white solid. MS (ESI, pos.): calc'd for C₄₉H₆₆N₆O₁₄, 962.5; found 963.4 (M+H). ¹H-NMR (500 MHz; DMSO-d₆): δ 8.65-8.61 (m, 1H), 8.43 (m, 1H), 8.22-8.06 (m, 3H), 7.83 (dd, J=15.5, 11.7 Hz, 1H), 7.25-7.21 (m, 4H), 7.16 (t, J=6.6 Hz, 1H), 6.84 (t, J=11.4 Hz, 1H), 6.30 (d, J=11.1 Hz, 1H), 6.21 (d, J=15.5 Hz, 1H), 5.80 (dd, J=7.9, 3.7 Hz, 1H), 5.29 (d, J=4.4 Hz, 1H), 4.60 (dd, J=10.3, 8.3 Hz, 1H), 4.52-4.45 (m, 3H), 4.30 (d, J=10.0 Hz, 1H), 4.05 (t, J=5.5 Hz, 2H), 3.91 (t, J=5.8 Hz, 1H), 3.75-3.62 (m, 7H), 3.58-3.53 (m, 1H), 3.04 (dd, J=13.5, 3.5 Hz, 2H), 2.94-2.88 (m, 3H), 2.80 (d, J=13.1 Hz, 1H), 2.64 (t, J=5.0 Hz, 1H), 2.26-2.21 (m, 1H), 2.12 (q, J=7.2 Hz, 2H), 1.95 (dt, J=14.9, 4.5 Hz, 1H), 1.88-1.85 (m, 2H), 1.81 (s, 2H), 1.76 (d, J=13.1 Hz, 2H), 1.64 (s, 3H), 1.55 (t, J=12.7 Hz, 1H), 1.48 (dt, J=14.3, 7.2 Hz, 2H), 1.34 (q, J=6.8 Hz, 2H), 1.24 (dt, J=15.1, 7.4 Hz, 3H), 0.76 (d, J=6.8 Hz, 3H), 0.69 (s, 3H).

### Example 43

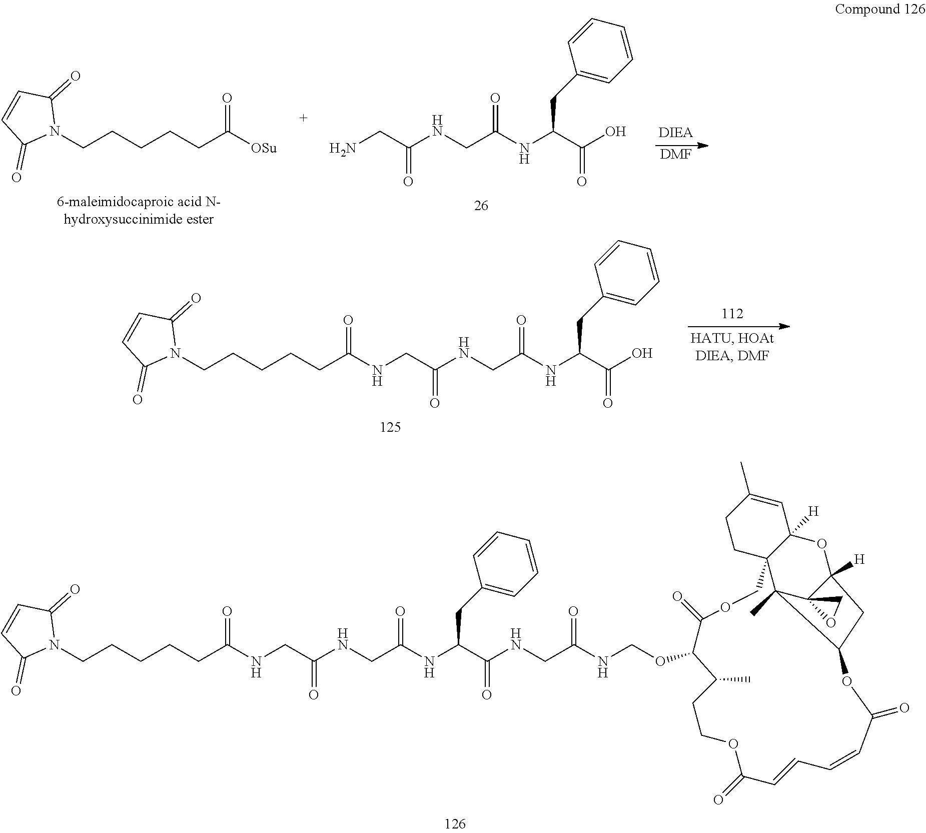

Compound 125 was prepared using the same method as for compound 27 except starting with 6-maleimidocaproic acid N-hydroxysuccinimide ester. MS (ESI, pos.): calc'd for C₂₃H₂₈N₄O₇, 472.2; found 473.3 (M+H).

Compound 126: To a solution of compound 112 (10 mg, 0.017 mmol) and compound 125 (9.5 mg, 0.020 mmol) in anhydrous DMF (1.5 mL), HATU (9.7 mg, 0.026 mmol), HOAt (1.5 mg, 0.017 mmol) and N,N-diisopropylethylamine (6.0 μL, 0.034 mmol) were added. After stirring for 30 minutes, the reaction was loaded onto an ISCO 30 g C18Aq column and eluted with 5-95% MeCN/H₂O (both containing 0.05% AcOH). Pure fractions were combined and lyophilized to obtain compound 126 (5.6 mg, 33%) as a fluffy off-white solid. MS (ESI, pos.): calc'd for C₅₃H₆₆N₆O₁₆, 1042.5; found 1043.4 (M+H). ¹H-NMR (500 MHz; DMSO-d₆): δ 8.54 (t, J=6.5 Hz, 1H), 8.34 (t, J=8.2 Hz, 1H), 8.13-8.06 (m, 2H), 7.99 (d, J=3.8 Hz, 1H), 7.83 (ddd, J=15.6, 11.6, 0.8 Hz, 1H), 7.25-7.20 (m, 4H), 7.18-7.15 (m, 1H), 6.98 (s, 2H), 6.83 (d, J=11.6 Hz, 1H), 6.30 (d, J=11.1 Hz, 1H), 6.21 (d, J=15.6 Hz, 1H), 5.80 (dd, J=7.9, 3.9 Hz, 1H), 5.29 (d, J=4.2 Hz, 1H), 4.60 (dd, J=10.7, 8.1 Hz, 1H), 4.52-4.45 (m, 3H), 4.32-4.28 (m, 1H), 4.06-4.04 (m, 2H), 3.92 (td, J=11.5, 3.2 Hz, 1H), 3.72 (t, J=11.3 Hz, 2H), 3.66 (m, 4H), 3.58 (td, J=18.4, 5.5 Hz, 2H), 3.04 (dd, J=13.3, 4.2 Hz, 1H), 2.92 (d, J=3.9 Hz, 1H), 2.78 (dd, J=13.6, 8.5 Hz, 1H), 2.63-2.61 (m, 1H), 2.41 (dd, J=13.4, 6.6 Hz, 2H), 2.26-2.21 (m, 1H), 2.10 (t, J=7.5 Hz, 2H), 1.95 (td, J=9.7, 5.7 Hz, 1H), 1.89-1.85 (m, 4H), 1.81-1.71 (m, 2H), 1.62-1.61 (s, 3H), 1.58-1.53 (m, 1H), 1.50-1.42 (m, 4H), 1.18 (dt, J=15.5, 7.7 Hz, 2H), 0.76 (d, J=6.9 Hz, 3H), 0.69 (s, 3H).

### Example 44

Anti-Hemagglutinin Antibody Drug Conjugate Synthesis

Anti-hemagglutinin (anti-HA) monoclonal antibody mAb11729 was mutated to introduce a consensus LLQGA pentapeptide sequence at the C-terminus of the heavy chain. A non-HA binding mAb (derived from an immunological antigen having no relation to infectious diseases) containing the same consensus sequence at the C-terminus of the heavy chain was used as a non-binding isotype control. The mutation allowed the antibodies to be enzymatically conjugated to a maximum loading of 2 on the heavy chains (one on each heavy chain).

A second anti-hemagglutinin (H3N2) monoclonal antibody F005-126 taken from the literature (Iba, et al., J. Virology 2014, vol 88, 7130-7144) was mutated to introduce a consensus ELQGP pentapeptide sequence at the C-terminus of the heavy chain. A non-HA binding mAb (derived from an immunological antigen having no relation to infectious diseases) containing the same consensus sequences at the C-terminus of the heavy chain or C-terminus of the light chain was used as a non-binding isotype control. The mutation allowed the antibodies to be enzymatically conjugated to a maximum loading of 2 (one on each heavy or light chain).

Antibodies with a conjugation site at the C-terminus of the heavy chain were conjugated at 1 mg/mL in PBS pH 7.4. Verrucarin A derivatives 10a, b, c; 19a, b, c; 46, 53, 101, 106, and 124 were added in a 10-40 fold molar excess over antibody and the enzymatic reaction was initiated by addition of 12 units of bacterial transglutaminase (Zedira or MilliporeSigma) per mg of antibody and incubated at 37° C. for 16 hours. The conjugates were purified using PBS with 5% glycerol by size exclusion chromatography (Superdex 200) and sterile filtered. Protein concentrations and drug to antibody ratios were determined by UV spectral analysis. Size-exclusion HPLC established that all conjugates were >90% monomeric. All conjugated antibodies were analyzed by mass spectroscopy for linker payload loading values. Drug to antibody ratios are reported in Table 3.

Native mAb11729 and the isotype control antibodies (1-10 mg/ml) in 50 mM HEPES, 150 mM NaCl, pH 7.5, were treated with 2.2 eq. of tris(2-carboxyethyl)phosphine at 37° C. for 90 min to reduce the interchain disulfide bonds. The maleimido verrucarin A derivatives 12a, b, c, 21a, b, c, and 97 (1.2 equivalents/SH group) in DMSO (10 mg/ml) was added to the reduced antibody and allowed to react at 22° C. for 1 hr. The conjugates were purified using PBS with 5% glycerol by size exclusion chromatography and sterile filtered. Protein concentrations and drug to antibody ratios were determined by UV spectral analysis. Size-exclusion HPLC established that all conjugates used were >95% monomeric. All conjugated antibodies were analyzed by mass spectroscopy for linker payload loading values. Drug to antibody ratios are reported in Table 3.

Table 3. Purity (by SEC) and DAR of conjugates.

| Antibody Drug Conjugate | Drug to | ESI-MS, m/z | Purity |
|---|---|---|---|
| | Antibody Ratio | (Conjug - Native) | (by |
| | (DAR, ESI-MS) | | SEC) |
| 11729-HC-Cterm- LLQGA-10a | 2.2 | | >92% |
| Isotype Control-HC-Cterm- LLQGA- | 2.0 | | >91% |
| 10a | | | |
| 11729-HC-Cterm- LLQGA-10b | 2.2 | 151041-149075 | >95% |
| Isotype Control-HC-Cterm- LLQGA- | 2.1 | 151404-149264 | >95% |
| 10b | | | |
| 11729-HC-Cterm- LLQGA-10C | 2.1 | | >93% |
| Isotype Control-HC-Cterm- LLQGA- | 2.1 | | >93% |
| 10c | | | |
| 11729-12b | 8.0 | LC 24694-23523 | >95% |
| | | HC 52522-50613 | |
| Isotype Control-12b | 8.0 | LC 24669-23498 | >98% |
| | | HC 52728-50660 | |
| 11729-HC-Cterm- LLQGA-19a | 2.0 | | >93% |
| Isotype Control-HC-Cterm- LLQGA- | 1.9 | | >92% |
| 19a | | | |
| 11729-HC-Cterm- LLQGA-19b | 2.1 | 151076-149075 | >91% |
| Isotype Control-HC-Cterm- LLQGA- | 2.0 | 151439-149264 | >91% |
| 19b | | | |
| 11729-HC-Cterm- LLQGA-19C | 2.1 | | >93% |
| Isotype Control-HC-Cterm- LLQGA- | 2.0 | | >92% |
| 19c | | | |
| 11729-21a | 8.0 | | >99% |
| Isotype Control-21 a | 8.0 | | >99% |
| 11729-21 b | 8.0 | | >99% |
| Isotype Control-21 b | 8.0 | | >99% |
| F005-126-HC-Cterm-ELQRP-10b | 1.9 | | >96% |
| Isotype Control-HC-Cterm-ELQRP- | 1.8 | | >95% |
| 10b | | | |
| F005-126-HC-Cterm-ELQRP-19b | 1.8 | | >95% |
| Isotype Control-HC-Cterm-ELQRP- | 1.8 | | >95% |
| 19b | | | |
| 10987-12b | 8.0 | LC 23935-22765 | >99% |
| | | HC 54108-50597 | |
| 10985-12b | 8.0 | LC 24027-22856 | >98% |
| | | HC 54840-51329 | |
| 10987-HC-Cterm- LLQGA-10b | 2.0 | 149849-147689 | >96% |
| Isotype Control-HC-Cterm- LLQGA- | 2.0 | 151436-149264 | >96% |
| 10b | | | |
| Antibody Drug Conjugate | Drug to | ESI-MS, m/z | Purity |
| | Antibody Ratio | (Conjug - Native) | (by |
| | (DAR, ESI-MS) | | SEC) |
| 10985-HC-Cterm- LLQGA-10b | 2.0 | 151505-149343 | >95% |
| 3471- HC-Cterm- LLQGA-10b | 2.0 | 151940-149792 | >96% |
| Clone 4A8-HC-Cterm- LLQGA-10b | 2.0 | 153825-151678 | >96% |
| 11729-HC-Cterm- LLQGA-46 | | | |
| Isotype Control-HC-Cterm- LLQGA- | | | |
| 46 | | | |
| 11729-HC-Cterm- LLQGA-53 | | | |
| Isotype Control-HC-Cterm- LLQGA- | | | |
| 53 | | | |
| 11729-97 | 8.0 | | >95% |
| Isotype Control-97 | 8.0 | | >95% |
| 11729-HC-Cterm- LLQGA-101 | 1.4 | | >98% |
| Isotype Control-HC-Cterm- LLQGA- | 1.4 | | >98% |
| 101 | | | |
| 11729-HC-Cterm- LLQGA-106 | 0.9 | | >98% |
| Isotype Control-HC-Cterm- LLQGA- | 1.5 | | >99% |
| 106 | | | |
| 11729-HC-Cterm- LLQGA-124 | 1.7 | | >99% |
| Isotype Control-HC-Cterm- LLQGA- | 1.9 | | >99% |
| 124 | | | |

Characterization of Conjugates by Liquid Chromatography-Mass Spectrometry

To determine the loading of the linker-payloads on the antibody, the conjugates were reduced to heavy and light chain by 50 mM dithiothreitol, if conjugates were glycosylated then further deglycosylated with PNGase and then analyzed by LC-MS. The resulting molecular ions, when weighted according to intensities, corresponded to the loadings listed in Table 3.

The conjugates were analyzed by ESI-MS for the determination of the drug:antibody ratio (DAR) using a Waters Acquity UPLC interfaced to Xevo G2-S QT of Mass Spectrometer. The chromatographic separation was achieved on a C4 column (2.1×50 mm ACQUITY UPLC BEH protein C4, 1.7 um, 300 A) in a 10 min gradient (minute:percentage of mobile phase B; 0:10%, 1:10%, 5:90%, 7:90%, 7.2:10%, 10:10%). The mobile phase A was 0.1% formic acid in water, and mobile phase B was 0.1% formic acid in acetonitrile. The flow rate was set at 0.3 mL/min. The detector TOF scan was set from m/z 500-4500 with major parameters as listed (Capillary voltage 3.0 kV; Sampling Cone 80V; Source Offset at 100V; Source temperatures 150° C.; Desolvation temperature 450° C.; Cone gas 0 L/hr; Desolvation gas 800 L/hr). The spectra were deconvoluted with MaxEnt function within MassLynx software. The resulting molecular ions, when weighted according to intensities, corresponded to the loadings listed in Tables 3.

Alternatively, to determine the loading of the drugs on the antibody, the conjugates were run on Agilent 1260 using a TSK-NPR Butyl HIC (Hydrophobic Interaction Chromatography) column using a linear gradient of 25 mM sodium phosphate with 1.5 M ammonium sulfate pH 6.8 to 25 mM sodium phosphate pH 6.8 over 18 min. The payload loading was determined by integration of peak areas corresponding to the species of conjugated and unconjugated antibody.

The mass spectrometry spectra were deconvoluted using Masslynx software and the drug to antibody ratio (DAR) was calculated using the following equations.

Dn %=PIn/Σ(PI0+PI1+PI2+PIi)×100 1. Relative percentage (%) of drug (Dn) by peak intensity (PI) distribution:

(n=0, 1, 2, 3, . . . , i)

DAR=Σ(1×D1%+2×D2%+3×D3%+ . . . +i×Di %) 2. Average DAR calculation:

### Example 45

Background

Antibodies targeting the membrane-proximal stem domain of influenza HA generally display increased breadth compared to antibodies targeting the globular head of this molecule. However, this increased breadth is coupled with a decrease in potency. Therefore, it is of interest whether the activity of these antibodies can be enhanced through conjugation with antiviral small molecules. mAb11729 is a monoclonal antibody that binds the stem domain of group 1 influenza HA molecules, and which displays antiviral activity against H1N1 in vitro. Verrucarin A is a broad antiviral molecule, which in this case has been modified to be cell-impermeable, and has been conjugated to mAb11729.

### Experimental Procedure, Results and Conclusions

TABLE 4

| Reagents Used | |
|---|---|
| Reagent | Vendor |
| MDCK London | IRR |
| DMEM (1X) | Life Technologies |
| Sodium Pyruvate (100mM) | Life Technologies |
| Fetal Bovine Serum, qualified, US origin | Life Technologies |
| Gentamicin | Life Technologies |
| 30% Low IgG BSA Solution | Sigma |
| PBS, pH 7.2 (1X) | Life Technologies |
| 0.25% Trypsin-EDTA | Life Technologies |
| Trypan Blue | Life Technologies |
| Influenza H1N1 A/Puerto Rico/08/1934-GFP | |

Antiviral Activity

To test antiviral efficacy, mAb11729 and ADCs 10b, 19a, 19b and 19c were assayed for their ability to suppress the infection of cells by influenza virus. MDCK London cells were seeded at 20,000 cells/well in 100 μL of growth media (DMEM containing 1% sodium pyruvate, 10% Fetal Bovine Serum and 0.5% Gentamicin) in a 96-well plate. The cells were incubated at 37° C. and 5% CO₂ for 18 hours. The following day, all antibodies were diluted to a starting concentration of 500 μg/mL in Trypsin infection media (DMEM containing 1% sodium pyruvate, 0.21% Low IgG BSA solution, 1 mg/mL Trypsin TPCK-Treated and 0.5% Gentamicin) and titrated 1:3 to a final concentration of 8.5×10⁻³ μg/mL. H1N1 A/Puerto Rico/08/1934 influenza virus that was engineered to express GFP in cells that it infects (“H1N1 A/Puerto Rico/08/1934-GFP”) was diluted to an MOI of one in Trypsin infection media (Life Technologies) and mixed 1:1 with diluted antibody or ADC. Growth Media was removed from seeded 96-well plates and virus-antibody or virus-ADC mixture was added onto cells at 100 μL per well. Plates were lightly tapped and returned to 37° C. 5% CO2 for 20 hours. Subsequently, plates were washed once with PBS and overlayed with 50 μL of PBS. Plates were read immediately for GFP signal on a Molecular Devices Spectramax i3x plate reader. The ADCs displayed reliably enhanced anti-viral potency against influenza A infection compared to the unconjugated antibody.

TABLE 5
ADC Antiviral Activity

| | | | |
|---|---|---|---|
| ADC | ADC IC₅₀ log(M) | Parental Antibody | Fold Increase over |
| | | IC₅₀ log(M) | Parental Antibody |
| 11729-HC- | 2.382E-09 | 1.658E-08 | 6.96 |
| Cterm- | | | |
| LLQGA-10b | | | |
| 11729-HC- | 1.122E-09 | 1.768E-08 | 15.76 |
| Cterm- | | | |
| LLQGA-19b | | | |
| 11729-HC- | 1.616E-09 | 1.253E-08 | 7.75 |
| Cterm- | | | |
| LLQGA-19a | | | |

| | | | |
|---|---|---|---|
| ADC | ADC IC₅₀ log(M) | Parental Antibody | Fold Increase over |
| | | IC₅₀ log(M) | Parental Antibody |
| 11729-HC- | 5.826E-10 | 1.677E-08 | 28.78 |
| Cterm- | | | |
| LLQGA-19C | | | |

### Example 46

To assess its potential as a broad antiviral payload, the cell-impermeable verrucarin A has been conjugated to mAb10987, mAb10985, and clone 4A8. mAb10987 and mAb10985 are monoclonal antibodies that bind distinct epitopes on the receptor-binding domain of SARS-CoV-2 and clone 4A8 binds outside of the RBD on the SARS-CoV-2 spike protein.

To test antiviral efficacy against SARS-CoV-2, mAb10987, mAb10985 and clone 4A8 and ADCs mAb10987-12b, mAb10985-12b, mAb10987-10b, mAb10985-10b, and clone 4A8-10b were assayed for their ability to suppress the infection of cells by VSVAG expressing the spike protein of SARS-CoV-2 on its surface. Vero cells were seeded at 20,000 cells/well in 100 μL of growth media (DMEM containing 10% Fetal Bovine Serum and 1% Penn-Strep Glutamine) in a 96-well plate. The cells were incubated at 37° C. and 5% CO₂ for 18-24 hours. The following day, all antibodies were diluted to a starting concentration of 10 μg/mL in infection media (DMEM containing 3% FBS, and 1% PSG) and titrated 1:3 to a final concentration of 1.69×10⁻⁴ μg/mL. VSV-spike virus was diluted to 4×10⁴ PFU/mL in infection media and mixed 1:1 with diluted antibody or ADC. The virus-antibody mix was incubated together for 30 minutes. Growth media was then removed from seeded 96-well plates and virus-antibody or virus-ADC mixture was added onto cells at 100 μL per well. Plates were lightly tapped and returned to 37° C. 5% CO₂ for 20 hours. Subsequently, plates were removed from the incubator and fixed with 2% PFA for 30 min. 2% PFA was then removed, and blocking/permeabilization buffer (PBS with 3% BSA and 0.1% Triton-X100) was added to cells to 1 hr at room temp. Blocking buffer was then replaced with the primary antibody solution (rabbit anti-VSV from Imanis diluted 1:100 in blocking buffer). Plates were incubated for 2 hrs at room temp. Plates were then washed 3× with PBS. Secondary antibody solution (goat anti-rabbit AF488 diluted 1:100 in blocking buffer) was added to cells for 1 hr at 37° C. Plates were then washed 3× with PBS and overlayed with 100 μL PBS. AF488 fluorescence was then read on a Molecular Devices Spectramax i3x plate reader. The anti-spike antibodies conjugated to the verrucarin-A payload displayed enhanced anti-viral potency against VSV-spike infection compared to the unconjugated antibody (Table 6).

TABLE 6
Anti-viral efficacy against SARS-CoV-2

| | ADC IC50 | Parental IC50 | Fold |
|---|---|---|---|
| | log(M) | log(M) | Increase |
| | | | over |
| | | | Parental |
| | | | Antibody |
| 10987-12b | 1.42E-11 | 4.97E-11 | 3.5 |
| 10987-HC-Cterm-LLQGA-10b | 1.97E-11 | 4.97E-11 | 2.5 |
| 10985-12b | 2.27E-10 | 5.03E-10 | 2.2 |
| 10985-HC-Cterm-LLQGA-10b | 1.99E-10 | 5.03E-10 | 2.5 |
| 4A8-HC-Cterm-LLQGA-1 Ob | 2.90E-11 | 8.05E-11 | 2.8 |

### Example 47

To further assess its potential as a broad antiviral payload, the cell-impermeable verrucarin A has been conjugated to mAb3471, a monoclonal antibody that binds the Ebolavirus (EBOV) glycoprotein.

To test antiviral efficacy against EBOV, mAb3471 and ADB mAb3471-10b were assayed for their ability to suppress the infection of cells by VSVΔG expressing the EBOV glycoprotein (GP) on its surface. Vero cells were seeded at 20,000 cells/well in 100 μL of growth media (DMEM containing 10% Fetal Bovine Serum and 1% Penn-Strep Glutamine) in a 96-well plate. The cells were incubated at 37° C. and 5% CO₂ for 18-24 hours. The following day, all antibodies were diluted to a starting concentration of 200 μg/mL in infection media (DMEM containing 3% FBS, and 1% PSG) and titrated 1:3 to a final concentration of 3.39×10⁻³ μg/mL. VSV-EBOV-GP virus was diluted to 4×10⁴ PFU/mL in infection media and mixed 1:1 with diluted antibody or ADC. The virus-antibody mix was incubated together for 30 minutes. Growth media was then removed from seeded 96-well plates and virus-antibody or virus-ADC mixture was added onto cells at 100 μL per well. Plates were lightly tapped and returned to 37° C. 5% CO₂ for 20 hours. Subsequently, plates were removed from the incubator and fixed with 2% PFA for 30 min. 2% PFA was then removed, and blocking/permeabilization buffer (PBS with 3% BSA and 0.1% Triton-X100) was added to cells to 1 hr at room temp. Blocking buffer was then replaced with the primary antibody solution (rabbit anti-VSV from Imanis diluted 1:100 in blocking buffer). Plates were incubated for 2 hrs at room temp. Plates were then washed 3× with PBS. Secondary antibody solution (goat anti-rabbit AF488 diluted 1:100 in blocking buffer) was added to cells for 1 hr at 37° C. Plates were then washed 3× with PBS and overlayed with 100 μL PBS. AF488 fluorescence was then read on a Molecular Devices Spectramax i3x plate reader. mAb3471 conjugated to the verrucarin-A payload displayed enhanced anti-viral potency against VSV-EBOV-GP infection compared to the unconjugated antibody (Table 7).

TABLE 7
Anti-viral efficacy against EBOV

| | ADC IC50 | Parental IC50 | Fold |
|---|---|---|---|
| | log(M) | log(M) | Increase |
| | | | over |
| | | | Parental |
| | | | Antibody |
| 3471 -HC-Cterm-LLQGA-10b | 2.22E-09 | 9.57E-09 | 4.3 |

This disclosure is not to be limited in scope by the embodiments disclosed in the examples which are intended as single illustrations of individual aspects, and any equivalents are within the scope of this disclosure. Various modifications in addition to those shown and described herein will become apparent to those skilled in the art from the foregoing description. Such modifications are intended to fall within the scope of the appended claims.

Various references such as patents, patent applications, and publications are cited herein, the disclosures of which are hereby incorporated by reference herein in their entireties.

1. A compound of Formula I: 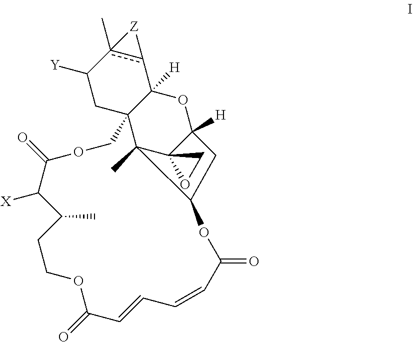
or a pharmaceutically acceptable derivative thereof, wherein:
X is NR¹R², OR³ or SR⁴;
R¹ and R² are each independently H, alkyl, OR⁵ or COR⁶, or together with the nitrogen atom to which they are attached form heterocycloalkyl;
R³ is alkyl or COR⁷;
R⁴ is H, alkyl or COR⁸;
R⁵ is H or alkyl;
R⁶ is R⁹, OR¹⁰ or NR¹¹R¹²;
R⁷-R⁹ are each independently alkyl or aralkyl;
R¹⁰-R¹² are each independently H, alkyl or aralkyl;
Y is H or OH; and
(i) Z is O and [symbol] is a single bond; or (ii) Z is absent and [symbol] is a double bond.

2. (canceled)

3. (canceled)

4. The compound of claim 1, wherein R¹ and R² are each independently H, methyl, ethyl, C(O)—(C₂₋₄alkylene)-CO—W where W is OR¹³ or NR¹⁴R¹⁵, or C(O)—CH(V)—CH₃ where V is OR¹⁶ or NR¹⁷R¹⁸, or together with the nitrogen atom to which they are attached form piperazinyl; R¹³ and R¹⁶ are each independently H or methyl; and R¹⁴, R¹⁵, R¹⁷ and R¹⁸ are each independently H, methyl, hydroxy or methoxy.

5. The compound of claim 1, wherein R¹ is H, methyl, ethyl, C(O)CH₂CH₂COOH, C(O)CH₂CH₂CH₂COOH, C(O)CH₂CH₂CH₂CH₂COOH, C(O)CH₂CH₂CH₂COOMe, C(O)CH₂CH₂CH₂CONHOH, C(O)CH₂CH₂CH₂CONHOMe, C(O)—CH(OH)—CH₃, C(O)—CH(NH₂)—CH₃ or C(O)—CH(NMe₂)-CH₃and R² is H or methyl; and R¹ and R² together with the nitrogen atom to whch they are attached form 4-methyl-1-piperazin.

6. (canceled)

7. (canceled)

8. The compound of claim 1, wherein R³ is methyl or C(O)-alkyl, where the alkyl is optionally substituted.

9. (canceled)

10. The compound of claim 1, wherein R³ is methyl, C(O)—CH(NHMe)-CH₃ or C(O)—CH(NHAc)—CH₃.

11. (canceled)

12. The compound of claim 1, wherein R⁴ is C(O)Me, and R⁵ is H or methyl.

13. (canceled)

14. (canceled)

15. The compound of claim 1, wherein R⁶ is CH₂CH₂COOH, CH₂CH₂CH₂COOH, CH₂CH₂CH₂CH₂COOH, CH₂CH₂CH₂COOMe, CH₂CH₂CH₂CONHOH or CH₂CH₂CH₂CONHOMe.

16. (canceled)

17. The compound of claim 1, wherein R⁷ and R⁸ are each independently methyl.

18. (canceled)

19. The compound of claim 1, wherein R¹⁰-R¹² are each independently H or methyl.

20. The compound of claim 1, wherein Y is H, Z is absent and [symbol] is a double bond.

21. The compound of claim 1, wherein Y is OH, Z is absent and [symbol] is a double bond.

22. The compound of claim 1, wherein Y is H, Z is O and [symbol] is a single bond.

23. The compound of claim 1 having Formula II: 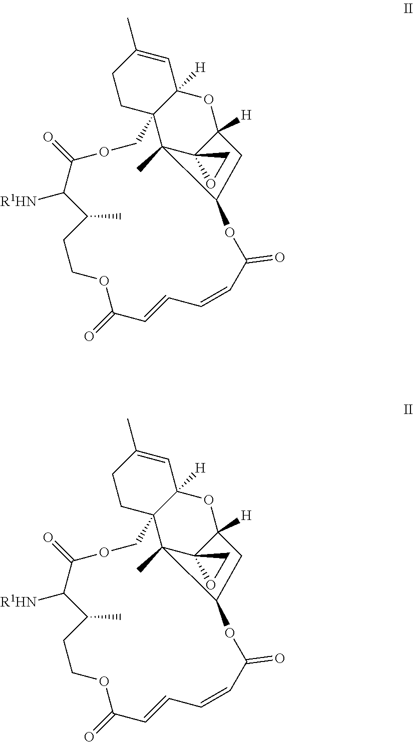
or a pharmaceutically acceptable derivative thereof, wherein:
R¹ is H or —COR⁶; and
R⁶ is -alkylene-COOH.

24. (canceled)

25. The compound of claim 23, wherein R¹ is —COR⁶.

26. The compound of claim 23, wherein R⁶ is —(CR²¹R²²)ₘCOOH, where R²¹ and R²² are each independently H or alkyl; and m is an integer from 0-6.

27. (canceled)

28. (canceled)

29. (canceled)

30. (canceled)

31. (canceled)

32. (canceled)

33. The compound of claim 1 selected from:

| Compound | Structure |
|---|---|
| 4 (R)-amino-verrucarin A | 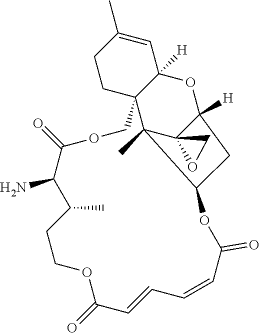 [SMILES: CC1=C[C@H]2O[C@@H]3C[C@H]4OC(=O)/C=C\C=C\C(=O)OCC[C@@H](C)[C@@H](N)C(=O)OC[C@@]2(CC1)[C@]4(C)[C@]31CO1] |
| 5 | 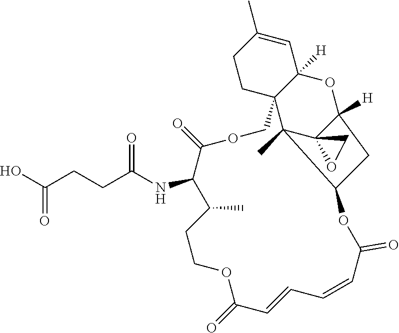 [SMILES: CC1=C[C@H]2O[C@@H]3C[C@H]4OC(=O)/C=C\C=C\C(=O)OCC[C@@H](C)[C@@H](NC(=O)CCC(=O)O)C(=O)OC[C@@]2(CC1)[C@]4(C)[C@]31CO1] |
| 6 | 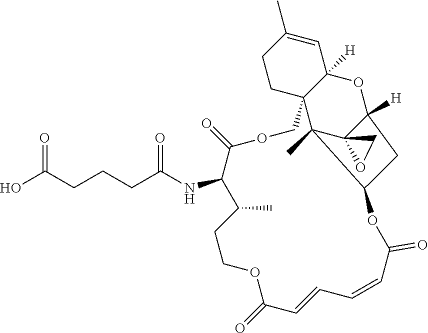 [SMILES: CC1=C[C@H]2O[C@@H]3C[C@H]4OC(=O)/C=C\C=C\C(=O)OCC[C@@H](C)[C@@H](NC(=O)CCCC(=O)O)C(=O)OC[C@@]2(CC1)[C@]4(C)[C@]31CO1] |
| 7 | 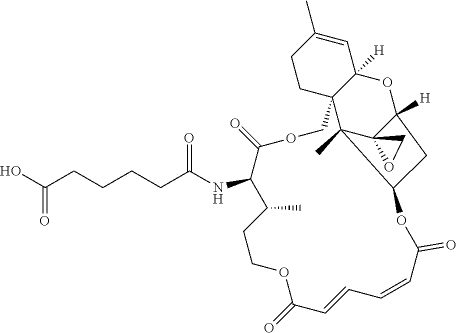 [SMILES: CC1=C[C@H]2O[C@@H]3C[C@H]4OC(=O)/C=C\C=C\C(=O)OCC[C@@H](C)[C@@H](NC(=O)CCCCC(=O)O)C(=O)OC[C@@]2(CC1)[C@]4(C)[C@]31CO1] |
| 22 | 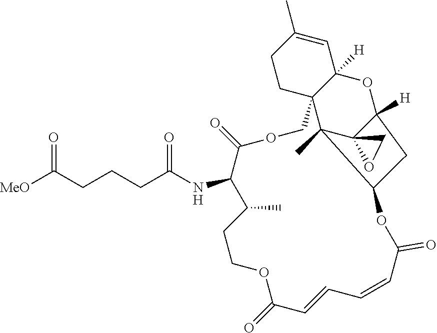 [SMILES: COC(=O)CCCC(=O)N[C@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1] |
| 24 | 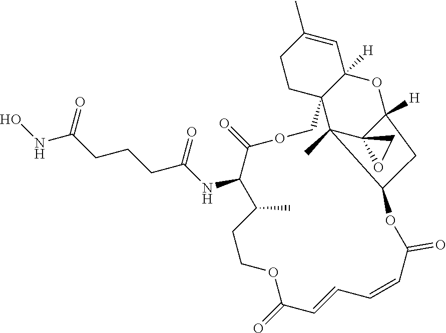 [SMILES: CC1=C[C@H]2O[C@@H]3C[C@H]4OC(=O)/C=C\C=C\C(=O)OCC[C@@H](C)[C@@H](NC(=O)CCCC(=O)NO)C(=O)OC[C@@]2(CC1)[C@]4(C)[C@]31CO1] |
| 25 | 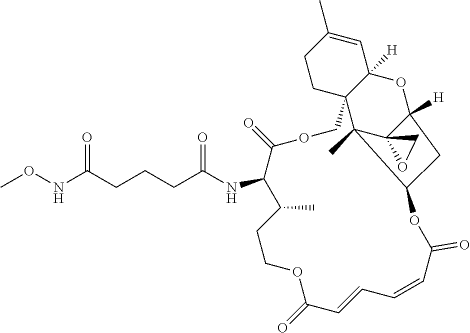 [SMILES: CONC(=O)CCCC(=O)N[C@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1] |
| 52 | 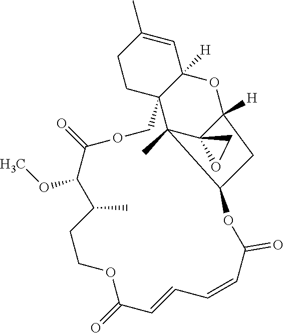 [SMILES: CO[C@@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1] |
| 53 | 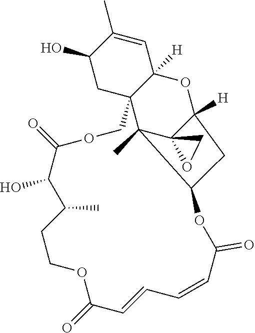 [SMILES: CC1=C[C@H]2O[C@@H]3C[C@H]4OC(=O)/C=C\C=C\C(=O)OCC[C@@H](C)[C@H](O)C(=O)OC[C@@]2(C[C@H]1O)[C@]4(C)[C@]31CO1] |
| 54 | 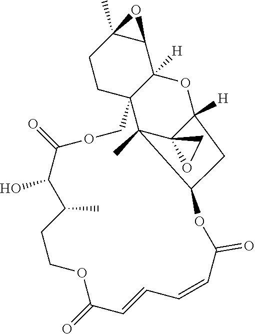 [SMILES: C[C@@H]1CCOC(=O)/C=C/C=C\C(=O)O[C@@H]2C[C@H]3O[C@@H]4[C@@H]5O[C@]5(C)CC[C@]4(COC(=O)[C@H]1O)[C@]2(C)[C@]31CO1] |
| 55 | 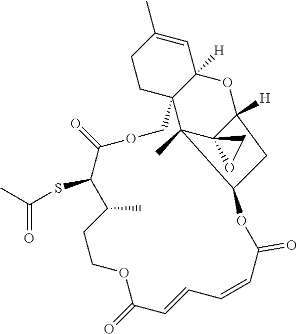 [SMILES: CC(=O)S[C@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1] |
| 56 | 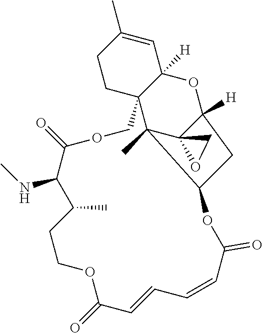 [SMILES: CN[C@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1] |
| 57 | 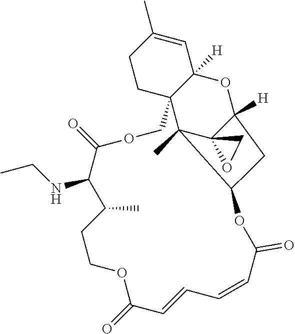 [SMILES: CCN[C@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1] |
| 58 | 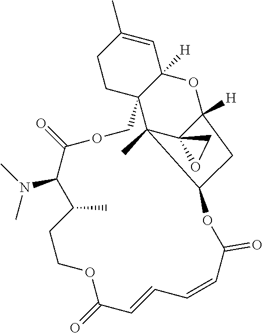 [SMILES: CC1=C[C@H]2O[C@@H]3C[C@H]4OC(=O)/C=C\C=C\C(=O)OCC[C@@H](C)[C@@H](N(C)C)C(=O)OC[C@@]2(CC1)[C@]4(C)[C@]31CO1] |
| 59 | 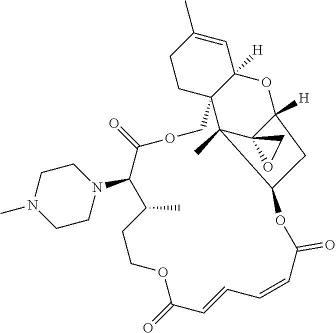 [SMILES: CC1=C[C@H]2O[C@@H]3C[C@H]4OC(=O)/C=C\C=C\C(=O)OCC[C@@H](C)[C@@H](N5CCN(C)CC5)C(=O)OC[C@@]2(CC1)[C@]4(C)[C@]31CO1] |
| 62 | 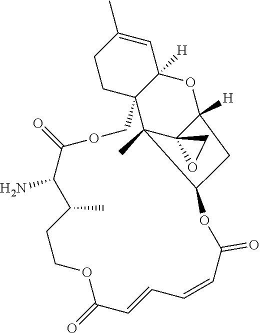 [SMILES: CC1=C[C@H]2O[C@@H]3C[C@H]4OC(=O)/C=C\C=C\C(=O)OCC[C@@H](C)[C@H](N)C(=O)OC[C@@]2(CC1)[C@]4(C)[C@]31CO1] |
| 64 | 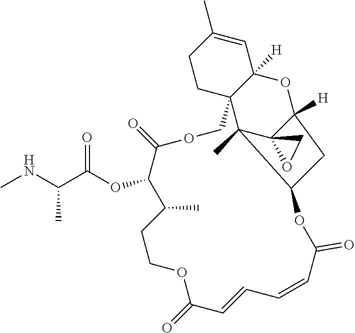 [SMILES: CN[C@@H](C)C(=O)O[C@@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1] |
| 65 | 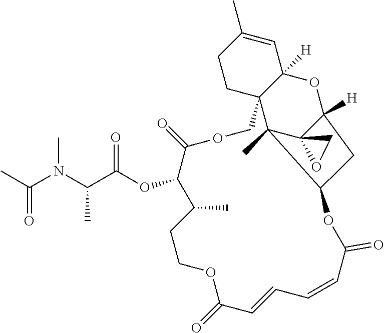 [SMILES: CC(=O)N(C)[C@@H](C)C(=O)O[C@@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1] |
| 77 | 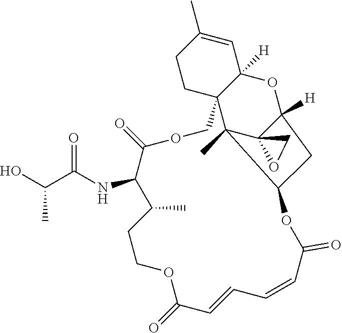 [SMILES: CC1=C[C@H]2O[C@@H]3C[C@H]4OC(=O)/C=C\C=C\C(=O)OCC[C@@H](C)[C@@H](NC(=O)[C@H](C)O)C(=O)OC[C@@]2(CC1)[C@]4(C)[C@]31CO1] |
| 78 | 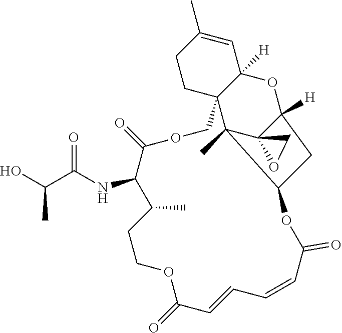 [SMILES: CC1=C[C@H]2O[C@@H]3C[C@H]4OC(=O)/C=C\C=C\C(=O)OCC[C@@H](C)[C@@H](NC(=O)[C@@H](C)O)C(=O)OC[C@@]2(CC1)[C@]4(C)[C@]31CO1] |
| 80 | 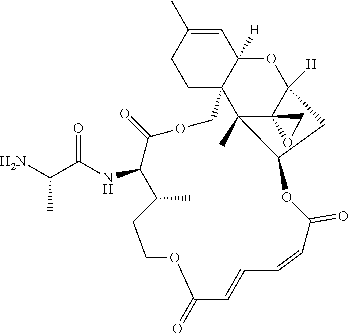 [SMILES: CC1=C[C@H]2O[C@@H]3C[C@H]4OC(=O)/C=C\C=C\C(=O)OCC[C@@H](C)[C@@H](NC(=O)[C@H](C)N)C(=O)OC[C@@]2(CC1)[C@]4(C)[C@]31CO1] |
| 81 | 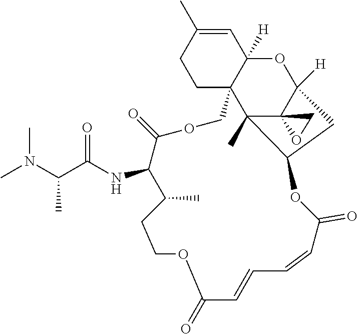 [SMILES: CC1=C[C@H]2O[C@@H]3C[C@H]4OC(=O)/C=C\C=C\C(=O)OCC[C@@H](C)[C@@H](NC(=O)[C@H](C)N(C)C)C(=O)OC[C@@]2(CC1)[C@]4(C)[C@]31CO1] |

34. The compound of claim 1 selected from:

| Compound | Structure |
|---|---|
| 4 (R)-amino-verrucarin A | 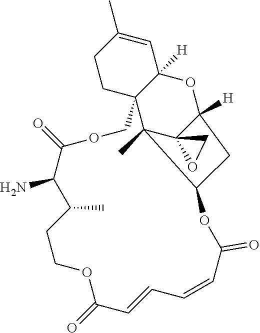 [SMILES: CC1=C[C@H]2O[C@@H]3C[C@H]4OC(=O)/C=C\C=C\C(=O)OCC[C@@H](C)[C@@H](N)C(=O)OC[C@@]2(CC1)[C@]4(C)[C@]31CO1] |
| 5 | 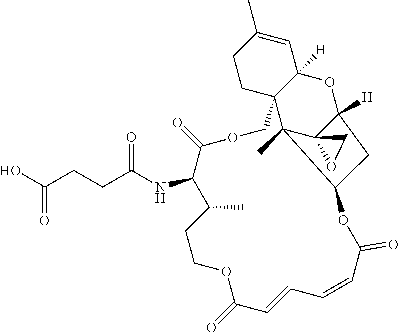 [SMILES: CC1=C[C@H]2O[C@@H]3C[C@H]4OC(=O)/C=C\C=C\C(=O)OCC[C@@H](C)[C@@H](NC(=O)CCC(=O)O)C(=O)OC[C@@]2(CC1)[C@]4(C)[C@]31CO1] |
| 6 | 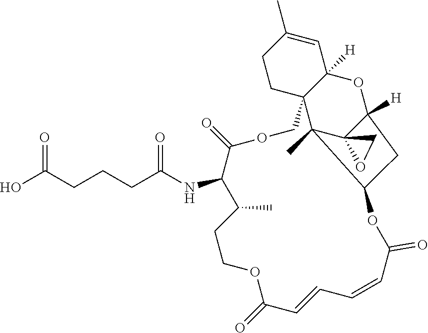 [SMILES: CC1=C[C@H]2O[C@@H]3C[C@H]4OC(=O)/C=C\C=C\C(=O)OCC[C@@H](C)[C@@H](NC(=O)CCCC(=O)O)C(=O)OC[C@@]2(CC1)[C@]4(C)[C@]31CO1] |
| 7 | 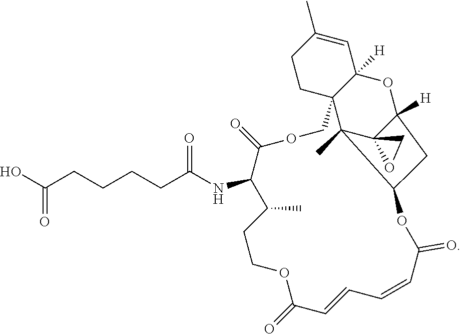 [SMILES: CC1=C[C@H]2O[C@@H]3C[C@H]4OC(=O)/C=C\C=C\C(=O)OCC[C@@H](C)[C@@H](NC(=O)CCCCC(=O)O)C(=O)OC[C@@]2(CC1)[C@]4(C)[C@]31CO1] |

35. A compound of Formula III: 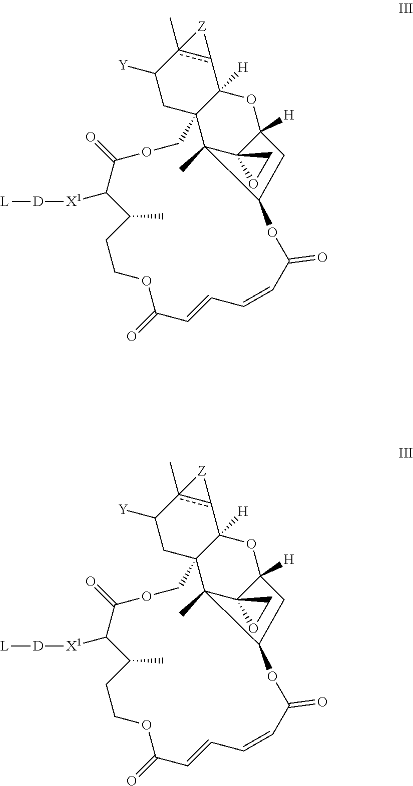
or a pharmaceutically acceptable derivative thereof, wherein X¹ is O or NH; D is absent or is —C(O)—(CH₂)₂₋₅—C(O)— or —O—NH—C(O)—(CH₂)₂₋₅—C(O)—; L is a linking group; Y is H or OH; and (i) Z is O and [symbol] is a single bond; or (ii) Z is absent and [symbol] is a double bond.

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. The compound of claim 35 that has Formula IV: 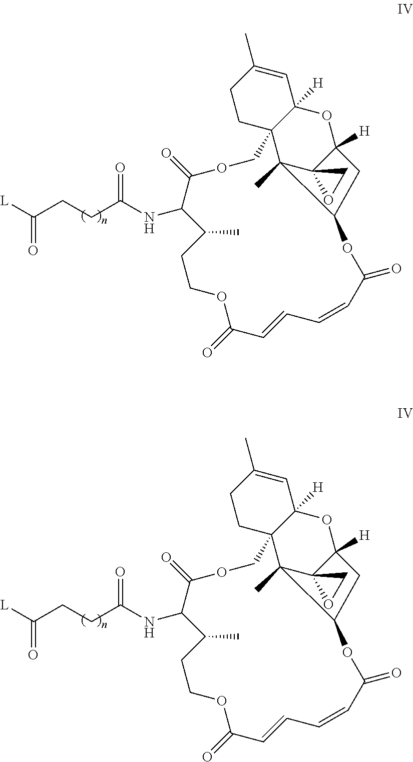
or a pharmaceutically acceptable derivative thereof, wherein L is a linking group and n is an integer from 1 to 4.

49. The compound of claim 48, wherein n is 1, 2 or 3.

50. The compound of claim 35, wherein L is a cleavable linker.

51. The compound of claim 35, wherein L is an acid-labile linker, a hydrolysis-labile linker, an enzymatically cleavable linker, a reduction labile linker, or a self-immolative linker.

52. (canceled)

53. (canceled)

54. (canceled)

55. (canceled)

56. (canceled)

57. (canceled)

58. (canceled)

59. (canceled)

60. The compound of claim 35, wherein L comprises or consists of MC-val-cit-PAB (6-maleimidocaproyl-valine-citrulline-p-aminobenzyloxy).

61. The compound of claim 35, wherein L comprises or consists of AC-val-cit-PAB (6-aminocaproyl-valine-citrulline-p-aminobenzyloxy).

62. The compound of claim 35, wherein L comprises or consists of MC-val-cit-MePAB (6-maleimidocaproyl-valine-citrulline-p-amino-α-methylbenzyloxy).

63. The compound of claim 35, wherein L comprises or consists of AC-val-cit-MePAB (6-aminocaproyl-valine-citrulline-p-amino-α-methylbenzyloxy).

64. The compound of claim 35, wherein L comprises or consists of AC-GGFG-CH₂- (6-aminocaproyl-Gly-Gly-Phe-Gly-CH₂-).

65. The compound of claim 35, having one of the formulae:

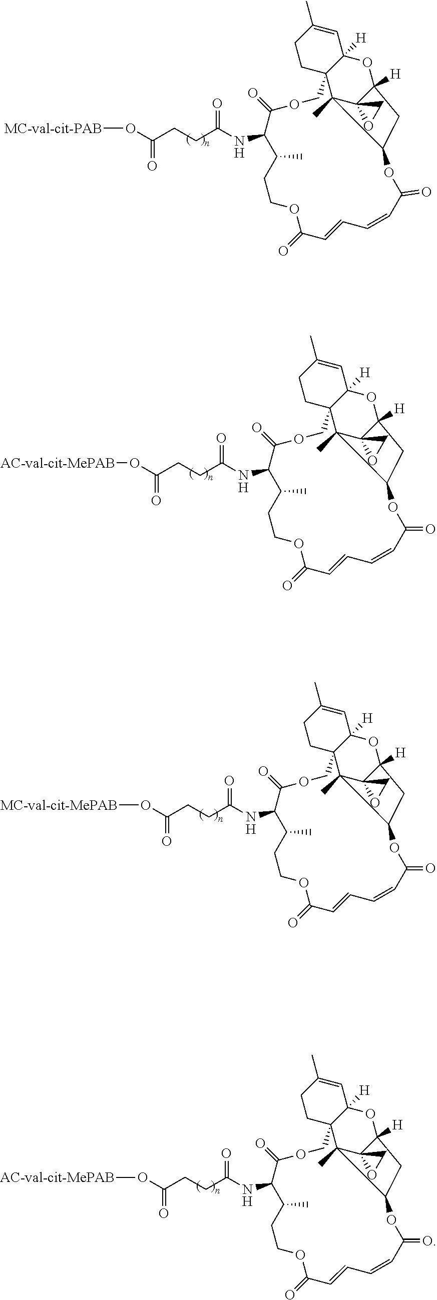

66. The compound of claim 35, wherein L is a non-cleavable linker.

67. The compound of claim 35 selected from:

| Compound | Structure |
|---|---|
| 10a (n = 1) | 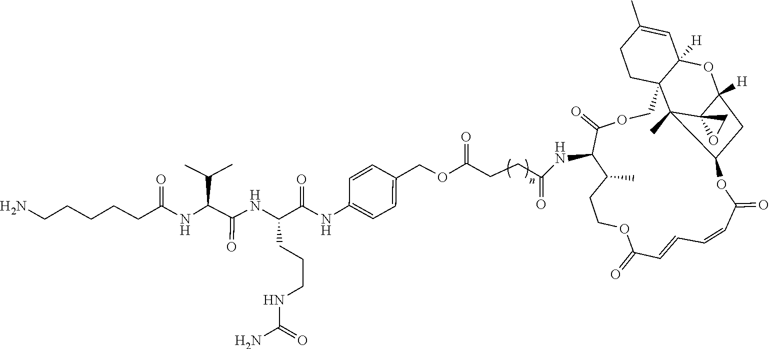 |
| 10b (n = 2) | 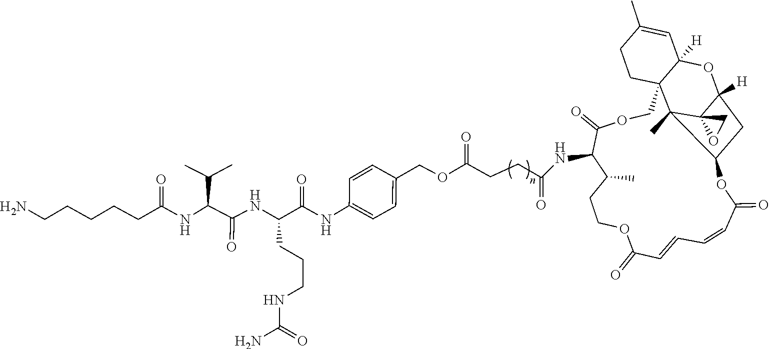 |
| 10c (n = 3) | 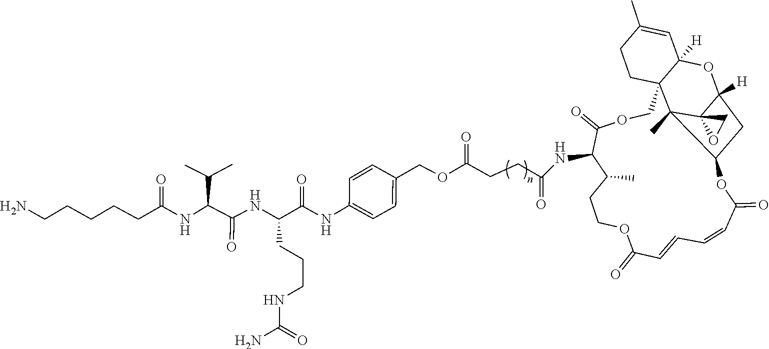 |
| 12a (n = 1) | 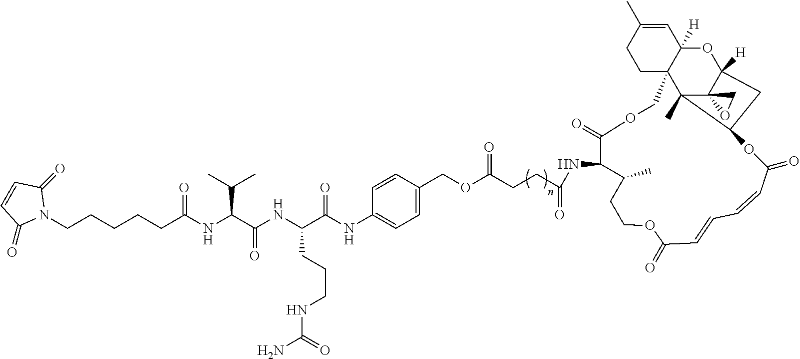 |
| 12b (n = 2) | 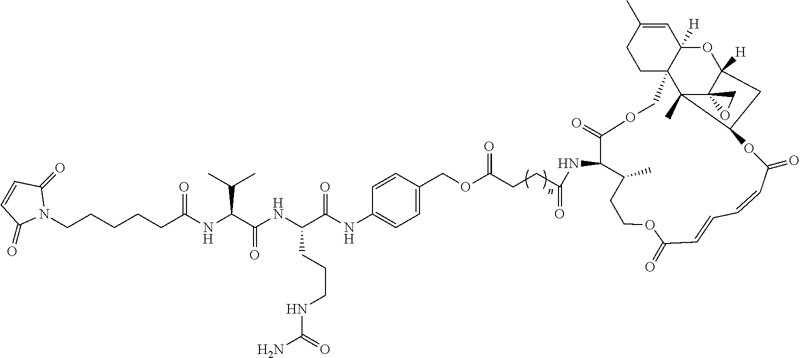 |
| 12c (n = 3) | 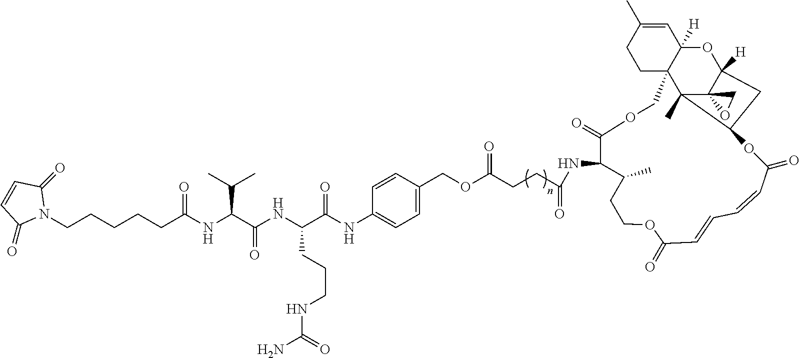 |
| 19a (n = 1) | 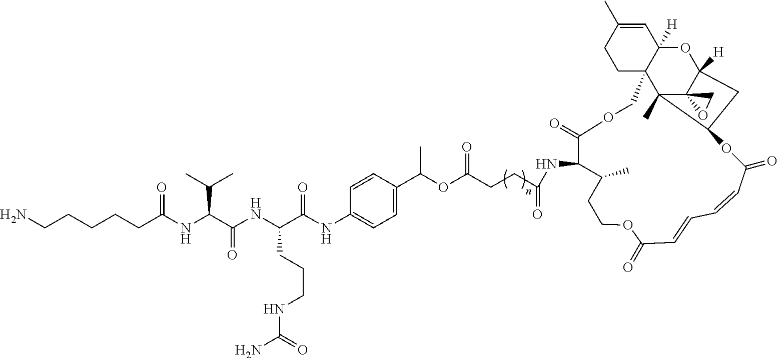 |
| 19b (n = 2) | 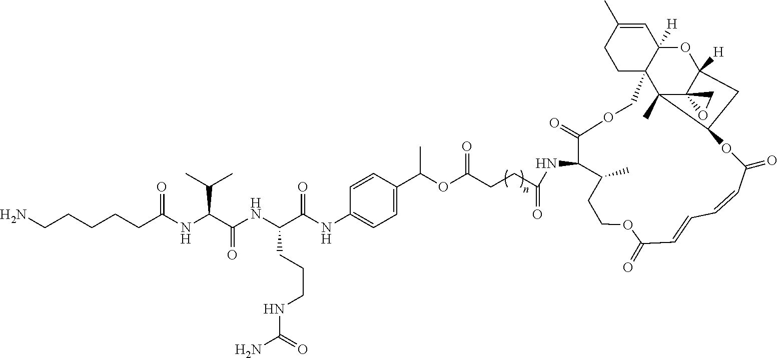 |
| 19c (n = 3) | 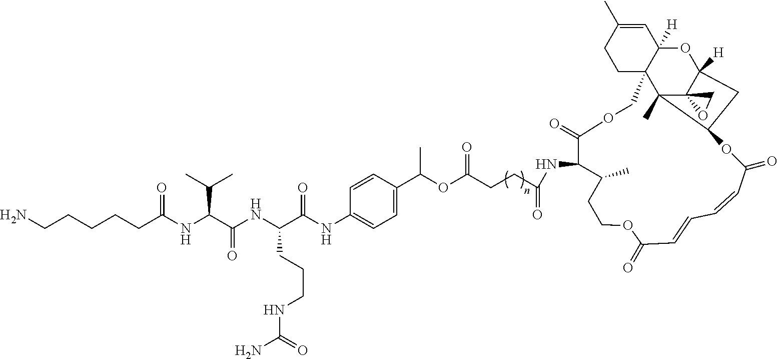 |
| 21a (n = 1) | 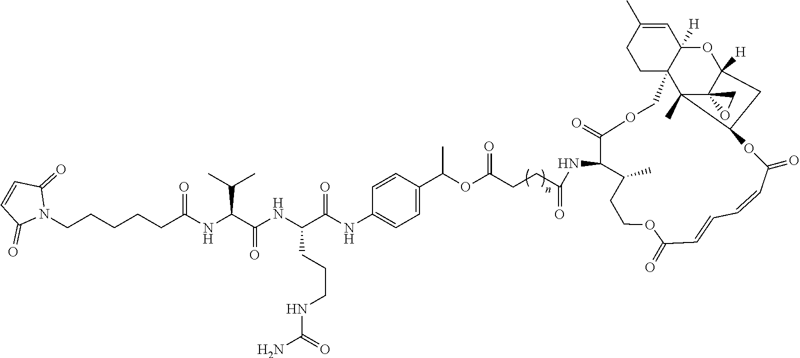 |
| 21b (n = 2) | 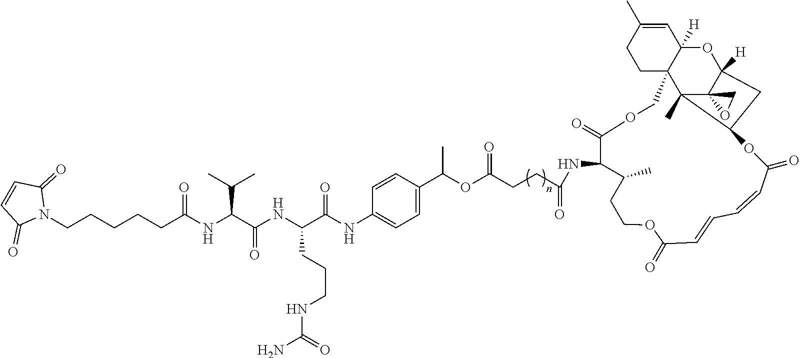 |
| 21c (n = 3) | 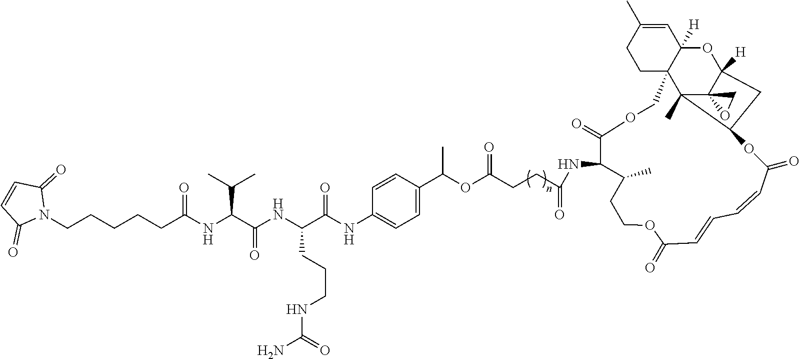 |
| 37 | 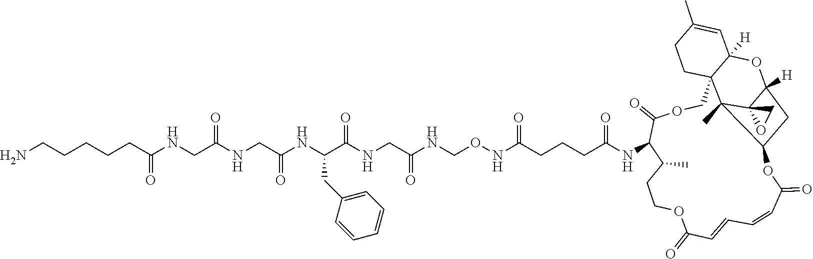 [SMILES: CC1=C[C@H]2O[C@@H]3C[C@H]4OC(=O)/C=C\C=C\C(=O)OCC[C@@H](C)[C@@H](NC(=O)CCCC(=O)NOCNC(=O)CNC(=O)[C@H](Cc5ccccc5)NC(=O)CNC(=O)CNC(=O)CCCCCN)C(=O)OC[C@@]2(CC1)[C@]4(C)[C@]31CO1] |
| 46 | 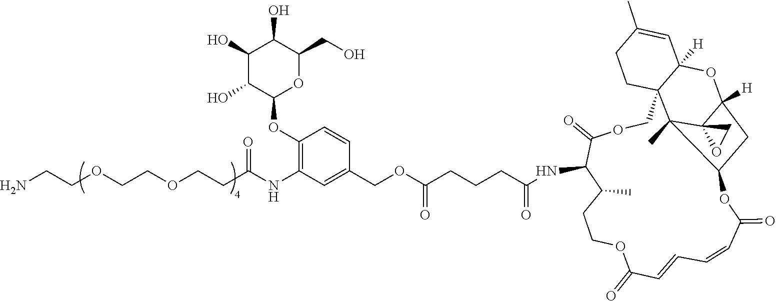 |
| 53 | 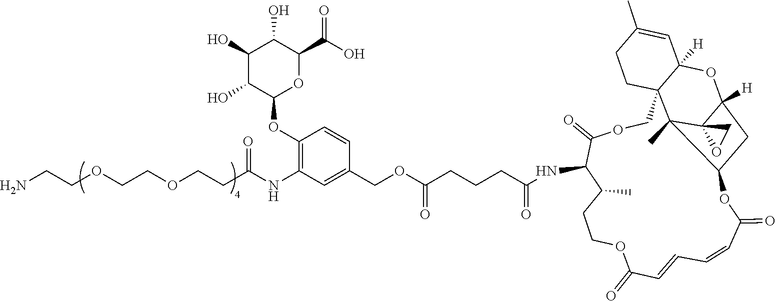 |
| 70a | 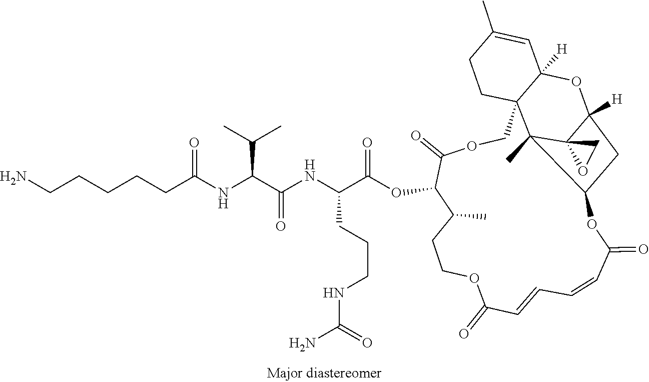 [SMILES: CC1=C[C@H]2O[C@@H]3C[C@H]4OC(=O)/C=C\C=C\C(=O)OCC[C@@H](C)[C@H](OC(=O)[C@H](CCCNC(N)=O)NC(=O)[C@@H](NC(=O)CCCCCN)C(C)C)C(=O)OC[C@@]2(CC1)[C@]4(C)[C@]31CO1] |
| 70b | 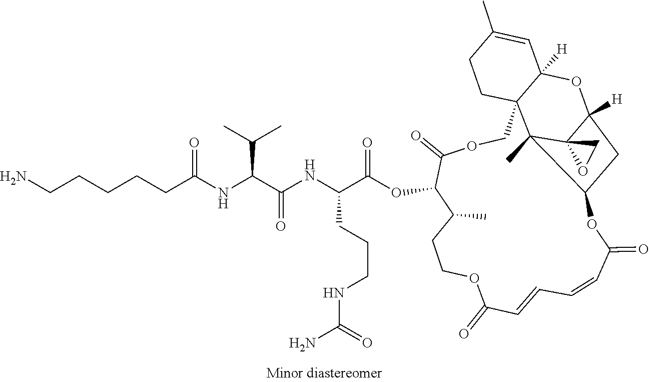 [SMILES: CC1=C[C@H]2O[C@@H]3C[C@H]4OC(=O)/C=C\C=C\C(=O)OCC[C@@H](C)[C@H](OC(=O)[C@H](CCCNC(N)=O)NC(=O)[C@@H](NC(=O)CCCCCN)C(C)C)C(=O)OC[C@@]2(CC1)[C@]4(C)[C@]31CO1] |
| 74 | 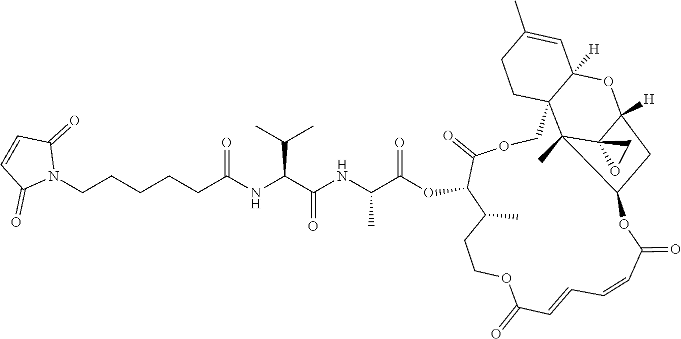 [SMILES: CC1=C[C@H]2O[C@@H]3C[C@H]4OC(=O)/C=C\C=C\C(=O)OCC[C@@H](C)[C@H](OC(=O)[C@H](C)NC(=O)[C@@H](NC(=O)CCCCCN5C(=O)C=CC5=O)C(C)C)C(=O)OC[C@@]2(CC1)[C@]4(C)[C@]31CO1] |
| 75 | 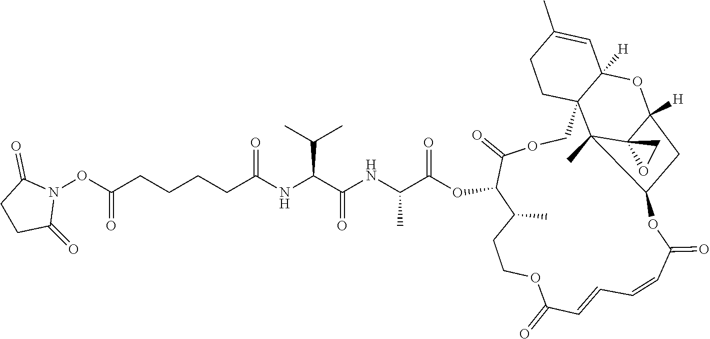 [SMILES: CC1=C[C@H]2O[C@@H]3C[C@H]4OC(=O)/C=C\C=C\C(=O)OCC[C@@H](C)[C@H](OC(=O)[C@H](C)NC(=O)[C@@H](NC(=O)CCCCC(=O)ON5C(=O)CCC5=O)C(C)C)C(=O)OC[C@@]2(CC1)[C@]4(C)[C@]31CO1] |
| 76 | 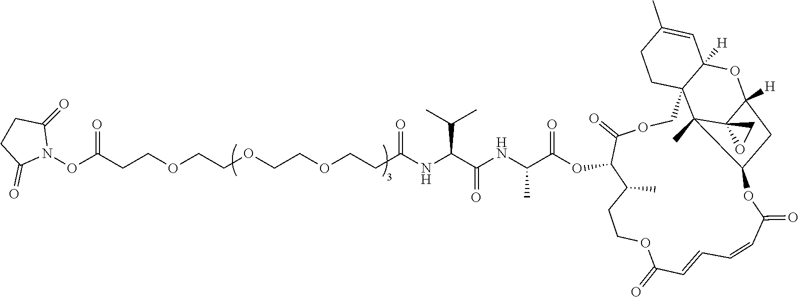 |
| 88 | 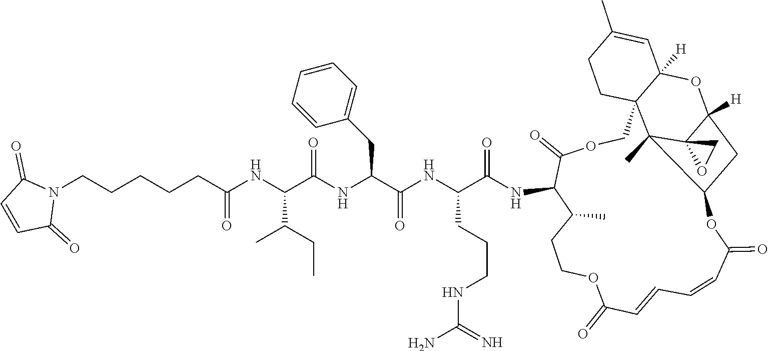 [SMILES: CCC(C)[C@H](NC(=O)CCCCCN1C(=O)C=CC1=O)C(=O)N[C@@H](Cc1ccccc1)C(=O)N[C@@H](CCCNC(=N)N)C(=O)N[C@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1] |
| 91 | 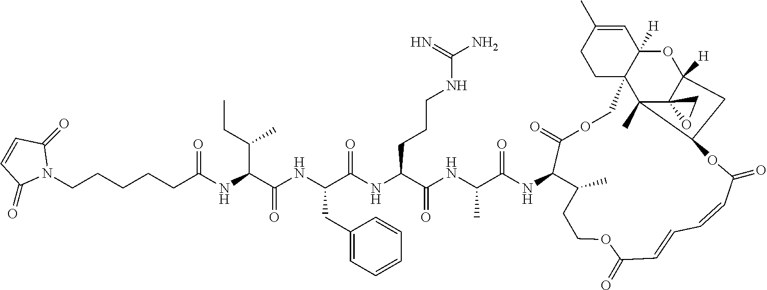 [SMILES: CC[C@H](C)[C@H](NC(=O)CCCCCN1C(=O)C=CC1=O)C(=O)N[C@@H](Cc1ccccc1)C(=O)N[C@@H](CCCNC(=N)N)C(=O)N[C@@H](C)C(=O)N[C@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1] |
| 97 | 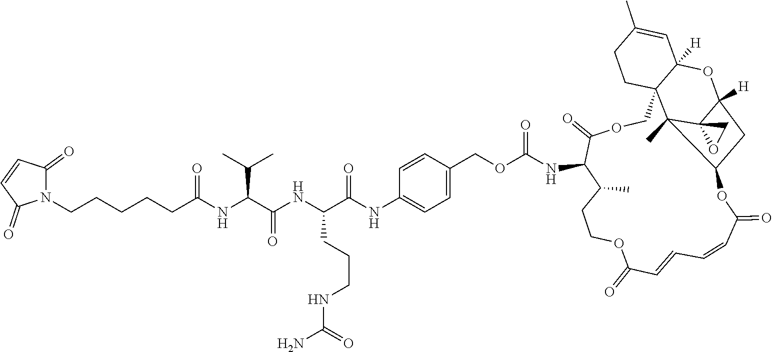 [SMILES: CC1=C[C@H]2O[C@@H]3C[C@H]4OC(=O)/C=C\C=C\C(=O)OCC[C@@H](C)[C@@H](NC(=O)OCc5ccc(NC(=O)[C@H](CCCNC(N)=O)NC(=O)[C@@H](NC(=O)CCCCCN6C(=O)C=CC6=O)C(C)C)cc5)C(=O)OC[C@@]2(CC1)[C@]4(C)[C@]31CO1] |
| 95 | 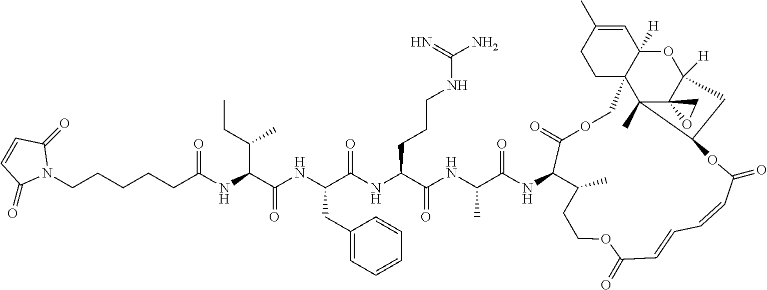 [SMILES: CC[C@H](C)[C@H](NC(=O)CCCCCN1C(=O)C=CC1=O)C(=O)N[C@@H](Cc1ccccc1)C(=O)N[C@@H](CCCNC(=N)N)C(=O)N[C@@H](C)C(=O)N[C@H]1C(=O)OC[C@]23CCC(C)=C[C@H]2O[C@@H]2C[C@@H](OC(=O)/C=C\C=C\C(=O)OCC[C@H]1C)[C@@]3(C)[C@]21CO1] |
| 101 | 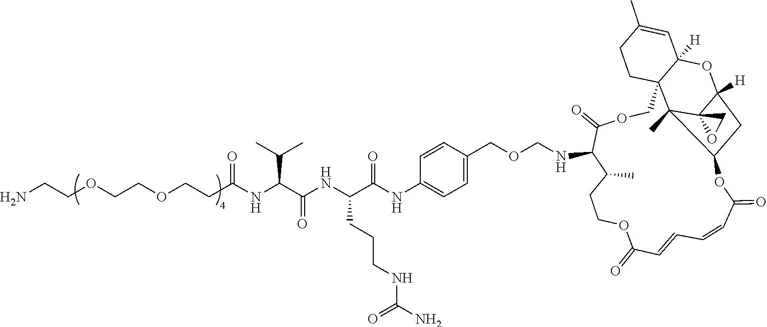 |
| 106 | 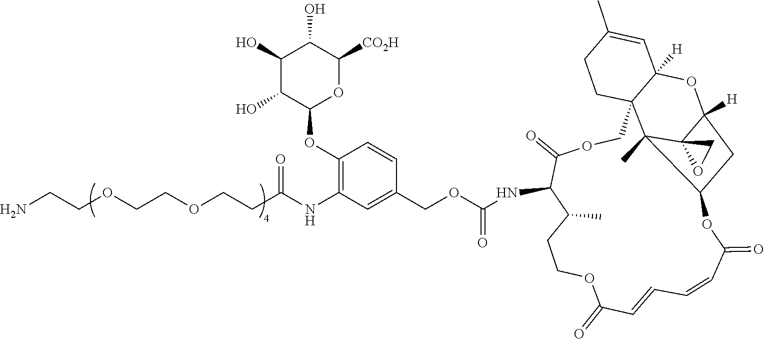 |
| 109 | 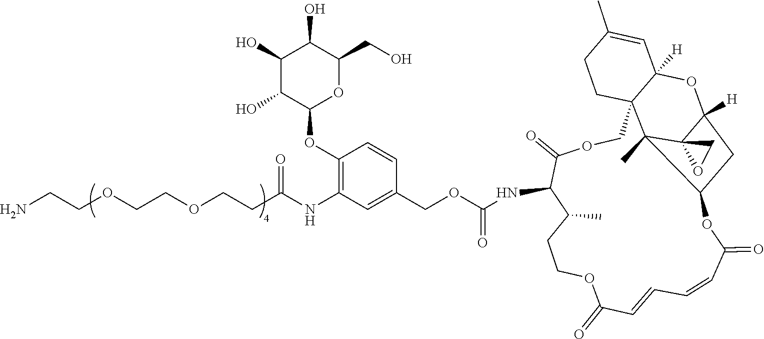 |
| 114 | 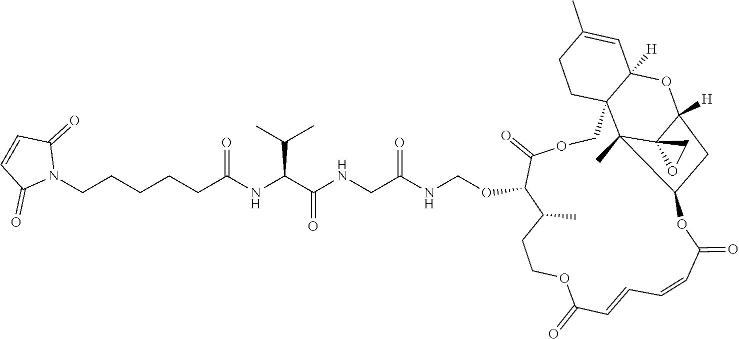 [SMILES: CC1=C[C@H]2O[C@@H]3C[C@H]4OC(=O)/C=C\C=C\C(=O)OCC[C@@H](C)[C@H](OCNC(=O)CNC(=O)[C@@H](NC(=O)CCCCCN5C(=O)C=CC5=O)C(C)C)C(=O)OC[C@@]2(CC1)[C@]4(C)[C@]31CO1] |
| 118 | 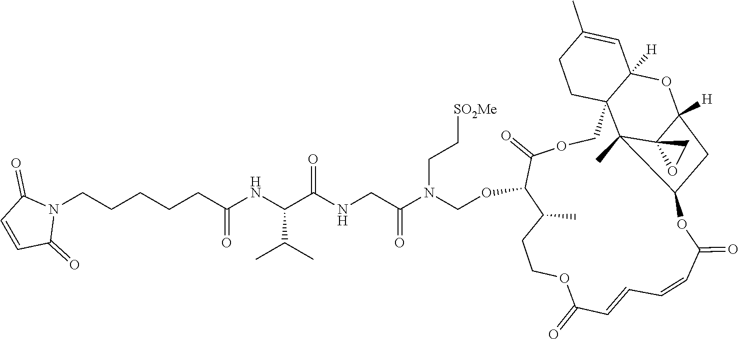 [SMILES: CC1=C[C@H]2O[C@@H]3C[C@H]4OC(=O)/C=C\C=C\C(=O)OCC[C@@H](C)[C@H](OCN(CCS(C)(=O)=O)C(=O)CNC(=O)[C@@H](NC(=O)CCCCCN5C(=O)C=CC5=O)C(C)C)C(=O)OC[C@@]2(CC1)[C@]4(C)[C@]31CO1] |
| 121 | 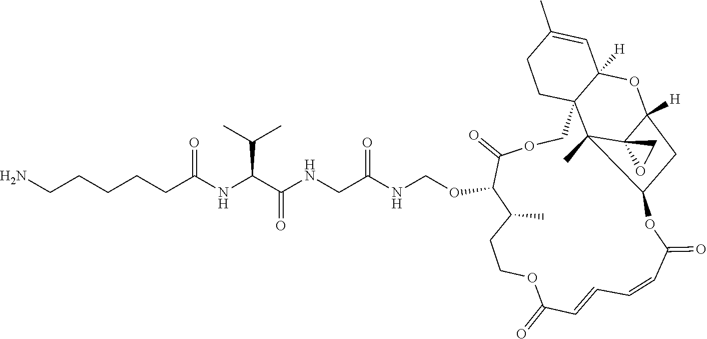 [SMILES: CC1=C[C@H]2O[C@@H]3C[C@H]4OC(=O)/C=C\C=C\C(=O)OCC[C@@H](C)[C@H](OCNC(=O)CNC(=O)[C@@H](NC(=O)CCCCCN)C(C)C)C(=O)OC[C@@]2(CC1)[C@]4(C)[C@]31CO1] |
| 124 | 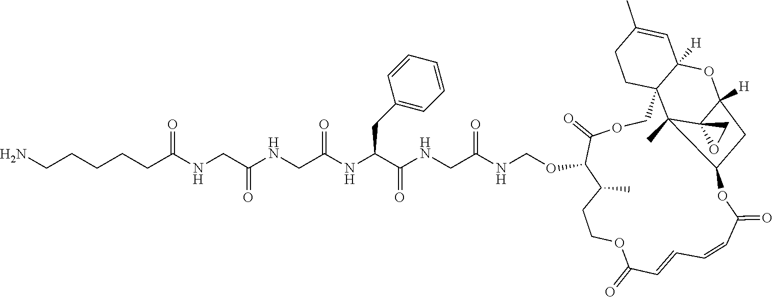 [SMILES: CC1=C[C@H]2O[C@@H]3C[C@H]4OC(=O)/C=C\C=C\C(=O)OCC[C@@H](C)[C@H](OCNC(=O)CNC(=O)[C@H](Cc5ccccc5)NC(=O)CNC(=O)CNC(=O)CCCCCN)C(=O)OC[C@@]2(CC1)[C@]4(C)[C@]31CO1] |
| 126 | 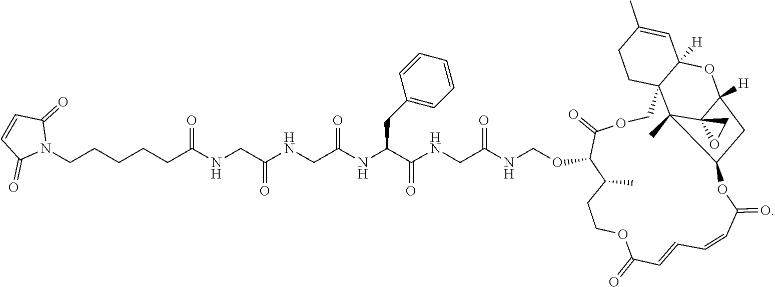 [SMILES: CC1=C[C@H]2O[C@@H]3C[C@H]4OC(=O)/C=C\C=C\C(=O)OCC[C@@H](C)[C@H](OCNC(=O)CNC(=O)[C@H](Cc5ccccc5)NC(=O)CNC(=O)CNC(=O)CCCCCN5C(=O)C=CC5=O)C(=O)OC[C@@]2(CC1)[C@]4(C)[C@]31CO1] |

68. The compound of claim 35 selected from:

| Compound | Structure |
|---|---|
| 10a (n = 1) | 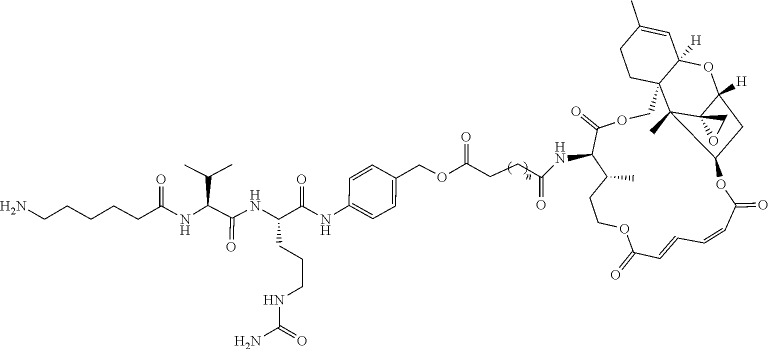 |
| 10b (n = 2) | 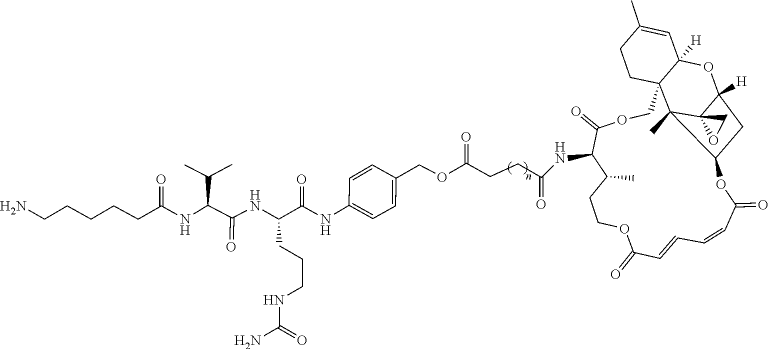 |
| 10c (n = 3) | 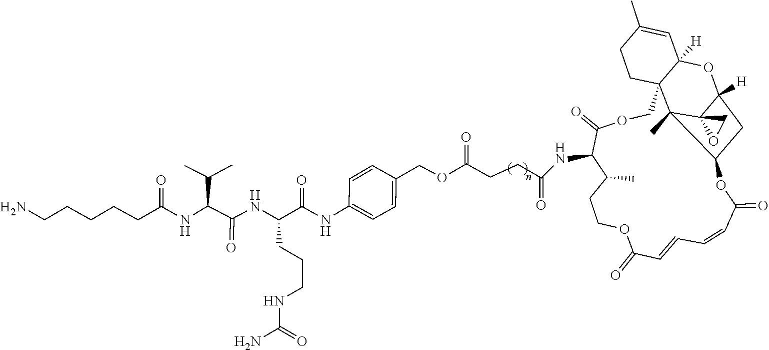 |
| 12a (n = 1) | 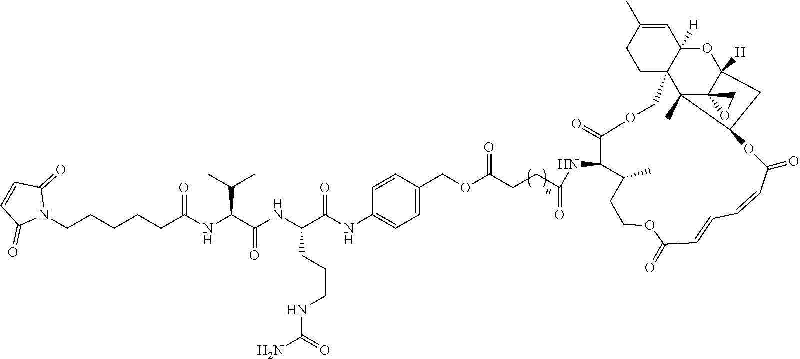 |
| 12b (n = 2) | 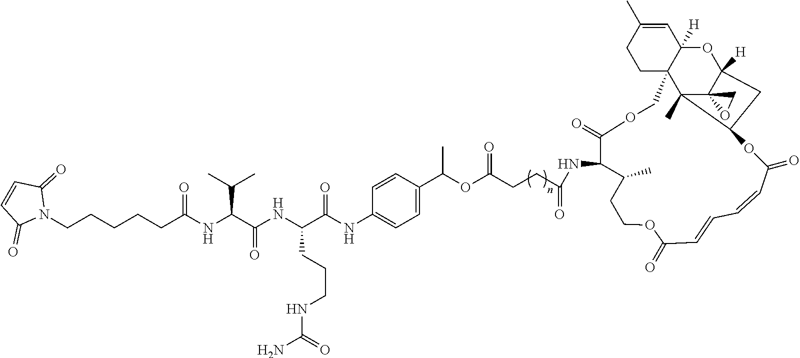 |
| 12c (n = 3) | 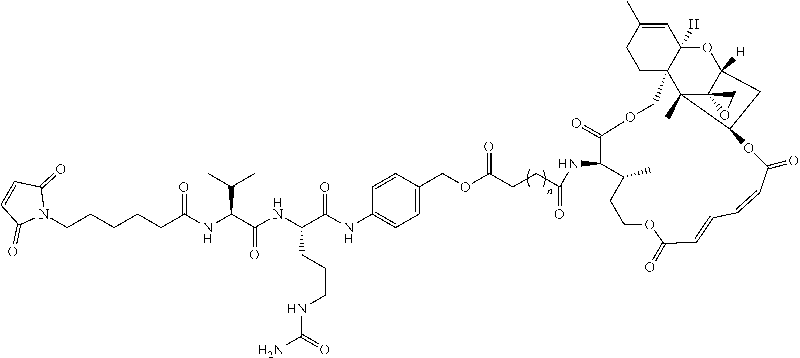 |
| 19a (n = 1) | 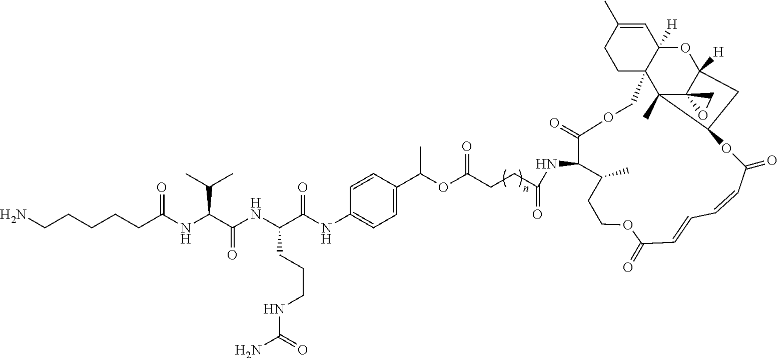 |
| 19b (n = 2) | 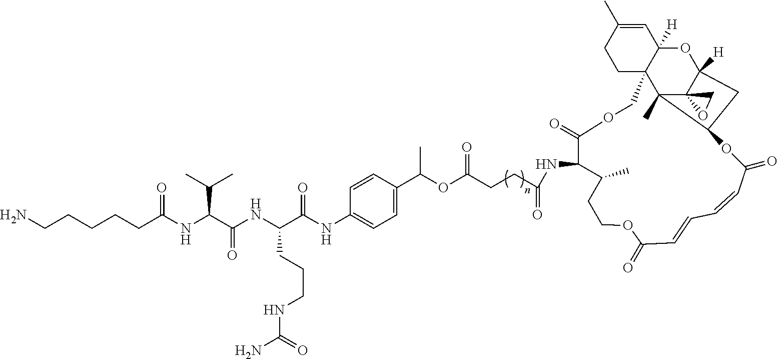 |
| 19c (n = 3) | 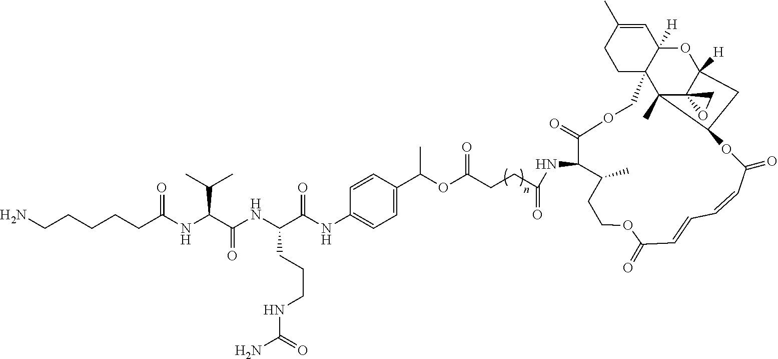 |
| 21a (n = 1) | 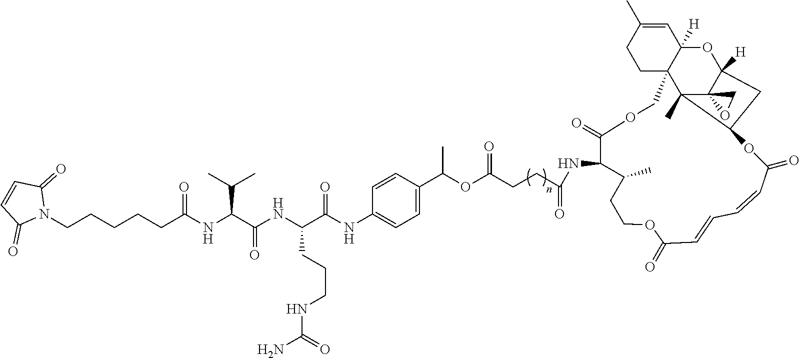 |
| 21b (n = 2) | 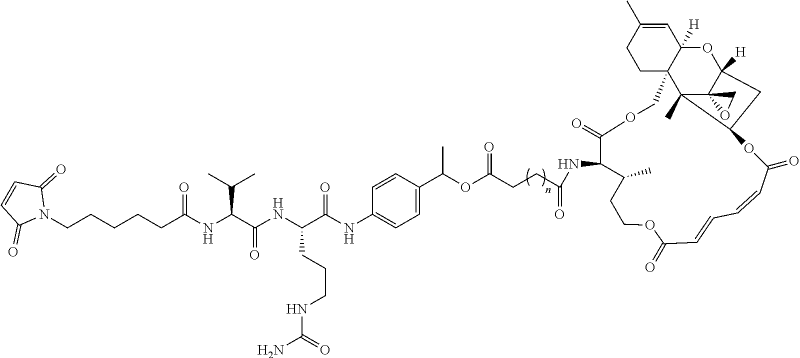 |
| 21c (n = 3) | 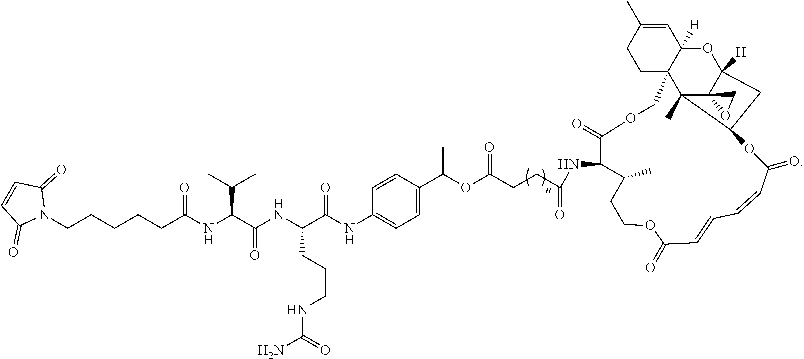 |

69. A compound of Formula V:
Z-(L-X)ᵥ V
or a pharmaceutically acceptable derivative thereof, wherein:
Z is an anti-influenza antigen-binding domain;
L is a linking group;
X is a verrucarin A derivative; and
v is an integer from 1 to 12.

70. A compound of Formula V:
Z-(L-X)ᵥ V
or a pharmaceutically acceptable derivative thereof, wherein:
Z is an anti-SARS-CoV-2 antigen-binding domain;
L is a linking group;
X is a verrucarin A derivative; and
v is an integer from 1 to 12.

71. A compound of Formula V:
Z-(L-X)v V
or a pharmaceutically acceptable derivative thereof, wherein:
Z is an anti-ebola virus antigen-binding domain;
L is a linking group;
X is a verrucarin A derivative; and
v is an integer from 1 to 12.

72. (canceled)

73. (canceled)

74. (canceled)

75. The compound of claim 69 wherein -L-X is radical formed by removal of an H from a compound of claim 35.

76. The compound of claim 70 wherein -L-X is radical formed by removal of an H from a compound of claim 35.

77. The compound of claim 71 wherein -L-X is radical formed by removal of an H from a compound of claim 35.

78. (canceled)

79. (canceled)

80. (canceled)

81. (canceled)

82. The compound of claim 69, wherein v is 1, 2, 3, 4, 5, 6, 7 or 8.

83. (canceled)

84. (canceled)

85. (canceled)

86. (canceled)

87. (canceled)

88. The compound of claim 69, wherein Z comprises an antibody heavy chain and further includes a peptide tag at the C-terminus of the antibody heavy chain, wherein the peptide tag is ELQRP, LLQG, LLQGG, LLQLLQG, LLQYQG, LLQGA, LLQGSG, SLLQG, LQG, LLQLQ, LLQLLQ, LLQGR, LLQYQGA, LQGG, LGQG or LLQLLQGA.

89. The compound of claim 69, wherein Z comprises two antibody heavy chains and a peptide tag at the C-terminus of each antibody heavy chain.

90. The compound of claim 89, wherein the peptide tag is the pentapeptide sequence LLQGA.

91. The compound of claim 89, wherein the peptide tag is the pentapeptide sequence ELQGP.

92. The compound of claim 69, having one of the following formulae:

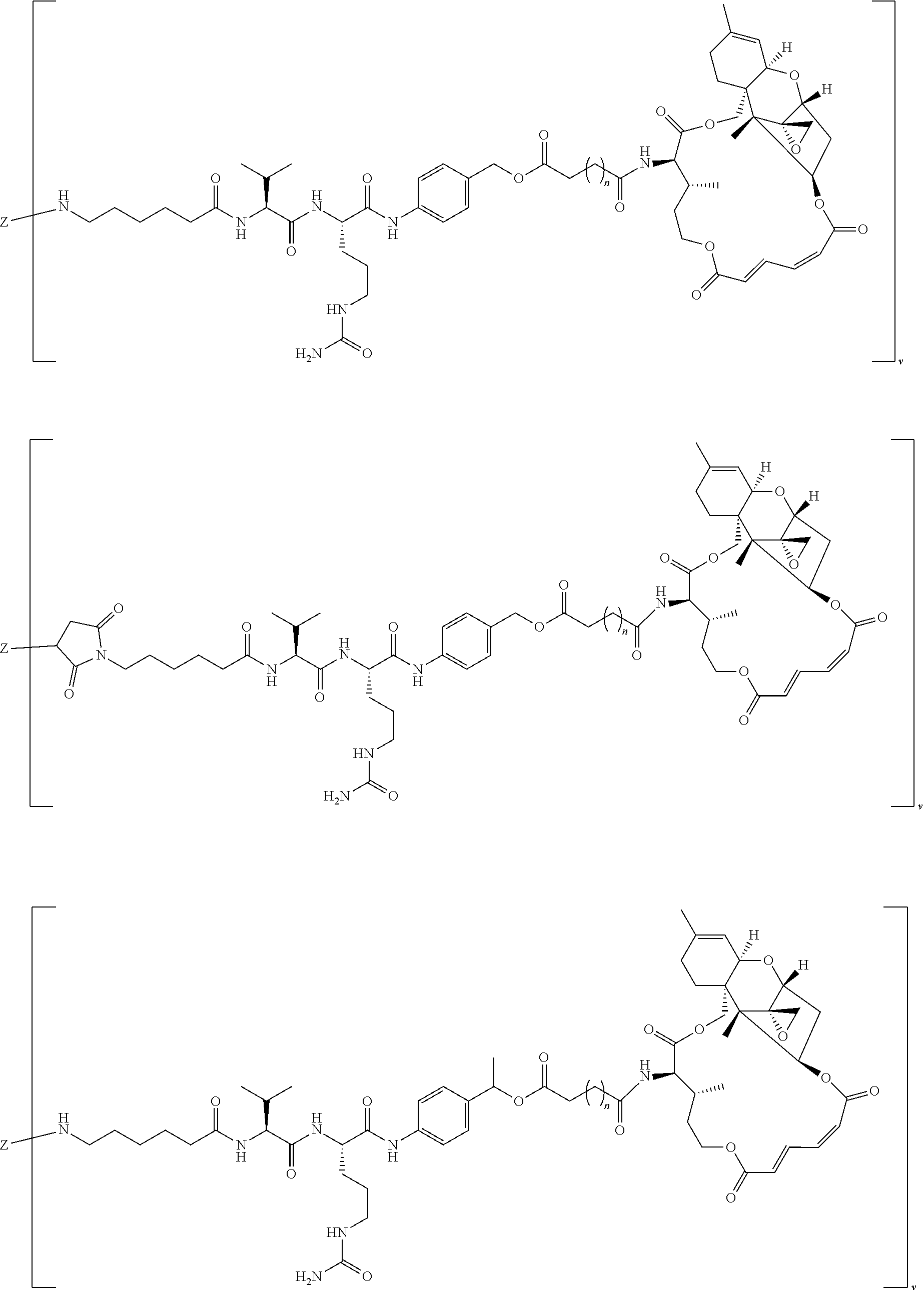

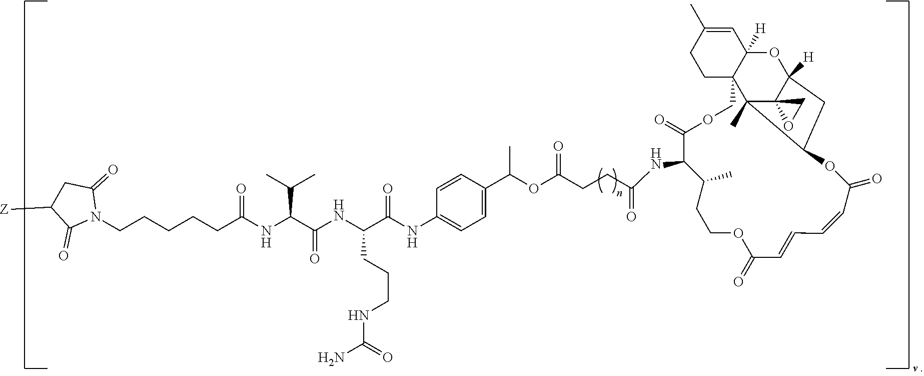

93. (canceled)

94. A pharmaceutical composition, comprising a compound of claim 1 and a pharmaceutically acceptable carrier.

95. A method of treating influenza infection in a subject, comprising administering to the subject a compound of claim 69.

96. The method of claim 95, wherein the influenza is influenza A.

97. A method of treating SARS-CoV-2 infection in a subject, comprising administering to the subject a compound of claim 70.

98. The method of claim 97, wherein the SARS-CoV-2 is an omicron variant.

99. A method of treating ebola infection in a subject, comprising administering to the subject a compound of claim 71.


SEQUENCE LISTING (378 sequences)

SEQ ID NO: 1 - DNA, 375 bp, synthetic construct
  misc_feature 1..375: note = synthetic
  source 1..375: mol_type = other DNA
caggtgcagctggtgcagtctggggctgaggtgaagaagcctgggtcctcggtgaaggtctcctgcaaggcttctggaggcaccttcagcaactatgctatcagctgggtgcgacaggcccctggacaagggcttgagaggatgggagggatcatccctatctttggtacagcaaactacgcacagaagttccagggcagagtcacgattaccacggacgaatccacgagcacagcctacatggagctgggcagcctgagatctgaggacacggccgtgtattactgtgcgagagggaaggatttctatggttcggggagttattataacctctttgactactggggccagggaaccctggtcaccgtctcctca

SEQ ID NO: 2 - protein, 125 aa, synthetic construct
  REGION 1..125: note = synthetic
QVQLVQSGAEVKKPGSSVKVSCKASGGTFSNYAISWVRQAPGQGLERMGGIIPIFGTANYAQKFQGRVTITTDESTSTAYMELGSLRSEDTAVYYCARGKDFYGSGSYYNLFDYWGQGTLVTVSS

SEQ ID NO: 3 - DNA, 24 bp, synthetic construct
  misc_feature 1..24: note = synthetic
  source 1..24: mol_type = other DNA
ggaggcaccttcagcaactatgct

SEQ ID NO: 4 - protein, 8 aa, synthetic construct
  REGION 1..8: note = synthetic
GGTFSNYA

SEQ ID NO: 5 - DNA, 24 bp, synthetic construct
  misc_feature 1..24: note = synthetic
  source 1..24: mol_type = other DNA
atcatccctatctttggtacagca

SEQ ID NO: 6 - protein, 8 aa, synthetic construct
  REGION 1..8: note = synthetic
IIPIFGTA

SEQ ID NO: 7 - DNA, 54 bp, synthetic construct
  misc_feature 1..54: note = synthetic
  source 1..54: mol_type = other DNA
gcgagagggaaggatttctatggttcggggagttattataacctctttgactac

SEQ ID NO: 8 - protein, 18 aa, synthetic construct
  REGION 1..18: note = synthetic
ARGKDFYGSGSYYNLFDY

SEQ ID NO: 9 - DNA, 321 bp, synthetic construct
  misc_feature 1..321: note = synthetic
  source 1..321: mol_type = other DNA
gacatccagatgacccagtctccttccaccctgtctgcatctgtaggagacatagtcaccatcacttgccgggccagtcagagtattagtagctggttggcctggtatcagcagaaaccagggaaagcccctaagctcctgatctataaggcgtctagtttacaaagtggggtcccatcaaggttcagcggcagtggatctgggacagaattcactctcaccatcagcagcctgcagcctgatgattttgcaactttttactgccaacactataatacttattcgtggacgttcggccaagggaccaaggtggaaatcaaa

SEQ ID NO: 10 - protein, 107 aa, synthetic construct
  REGION 1..107: note = synthetic
DIQMTQSPSTLSASVGDIVTITCRASQSISSWLAWYQQKPGKAPKLLIYKASSLQSGVPSRFSGSGSGTEFTLTISSLQPDDFATFYCQHYNTYSWTFGQGTKVEIK

SEQ ID NO: 11 - DNA, 18 bp, synthetic construct
  misc_feature 1..18: note = synthetic
  source 1..18: mol_type = other DNA
cagagtattagtagctgg

SEQ ID NO: 12 - protein, 6 aa, synthetic construct
  REGION 1..6: note = synthetic
QSISSW

SEQ ID NO: 13-14 - skipped (2 entries)

SEQ ID NO: 15 - DNA, 27 bp, synthetic construct
  misc_feature 1..27: note = synthetic
  source 1..27: mol_type = other DNA
caacactataatacttattcgtggacg

SEQ ID NO: 16 - protein, 9 aa, synthetic construct
  REGION 1..9: note = synthetic
QHYNTYSWT

SEQ ID NO: 17 - DNA, 360 bp, synthetic construct
  misc_feature 1..360: note = synthetic
  source 1..360: mol_type = other DNA
caggtgcagctggtgcagtctggggctgaggtgaagaagtctgggtcctcggtgaaggtctcctgcaaggcttctggaggcaccttcagcagctatgctattagctgggtgcgccaggcccctggacaagggcttgagtggatgggagggatcatccctatttttggtacaccaagctatgcacagaagttccaggacagagtcacgattaccacggacgaatccacgagcacagtttacatggagctgagcagcctgagatctgaagacacggccgtgtattactgtgcgagacagcagcccgtctaccagtacaatatggacgtctggggccaagggaccacggtcaccgtctcctca

SEQ ID NO: 18 - protein, 120 aa, synthetic construct
  REGION 1..120: note = synthetic
QVQLVQSGAEVKKSGSSVKVSCKASGGTFSSYAISWVRQAPGQGLEWMGGIIPIFGTPSYAQKFQDRVTITTDESTSTVYMELSSLRSEDTAVYYCARQQPVYQYNMDVWGQGTTVTVSS

SEQ ID NO: 19 - DNA, 24 bp, synthetic construct
  misc_feature 1..24: note = synthetic
  source 1..24: mol_type = other DNA
ggaggcaccttcagcagctatgct

SEQ ID NO: 20 - protein, 8 aa, synthetic construct
  REGION 1..8: note = synthetic
GGTFSSYA

SEQ ID NO: 21 - DNA, 24 bp, synthetic construct
  misc_feature 1..24: note = synthetic
  source 1..24: mol_type = other DNA
atcatccctatttttggtacacca

SEQ ID NO: 22 - protein, 8 aa, synthetic construct
  REGION 1..8: note = synthetic
IIPIFGTP

SEQ ID NO: 23 - DNA, 39 bp, synthetic construct
  misc_feature 1..39: note = synthetic
  source 1..39: mol_type = other DNA
gcgagacagcagcccgtctaccagtacaatatggacgtc

SEQ ID NO: 24 - protein, 13 aa, synthetic construct
  REGION 1..13: note = synthetic
ARQQPVYQYNMDV

SEQ ID NO: 25 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccgggcaagtcagggcattagaaataatttaggctggtatcagcagaaaccactgaaagcccctaagcgcctgatctatgctgcgtccagtttgcaaagtggggtcccatcaaggttcagcggcagtggatctgggacagaattcactctcacaatcagcagcctgcagcctgaagattttgcaacttattactgtctacaatataataattacccgtggacgttcggccaagggaccaaggtggaaatcaaa

SEQ ID NO: 26 - protein, 107 aa, synthetic construct
  REGION 1..107: note = synthetic
DIQMTQSPSSLSASVGDRVTITCRASQGIRNNLGWYQQKPLKAPKRLIYAASSLQSGVPSRFSGSGSGTEFTLTISSLQPEDFATYYCLQYNNYPWTFGQGTKVEIK

SEQ ID NO: 27 - DNA, 18 bp, synthetic construct
  misc_feature 1..18: note = synthetic
  source 1..18: mol_type = other DNA
cagggcattagaaataat

SEQ ID NO: 28 - protein, 6 aa, synthetic construct
  REGION 1..6: note = synthetic
QGIRNN

SEQ ID NO: 29-30 - skipped (2 entries)

SEQ ID NO: 31 - DNA, 27 bp, synthetic construct
  misc_feature 1..27: note = synthetic
  source 1..27: mol_type = other DNA
ctacaatataataattacccgtggacg

SEQ ID NO: 32 - protein, 9 aa, synthetic construct
  REGION 1..9: note = synthetic
LQYNNYPWT

SEQ ID NO: 33 - DNA, 366 bp, synthetic construct
  misc_feature 1..366: note = synthetic
  source 1..366: mol_type = other DNA
caggtgcagctggtgcagtctggggctgaagtgaagaagcctgggtcctcggtgaaggtctcctgcaaggcttctggaggcaccttcaacagctatgctatcagctgggtgcgacaggcccctggacaagggcttgagtggatgggaggcatcatccctatctttgctacaacaaacttcgcacagaagttccagggcagagtcacgattacctcggacgaatccacgaacacagcctacatggagctgaccagcctgagatctgaggacacggccgcgtattactgtgcaagggggggttggtataactggcagtacgtggggtttgactcctggggccaagggaccacggtcaccgtctcctca

SEQ ID NO: 34 - protein, 122 aa, synthetic construct
  REGION 1..122: note = synthetic
QVQLVQSGAEVKKPGSSVKVSCKASGGTFNSYAISWVRQAPGQGLEWMGGIIPIFATTNFAQKFQGRVTITSDESTNTAYMELTSLRSEDTAAYYCARGGWYNWQYVGFDSWGQGTTVTVSS

SEQ ID NO: 35 - DNA, 24 bp, synthetic construct
  misc_feature 1..24: note = synthetic
  source 1..24: mol_type = other DNA
ggaggcaccttcaacagctatgct

SEQ ID NO: 36 - protein, 8 aa, synthetic construct
  REGION 1..8: note = synthetic
GGTFNSYA

SEQ ID NO: 37 - DNA, 24 bp, synthetic construct
  misc_feature 1..24: note = synthetic
  source 1..24: mol_type = other DNA
atcatccctatctttgctacaaca

SEQ ID NO: 38 - protein, 8 aa, synthetic construct
  REGION 1..8: note = synthetic
IIPIFATT

SEQ ID NO: 39 - DNA, 45 bp, synthetic construct
  misc_feature 1..45: note = synthetic
  source 1..45: mol_type = other DNA
gcaagggggggttggtataactggcagtacgtggggtttgactcc

SEQ ID NO: 40 - protein, 15 aa, synthetic construct
  REGION 1..15: note = synthetic
ARGGWYNWQYVGFDS

SEQ ID NO: 41 - DNA, 321 bp, synthetic construct
  misc_feature 1..321: note = synthetic
  source 1..321: mol_type = other DNA
gacatccagatgacccagtctccttccaccctgtctgcatctgtaggagacagagtcgccatcacttgccgggccagtcagagtattagtacctggttggcctggtatcagcagaaaccagggaaagcccctaagctcctgatctataaggcgtctagtttagaaaatggggtcccttcaaggttcagcggcagtggatctgggacagatttcactctcaccatcagcagcctgcaggctgatgattttgcaacttattactgccaaaagtataatagtaattcgtggacgttcggccaagggaccaaggtggatatcaaa

SEQ ID NO: 42 - protein, 107 aa, synthetic construct
  REGION 1..107: note = synthetic
DIQMTQSPSTLSASVGDRVAITCRASQSISTWLAWYQQKPGKAPKLLIYKASSLENGVPSRFSGSGSGTDFTLTISSLQADDFATYYCQKYNSNSWTFGQGTKVDIK

SEQ ID NO: 43 - DNA, 18 bp, synthetic construct
  misc_feature 1..18: note = synthetic
  source 1..18: mol_type = other DNA
cagagtattagtacctgg

SEQ ID NO: 44 - protein, 6 aa, synthetic construct
  REGION 1..6: note = synthetic
QSISTW

SEQ ID NO: 45-46 - skipped (2 entries)

SEQ ID NO: 47 - DNA, 27 bp, synthetic construct
  misc_feature 1..27: note = synthetic
  source 1..27: mol_type = other DNA
caaaagtataatagtaattcgtggacg

SEQ ID NO: 48 - protein, 9 aa, synthetic construct
  REGION 1..9: note = synthetic
QKYNSNSWT

SEQ ID NO: 49 - DNA, 360 bp, synthetic construct
  misc_feature 1..360: note = synthetic
  source 1..360: mol_type = other DNA
caggtccacctggtgcagtctgggccagaggtgaagaagcctgggtcctcggtgaaggtctcctgcaaggcttctggagtcaccttcatcagtcatgctatcagctgggtgcgacaggcccctggacaagggcttgaatgggtgggaggaatcatcgctatctttggtacaacaaactacgcacagaagttccagggcagagtcacggttacaacggacaaatccacgaacacagtctacatggaattgagcagactgagatctgaggacacggccatttattactgtgcgcgaggtgagacctactacgagggaaactttgacttctggggccagggaaccctggtcaccgtctcctca

SEQ ID NO: 50 - protein, 120 aa, synthetic construct
  REGION 1..120: note = synthetic
QVHLVQSGPEVKKPGSSVKVSCKASGVTFISHAISWVRQAPGQGLEWVGGIIAIFGTTNYAQKFQGRVTVTTDKSTNTVYMELSRLRSEDTAIYYCARGETYYEGNFDFWGQGTLVTVSS

SEQ ID NO: 51 - DNA, 24 bp, synthetic construct
  misc_feature 1..24: note = synthetic
  source 1..24: mol_type = other DNA
ggagtcaccttcatcagtcatgct

SEQ ID NO: 52 - protein, 8 aa, synthetic construct
  REGION 1..8: note = synthetic
GVTFISHA

SEQ ID NO: 53 - DNA, 24 bp, synthetic construct
  misc_feature 1..24: note = synthetic
  source 1..24: mol_type = other DNA
atcatcgctatctttggtacaaca

SEQ ID NO: 54 - protein, 8 aa, synthetic construct
  REGION 1..8: note = synthetic
IIAIFGTT

SEQ ID NO: 55 - DNA, 39 bp, synthetic construct
  misc_feature 1..39: note = synthetic
  source 1..39: mol_type = other DNA
gcgcgaggtgagacctactacgagggaaactttgacttc

SEQ ID NO: 56 - protein, 13 aa, synthetic construct
  REGION 1..13: note = synthetic
ARGETYYEGNFDF

SEQ ID NO: 57 - DNA, 324 bp, synthetic construct
  misc_feature 1..324: note = synthetic
  source 1..324: mol_type = other DNA
gtcatccagatgacccagtctccatcctccctgtctgcatctgtaggagacagagtcaccatcacttgccgggccagtcagagcgttaggagtaatttaaattggtatcagcagacaccagggaaagcccctaggctcctgatctatgctgcatccagtttgcaaagtggggtcccatcaaggttcagtggcagtggatctgggacagatttcactctcaccatcagcagtctgcaacctgaagattttgcaacttattactgtcaacagagttacagtacccctccgatcaccttcggccaagggacacgactggagattaaa

SEQ ID NO: 58 - protein, 108 aa, synthetic construct
  REGION 1..108: note = synthetic
VIQMTQSPSSLSASVGDRVTITCRASQSVRSNLNWYQQTPGKAPRLLIYAASSLQSGVPSRFSGSGSGTDFTLTISSLQPEDFATYYCQQSYSTPPITFGQGTRLEIK

SEQ ID NO: 59 - DNA, 18 bp, synthetic construct
  misc_feature 1..18: note = synthetic
  source 1..18: mol_type = other DNA
cagagcgttaggagtaat

SEQ ID NO: 60 - protein, 6 aa, synthetic construct
  REGION 1..6: note = synthetic
QSVRSN

SEQ ID NO: 61-62 - skipped (2 entries)

SEQ ID NO: 63 - DNA, 30 bp, synthetic construct
  misc_feature 1..30: note = synthetic
  source 1..30: mol_type = other DNA
caacagagttacagtacccctccgatcacc

SEQ ID NO: 64 - protein, 10 aa, synthetic construct
  REGION 1..10: note = synthetic
QQSYSTPPIT

SEQ ID NO: 65 - DNA, 324 bp, synthetic construct
  misc_feature 1..324: note = synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 66 - protein, 108 aa, synthetic construct
  REGION 1..108: note = synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 67 - DNA, 18 bp, synthetic construct
  misc_feature 1..18: note = synthetic
  source 1..18: mol_type = other DNA
cagagcattagcagctat

SEQ ID NO: 68 - protein, 6 aa, synthetic construct
  REGION 1..6: note = synthetic
QSISSY

SEQ ID NO: 69-70 - skipped (2 entries)

SEQ ID NO: 71 - DNA, 30 bp, synthetic construct; identical to SEQ ID NO: 63
  misc_feature 1..30: note = synthetic
  source 1..30: mol_type = other DNA

SEQ ID NO: 72 - protein, 10 aa, synthetic construct
  REGION 1..10: note = synthetic
QQSYSTPPIT

SEQ ID NO: 73 - DNA, 369 bp, synthetic construct
  misc_feature 1..369: note = synthetic
  source 1..369: mol_type = other DNA
caggtgcagctggtgcagtctggagcagaggtgaggaagcctgggtcctcggtgaaggtctcctgcaaggcttctggaggaaccttcaccggccatgctatcagctgggtgcgacaggcccctggacaaggccttgagtggatgggaggggtcgtccctatctttggttcagcaaactacgcacagaagttccagggcagagtcacgatgaccatggacgaatccacgagtacagcctacatggacctgagcagcctgagatctgaggacacggccgtttattattgtgtgagagatccgggcaactcgggatactactactacggtatggacgtctggggccaagggaccacggtcaccgtctcctca

SEQ ID NO: 74 - protein, 123 aa, synthetic construct
  REGION 1..123: note = synthetic
QVQLVQSGAEVRKPGSSVKVSCKASGGTFTGHAISWVRQAPGQGLEWMGGVVPIFGSANYAQKFQGRVTMTMDESTSTAYMDLSSLRSEDTAVYYCVRDPGNSGYYYYGMDVWGQGTTVTVSS

SEQ ID NO: 75 - DNA, 24 bp, synthetic construct
  misc_feature 1..24: note = synthetic
  source 1..24: mol_type = other DNA
ggaggaaccttcaccggccatgct

SEQ ID NO: 76 - protein, 8 aa, synthetic construct
  REGION 1..8: note = synthetic
GGTFTGHA

SEQ ID NO: 77 - DNA, 24 bp, synthetic construct
  misc_feature 1..24: note = synthetic
  source 1..24: mol_type = other DNA
gtcgtccctatctttggttcagca

SEQ ID NO: 78 - protein, 8 aa, synthetic construct
  REGION 1..8: note = synthetic
VVPIFGSA

SEQ ID NO: 79 - DNA, 48 bp, synthetic construct
  misc_feature 1..48: note = synthetic
  source 1..48: mol_type = other DNA
gtgagagatccgggcaactcgggatactactactacggtatggacgtc

SEQ ID NO: 80 - protein, 16 aa, synthetic construct
  REGION 1..16: note = synthetic
VRDPGNSGYYYYGMDV

SEQ ID NO: 81 - DNA, 324 bp, synthetic construct
  misc_feature 1..324: note = synthetic
  source 1..324: mol_type = other DNA
gacatccagatgacccagtctccatcctccctgtctgcatctgtaggagacagagtcaccatcacttgccgggcaagtcagagcattagcagctatttaaattggtttcagcagaaaccagggaaagcccctaagctcctgatctatactgcatccagtttgcaaagtggggtcccatcaaggttcagtggcagtggatctgggacagatttcactctcaccatcagcagtctgcaacctgaagattttgcaacttactactgtcaacagagttacagttcccctccgatcaccttcggccaagggacacgactggagattaaa

SEQ ID NO: 82 - protein, 108 aa, synthetic construct
  REGION 1..108: note = synthetic
DIQMTQSPSSLSASVGDRVTITCRASQSISSYLNWFQQKPGKAPKLLIYTASSLQSGVPSRFSGSGSGTDFTLTISSLQPEDFATYYCQQSYSSPPITFGQGTRLEIK

SEQ ID NO: 83 - DNA, 18 bp, synthetic construct
  misc_feature 1..18: note = synthetic
  source 1..18: mol_type = other DNA
cagagcattagcagctat

SEQ ID NO: 84 - protein, 6 aa, synthetic construct
  REGION 1..6: note = synthetic
QSISSY

SEQ ID NO: 85-86 - skipped (2 entries)

SEQ ID NO: 87 - DNA, 30 bp, synthetic construct
  misc_feature 1..30: note = synthetic
  source 1..30: mol_type = other DNA
caacagagttacagttcccctccgatcacc

SEQ ID NO: 88 - protein, 10 aa, synthetic construct
  REGION 1..10: note = synthetic
QQSYSSPPIT

SEQ ID NO: 89 - DNA, 369 bp, synthetic construct
  misc_feature 1..369: note = synthetic
  source 1..369: mol_type = other DNA
caggtccacctggtgcagtctggggctgaggtgaagaagcctgggtcgtcggtgaaggtctcctgcaaggcttctggaggcaccttcagcacctatgctatcacctgggtgcgacaggcccctggacaagggcttgagtggataggagggatcagccgtttctttggttcagcaaactacgcacagaagtttcagggcagagtcacaattaccacggacgaatccacgaacacagcctacatggaactaagcagcctgagatctgaggacacggccgtatattattgtgcgagagatcctggaaacacgggctattatttttacggtatggacgtctggggccaagggaccacggtcaccgtctcctca

SEQ ID NO: 90 - protein, 123 aa, synthetic construct
  REGION 1..123: note = synthetic
QVHLVQSGAEVKKPGSSVKVSCKASGGTFSTYAITWVRQAPGQGLEWIGGISRFFGSANYAQKFQGRVTITTDESTNTAYMELSSLRSEDTAVYYCARDPGNTGYYFYGMDVWGQGTTVTVSS

SEQ ID NO: 91 - DNA, 24 bp, synthetic construct
  misc_feature 1..24: note = synthetic
  source 1..24: mol_type = other DNA
ggaggcaccttcagcacctatgct

SEQ ID NO: 92 - protein, 8 aa, synthetic construct
  REGION 1..8: note = synthetic
GGTFSTYA

SEQ ID NO: 93 - DNA, 24 bp, synthetic construct
  misc_feature 1..24: note = synthetic
  source 1..24: mol_type = other DNA
atcagccgtttctttggttcagca

SEQ ID NO: 94 - protein, 8 aa, synthetic construct
  REGION 1..8: note = synthetic
ISRFFGSA

SEQ ID NO: 95 - DNA, 48 bp, synthetic construct
  misc_feature 1..48: note = synthetic
  source 1..48: mol_type = other DNA
gcgagagatcctggaaacacgggctattatttttacggtatggacgtc

SEQ ID NO: 96 - protein, 16 aa, synthetic construct
  REGION 1..16: note = synthetic
ARDPGNTGYYFYGMDV

SEQ ID NO: 97 - DNA, 324 bp, synthetic construct
  misc_feature 1..324: note = synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatccagtttgcaaagtggggtcccatcaaggttcagtggcagtggatctgggacagatttcactctcaccatcagcagtctgcaacctgaagattttgcaacttactactgtcaacagagtaagagtaattctccgatcaccttcggccaagggacacgactggagattaaa

SEQ ID NO: 98 - protein, 108 aa, synthetic construct
  REGION 1..108: note = synthetic
DIQMTQSPSSLSASVGDRVTITCRASQSISSYLNWYQQKPGKAPKLLIYGASSLQSGVPSRFSGSGSGTDFTLTISSLQPEDFATYYCQQSKSNSPITFGQGTRLEIK

SEQ ID NO: 99 - DNA, 18 bp, synthetic construct
  misc_feature 1..18: note = synthetic
  source 1..18: mol_type = other DNA
cagagcattagcagctat

SEQ ID NO: 100 - protein, 6 aa, synthetic construct
  REGION 1..6: note = synthetic
QSISSY

SEQ ID NO: 101-102 - skipped (2 entries)

SEQ ID NO: 103 - DNA, 30 bp, synthetic construct
  misc_feature 1..30: note = synthetic
  source 1..30: mol_type = other DNA
caacagagtaagagtaattctccgatcacc

SEQ ID NO: 104 - protein, 10 aa, synthetic construct
  REGION 1..10: note = synthetic
QQSKSNSPIT

SEQ ID NO: 105 - DNA, 369 bp, synthetic construct
  misc_feature 1..369: note = synthetic
  source 1..369: mol_type = other DNA
caggtccagctggtgcagtctggggctgaggtgaagaagcctgggtcctcggtgaaggtctcctgcaaggcttctggaggcaccttcatcacctatgctatcagctgggtgcgacaggcccctggacaagggcttgagtggatgggaggtgtcatccctatctttggtactccaaggtacgcacagaagttccagggcagagtcacgattaccacggacgattccacgaccacagcctacatggagctgagcagcctgagatctgacgacacggccgtgtattattgtgcgacaaggagcagctcgtctccctattattactacggtatggacgtctggggccaagggaccacggtcaccgtctcctca

SEQ ID NO: 106 - protein, 123 aa, synthetic construct
  REGION 1..123: note = synthetic
QVQLVQSGAEVKKPGSSVKVSCKASGGTFITYAISWVRQAPGQGLEWMGGVIPIFGTPRYAQKFQGRVTITTDDSTTTAYMELSSLRSDDTAVYYCATRSSSSPYYYYGMDVWGQGTTVTVSS

SEQ ID NO: 107 - DNA, 24 bp, synthetic construct
  misc_feature 1..24: note = synthetic
  source 1..24: mol_type = other DNA
ggaggcaccttcatcacctatgct

SEQ ID NO: 108 - protein, 8 aa, synthetic construct
  REGION 1..8: note = synthetic
GGTFITYA

SEQ ID NO: 109 - DNA, 24 bp, synthetic construct
  misc_feature 1..24: note = synthetic
  source 1..24: mol_type = other DNA
gtcatccctatctttggtactcca

SEQ ID NO: 110 - protein, 8 aa, synthetic construct
  REGION 1..8: note = synthetic
VIPIFGTP

SEQ ID NO: 111 - DNA, 48 bp, synthetic construct
  misc_feature 1..48: note = synthetic
  source 1..48: mol_type = other DNA
gcgacaaggagcagctcgtctccctattattactacggtatggacgtc

SEQ ID NO: 112 - protein, 16 aa, synthetic construct
  REGION 1..16: note = synthetic
ATRSSSSPYYYYGMDV

SEQ ID NO: 113 - DNA, 336 bp, synthetic construct
  misc_feature 1..336: note = synthetic
  source 1..336: mol_type = other DNA
gatattgtgatgacccagactccactctcctcacctgtcacccttggacagccggcctccatctcctgcaggtctagtcaaagcctcgtacacagtgatggaaacacctatttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcgggatttattactgcattcaagctacacaatttccgtacacttttggccaggggaccaagctggagatcaaa

SEQ ID NO: 114 - protein, 112 aa, synthetic construct
  REGION 1..112: note = synthetic
DIVMTQTPLSSPVTLGQPASISCRSSQSLVHSDGNTYLSWLQQRPGQPPRLLIYKISNRFSGVPDRFSGSGAGTDFTLKISRVEAEDVGIYYCIQATQFPYTFGQGTKLEIK

SEQ ID NO: 115 - DNA, 33 bp, synthetic construct
  misc_feature 1..33: note = synthetic
  source 1..33: mol_type = other DNA
caaagcctcgtacacagtgatggaaacacctat

SEQ ID NO: 116 - protein, 11 aa, synthetic construct
  REGION 1..11: note = synthetic
QSLVHSDGNTY

SEQ ID NO: 117-118 - skipped (2 entries)

SEQ ID NO: 119 - DNA, 27 bp, synthetic construct
  misc_feature 1..27: note = synthetic
  source 1..27: mol_type = other DNA
attcaagctacacaatttccgtacact

SEQ ID NO: 120 - protein, 9 aa, synthetic construct
  REGION 1..9: note = synthetic
IQATQFPYT

SEQ ID NO: 121 - DNA, 372 bp, synthetic construct
  misc_feature 1..372: note = synthetic
  source 1..372: mol_type = other DNA
caggtgcagctggtgcagtctggggctgaggtgaagaagcctggggcctcagtgacggtctcctgcaaggcttctggatacaccttcaccaattatgatatcaactgggtgcgacaggccactggacaaggacttgagtggttgggatggatgagccctaacagtggtaacaaaggctatgcacagaagttccagggcagagtctccatgactttgaataccgccttaagcacagcctacatggaactgagcagcctgagatctgaggacacggccgtgtattactgtgcgagatggtattgtagtgatgccagttgctatcccgatgcttttgatatctggggccaagggacaatggtcaccgtctcttca

SEQ ID NO: 122 - protein, 124 aa, synthetic construct
  REGION 1..124: note = synthetic
QVQLVQSGAEVKKPGASVTVSCKASGYTFTNYDINWVRQATGQGLEWLGWMSPNSGNKGYAQKFQGRVSMTLNTALSTAYMELSSLRSEDTAVYYCARWYCSDASCYPDAFDIWGQGTMVTVSS

SEQ ID NO: 123 - DNA, 24 bp, synthetic construct
  misc_feature 1..24: note = synthetic
  source 1..24: mol_type = other DNA
ggatacaccttcaccaattatgat

SEQ ID NO: 124 - protein, 8 aa, synthetic construct
  REGION 1..8: note = synthetic
GYTFTNYD

SEQ ID NO: 125 - DNA, 24 bp, synthetic construct
  misc_feature 1..24: note = synthetic
  source 1..24: mol_type = other DNA
atgagccctaacagtggtaacaaa

SEQ ID NO: 126 - protein, 8 aa, synthetic construct
  REGION 1..8: note = synthetic
MSPNSGNK

SEQ ID NO: 127 - DNA, 51 bp, synthetic construct
  misc_feature 1..51: note = synthetic
  source 1..51: mol_type = other DNA
gcgagatggtattgtagtgatgccagttgctatcccgatgcttttgatatc

SEQ ID NO: 128 - protein, 17 aa, synthetic construct
  REGION 1..17: note = synthetic
ARWYCSDASCYPDAFDI

SEQ ID NO: 129 - DNA, 321 bp, synthetic construct
  misc_feature 1..321: note = synthetic
  source 1..321: mol_type = other DNA
gacatccagatgacccagtctccatcttccgtgtctgcatctgtaggagacagagtcaccatcacttgtcgggcgagtcaggatttttccgactggttagcctggtatcagcagaaacctgggaaagcccctgagctcctgatctatgctacatccagtttgcatactggggtcccatcaaggttcagcggcagtggatctgggacagatttcactctcaccatcagcaccctgcagcctgaagattttgctacttactattgtctacaggctaacaatttcccgctcactttcggcggagggaccaaggtggagatcaaa

SEQ ID NO: 130 - protein, 107 aa, synthetic construct
  REGION 1..107: note = synthetic
DIQMTQSPSSVSASVGDRVTITCRASQDFSDWLAWYQQKPGKAPELLIYATSSLHTGVPSRFSGSGSGTDFTLTISTLQPEDFATYYCLQANNFPLTFGGGTKVEIK

SEQ ID NO: 131 - DNA, 18 bp, synthetic construct
  misc_feature 1..18: note = synthetic
  source 1..18: mol_type = other DNA
caggatttttccgactgg

SEQ ID NO: 132 - protein, 6 aa, synthetic construct
  REGION 1..6: note = synthetic
QDFSDW

SEQ ID NO: 133-134 - skipped (2 entries)

SEQ ID NO: 135 - DNA, 27 bp, synthetic construct
  misc_feature 1..27: note = synthetic
  source 1..27: mol_type = other DNA
ctacaggctaacaatttcccgctcact

SEQ ID NO: 136 - protein, 9 aa, synthetic construct
  REGION 1..9: note = synthetic
LQANNFPLT

SEQ ID NO: 137 - DNA, 366 bp, synthetic construct
  misc_feature 1..366: note = synthetic
  source 1..366: mol_type = other DNA
caggtgcagctggtggagtctgggggaggcgtggcccagcctgggaggtccctgagactctcctgtgcagcctctggaatcaccttcaataactatggcatgcactgggtccgccaggctccaggcaaggggctggagtgggtggcatttatttcagatgaaggaagaaataaacactatggagactccgtgaagggccgattcaccatcgacagagacaattccaagaacacactgtatctgcaaatgaatagcctgagagctgaggacacggctgtatattactgtgcgaaattgggggataataggaatcaccactacggtttggacgtctggggccaagggaccacggtcaccgtctcctca

SEQ ID NO: 138 - protein, 122 aa, synthetic construct
  REGION 1..122: note = synthetic
QVQLVESGGGVAQPGRSLRLSCAASGITFNNYGMHWVRQAPGKGLEWVAFISDEGRNKHYGDSVKGRFTIDRDNSKNTLYLQMNSLRAEDTAVYYCAKLGDNRNHHYGLDVWGQGTTVTVSS

SEQ ID NO: 139 - DNA, 24 bp, synthetic construct
  misc_feature 1..24: note = synthetic
  source 1..24: mol_type = other DNA
ggaatcaccttcaataactatggc

SEQ ID NO: 140 - protein, 8 aa, synthetic construct
  REGION 1..8: note = synthetic
GITFNNYG

SEQ ID NO: 141 - DNA, 24 bp, synthetic construct
  misc_feature 1..24: note = synthetic
  source 1..24: mol_type = other DNA
atttcagatgaaggaagaaataaa

SEQ ID NO: 142 - protein, 8 aa, synthetic construct
  REGION 1..8: note = synthetic
ISDEGRNK

SEQ ID NO: 143 - DNA, 45 bp, synthetic construct
  misc_feature 1..45: note = synthetic
  source 1..45: mol_type = other DNA
gcgaaattgggggataataggaatcaccactacggtttggacgtc

SEQ ID NO: 144 - protein, 15 aa, synthetic construct
  REGION 1..15: note = synthetic
AKLGDNRNHHYGLDV

SEQ ID NO: 145 - DNA, 321 bp, synthetic construct
  misc_feature 1..321: note = synthetic
  source 1..321: mol_type = other DNA
gacatccagttgacccagtctccatccttcctgtctgcatctgtaggagacagagtcaccatcacttgctgggccagtcagggcattaccaattatttagcctggtatcagcaaaaaccagggaaagcccctagcctcctgatctatgctgcatccactttgcaaagtggggtcccatcaaggttcagcggcagtggacatgggacagaattcactctcacagtcagcagcctgcagcctgaagattttgtaacttattactgtcaacagtttaatagttacccgatcaccttcggccaagggacacgactggagattaaa

SEQ ID NO: 146 - protein, 107 aa, synthetic construct
  REGION 1..107: note = synthetic
DIQLTQSPSFLSASVGDRVTITCWASQGITNYLAWYQQKPGKAPSLLIYAASTLQSGVPSRFSGSGHGTEFTLTVSSLQPEDFVTYYCQQFNSYPITFGQGTRLEIK

SEQ ID NO: 147 - DNA, 18 bp, synthetic construct
  misc_feature 1..18: note = synthetic
  source 1..18: mol_type = other DNA
cagggcattaccaattat

SEQ ID NO: 148 - protein, 6 aa, synthetic construct
  REGION 1..6: note = synthetic
QGITNY

SEQ ID NO: 149-150 - skipped (2 entries)

SEQ ID NO: 151 - DNA, 27 bp, synthetic construct
  misc_feature 1..27: note = synthetic
  source 1..27: mol_type = other DNA
caacagtttaatagttacccgatcacc

SEQ ID NO: 152 - protein, 9 aa, synthetic construct
  REGION 1..9: note = synthetic
QQFNSYPIT

SEQ ID NO: 153 - DNA, 366 bp, synthetic construct
  misc_feature 1..366: note = synthetic
  source 1..366: mol_type = other DNA
caggtccagctggtgcagtctggggctgaggtgaagaagcctgggtcctcggtgaaggtctcctgtaaggcttctggaggcaccttcagcaactatgatatcagctgggtgcgacaggcccctggacaaggacttgagtggatgggagggatcatccccatcattggtacagcaaactacgcacagaagttccagggcagagtcacgattacaacggacgaatccacgagcacagcctacatggagctgagcagcctgagatctgaggacacggccgtgtattactgtgcgagagatccgggtatagcagtggctggttcgagctttgactactggggccagggaaccctggtcaccgtctcctca

SEQ ID NO: 154 - protein, 122 aa, synthetic construct
  REGION 1..122: note = synthetic
QVQLVQSGAEVKKPGSSVKVSCKASGGTFSNYDISWVRQAPGQGLEWMGGIIPIIGTANYAQKFQGRVTITTDESTSTAYMELSSLRSEDTAVYYCARDPGIAVAGSSFDYWGQGTLVTVSS

SEQ ID NO: 155 - DNA, 24 bp, synthetic construct
  misc_feature 1..24: note = synthetic
  source 1..24: mol_type = other DNA
ggaggcaccttcagcaactatgat

SEQ ID NO: 156 - protein, 8 aa, synthetic construct
  REGION 1..8: note = synthetic
GGTFSNYD

SEQ ID NO: 157 - DNA, 24 bp, synthetic construct
  misc_feature 1..24: note = synthetic
  source 1..24: mol_type = other DNA
atcatccccatcattggtacagca

SEQ ID NO: 158 - protein, 8 aa, synthetic construct
  REGION 1..8: note = synthetic
IIPIIGTA

SEQ ID NO: 159 - DNA, 45 bp, synthetic construct
  misc_feature 1..45: note = synthetic
  source 1..45: mol_type = other DNA
gcgagagatccgggtatagcagtggctggttcgagctttgactac

SEQ ID NO: 160 - protein, 15 aa, synthetic construct
  REGION 1..15: note = synthetic
ARDPGIAVAGSSFDY

SEQ ID NO: 161 - DNA, 321 bp, synthetic construct
  misc_feature 1..321: note = synthetic
  source 1..321: mol_type = other DNA
gccatccagatgacccagtctccatcctccctgtctgcatctgttggagacagagtcaccatcacttgccgggcaagtcagggcattagacatgatttaggctggtatcagcagaaaccagggaaagcccctaagctcctgatctatgctgcatccaatttacaaagtggggtcccatcaaggttcagcggcagtggatctggcacagatttcactctcaccatcagcagcctgcagcctgaagattttgcaacttattactgtctacaagattacaattaccctcggacgttcggccaagggaccaaggtggaaatcaaa

SEQ ID NO: 162 - protein, 107 aa, synthetic construct
  REGION 1..107: note = synthetic
AIQMTQSPSSLSASVGDRVTITCRASQGIRHDLGWYQQKPGKAPKLLIYAASNLQSGVPSRFSGSGSGTDFTLTISSLQPEDFATYYCLQDYNYPRTFGQGTKVEIK

SEQ ID NO: 163 - DNA, 18 bp, synthetic construct
  misc_feature 1..18: note = synthetic
  source 1..18: mol_type = other DNA
cagggcattagacatgat

SEQ ID NO: 164 - protein, 6 aa, synthetic construct
  REGION 1..6: note = synthetic
QGIRHD

SEQ ID NO: 165-166 - skipped (2 entries)

SEQ ID NO: 167 - DNA, 27 bp, synthetic construct
  misc_feature 1..27: note = synthetic
  source 1..27: mol_type = other DNA
ctacaagattacaattaccctcggacg

SEQ ID NO: 168 - protein, 9 aa, synthetic construct
  REGION 1..9: note = synthetic
LQDYNYPRT

SEQ ID NO: 169 - DNA, 348 bp, synthetic construct
  misc_feature 1..348: note = synthetic
  source 1..348: mol_type = other DNA
caggtgcagctggtgcagtctggggctgaggtgaggacgcctgggtcctcggtgaaggtctcctgcaaggcttctggaggcaccttcagcagctatgctgtcacctgggtgcgacaggcccctggacaagggcttgagtggatgggagggctcatccctttctttggtccagcaaactacgcacagagattccagggcagagtctcgattaccacggacgaatccacgaacatagcctacttggagctgagcagcctgagatccgaggactcggccgtttattactgtgcgagagggcggaacttcggtgactactggggccagggaaccctggtcaccgtctcctca

SEQ ID NO: 170 - protein, 116 aa, synthetic construct
  REGION 1..116: note = synthetic
QVQLVQSGAEVRTPGSSVKVSCKASGGTFSSYAVTWVRQAPGQGLEWMGGLIPFFGPANYAQRFQGRVSITTDESTNIAYLELSSLRSEDSAVYYCARGRNFGDYWGQGTLVTVSS

SEQ ID NO: 171 - DNA, 24 bp, synthetic construct
  misc_feature 1..24: note = synthetic
  source 1..24: mol_type = other DNA
ggaggcaccttcagcagctatgct

SEQ ID NO: 172 - protein, 8 aa, synthetic construct
  REGION 1..8: note = synthetic
GGTFSSYA

SEQ ID NO: 173 - DNA, 24 bp, synthetic construct
  misc_feature 1..24: note = synthetic
  source 1..24: mol_type = other DNA
ctcatccctttctttggtccagca

SEQ ID NO: 174 - protein, 8 aa, synthetic construct
  REGION 1..8: note = synthetic
LIPFFGPA

SEQ ID NO: 175 - DNA, 27 bp, synthetic construct
  misc_feature 1..27: note = synthetic
  source 1..27: mol_type = other DNA
gcgagagggcggaacttcggtgactac

SEQ ID NO: 176 - protein, 9 aa, synthetic construct
  REGION 1..9: note = synthetic
ARGRNFGDY

SEQ ID NO: 177 - DNA, 321 bp, synthetic construct
  misc_feature 1..321: note = synthetic
  source 1..321: mol_type = other DNA
gacatccagatgacccagtctccatcctcactgtctgcatctataggagacagagtcaccatcacttgtcgggcgagtcaggacattagcaattatttagcctggtttcagcagaaaccagggaaagtccctaagtccctgatctatgctgcatccaatttgcaaagtggggtcccatcaaagttcagcggcagtggatctgggacagatttcactctcaccatcagtaccctgcagcctgaagattttgcaacttattactgccaacagtatagtagttatccattcactttcggccctgggaccaaagtggatatcaaa

SEQ ID NO: 178 - protein, 107 aa, synthetic construct
  REGION 1..107: note = synthetic
DIQMTQSPSSLSASIGDRVTITCRASQDISNYLAWFQQKPGKVPKSLIYAASNLQSGVPSKFSGSGSGTDFTLTISTLQPEDFATYYCQQYSSYPFTFGPGTKVDIK

SEQ ID NO: 179 - DNA, 18 bp, synthetic construct
  misc_feature 1..18: note = synthetic
  source 1..18: mol_type = other DNA
caggacattagcaattat

SEQ ID NO: 180 - protein, 6 aa, synthetic construct
  REGION 1..6: note = synthetic
QDISNY

SEQ ID NO: 181-182 - skipped (2 entries)

SEQ ID NO: 183 - DNA, 27 bp, synthetic construct
  misc_feature 1..27: note = synthetic
  source 1..27: mol_type = other DNA
caacagtatagtagttatccattcact

SEQ ID NO: 184 - protein, 9 aa, synthetic construct
  REGION 1..9: note = synthetic
QQYSSYPFT

SEQ ID NO: 185 - DNA, 351 bp, synthetic construct
  misc_feature 1..351: note = synthetic
  source 1..351: mol_type = other DNA
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacaacaaactacgcacagaagttccagggcagagtcacgattaccacggacgaatccacgagcacagcctacatggagctgagcagcctgagatctgaggacacggccgtgtattactgtgcgagaaggcacggtatatcctttgactactggggccagggaaccctggtcaccgtctcctca

SEQ ID NO: 186 - protein, 117 aa, synthetic construct
  REGION 1..117: note = synthetic
QVQLVQSGAEVKKPGSSVKVSCKASGGTFSSYAISWVRQAPGQGLEWMGGIIPIFGTTNYAQKFQGRVTITTDESTSTAYMELSSLRSEDTAVYYCARRHGISFDYWGQGTLVTVSS

SEQ ID NO: 187 - DNA, 24 bp, synthetic construct
  misc_feature 1..24: note = synthetic
  source 1..24: mol_type = other DNA
ggaggcaccttcagcagctatgct

SEQ ID NO: 188 - protein, 8 aa, synthetic construct
  REGION 1..8: note = synthetic
GGTFSSYA

SEQ ID NO: 189 - DNA, 24 bp, synthetic construct
  misc_feature 1..24: note = synthetic
  source 1..24: mol_type = other DNA
atcatccctatctttggtacaaca

SEQ ID NO: 190 - protein, 8 aa, synthetic construct
  REGION 1..8: note = synthetic
IIPIFGTT

SEQ ID NO: 191 - DNA, 30 bp, synthetic construct
  misc_feature 1..30: note = synthetic
  source 1..30: mol_type = other DNA
gcgagaaggcacggtatatcctttgactac

SEQ ID NO: 192 - protein, 10 aa, synthetic construct
  REGION 1..10: note = synthetic
ARRHGISFDY

SEQ ID NO: 193 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccgtcacttgccgggcaagtcagggcattagaaatgatttggcctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttatccgtggacgttcggcctagggaccaaggtggaaatcaaa

SEQ ID NO: 194 - protein, 107 aa, synthetic construct
  REGION 1..107: note = synthetic
DIQMTQSPSSLSASVGDRVTVTCRASQGIRNDLAWYQQKPGKAPKRLIYAASSLQSGVPSRFSGSGSGTEFTLTISSLQPEDFATYYCLQHNSYPWTFGLGTKVEIK

SEQ ID NO: 195 - DNA, 18 bp, synthetic construct
  misc_feature 1..18: note = synthetic
  source 1..18: mol_type = other DNA
cagggcattagaaatgat

SEQ ID NO: 196 - protein, 6 aa, synthetic construct
  REGION 1..6: note = synthetic
QGIRND

SEQ ID NO: 197-198 - skipped (2 entries)

SEQ ID NO: 199 - DNA, 27 bp, synthetic construct
  misc_feature 1..27: note = synthetic
  source 1..27: mol_type = other DNA
ctacagcataatagttatccgtggacg

SEQ ID NO: 200 - protein, 9 aa, synthetic construct
  REGION 1..9: note = synthetic
LQHNSYPWT

SEQ ID NO: 201 - DNA, 366 bp, synthetic construct
  misc_feature 1..366: note = synthetic
  source 1..366: mol_type = other DNA
caggtgcagctggtgcagtctggggctgaggtgaagaagcctgggtcctcggtgaaggtctcctgcaaggcttctggaggcaccttcagcagctatactatcagctgggttcgacaggcccctggacaagggcttgagtggatgggagggatcagccctatctttggtacagcaaactacgcacagaagttccagggcagagtcacgattaccacggacgaatccacgagcacagcctacatggagctgagcagcctgagatctgaggacacggccgtgtattactgtgcgagagagtccccgtataactggaaccaaaaatacttccagtattggggccagggcaccctggtcaccgtctcctca

SEQ ID NO: 202 - protein, 122 aa, synthetic construct
  REGION 1..122: note = synthetic
QVQLVQSGAEVKKPGSSVKVSCKASGGTFSSYTISWVRQAPGQGLEWMGGISPIFGTANYAQKFQGRVTITTDESTSTAYMELSSLRSEDTAVYYCARESPYNWNQKYFQYWGQGTLVTVSS

SEQ ID NO: 203 - DNA, 24 bp, synthetic construct
  misc_feature 1..24: note = synthetic
  source 1..24: mol_type = other DNA
ggaggcaccttcagcagctatact

SEQ ID NO: 204 - protein, 8 aa, synthetic construct
  REGION 1..8: note = synthetic
GGTFSSYT

SEQ ID NO: 205 - DNA, 24 bp, synthetic construct
  misc_feature 1..24: note = synthetic
  source 1..24: mol_type = other DNA
atcagccctatctttggtacagca

SEQ ID NO: 206 - protein, 8 aa, synthetic construct
  REGION 1..8: note = synthetic
ISPIFGTA

SEQ ID NO: 207 - DNA, 45 bp, synthetic construct
  misc_feature 1..45: note = synthetic
  source 1..45: mol_type = other DNA
gcgagagagtccccgtataactggaaccaaaaatacttccagtat

SEQ ID NO: 208 - protein, 15 aa, synthetic construct
  REGION 1..15: note = synthetic
ARESPYNWNQKYFQY

SEQ ID NO: 209 - DNA, 321 bp, synthetic construct
  misc_feature 1..321: note = synthetic
  source 1..321: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattcgtacacttttggccaggggaccaagctggagatcaaa

SEQ ID NO: 210 - protein, 107 aa, synthetic construct
  REGION 1..107: note = synthetic
DIQMTQSPSTLSASVGDRVTITCRASQSISSWLAWYQQKPGKAPKLLIYKASSLESGVPSRFSGSGSGTEFTLTISSLQPDDFATYYCQQYNSYSYTFGQGTKLEIK

SEQ ID NO: 211 - DNA, 18 bp, synthetic construct
  misc_feature 1..18: note = synthetic
  source 1..18: mol_type = other DNA
cagagtattagtagctgg

SEQ ID NO: 212 - protein, 6 aa, synthetic construct
  REGION 1..6: note = synthetic
QSISSW

SEQ ID NO: 213-214 - skipped (2 entries)

SEQ ID NO: 215 - DNA, 27 bp, synthetic construct
  misc_feature 1..27: note = synthetic
  source 1..27: mol_type = other DNA
caacagtataatagttattcgtacact

SEQ ID NO: 216 - protein, 9 aa, synthetic construct
  REGION 1..9: note = synthetic
QQYNSYSYT

SEQ ID NO: 217 - DNA, 363 bp, synthetic construct
  misc_feature 1..363: note = synthetic
  source 1..363: mol_type = other DNA
caggtccagctggtgcagtctgggtctgaggtgaagaagcctgggtcctcggtgaaggtctcctgcaaggcgtctggagggaccctcagcctctatgctgtcagctgggtgcgacaggcccctggacaggggcttgagtggatgggggggatcatccctatctttggtacaacaaaatacgcacaggagttccagggcagagtcacgttttccacggacgagtccacgagcacagcctacatggagctgaacagcctgcgatctgaggacacggccgtgtattactgtgcgagacaatggactatgactagaaccttggattttgacatctggggccagggaaccctggtcaccgtctcctca

SEQ ID NO: 218 - protein, 121 aa, synthetic construct
  REGION 1..121: note = synthetic
QVQLVQSGSEVKKPGSSVKVSCKASGGTLSLYAVSWVRQAPGQGLEWMGGIIPIFGTTKYAQEFQGRVTFSTDESTSTAYMELNSLRSEDTAVYYCARQWTMTRTLDFDIWGQGTLVTVSS

SEQ ID NO: 219 - DNA, 24 bp, synthetic construct
  misc_feature 1..24: note = synthetic
  source 1..24: mol_type = other DNA
ggagggaccctcagcctctatgct

SEQ ID NO: 220 - protein, 8 aa, synthetic construct
  REGION 1..8: note = synthetic
GGTLSLYA

SEQ ID NO: 221 - DNA, 24 bp, synthetic construct
  misc_feature 1..24: note = synthetic
  source 1..24: mol_type = other DNA
atcatccctatctttggtacaaca

SEQ ID NO: 222 - protein, 8 aa, synthetic construct
  REGION 1..8: note = synthetic
IIPIFGTT

SEQ ID NO: 223 - DNA, 42 bp, synthetic construct
  misc_feature 1..42: note = synthetic
  source 1..42: mol_type = other DNA
gcgagacaatggactatgactagaaccttggattttgacatc

SEQ ID NO: 224 - protein, 14 aa, synthetic construct
  REGION 1..14: note = synthetic
ARQWTMTRTLDFDI

SEQ ID NO: 225 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccggacaagtcagggcattagaaatgatttaggctggtatcagcagaaaccggggaaagcccctaagcgcctgatctatggtgcatccagtttgcaaagtggggtcccatcaagattcagcggcagtggatctgggacagaattcactctcacaatcagcagcctgcagcctgaagattttgcaagttattactgtctacaacataataattatccgtggacgttcggccaagggaccaaggtggaaatcaaa

SEQ ID NO: 226 - protein, 107 aa, synthetic construct
  REGION 1..107: note = synthetic
DIQMTQSPSSLSASVGDRVTITCRTSQGIRNDLGWYQQKPGKAPKRLIYGASSLQSGVPSRFSGSGSGTEFTLTISSLQPEDFASYYCLQHNNYPWTFGQGTKVEIK

SEQ ID NO: 227 - DNA, 18 bp, synthetic construct
  misc_feature 1..18: note = synthetic
  source 1..18: mol_type = other DNA
cagggcattagaaatgat

SEQ ID NO: 228 - protein, 6 aa, synthetic construct
  REGION 1..6: note = synthetic
QGIRND

SEQ ID NO: 229-230 - skipped (2 entries)

SEQ ID NO: 231 - DNA, 27 bp, synthetic construct
  misc_feature 1..27: note = synthetic
  source 1..27: mol_type = other DNA
ctacaacataataattatccgtggacg

SEQ ID NO: 232 - protein, 9 aa, synthetic construct
  REGION 1..9: note = synthetic
LQHNNYPWT

SEQ ID NO: 233 - DNA, 369 bp, synthetic construct
  misc_feature 1..369: note = synthetic
  source 1..369: mol_type = other DNA
caggtgcagctggtggagtctgggggaggcgtggtccagcctgggaggtccctgagactctcctgtgtagcctccggattcaccttcagtgtctatggcatgaactgggtccgccaggctccaggcaagggtctggactgggtggcagtcatatcaaatgatggaagttataaatactatgcggactccgtgaagggccgattcaccatctccagagacaattccaagaacacggtgtatctgcaaatgaacagcctgagaggtgaggacacggctatttattactgtgcgaaaaggaatgaatgggagctagaggaatactacggtatggacgtctggggccaagggaccacggtcaccgtctcctca

SEQ ID NO: 234 - protein, 123 aa, synthetic construct
  REGION 1..123: note = synthetic
QVQLVESGGGVVQPGRSLRLSCVASGFTFSVYGMNWVRQAPGKGLDWVAVISNDGSYKYYADSVKGRFTISRDNSKNTVYLQMNSLRGEDTAIYYCAKRNEWELEEYYGMDVWGQGTTVTVSS

SEQ ID NO: 235 - DNA, 24 bp, synthetic construct
  misc_feature 1..24: note = synthetic
  source 1..24: mol_type = other DNA
ggattcaccttcagtgtctatggc

SEQ ID NO: 236 - protein, 8 aa, synthetic construct
  REGION 1..8: note = synthetic
GFTFSVYG

SEQ ID NO: 237 - DNA, 24 bp, synthetic construct
  misc_feature 1..24: note = synthetic
  source 1..24: mol_type = other DNA
atatcaaatgatggaagttataaa

SEQ ID NO: 238 - protein, 8 aa, synthetic construct
  REGION 1..8: note = synthetic
ISNDGSYK

SEQ ID NO: 239 - DNA, 48 bp, synthetic construct
  misc_feature 1..48: note = synthetic
  source 1..48: mol_type = other DNA
gcgaaaaggaatgaatgggagctagaggaatactacggtatggacgtc

SEQ ID NO: 240 - protein, 16 aa, synthetic construct
  REGION 1..16: note = synthetic
AKRNEWELEEYYGMDV

SEQ ID NO: 241 - DNA, 363 bp, synthetic construct
  misc_feature 1..363: note = synthetic
  source 1..363: mol_type = other DNA
gaggtgcagctggtggagtctgggggaggattggtacagtctggagggtccctgagactctcctgtgtagcctctggattcaccttcaataattatgagatgaattgggtccgccaggctccagggaaggggctggagtggatttcatacattgatattaatggtggaagtaccatctactatgcagactctgtgaagggccgattcaccatctccagagacaatgccaagaagtcactgtatctgcaaatgaacagcctgagagccgaggacacggctatttactactgtgcgagcgcctttggttcgggtggttttctttttgactattggggccagggaaccctggtcactgtctcctca

SEQ ID NO: 242 - protein, 121 aa, synthetic construct
  REGION 1..121: note = synthetic
EVQLVESGGGLVQSGGSLRLSCVASGFTFNNYEMNWVRQAPGKGLEWISYIDINGGSTIYYADSVKGRFTISRDNAKKSLYLQMNSLRAEDTAIYYCASAFGSGGFLFDYWGQGTLVTVSS

SEQ ID NO: 243 - DNA, 24 bp, synthetic construct
  misc_feature 1..24: note = synthetic
  source 1..24: mol_type = other DNA
ggattcaccttcaataattatgag

SEQ ID NO: 244 - protein, 8 aa, synthetic construct
  REGION 1..8: note = synthetic
GFTFNNYE

SEQ ID NO: 245 - DNA, 27 bp, synthetic construct
  misc_feature 1..27: note = synthetic
  source 1..27: mol_type = other DNA
attgatattaatggtggaagtaccatc

SEQ ID NO: 246 - protein, 9 aa, synthetic construct
  REGION 1..9: note = synthetic
IDINGGSTI

SEQ ID NO: 247 - DNA, 39 bp, synthetic construct
  misc_feature 1..39: note = synthetic
  source 1..39: mol_type = other DNA
gcgagcgcctttggttcgggtggttttctttttgactat

SEQ ID NO: 248 - protein, 13 aa, synthetic construct
  REGION 1..13: note = synthetic
ASAFGSGGFLFDY

SEQ ID NO: 249 - DNA, 366 bp, synthetic construct
  misc_feature 1..366: note = synthetic
  source 1..366: mol_type = other DNA
caggtgcagctggtggagtctgggggaggcgtggtccagcctgggatgtccctgagactctcctgtatagcgtctggattcaccttcagtgactatgtcatacactgggtccgccaggctccaggcaaggggctggagtgggtggcaattatatggtatgatggaagtaataaatactatgcagactccgtgaagggccgattcaccatctccagagacaattccaagaacacgctgtatcttgaaatgaatagactgagagtcgaggacacggctgtgttttactgtgcgaggggtctggggatcgaagattacaattacggtatggacgtctggggccaagggaccacggtcaccgtctcctca

SEQ ID NO: 250 - protein, 122 aa, synthetic construct
  REGION 1..122: note = synthetic
QVQLVESGGGVVQPGMSLRLSCIASGFTFSDYVIHWVRQAPGKGLEWVAIIWYDGSNKYYADSVKGRFTISRDNSKNTLYLEMNRLRVEDTAVFYCARGLGIEDYNYGMDVWGQGTTVTVSS

SEQ ID NO: 251 - DNA, 24 bp, synthetic construct
  misc_feature 1..24: note = synthetic
  source 1..24: mol_type = other DNA
ggattcaccttcagtgactatgtc

SEQ ID NO: 252 - protein, 8 aa, synthetic construct
  REGION 1..8: note = synthetic
GFTFSDYV

SEQ ID NO: 253 - DNA, 24 bp, synthetic construct
  misc_feature 1..24: note = synthetic
  source 1..24: mol_type = other DNA
atatggtatgatggaagtaataaa

SEQ ID NO: 254 - protein, 8 aa, synthetic construct
  REGION 1..8: note = synthetic
IWYDGSNK

SEQ ID NO: 255 - DNA, 45 bp, synthetic construct
  misc_feature 1..45: note = synthetic
  source 1..45: mol_type = other DNA
gcgaggggtctggggatcgaagattacaattacggtatggacgtc

SEQ ID NO: 256 - protein, 15 aa, synthetic construct
  REGION 1..15: note = synthetic
ARGLGIEDYNYGMDV

SEQ ID NO: 257 - DNA, 357 bp, synthetic construct
  misc_feature 1..357: note = synthetic
  source 1..357: mol_type = other DNA
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacactggaccgacgaggatgcttttgatatctggggccaagggacaatggtcaccgtctcttca

SEQ ID NO: 258 - protein, 119 aa, synthetic construct
  REGION 1..119: note = synthetic
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHWTDEDAFDIWGQGTMVTVSS

SEQ ID NO: 259 - DNA, 24 bp, synthetic construct
  misc_feature 1..24: note = synthetic
  source 1..24: mol_type = other DNA
ggatacagctttaccagctactgg

SEQ ID NO: 260 - protein, 8 aa, synthetic construct
  REGION 1..8: note = synthetic
GYSFTSYW

SEQ ID NO: 261 - DNA, 24 bp, synthetic construct
  misc_feature 1..24: note = synthetic
  source 1..24: mol_type = other DNA
atctatcctggtgactctgatacc

SEQ ID NO: 262 - protein, 8 aa, synthetic construct
  REGION 1..8: note = synthetic
IYPGDSDT

SEQ ID NO: 263 - DNA, 36 bp, synthetic construct
  misc_feature 1..36: note = synthetic
  source 1..36: mol_type = other DNA
gcgagacactggaccgacgaggatgcttttgatatc

SEQ ID NO: 264 - protein, 12 aa, synthetic construct
  REGION 1..12: note = synthetic
ARHWTDEDAFDI

SEQ ID NO: 265 - DNA, 369 bp, synthetic construct
  misc_feature 1..369: note = synthetic
  source 1..369: mol_type = other DNA
caggtccagctggtgcagtctgggactgaggtgaagaagcctgggtcttcggtgaaggtctcctgcaaggcttctggaggcaccttcggcagttatcctatcagctgggtgcgacaggcccctggacaagggcttgagtggatgggagggatcatccctatctttggtacagcaaactccgcacagaagttccagggcagagtcacgattaccacggacgaacccacgagcacagccttcatggagctgagcagcctgagatctgaggacacggccgtgtattactgtgcgagaccgtattacgatattttgactgactactacggtatggacgtctggggtcaagggaccacggtcaccgtctcctca

SEQ ID NO: 266 - protein, 123 aa, synthetic construct
  REGION 1..123: note = synthetic
QVQLVQSGTEVKKPGSSVKVSCKASGGTFGSYPISWVRQAPGQGLEWMGGIIPIFGTANSAQKFQGRVTITTDEPTSTAFMELSSLRSEDTAVYYCARPYYDILTDYYGMDVWGQGTTVTVSS

SEQ ID NO: 267 - DNA, 24 bp, synthetic construct
  misc_feature 1..24: note = synthetic
  source 1..24: mol_type = other DNA
ggaggcaccttcggcagttatcct

SEQ ID NO: 268 - protein, 8 aa, synthetic construct
  REGION 1..8: note = synthetic
GGTFGSYP

SEQ ID NO: 269 - DNA, 24 bp, synthetic construct
  misc_feature 1..24: note = synthetic
  source 1..24: mol_type = other DNA
atcatccctatctttggtacagca

SEQ ID NO: 270 - protein, 8 aa, synthetic construct
  REGION 1..8: note = synthetic
IIPIFGTA

SEQ ID NO: 271 - DNA, 48 bp, synthetic construct
  misc_feature 1..48: note = synthetic
  source 1..48: mol_type = other DNA
gcgagaccgtattacgatattttgactgactactacggtatggacgtc

SEQ ID NO: 272 - protein, 16 aa, synthetic construct
  REGION 1..16: note = synthetic
ARPYYDILTDYYGMDV

SEQ ID NO: 273 - DNA, 354 bp, synthetic construct
  misc_feature 1..354: note = synthetic
  source 1..354: mol_type = other DNA
caggtccagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccacggacgaatccacgagcacagcctacatggagctgagcagtctgagatctgaggacacggccgtgtattactgtgcgacagaagggtacaactggaactatgactattggggccagggaaccctggtcaccgtctcctca

SEQ ID NO: 274 - protein, 118 aa, synthetic construct
  REGION 1..118: note = synthetic
QVQLVQSGAEVKKPGSSVKVSCKASGGTFSSYAISWVRQAPGQGLEWMGGIIPIFGTANYAQKFQGRVTITTDESTSTAYMELSSLRSEDTAVYYCATEGYNWNYDYWGQGTLVTVSS

SEQ ID NO: 275 - DNA, 24 bp, synthetic construct
  misc_feature 1..24: note = synthetic
  source 1..24: mol_type = other DNA
ggaggcaccttcagcagctatgct

SEQ ID NO: 276 - protein, 8 aa, synthetic construct
  REGION 1..8: note = synthetic
GGTFSSYA

SEQ ID NO: 277 - DNA, 24 bp, synthetic construct
  misc_feature 1..24: note = synthetic
  source 1..24: mol_type = other DNA
atcatccctatctttggtacagca

SEQ ID NO: 278 - protein, 8 aa, synthetic construct
  REGION 1..8: note = synthetic
IIPIFGTA

SEQ ID NO: 279 - DNA, 33 bp, synthetic construct
  misc_feature 1..33: note = synthetic
  source 1..33: mol_type = other DNA
gcgacagaagggtacaactggaactatgactat

SEQ ID NO: 280 - protein, 11 aa, synthetic construct
  REGION 1..11: note = synthetic
ATEGYNWNYDY

SEQ ID NO: 281 - DNA, 363 bp, synthetic construct
  misc_feature 1..363: note = synthetic
  source 1..363: mol_type = other DNA
caggtccagctggtgcagtctggggctgaggtgaagaagcctgggtcctcggtgaaggtctcctgcaaggcttctggaggcaccttcagcagctatgttatcacctgggtgcgacaggtccctggacaagggcttgagtggatgggagggatcatccctatctttggtacagcaaactacgcacagaagttccagggcagagtcacgattaccacggacgaatccacgagcacagcctacatggacctgagcagcctgagatctgaggacacggccttttattattgtgcgagaaggaggtctaactggggatctcatgcttttgatatctggggccaagggacaatggtcaccgtctcttca

SEQ ID NO: 282 - protein, 121 aa, synthetic construct
  REGION 1..121: note = synthetic
QVQLVQSGAEVKKPGSSVKVSCKASGGTFSSYVITWVRQVPGQGLEWMGGIIPIFGTANYAQKFQGRVTITTDESTSTAYMDLSSLRSEDTAFYYCARRRSNWGSHAFDIWGQGTMVTVSS

SEQ ID NO: 283 - DNA, 24 bp, synthetic construct
  misc_feature 1..24: note = synthetic
  source 1..24: mol_type = other DNA
ggaggcaccttcagcagctatgtt

SEQ ID NO: 284 - protein, 8 aa, synthetic construct
  REGION 1..8: note = synthetic
GGTFSSYV

SEQ ID NO: 285 - DNA, 24 bp, synthetic construct
  misc_feature 1..24: note = synthetic
  source 1..24: mol_type = other DNA
atcatccctatctttggtacagca

SEQ ID NO: 286 - protein, 8 aa, synthetic construct
  REGION 1..8: note = synthetic
IIPIFGTA

SEQ ID NO: 287 - DNA, 42 bp, synthetic construct
  misc_feature 1..42: note = synthetic
  source 1..42: mol_type = other DNA
gcgagaaggaggtctaactggggatctcatgcttttgatatc

SEQ ID NO: 288 - protein, 14 aa, synthetic construct
  REGION 1..14: note = synthetic
ARRRSNWGSHAFDI

SEQ ID NO: 289 - DNA, 387 bp, synthetic construct
  misc_feature 1..387: note = synthetic
  source 1..387: mol_type = other DNA
gaagtgcagctggtggagtctgggggaggcttggtacagcctggcaggtccctgagactctcctgtgcagcctctggaatcacctttgatgattatgccatgtactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagttgaggacacggccttgtattattgtgcaaaagataaggggtattacgatattttgactggagattactactactactacggtatggacgtctggggccaagggaccacggtcaccgtctcctca

SEQ ID NO: 290 - protein, 129 aa, synthetic construct
  REGION 1..129: note = synthetic
EVQLVESGGGLVQPGRSLRLSCAASGITFDDYAMYWVRQAPGKGLEWVSGISWNSGSIGYADSVKGRFTISRDNAKNSLYLQMNSLRVEDTALYYCAKDKGYYDILTGDYYYYYGMDVWGQGTTVTVSS

SEQ ID NO: 291 - DNA, 24 bp, synthetic construct
  misc_feature 1..24: note = synthetic
  source 1..24: mol_type = other DNA
ggaatcacctttgatgattatgcc

SEQ ID NO: 292 - protein, 8 aa, synthetic construct
  REGION 1..8: note = synthetic
GITFDDYA

SEQ ID NO: 293 - DNA, 24 bp, synthetic construct
  misc_feature 1..24: note = synthetic
  source 1..24: mol_type = other DNA
attagttggaatagtggtagcata

SEQ ID NO: 294 - protein, 8 aa, synthetic construct
  REGION 1..8: note = synthetic
ISWNSGSI

SEQ ID NO: 295 - DNA, 66 bp, synthetic construct
  misc_feature 1..66: note = synthetic
  source 1..66: mol_type = other DNA
gcaaaagataaggggtattacgatattttgactggagattactactactactacggtatggacgtc

SEQ ID NO: 296 - protein, 22 aa, synthetic construct
  REGION 1..22: note = synthetic
AKDKGYYDILTGDYYYYYGMDV

SEQ ID NO: 297 - protein, 5 aa, synthetic construct
  REGION 1..5: note = Synthetic
LLQGA

SEQ ID NO: 298 - protein, 126 aa, synthetic construct
  REGION 1..126: note = Synthetic
QVQLVQSGAEVKKPGASVTVSCQVSGYTLTSYGLSWVRQAPGQGLEWVGWINTYDGQTKYVKKFQGRVTMTTHTGTNTAYMEMKSLRSDDTAVYYCARVEGVRGVMGFHYYPMDVWGQGTMVTVSS

SEQ ID NO: 299 - protein, 8 aa, synthetic construct
  REGION 1..8: note = Synthetic
GYTLTSYG

SEQ ID NO: 300 - protein, 8 aa, synthetic construct
  REGION 1..8: note = Synthetic
INTYDGQT

SEQ ID NO: 301 - protein, 19 aa, synthetic construct
  REGION 1..19: note = Synthetic
ARVEGVRGVMGFHYYPMDV

SEQ ID NO: 302 - protein, 111 aa, synthetic construct
  REGION 1..111: note = Synthetic
QSVLTQPPSVSGAPGQRVTISCTGSSSNIGAGYAVHWYQQLPGTAPKLLISGNSNRPSGVPDRFSGSKSGTSASLAITGLQAEDEADYYCQSYDSSLSGSVFGGGTKLTVL

SEQ ID NO: 303 - protein, 9 aa, synthetic construct
  REGION 1..9: note = Synthetic
SSNIGAGYA

SEQ ID NO: 304 - skipped

SEQ ID NO: 305 - protein, 11 aa, synthetic construct
  REGION 1..11: note = Synthetic
QSYDSSLSGSV

SEQ ID NO: 306 - protein, 5 aa, synthetic construct
  REGION 1..5: note = Synthetic
GGGGS

SEQ ID NO: 307 - protein, 5 aa, synthetic construct
  REGION 1..5: note = Synthetic
GGGGG

SEQ ID NO: 308 - protein, 5 aa, synthetic construct
  REGION 1..5: note = Synthetic
GGGGK

SEQ ID NO: 309 - protein, 4 aa, synthetic construct
  REGION 1..4: note = Synthetic
GFGG

SEQ ID NO: 310 - protein, 5 aa, synthetic construct
  REGION 1..5: note = Synthetic
ELQGP

SEQ ID NO: 311 - protein, 11 aa, synthetic construct
  REGION 1..11: note = Synthetic
GGGGSGELQRP

SEQ ID NO: 312 - DNA, 384 bp, synthetic construct
  misc_feature 1..384: note = Synthetic
  source 1..384: mol_type = other DNA
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaataggggtagcataggctatgcggactctgtgaagggccgattcaccatctccagagacaacgccaagaactccctgtatctgcaaatgagcagtctgagagctgaggacacggccttgtattactgcgcaaaagatggcgagagatgggatagtgtagtagtaccatctgctaggaacggtatggacgtctggggccaagggaccacggtcaccgtctcctca

SEQ ID NO: 313 - protein, 128 aa, synthetic construct
  REGION 1..128: note = Synthetic
EVQLVESGGGLVQPGRSLRLSCAASGFTFDDYAMHWVRQAPGKGLEWVSGISWNRGSIGYADSVKGRFTISRDNAKNSLYLQMSSLRAEDTALYYCAKDGERWDSVVVPSARNGMDVWGQGTTVTVSS

SEQ ID NO: 314 - DNA, 24 bp, synthetic construct
  misc_feature 1..24: note = Synthetic
  source 1..24: mol_type = other DNA
ggattcacctttgatgattatgcc

SEQ ID NO: 315 - protein, 8 aa, synthetic construct
  REGION 1..8: note = Synthetic
GFTFDDYA

SEQ ID NO: 316 - DNA, 24 bp, synthetic construct
  misc_feature 1..24: note = Synthetic
  source 1..24: mol_type = other DNA
attagttggaataggggtagcata

SEQ ID NO: 317 - protein, 8 aa, synthetic construct
  REGION 1..8: note = Synthetic
ISWNRGSI

SEQ ID NO: 318 - DNA, 63 bp, synthetic construct
  misc_feature 1..63: note = Synthetic
  source 1..63: mol_type = other DNA
gcaaaagatggcgagagatgggatagtgtagtagtaccatctgctaggaacggtatggacgtc

SEQ ID NO: 319 - protein, 21 aa, synthetic construct
  REGION 1..21: note = Synthetic
AKDGERWDSVVVPSARNGMDV

SEQ ID NO: 320 - DNA, 336 bp, synthetic construct
  misc_feature 1..336: note = Synthetic
  source 1..336: mol_type = other DNA
cagtctgtgctgacgcagccgccctcagtgtctggggccccagggcagagggtcaccatctcctgcactgggagcagctccaacatcggggcaggttatgatgtacattggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagcctgagtggctcttatgtcttcggaactgggaccaaggtcaccgtccta

SEQ ID NO: 321 - protein, 112 aa, synthetic construct
  REGION 1..112: note = Synthetic
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSYVFGTGTKVTVL

SEQ ID NO: 322 - DNA, 27 bp, synthetic construct
  misc_feature 1..27: note = Synthetic
  source 1..27: mol_type = other DNA
agctccaacatcggggcaggttatgat

SEQ ID NO: 323 - protein, 9 aa, synthetic construct
  REGION 1..9: note = Synthetic
SSNIGAGYD

SEQ ID NO: 324-325 - skipped (2 entries)

SEQ ID NO: 326 - DNA, 36 bp, synthetic construct
  misc_feature 1..36: note = Synthetic
  source 1..36: mol_type = other DNA
cagtcctatgacagcagcctgagtggctcttatgtc

SEQ ID NO: 327 - protein, 12 aa, synthetic construct
  REGION 1..12: note = Synthetic
QSYDSSLSGSYV

SEQ ID NO: 328 - DNA, 1377 bp, synthetic construct
  misc_feature 1..1377: note = Synthetic
  source 1..1377: mol_type = other DNA
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaataggggtagcataggctatgcggactctgtgaagggccgattcaccatctccagagacaacgccaagaactccctgtatctgcaaatgagcagtctgagagctgaggacacggccttgtattactgcgcaaaagatggcgagagatgggatagtgtagtagtaccatctgctaggaacggtatggacgtctggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 329 - protein, 458 aa, synthetic construct
  REGION 1..458: note = Synthetic
EVQLVESGGGLVQPGRSLRLSCAASGFTFDDYAMHWVRQAPGKGLEWVSGISWNRGSIGYADSVKGRFTISRDNAKNSLYLQMSSLRAEDTALYYCAKDGERWDSVVVPSARNGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 330 - DNA, 657 bp, synthetic construct
  misc_feature 1..657: note = Synthetic
  source 1..657: mol_type = other DNA
cagtctgtgctgacgcagccgccctcagtgtctggggccccagggcagagggtcaccatctcctgcactgggagcagctccaacatcggggcaggttatgatgtacattggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagcctgagtggctcttatgtcttcggaactgggaccaaggtcaccgtcctaggccagcccaaggccgccccctccgtgaccctgttccccccctcctccgaggagctgcaggccaacaaggccaccctggtgtgcctgatctccgacttctaccccggcgccgtgaccgtggcctggaaggccgactcctcccccgtgaaggccggcgtggagaccaccaccccctccaagcagtccaacaacaagtacgccgcctcctcctacctgtccctgacccccgagcagtggaagtcccaccggtcctactcctgccaggtgacccacgagggctccaccgtggagaagaccgtggcccccaccgagtgctcctga

SEQ ID NO: 331 - protein, 218 aa, synthetic construct
  REGION 1..218: note = Synthetic
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 332 - DNA, 360 bp, synthetic construct
  misc_feature 1..360: note = Synthetic
  source 1..360: mol_type = other DNA
caggtgcagctggtggagtctgggggaggcgtggtccagcctgggaggtccctgagactctcctgtgcagcctctggattcaccttcagtaactatgctatgtactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagaactgaggacacggctgtgtattactgtgcgagtggctccgactacggtgactacttattggtttactggggccagggaaccctggtcaccgtctcctca

SEQ ID NO: 333 - protein, 120 aa, synthetic construct
  REGION 1..120: note = Synthetic
QVQLVESGGGVVQPGRSLRLSCAASGFTFSNYAMYWVRQAPGKGLEWVAVISYDGSNKYYADSVKGRFTISRDNSKNTLYLQMNSLRTEDTAVYYCASGSDYGDYLLVYWGQGTLVTVSS

SEQ ID NO: 334 - DNA, 24 bp, synthetic construct
  misc_feature 1..24: note = Synthetic
  source 1..24: mol_type = other DNA
ggattcaccttcagtaactatgct

SEQ ID NO: 335 - protein, 8 aa, synthetic construct
  REGION 1..8: note = Synthetic
GFTFSNYA

SEQ ID NO: 336 - DNA, 24 bp, synthetic construct
  misc_feature 1..24: note = Synthetic
  source 1..24: mol_type = other DNA
atatcatatgatggaagtaataaa

SEQ ID NO: 337 - protein, 8 aa, synthetic construct
  REGION 1..8: note = Synthetic
ISYDGSNK

SEQ ID NO: 338 - DNA, 39 bp, synthetic construct
  misc_feature 1..39: note = Synthetic
  source 1..39: mol_type = other DNA
gcgagtggctccgactacggtgactacttattggtttac

SEQ ID NO: 339 - protein, 13 aa, synthetic construct
  REGION 1..13: note = Synthetic
ASGSDYGDYLLVY

SEQ ID NO: 340 - DNA, 330 bp, synthetic construct
  misc_feature 1..330: note = Synthetic
  source 1..330: mol_type = other DNA
cagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaagcggccctcaggggtttctaatcgcttctctggctccaagtctggcaacacggcctccctgaccatctctgggctccagtctgaggacgaggctgattattactgcaactctttgacaagcatcagcacttgggtgttcggcggagggaccaagctgaccgtccta

SEQ ID NO: 341 - protein, 110 aa, synthetic construct
  REGION 1..110: note = Synthetic
QSALTQPASVSGSPGQSITISCTGTSSDVGGYNYVSWYQQHPGKAPKLMIYDVSKRPSGVSNRFSGSKSGNTASLTISGLQSEDEADYYCNSLTSISTWVFGGGTKLTVL

SEQ ID NO: 342 - DNA, 27 bp, synthetic construct
  misc_feature 1..27: note = Synthetic
  source 1..27: mol_type = other DNA
agcagtgacgttggtggttataactat

SEQ ID NO: 343 - protein, 9 aa, synthetic construct
  REGION 1..9: note = Synthetic
SSDVGGYNY

SEQ ID NO: 344-345 - skipped (2 entries)

SEQ ID NO: 346 - DNA, 30 bp, synthetic construct
  misc_feature 1..30: note = Synthetic
  source 1..30: mol_type = other DNA
aactctttgacaagcatcagcacttgggtg

SEQ ID NO: 347 - protein, 10 aa, synthetic construct
  REGION 1..10: note = Synthetic
NSLTSISTWV

SEQ ID NO: 348 - DNA, 1353 bp, synthetic construct
  misc_feature 1..1353: note = Synthetic
  source 1..1353: mol_type = other DNA
caggtgcagctggtggagtctgggggaggcgtggtccagcctgggaggtccctgagactctcctgtgcagcctctggattcaccttcagtaactatgctatgtactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagaactgaggacacggctgtgtattactgtgcgagtggctccgactacggtgactacttattggtttactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 349 - protein, 450 aa, synthetic construct
  REGION 1..450: note = Synthetic
QVQLVESGGGVVQPGRSLRLSCAASGFTFSNYAMYWVRQAPGKGLEWVAVISYDGSNKYYADSVKGRFTISRDNSKNTLYLQMNSLRTEDTAVYYCASGSDYGDYLLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 350 - DNA, 651 bp, synthetic construct
  misc_feature 1..651: note = Synthetic
  source 1..651: mol_type = other DNA
cagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaagcggccctcaggggtttctaatcgcttctctggctccaagtctggcaacacggcctccctgaccatctctgggctccagtctgaggacgaggctgattattactgcaactctttgacaagcatcagcacttgggtgttcggcggagggaccaagctgaccgtcctaggccagcccaaggccgccccctccgtgaccctgttccccccctcctccgaggagctgcaggccaacaaggccaccctggtgtgcctgatctccgacttctaccccggcgccgtgaccgtggcctggaaggccgactcctcccccgtgaaggccggcgtggagaccaccaccccctccaagcagtccaacaacaagtacgccgcctcctcctacctgtccctgacccccgagcagtggaagtcccaccggtcctactcctgccaggtgacccacgagggctccaccgtggagaagaccgtggcccccaccgagtgctcctga

SEQ ID NO: 351 - protein, 216 aa, synthetic construct
  REGION 1..216: note = Synthetic
QSALTQPASVSGSPGQSITISCTGTSSDVGGYNYVSWYQQHPGKAPKLMIYDVSKRPSGVSNRFSGSKSGNTASLTISGLQSEDEADYYCNSLTSISTWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 352 - DNA, 354 bp, synthetic construct
  source 1..354: mol_type = other DNA
gaggtgcagctggtggagtctgggggaggcttggtacagcctggggggtccctgagactctcctgtgcagcctctggattcaccttcagtagctacgacatgcactgggtccgccaagctacaggaaaaggtctggagtgggtctcagctattggtactgctggtgacacatactatccaggctccgtgaagggccgattcaccatctccagagaaaatgccaaaaactccttgtatcttcaaatgaacagcctgagagccggggacacggctgtgtattactgtgcaagaacatggttcggggagctttactttgactactggggccagggaaccctggtcaccgtctcctca

SEQ ID NO: 353 - protein, 118 aa, synthetic construct
EVQLVESGGGLVQPGGSLRLSCAASGFTFSSYDMHWVRQATGKGLEWVSAIGTAGDTYYPGSVKGRFTISRENAKNSLYLQMNSLRAGDTAVYYCARTWFGELYFDYWGQGTLVTVSS

SEQ ID NO: 354 - DNA, 24 bp, synthetic construct
  source 1..24: mol_type = other DNA
ggattcaccttcagtagctacgac

SEQ ID NO: 355 - protein, 8 aa, synthetic construct
GFTFSSYD

SEQ ID NO: 356 - DNA, 21 bp, synthetic construct
  source 1..21: mol_type = other DNA
attggtactgctggtgacaca

SEQ ID NO: 357 - protein, 7 aa, synthetic construct
IGTAGDT

SEQ ID NO: 358 - DNA, 36 bp, synthetic construct
  source 1..36: mol_type = other DNA
gcaagaacatggttcggggagctttactttgactac

SEQ ID NO: 359 - protein, 12 aa, synthetic construct
ARTWFGELYFDY

SEQ ID NO: 360 - DNA, 339 bp, synthetic construct
  source 1..339: mol_type = other DNA
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagaattcactctcaccatcaccagcctgcaggctgaagatgtggcagtttattactgtcagcaatattatagtagtccgctcactttcggcggagggaccaaggtggagatcaaa

SEQ ID NO: 361 - protein, 113 aa, synthetic construct
DIVMTQSPDSLAVSLGERATINCKSSQSVLYSSNNKNYLAWYQQKPGQPPKLLIYWASTRESGVPDRFSGSGSGTEFTLTITSLQAEDVAVYYCQQYYSSPLTFGGGTKVEIK

SEQ ID NO: 362 - DNA, 36 bp, synthetic construct
  source 1..36: mol_type = other DNA
cagagtgttttatacagctccaacaataagaactac

SEQ ID NO: 363 - protein, 12 aa, synthetic construct
QSVLYSSNNKNY

SEQ ID NO: 364 - DNA, 27 bp, synthetic construct
  source 1..27: mol_type = other DNA
cagcaatattatagtagtccgctcact

SEQ ID NO: 365 - protein, 9 aa, synthetic construct
QQYYSSPLT

SEQ ID NO: 366 - DNA, 1347 bp, synthetic construct
  source 1..1347: mol_type = other DNA
gaggtgcagctggtggagtctgggggaggcttggtacagcctggggggtccctgagactctcctgtgcagcctctggattcaccttcagtagctacgacatgcactgggtccgccaagctacaggaaaaggtctggagtgggtctcagctattggtactgctggtgacacatactatccaggctccgtgaagggccgattcaccatctccagagaaaatgccaaaaactccttgtatcttcaaatgaacagcctgagagccggggacacggctgtgtattactgtgcaagaacatggttcggggagctttactttgactactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 367 - protein, 448 aa, synthetic construct
EVQLVESGGGLVQPGGSLRLSCAASGFTFSSYDMHWVRQATGKGLEWVSAIGTAGDTYYPGSVKGRFTISRENAKNSLYLQMNSLRAGDTAVYYCARTWFGELYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 368 - DNA, 663 bp, synthetic construct
  source 1..663: mol_type = other DNA
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagaattcactctcaccatcaccagcctgcaggctgaagatgtggcagtttattactgtcagcaatattatagtagtccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 369 - protein, 220 aa, synthetic construct
DIVMTQSPDSLAVSLGERATINCKSSQSVLYSSNNKNYLAWYQQKPGQPPKLLIYWASTRESGVPDRFSGSGSGTEFTLTITSLQAEDVAVYYCQQYYSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 370 - protein, 128 aa, synthetic construct
EVQLVESGAEVKKPGASVKVSCKVSGYTLTELSMHWVRQAPGKGLEWMGGFDPEDGETMYAQKFQGRVTMTEDTSTDTAYMELSSLRSEDTAVYYCATSTAVAGTPDLFDYYYGMDVWGQGTTVTVSS

SEQ ID NO: 371 - protein, 330 aa, synthetic construct [sequence omitted]

SEQ ID NO: 372 - protein, 112 aa, synthetic construct
EIVMTQSPLSSPVTLGQPASISCRSSQSLVHSDGNTYLSWLQQRPGQPPRLLIYKISNRFSGVPDRFSGSGAGTDFTLKISRVEAEDVGVYYCTQATQFPYTFGQGTKVDIK

SEQ ID NO: 373 - protein, 107 aa, synthetic construct [sequence omitted]

SEQ ID NO: 374 - DNA, 990 bp, synthetic construct
  source 1..990: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaa

SEQ ID NO: 375 - protein, 334 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGLLQGA

SEQ ID NO: 376 - DNA, 1002 bp, synthetic construct
  source 1..1002: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtctgctgcagggcgcc

SEQ ID NO: 377 - protein, 334 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGELQRP

SEQ ID NO: 378 - DNA, 1002 bp, synthetic construct
  source 1..1002: mol_type = other DNA
gcctctaccaagggccccagcgtttttcctctggcccctagctctaaatctacctctggcggcaccgccgctctgggctgtctggtcaaggactacttccctgagcctgtgactgtgtcttggaactctggagctctgaccagcggagtgcacaccttccccgctgtgctgcagtcctctggcctgtacagcctgtcttccgtggttaccgtgccttctagctccctcggcacacagacctacatctgcaacgtgaaccacaagccttctaacaccaaggtggacaagaaagtggaacccaagagttgcgacaaaacccatacctgtcctccttgccctgcccccgaactgctgggcggcccttccgtgtttctgtttcctccaaagcccaaggacacactgatgatctcccggacccctgaagtgacctgcgtggtggtggatgtgtctcacgaggatcctgaggtgaagttcaactggtacgtggatggcgtggaagtgcacaacgccaagaccaagcctagagaggaacagtacaactccacctaccgggtggtcagcgtgctgaccgtgctgcaccaggactggctgaacggcaaagagtacaagtgcaaggtgtccaacaaggctctgcctgctcctatcgagaagaccatctctaaggccaagggccagcctcgggaacctcaggtttataccctgcctccttctcgggacgagctgaccaagaaccaggtctctctgacctgcctggtgaagggattctacccatctgacatcgccgtggagtgggagtccaatggccagcccgagaacaactacaagactacacctcccgtgctggactccgacggctccttcttcctgtactccaagctgacagtggacaagtctagatggcagcagggcaacgtgttctcctgctccgtgatgcacgaggccctgcacaatcactatacacagaagtccctgtctctgtcccctggcgagctgcagcggcct